(19) United States
(12) Patent Application Publication
(10) Pub. No.: US 2024/0173352 A1
(43) Pub. Date: May 30, 2024

(54) CELL-SURFACE RECEPTORS RESPONSIVE TO LOSS OF HETEROZYGOSITY

(71) Applicant: A2 Biotherapeutics, Inc., Agoura Hills, CA (US)

(72) Inventors: Carl Alexander KAMB, Westlake Village, CA (US); Agnes HAMBURGER, Newbury Park, CA (US); Han XU, Agoura Hills, CA (US)

(21) Appl. No.: 17/633,358

(22) PCT Filed: Aug. 6, 2020
(86) PCT No.: PCT/US2020/045228

§ 371 Date: Feb. 7, 2022

Related U.S. Application Data
(60) Provisional application No. 62/885,093, filed on Aug. 9, 2019.
(60) Provisional application No. 63/005,670, filed on Apr. 6, 2020.

(51) Int. Cl. A61K 35/17; A61K 39/00; A61P 35/00; A61P 37/04; C07K 16/28; C12N 5/0783; C12N 15/85

(52) U.S. Cl. CPC A61K 35/17; A61K 39/4611; A61K 39/4631; A61K 39/464411; A61P 35/00; A61P 37/04; C07K 16/2833; C12N 5/0636; C12N 15/85; A61K 2239/13; A61K 2239/21

(57) ABSTRACT

The disclosure relates to systems of two engineered receptors each having a ligand binding domain, collectively designed to target cells identified by loss of heterozygosity and used to treat a disease or disorder, for example, cancer. The disclosure provides immune cells expressing two engineered receptors, methods of making same, and polynucleotides and vectors encoding same.

### CROSS REFERENCE TO RELATED APPLICATIONS

This application claims the benefit of priority to U.S. Provisional Patent Application Ser. No. 62/885,093 filed on Aug. 9, 2019 and U.S. Provisional Patent Application Ser. No. 63/005,670 filed on Apr. 6, 2020, the contents of each of which are hereby incorporated by reference in their entireties.

### INCORPORATION BY REFERENCE OF SEQUENCE LISTING

The present application is being filed with a Sequence Listing in electronic format. The Sequence Listing is provided as a file entitled A2BI-00903WO_SeqList.txt, created on Aug. 5, 2020 and is 404 kilobytes in size. The information in electronic format of the Sequence Listing is incorporated by reference in its entirety.

### BACKGROUND

Cell therapy is a powerful tool for the treatment of various diseases, particularly cancers. In conventional adoptive cell therapies, immune cells are engineered to express specific receptors, for example chimeric antigen receptors (CARs) or T Cell Receptors (TCRs), which direct the activity of the immune cells to cellular targets via interaction of the receptor with a ligand expressed by the target cell. Identification of suitable target molecules remains challenging. There is a need in the art for compositions and methods useful in the treatment of disease, particularly cancers, by cellular therapy.

### SUMMARY

The disclosure relates generally to a two-receptor system expressed in engineered immune cells, for example immune cells used in adoptive cell therapy, which can be used to target these immune cells to tumor cells exhibiting loss of heterozygosity. In this two receptor system, the first receptor acts to activate, or promote activation of the immune cells, while the second receptor acts to inhibit activation by the first receptor. Differential expression of ligands for the first and second receptors, for example through loss of heterozygosity of the locus encoding the inhibitory ligand, mediates activation of immune cells by target cells that express the first activator ligand but not the second inhibitory ligand.

The disclosure provides immune cells, comprising: (a) a first engineered receptor, the first engineered receptor comprising a transmembrane region and an extracellular region, the extracellular region comprising a first ligand binding domain capable of specifically binding a first ligand; and (b) a second engineered receptor, the second engineered receptor comprising a transmembrane region and an extracellular region, the extracellular region comprising a second ligand binding domain capable of specifically binding a second ligand, wherein binding of the first ligand binding domain to the first ligand activates or promotes activation of the immune cell by the receptor, and wherein binding of the second ligand binding domain to the second ligand inhibits activation of the immune cell by the first receptor.

In some embodiments of the immune cells of the disclosure, the second ligand not expressed in a target cell due to loss of heterozygosity of a gene encoding the second ligand. In some embodiments the second ligand is an HLA class I allele or a minor histocompatibility antigen (MiHA).

In some embodiments of the immune cells of the disclosure, the second ligand is a MiHA. In some embodiments, the MiHA is selected from the group of MiHAs in Tables 8 and 9. In some embodiments, the MiHA is HA-1.

In some embodiments of the immune cells of the disclosure, the second ligand is an HLA class I allele. In some embodiments, the HLA class I allele comprises HLA-A, HLA-B or HLA-C. In some embodiments, the HLA class I allele is an HLA-A*02 allele.

In some embodiments of the immune cells of the disclosure, the second ligand is not expressed in the target cell due to loss of Y chromosome. In some embodiments, the second ligand is encoded by a Y chromosome gene.

In some embodiments of the immune cells of the disclosure, the first ligand and second ligand are not the same. In some embodiments, the first ligand is expressed by target cells. In some embodiments, the first ligand is expressed by target cells and non-target cells. In some embodiments, the second ligand is not expressed by the target cells, and is expressed by a plurality of the non-target cells. In some embodiments, the plurality of non-target cells express both the first and second ligands.

In some embodiments, the target cells are cancer cells and the non-target cells are non-cancerous cells.

In some embodiments of the immune cells of the disclosure, the first ligand is selected from the group consisting of a cell adhesion molecule, a cell-cell signaling molecule, an extracellular domain, a molecule involved in chemotaxis, a glycoprotein, a G protein-coupled receptor, a transmembrane protein, a receptor for a neurotransmitter and a voltage gated ion channel. In some embodiments, the first ligand is selected from the group of antigens in Table 5. In some embodiments, the first ligand is selected from the group consisting of transferrin receptor (TFRC), epidermal growth factor receptor (EGFR), CEA cell adhesion molecule 5 (CEA), CD19 molecule (CD19), erb-b2 receptor tyrosine kinase 2 (HER2), and mesothelin (MSLN) or a peptide antigen thereof. In some embodiments, the first ligand comprises HLA-A, HLA-B, HLA-C, HLA-E, HLA-F, or HLA-G. In some embodiments, the first ligand is a pan-HLA ligand.

In some embodiments of the immune cells of the disclosure, the second ligand is selected from the group consisting of an HLA class I allele, a minor histocompatibility antigen (MiHA), and a Y chromosome gene. In some embodiments, expression of the second ligand has been lost in the target cell from loss of heterozygosity. In some embodiments, the MiHA is HA-1. In some embodiments, the HLA class I allele is an HLA-A*02 allele.

In some embodiments of the immune cells of the disclosure, the first engineered receptor is a T cell receptor (TCR) or a chimeric antigen receptor (CAR). In some embodiments, the second engineered receptor is a T cell receptor (TCR) or a chimeric antigen receptor (CAR).

In some embodiments of the immune cells of the disclosure, the first ligand binding domain comprises a single chain Fv antibody fragment (ScFv), a β chain variable domain (Vβ), TCR α chain variable domain and a TCR β chain variable domain, or a variable heavy chain (VH) domain and a variable light chain (VL) domain. In some embodiments, the second ligand binding domain comprises an ScFv, Vβ domain, a TCR α chain variable domain and a TCR β chain variable domain, or a variable heavy chain (VH) domain and a variable light chain (VL) domain.

In some embodiments of the immune cells of the disclosure, the first ligand is EGFR or a peptide antigen thereof. In some embodiments, first ligand binding domain comprises a sequence of SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, or SEQ ID NO: 391, or a sequence having at least 90%, at least 95% or at least 99% identity thereto. In some embodiments, the first ligand binding domain comprises CDRs selected from SEQ ID NOs: 131-166.

In some embodiments of the immune cells of the disclosure, the first ligand is MSLN or a peptide antigen thereof. In some embodiments, the first ligand binding domain comprises a sequence of SEQ ID NO: 86, SEQ ID NO: 88, SEQ ID NO: 90 or SEQ ID NO: 92, or a sequence having at least 90%, at least 95% or at least 99% identity thereto.

In some embodiments of the immune cells of the disclosure, the first ligand is CEA or a peptide antigen thereof. In some embodiments, the first ligand binding domain comprises SEQ ID NO: 94, SEQ ID NO: 96, SEQ ID NO: 98, SEQ ID NO: 100, SEQ ID NO: 282, SEQ ID NO: 284, or SEQ ID NO: 286, or a sequence having at least 90%, at least 95% or at least 99% identity thereto. In some embodiments, the first ligand binding domain comprises CDRs selected from SEQ ID NOs: 294-302.

In some embodiments of the immune cells of the disclosure, the first ligand is CD19 or a peptide antigen thereof, and the first ligand binding domain comprises SEQ ID NO: 275 or SEQ ID NO: 277, or a sequence having at least 90%, at least 95% or at least 99% identity thereto.

In some embodiments of the immune cells of the disclosure, the first ligand is a pan-HLA ligand. In some embodiments, the first ligand binding domain comprises a sequence of SEQ ID NO: 167, SEQ ID NO: 169, SEQ ID NO: 171, SEQ ID NO: 173, SEQ ID NO: 175, or SEQ ID NO: 177, or a sequence having at least 90%, at least 95% or at least 99% identity thereto.

In some embodiments of the immune cells of the disclosure, the second ligand comprises HA-1. In some embodiments, and wherein the second ligand binding domain comprises a TCR alpha variable domain comprising SEQ ID NO: 199 or a sequence having at least 90%, at least 95%, or at least 99% identity thereto, and a TCR beta variable domain comprising SEQ ID NO: 200 or a sequence having at least 90%, at least 95%, or at least 99% identity thereto. In some embodiments, the second ligand binding domain comprises a TCR alpha variable domain comprising SEQ ID NO: 199, and a TCR beta variable domain comprising SEQ ID NO: 200.

In some embodiments of the immune cells of the disclosure, the second ligand comprises an HLA-A*02 allele. In some embodiments, the second ligand binding domain comprises any one of SEQ ID NOs: 53-64 or a sequence having at least 90%, at least 95%, or at least 99% identity thereto. In some embodiments, the second ligand binding domain comprises CDRs selected from SEQ ID NOs: 41-52.

In some embodiments of the immune cells of the disclosure, the second engineered receptor comprises an immunoreceptor tyrosine-based inhibitory motif (ITIM).

In some embodiments of the immune cells of the disclosure, the second engineered receptor comprises a LILRB1 intracellular domain or a functional variant thereof. In some embodiments, the LILRB1 intracellular domain comprises a sequence at least 95% identical to SEQ ID NO: 76. In some embodiments, the second engineered receptor comprises a LILRB1 transmembrane domain or a functional variant thereof. In some embodiments, the LILRB1 transmembrane domain or a functional variant thereof comprises a sequence at least 95% identical to SEQ ID NO: 85. In some embodiments, the second engineered receptor comprises a LILRB1 hinge domain or functional fragment or variant thereof. In some embodiments, the LILRB1 hinge domain comprises a sequence at least 95% identical to SEQ ID NO: 84, SEQ ID NO: 77 or SEQ ID NO: 78. In some embodiments, the second engineered receptor comprises a LILRB1 intracellular domain and a LILRB1 transmembrane domain, or a functional variant thereof. In some embodiments, the LILRB1 intracellular domain and LILRB1 transmembrane domain comprises SEQ ID NO: 80 or a sequence at least 95% identical to SEQ ID NO: 80. In some embodiments, the second engineered receptor comprises a first polypeptide comprising SEQ ID NO: 80 or a sequence at least 95% identity thereto fused to a TCR alpha variable domain, and a second polypeptide comprising SEQ ID NO: 80 or a sequence at least 95% identity thereto fused to a TCR beta variable domain.

In some embodiments of the immune cells of the disclosure, the first and second receptors are expressed on the surface of the immune cell at a ratio between about 1:10 to 10:1 first receptor to second receptor. In some embodiments, the first and second receptors are expressed on the surface of the immune cell at a ratio between about 1:3 to 3:1 first receptor to second receptor. In some embodiments, the first and second receptors are expressed on the surface of the immune cell at a ratio of about 1:1.

In some embodiments of the immune cells of the disclosure, the immune cell is selected form the group consisting of T cells, B cells and Natural Killer (NK) cells. In some embodiments, the immune cell is non-natural. In some embodiments, the immune cell is isolated.

The disclosure provides immune cells expressing the two receptor system of the disclosure for use as a medicament. In some embodiments, the medicament is for use in the treatment of cancer.

The disclosure provides a pharmaceutical composition comprising the immune cells of the disclosure. In some embodiments, the pharmaceutical composition comprises a pharmaceutically acceptable carrier, diluent or excipient. In some embodiments, the pharmaceutical composition comprises a therapeutically effective amount of the immune cells.

The disclosure provides methods of increasing the specificity of an adoptive cell therapy in a subject, comprising administering to the subject a plurality of the immune cells or pharmaceutical compositions of the disclosure.

The disclosure provides methods of treating cancer in a subject in need thereof, comprising administering to the subject a therapeutically effective amount of the immune cells or pharmaceutical compositions of the disclosure.

In some embodiments of the methods of the disclosure, the subject has cancer. In some embodiments, the cells of the cancer express the first ligand. In some embodiments, the cells of the cancer do not express the second ligand due to loss of heterozygosity or loss of Y chromosome.

The disclosure provides methods of making the immune cells of the disclosure, comprising (a) providing a plurality of immune cells; and (b) transforming the immune cells with a vector encoding a first engineered receptor comprising a transmembrane region and an extracellular region, the extracellular region comprising a first ligand binding domain capable of specifically binding a first ligand, and a vector encoding a second engineered receptor comprising a transmembrane region and an extracellular region, the extracellular region comprising a second ligand binding domain capable of specifically binding a second ligand; wherein binding of the first ligand binding domain to the first ligand activates or promotes activation of the immune cell, and wherein binding of the second ligand binding domain to a second ligand inhibits activation of the immune cell by the first ligand.

The disclosure provides kits comprising the immune cells or pharmaceutical compositions of the disclosure.

The disclosure provides inhibitory receptors comprising an extracellular ligand binding domain capable of specifically binding an HA-1 minor histocompatibility antigen (MiHA) and an intracellular domain comprising at least one immunoreceptor tyrosine-based inhibitory motif (ITIM).

In some embodiments of the inhibitory receptors of the disclosure, the extracellular ligand binding domain has a higher affinity for an HA-1(H) peptide of VLHDDLLEA (SEQ ID NO: 191) than for an HA-1(R) peptide of VLRDDLLEA (SEQ ID NO: 266). In some embodiments, the inhibitory receptor is activated by the HA-1(H) peptide of VLHDDLLEA (SEQ ID NO: 191) and is not activated, or activated to a lesser extent, by the HA-1(R) peptide of VLRDDLLEA (SEQ ID NO: 266). In some embodiments, the extracellular ligand binding domain comprises a TCR alpha variable domain comprising SEQ ID NO: 199 or a sequence having at least 90%, at least 95%, or at least 99% identity thereto, and a TCR beta variable domain comprising SEQ ID NO: 200 or a sequence having at least 90%, at least 95%, or at least 99% identity thereto. In some embodiments, the extracellular ligand binding domain comprises a TCR alpha variable domain comprising SEQ ID NO: 199 and a TCR beta variable domain comprising SEQ ID NO: 200.

In some embodiments of the inhibitory receptors of the disclosure, the intracellular domain comprises a LILRB1 intracellular domain or a functional variant thereof. In some embodiments, the LILRB1 intracellular domain or functional variant thereof comprises a sequence at least 95% identical to SEQ ID NO: 76. In some embodiments, the inhibitory receptor comprises a LILRB1 transmembrane domain or a functional variant thereof. In some embodiments, the LILRB1 transmembrane domain or functional variant thereof comprises a sequence at least 95% identical to SEQ ID NO: 85. In some embodiments, the inhibitory receptor comprises a LILRB1 intracellular domain and a LILRB1 transmembrane domain, or a functional variant thereof. In some embodiments, the LILRB1 intracellular domain and LILRB1 transmembrane domain comprises SEQ ID NO: 80, or a sequence at least 95% identical to SEQ ID NO: 80. In some embodiments, the inhibitory receptor comprises a first polypeptide comprising SEQ ID NO: 80 or a sequence at least 95% identical thereto fused to a TCR alpha variable domain, and a second polypeptide comprising SEQ ID NO: 80 or a sequence at least 95% identical thereto fused to a TCR beta variable domain. In some embodiments, the inhibitory receptor comprises a polypeptide of SEQ ID NO: 195 or at least 95% identity thereto and a polypeptide of SEQ ID NO: 197 or at least 95% identity thereto.

The disclosure provides an immune cell, comprising: (a) a first engineered receptor, the first engineered receptor comprising a transmembrane region and an extracellular region, the extracellular region comprising a first ligand binding domain capable of specifically binding a CD19 ligand; and (b) a second engineered receptor, the second engineered receptor comprising a transmembrane region and an extracellular region, the extracellular region comprising a second ligand binding domain capable of specifically binding an HLA-A*02 allele, wherein binding of the first ligand binding domain to the CD19 ligand activates or promotes activation of the immune cell by the first receptor, and wherein binding of the second ligand binding domain to the HLA-A*02 allele inhibits activation of the immune cell by the first receptor.

The disclosure provides an immune cell, comprising: (a) a first engineered receptor, the first engineered receptor comprising a transmembrane region and an extracellular region, the extracellular region comprising a first ligand binding domain capable of specifically binding an EGFR ligand; and (b) a second engineered receptor, the second engineered receptor comprising a transmembrane region and an extracellular region, the extracellular region comprising a second ligand binding domain capable of specifically binding an HLA-A*02 allele, wherein binding of the first ligand binding domain to the EGFR ligand activates or promotes activation of the immune cell by the first receptor, and wherein binding of the second ligand binding domain to the HLA-A*02 allele inhibits activation of the immune cell by the first receptor.

The disclosure provides an immune cell, comprising (a) a first engineered receptor, the first engineered receptor comprising a transmembrane region and an extracellular region, the extracellular region comprising a first ligand binding domain capable of specifically binding a mesothelin (MSLN) ligand; and (b) a second engineered receptor, the second engineered receptor comprising a transmembrane region and an extracellular region, the extracellular region comprising a second ligand binding domain capable of specifically binding an HLA-A*02 allele, wherein binding of the first ligand binding domain to the MSLN ligand activates or promotes activation of the immune cell by the first receptor, and wherein binding of the second ligand binding domain to the HLA-A*02 allele inhibits activation of the immune cell by the first receptor.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 is a diagram illustrating hemizygous tumor cells forming a tumor against a background of heterozygous cells that compose normal tissue. The hemizygous tumor cells express only Target A and have lost Target B due to loss of heterozygosity (LOH), while the normal cells express both Target A and Target B. This genetic difference can be exploited to create tumor-selective cytotoxic therapeutics that are blocked by Target B and activated by Target A, thereby selectively killing tumors.

FIG. 2A is a diagram showing an exemplary architecture of a dual targeted therapeutic based on LOH in tumors. In this example, there is cell-based integration of activator and blocker signals.

FIG. 2B is a series of diagrams showing various activator and receptor formats and combinations.

FIG. 3A is a pair of diagrams that show exemplary dual receptor constructs of the disclosure in TCR format. In this example, activator and inhibitor (blocker) LBDs are each fused separately to the CD3 gamma subunit of the TCR

FIG. 3B is diagram and a table that show exemplary dual receptor constructs of the disclosure in CAR format. Exemplary ITIM and inhibitor domains of the inhibitor CAR are shown in the table at right.

FIG. 4A is a plot showing the RNA-Seq expression of the transferrin receptor (TFRC) in human tissues from the GTEx database. Transferrin receptor (TFRC) is a candidate for Target A (the activator). Expression of TFRC at the RNA level is ubiquitous and relatively even. TRFC is an essential gene: Loss-of-function homozygous mutations are embryonic lethal in mice.

FIG. 4B is a plot showing the RNA-Seq expression profiles of HLA-A and HLA-B.

FIG. 5A-5H show LIR-1 blocker is modular and mediates large EC50 shifts.

FIG. 5A shows schematic of T2-Jurkat experiments to evaluate blocker constructs.

FIG. 5B shows the effect of various NY-ESO-1 scFv LBD blocker modules (PD-1, CTLA-4, LIR-1) on EC50 of MAGE-A3 CAR activator (MP1-CAR) when loaded with NY-ESO-1 blocker peptide. Error bars indicate f SD (n=2).

FIG. 5C shows the effect of an LIR-1 blocker module with various scFv LBDs (ESO, MP1 LBD 1, MP1 LBD 2, HPV E6 LBD 1, HPV E6 LBD 2, HPV E7) on EC50 of MAGE-A3 CAR activator (MP1-CAR) when loaded with corresponding peptide. Error bars indicate±SD (n=2).

FIG. 5D shows the effect of an LIR-1 blocker module with NY-ESO-1 scFv LBD on EC50 of different MAGE-A3 CAR activators (MP1-CAR or MP2-CAR) when loaded with NY-ESO-1 blocker peptide. Error bars indicate f SD (n=2).

FIG. 5E shows the effect of an LIR-1 blocker module with NY-ESO-1 scFv LBD on EC50 of different TCR activators (MP1-TCR, MP2-TCR, HPV E6-TCR) when loaded with NY-ESO-1 blocker peptide. Error bars indicate±SD (n=2). Three different TCR activators are blocked by NY-ESO-LIR-1, which has an ESO scFv, LIR-1 hinge, LIR-1 TM and LIR-1 ICD.

FIG. 5F shows the effect of an LIR-1 blocker module with NY-ESO-1 Ftcr LBDs on EC50 of MAGE-A3 CAR and TCR activators (MP1-CAR, MP1-TCR). Error bars indicate t SD (n=2). Both a third generation CAR activator or a regular TCR activator can be blocked by NY-ESO-1 Ftcr-LIR-1, which has TCRa ECD, LIR-1 TM, a LIR-1 ICD and a TCRb ECD, LIR-1 TM and LIR-1 ICD.

FIG. 5G shows Jurkat cells transfected with either HPV E7-CAR or HPV E7-CAR & A2-LIR-1 co-cultured with beads displaying various ratios of activator (HPV E7) and blocker (NY-ESO-1) antigen demonstrates blocking in cis but not trans.

FIG. 5H shows that the A2-LIR-1 blocker module blocks CD19-CAR activator at various activator to blocker ratios. E:T ratio: effector:target ratio.

FIG. 6A, 6C-6E show that primary T cells expressing LIR-1 blocker selectively kill tumor cells with pMHC and non-pMHC proof-of-concept targets.

FIG. 6A shows primary T cells transduced with HPV E7-TCR activator and ESO-LIR-1 blocker shifts EC50˜100 fold in primary T cell killing assay. Error bars indicate +/−SD (n=2).

FIG. 6B shows that HLA-A*02-LIR-1 blocks NY-ESO-1 CAR activator at various activator:blocker DNA ratios in Jurkat cells.

FIG. 6C shows that primary T cells transduced with CD19 CAR activator and HLA-A*02 blocker distinguish “tumor” cells from “normal” cells in in vitro cytotoxicity assay and demonstrate selective killing of “tumor” cells in mixed target cell assay at 3:1 E:T. A2-LIR-1: LIR-1 based receptor with an HLA-A2*02 LBD.

FIGS. 6D-6E show that primary T cells transduced with CD19 CAR activator and HLA-A*02 blocker demonstrate reversible blockade (FIG. 6D) and activation (FIG. 6E) after 3 rounds of antigen exposure (AB-A-AB and A-AB-A) in an in vitro cytotoxicity assay at 3:1 E:T. The primary T cell cytotoxicity assay was reproduced with three HLA-A*02-negative donors.

FIGS. 7A-7E show that modified CAR-T cells (i.e., CAR-T cells expressing both an activator and a blocker receptor) selectively kill tumors in xenograft model.

FIG. 7A shows primary T cells transduced with CD19 CAR activator and HLA-A*02 blocker demonstrate ˜20-fold expansion with CD3/28 stimulation over 10 days.

FIG. 7B shows a schematic of in vivo study design: HLA-A*02 NSG mice were administered either “tumor cells” (A2-negative Raji cells) or “normal cells” (A2-positive Raji cells) subcutaneously and primary T cells (human, HLA-A*02-negative donor) were injected into the tail vein when Raji xenografts averaged ˜70 mm³.

FIGS. 7C-7E show readouts of tumor size by caliper measurement (FIG. 7C), human blood T cell count by flow cytometry (FIG. 7D), and survival (FIG. 7E). Error bars are standard error of the mean (s.e.m.). UTD: untransduced.

FIG. 8 shows that the peptide-loading shift of activation EC50 is typically less than −10×. The effect of blocker peptide loading (50 uM each of NY-ESO-1, MAGE-A3, HPV E6, and HPV E7) on activating MAGE-A3 CAR (MP2 CAR) is shown.

FIG. 9 shows that the LIR-1 blocker module is ligand dependent. The effect of NY-ESO-1-LIR-1 blocker on EC50 of activating MAGE-A3 CAR (MP1-CAR) when loaded with various concentrations of NY-ESO-1 blocker peptide is shown.

FIG. 10 shows that blockers without an ICD or with a mutated, non-functional ICD do not block activation. Effect of a modified LIR-1 blocker modules containing no ICD (blue) or a mutated ICD (purple) with NY-ESO-1 scFv LBD on EC50 of MAGE-A3 CAR activator (MP2-CAR) when loaded with 10 uM of NY-ESO-1 blocker peptide is shown.

FIG. 11 shows that CD19 activates & A2-LIR-1 blocks Jurkat activation in HLA-A*02+(A2+) Raji cells. Jurkat cells transfected with either CD19 or CD19 & A2-LIR-1 were co-cultured with either WT (A2−) Raji cells or A2+ Raji cells at various cell ratios.

FIG. 12 is four panels that show the correlation of hCD3+ T cells in mouse blood to tumor growth. Shown are graphs of hCD3+ T cells compared to tumor volume 10 days and 17 days after T cell injection with A2− and A2+ Raji cells.

FIG. 13 shows that Jurkat cells expressing an EGFR CAR activator and an HLA-A*02 LIR-1 blocker are activated by EGFR+/HLA-A*02− HeLa target cells but not EGFR+/HLA-A*02+ HeLa target cells.

FIG. 14A shows the expression of HLA-A*02 on HeLa cells transduced with HLA-A*02, and HCT116 cells. HeLa and HCT1116 cells were labeled with the anti-HLA-A2 antibody BB7.2 and FACs sorted. Green: unlabeled HeLa; orange: unlabeled HCT116; blue: wild type HCT116 labeled with BB7.2; red: HeLa cells transduced with HLA-A*02 and labeled with BB7.2.

FIG. 14B shows expression of EGFR on HeLa cells and HCT116 cells. HeLa and HCT1116 cells were labeled with anti-EGFR antibody and FACs sorted. Green: unlabeled HeLa; orange: unlabeled HCT1116; blue: wild type HCT116 labeled with anti-EGFR; red: HeLa cells transduced with HLA-A*02 and labeled with anti-EGFR

FIG. 15A shows EGFR CAR activation of Jurkat cells expressing an EGFR CAR, and HCT116 target cells.

FIG. 15B shows that EGFR CAR activation of Jurkat cells can be blocked by an HLA-A*02 LIR-1 inhibitory receptor. Co-expression of the EGFR CAR and HLA-A*02 LIR-1 inhibitory receptor by Jurkat cells leads to a shift in the CAR E_{MAX} of approximately 1.8× when Jurkat cells are presented with HCT116 target cells expressing EGFR and HLA-A*02.

FIG. 16A shows titration of activator antigen in a bead-based assay to determine the optimal ratio of activator to blocker antigen.

FIG. 16B shows titration of blocker (inhibitory) antigen in the presence of a constant amount of activator antigen in a bead based assay to determine the optimal ratio of activator to blocker antigen.

FIG. 17 is a diagram (left) and a plot (right) showing that a NY-ESO-1 ScFv LIR-1 based inhibitory receptor can inhibit activation of Jurkat cell activation by a MP MAGE-A3 TCR using the solid tumor cell line A375 as target cells.

FIG. 18 is a diagram (left) and a plot (right) showing that a pMHC HLA-A*02 ScFv LIR-1 based inhibitory receptor can inhibit activation of Jurkat cell activation by a CD19 ScFv CAR using the B cell leukemia line NALM6 as target cells.

FIG. 19 is a diagram (left) and a plot (right) showing that a pMHC HLA-A*02 ScFv LIR-1 based inhibitory receptor can inhibit activation of Jurkat cells by a NY-ESO-1 ScFv CAR activator in a dose dependent manner.

FIG. 20 shows that a pan HLA (pan class I) ScFv CAR is blocked by expression of an HAL-A*02 LIR-1 blocker with tunable strength when assayed in Jurkat cells using T2 target cells and a luciferase assay.

FIG. 21A shows that a pMHC HLA-A*02 ScFv LIR-1 based inhibitory receptor can inhibit activation of Jurkat cells in cis in a cell-free bead based assay.

FIG. 21B that a pMHC HLA-A*02 ScFv LIR-1 based inhibitory receptor can inhibit activation of Jurkat cells by a MSLN ScFv CAR using the leukemia cell line K562 as target cells.

FIG. 22 is a diagram (left) and a chart (right) showing that a pMHC HLA-A*02 ScFv LIR-1 based inhibitory receptor can inhibit activation of Jurkat cells, as measured by fold induction of IFNγ, by a MSLN ScFv CAR using a pMHC HLA-A*02 ScFv LIR-1 based inhibitory receptor and HLA-A*02+ HeLa and SiHa cells as target cells.

FIG. 23 shows that a pMHC HLA-A*02 ScFv LIR-1 based inhibitory receptor inhibits killing by MSLN CAR activators using HLA-A*02+ SiHa cells but not HLA-A*02− SiHa cells.

FIG. 24 shows that activation of Jurkat cells expressing an EGFR ScFv CAR using a bead based assay can be blocked by a pMHC HLA-A*02 ScFv LIR-1 based inhibitory receptor when the activator and inhibitor antigens are present on beads in cis, but not when the activator and inhibitor antigens are present on the beads in trans.

FIG. 25A shows that activation of Jurkat cells by an EGFR ScFv CAR can be blocked by a pMHC HLA-A*02 ScFv LIR-1 based inhibitory receptor using SiHa target cells expressing HLA-A*02 (SiHa A02), but not by SiHa cells that do not express HLA-A*02 (SiHa WT).

FIG. 25B shows that activation of Jurkat cells by an EGFR ScFv CAR can be blocked by a pMHC HLA-A*02 ScFv LIR-1 based inhibitory receptor using HeLa target cells expressing HLA-A*02 (HeLa A02), but not by HeLa cells that do not express HLA-A*02 (HeLa WT).

FIG. 26 shows that additional ScFvs fused to a LIR-1 inhibitory domain inhibit a constitutive CAR activator in a dose dependent manner. Jurkat-NFAT luciferase reporter cells were transfected with an activating CAR construct that exhibits high tonic signaling and an inhibitory construct recognizing various pMHCs. The effect on activation of NFAT-luciferase was measured by co-culturing transfected Jurkat cells with T2 cells loaded with varying amounts of inhibiting peptide.

FIG. 27 is a diagram (left) and a plot (right) showing that an inhibitory receptor comprising a MiHA-b surrogate ScFv ligand binding domain (KRAS G12V ScFv-blocker) inhibits Jurkat effector cell activation by an activator TCR targeting a MiHA-a surrogate (KRAS G12D TCR, C-891), using T2 target cells.

FIG. 28 is a diagram (left) and a plot (right) showing that an inhibitory receptor comprising a MiHA-b surrogate ScFv ligand binding domain fused a LIR-1 hinge, TM and ICD (KRAS G12D ScFv-blocker) inhibits Jurkat effector cell activation by a TCR targeting a MiHA-a surrogate (KRAS G12V TCR, C-913), using T2 target cells.

FIG. 29 is a diagram (left) and a plot (right) showing that an inhibitory receptor comprising a MiHA-b surrogate Ftcr binding domain fused to a LIR1 TM and ICD (KRAS G12V Ftcr-blocker) inhibits Jurkat effector cell activation by a TCR targeting a MiHA-a surrogate (KRAS G12D TCR), using T2 target cells.

FIG. 30 is a diagram (left) and a plot (right) showing that an inhibitory receptor comprising a MiHA-b surrogate Ftcr binding domain fused to a LIR-1 TM and ICD (KRAS G12D Ftcr-blocker) inhibits Jurkat effector cell activation by a TCR targeting a MiHA-a surrogate (KRAS G12V TCR), using T2 target cells.

FIG. 31A is a plot showing inhibition of Jurkat cell activation by a MiHA-a TCR using an inhibitory receptor comprising a MiHA-b ScFv ligand binding domain that binds one mutant KRAS peptide [KRAS G12D] and a LIR-1 hinge, transmembrane domain and intracellular domain (ICD) that binds another mutant KRAS peptide (KRAS G12V). Black: C-891 activator; Blue: C-891 activator, C-1761 inhibitor; Red: C-891 activator, C-2371 and C2369 inhibitor.

FIG. 31B is a plot showing inhibition of Jurkat cell activation by a MiHA-a TCR using an inhibitory receptor comprising a MiHA-b Ftcr ligand binding domain and a LIR-1 transmembrane domain and intracellular domain (ICD). Black: C-913 activator; Blue: C-913 activator, C-1761 inhibitor; Red: C-913 activator, C2365 and C2367 inhibitor.

FIG. 32 is a plot showing that mouse MiHA-Y TCRs can activate Jurkat effector cells.

FIG. 33A is a plot and a table showing that an HA-1 Ftcr can block NY-ESO-1 TCR specifically in the presence of HA-1(H) peptide.

FIG. 33B is a plot and a table showing that there is essentially no blocking of NY-ESO-1 TCR by the HA-1 Ftcr in the presence of the non-specific, allelic variant HA-1(R) peptide.

FIG. 34A is a plot and a table showing that an HA-1 Ftcr can block a KRAS TCR specifically in the presence of HA-1(H) blocker peptide.

FIG. 34B is a plot and at table showing that there is essentially no blocking of a KRAS TCR by the HA-1 Ftcr in the presence of the non-specific, allelic variant HA-1(R) peptide.

FIG. 35 is a plot comparing peptide loading of HA-1(R), HA-1(H) and NY-ESO-1 peptides in T2 cells by flow cytometry.

FIG. 36A is a plot and a table showing an activation dose response using a MAGE-A3 MP1 ScFv CAR and a NY-ESO-1 ScFv LIR1 blocker.

FIG. 36B is a plot and a table showing an inhibition dose response using a MAGE-A3 MP1 ScFv CAR and a NY-ESO-1 ScFv LIR1 blocker.

FIG. 36C is a plot showing the x-value blocker NY-ESO-1 peptide concentrations from FIG. 36B that were normalized to the constant activator MAGE peptide concentrations used for each curve and plotted on the x-axis. B: NY-ESO-1 LIR1 blocker, A: MAGE-A3 peptide 2 ScFv CAR.

FIG. 37 is a series of plots and a table that shows that a different degree of blocking is observed when an HLA-A*02 ScFv LIR1 inhibitor is used with different EGFR ScFv CAR activators.

FIG. 38A is a series of fluorescence activated cell sorting (FACS) plots showing expression of EGFR ScFv CAR activator receptor by T cells following incubation of T cells expressing different EGFR ScFv CAR and an HLA-A*02 ScFv LIR1 inhibitor with HeLa cells expressing EGFR activator alone (Target A), inhibitor target alone (Target B) or activator and inhibitor targets (Target AB).

FIG. 38B is a plot showing quantification activator receptor expression before exposure to target cells, and after 120 hours co-culture with target cells expressing activator ligand alone (Target A), or target cells expressing both activator and blocker ligands (Target AB).

FIG. 39A is a plot showing cell surface expression of the activator receptor on T cells expressing an EGFR ScFv CAR (CT-482) activator and HLA-A*02 ScFv LIR1 inhibitor (C1765) following co-culture with to populations of HeLa cells expressing EGFR (Target A), HLA-A*02 (Target B), a combination of EGFR and HLA-A*02 on the same cell (Target AB), a mixed population of HeLa cells expressing Target A and Target AB on different cells, or a mixed population of HeLa cells expressing Target B and Target AB on different cells.

FIG. 39B is a plot showing cell surface expression of the inhibitor receptor on T cells expressing an EGFR ScFv CAR (CT-482) activator and HLA-A*02 ScFv LIR1 inhibitor (C1765) following co-culture with to populations of HeLa cells expressing EGFR (Target A), HLA-A*02 (Target B), a combination of EGFR and HLA-A*02 on the same cell (Target AB), a mixed population of HeLa cells expressing Target A and Target AB on different cells, or a mixed population of HeLa cells expressing Target B and Target AB on different cells.

FIG. 40 is a diagram of an experiment to determine if loss of expression of activator receptor by T cells was reversible.

FIG. 41A is a series of plots showing that activator surface loss of expression is reversible and corresponds to T cell cytotoxicity. At top: percent killing of target HeLa cells by T cells is shown. At bottom: activator and inhibitor receptor expression as assayed by FACS.

FIG. 41B is a series of plots showing that activator surface loss of expression is reversible and corresponds to T cell cytotoxicity. At top: percent killing of target HeLa cells by T cells is shown. At bottom: activator and inhibitor receptor expression as assayed by FACS.

### DETAILED DESCRIPTION

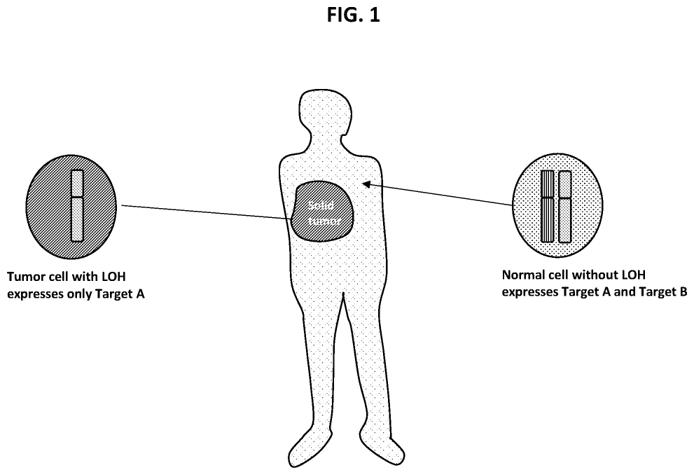

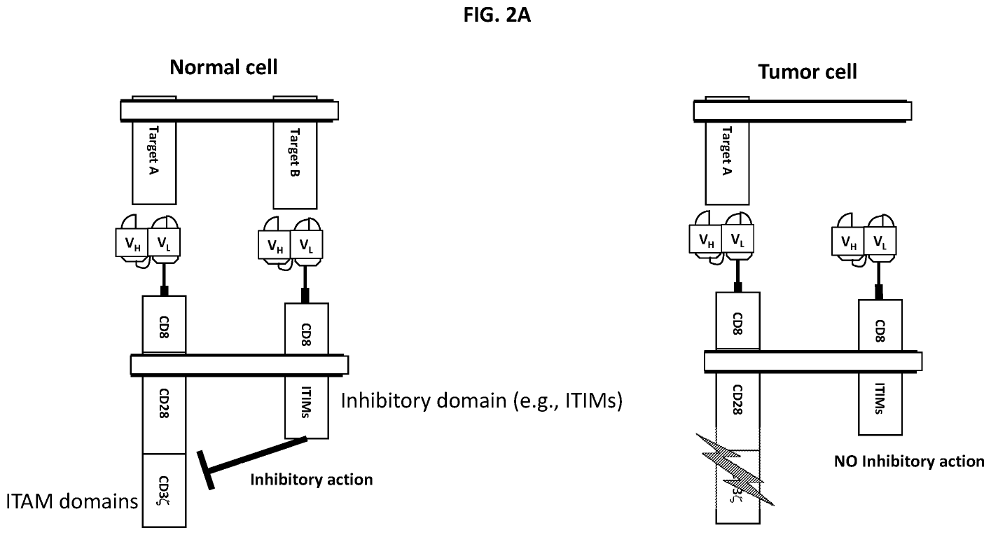

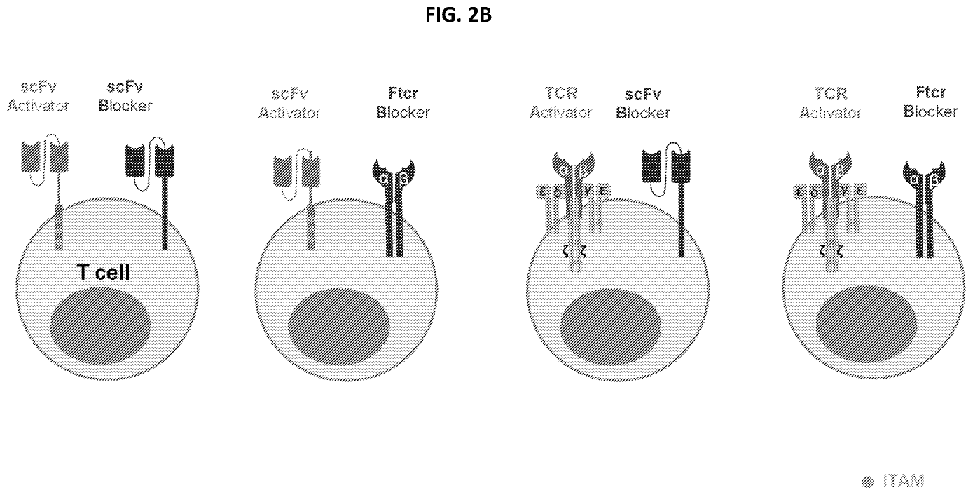

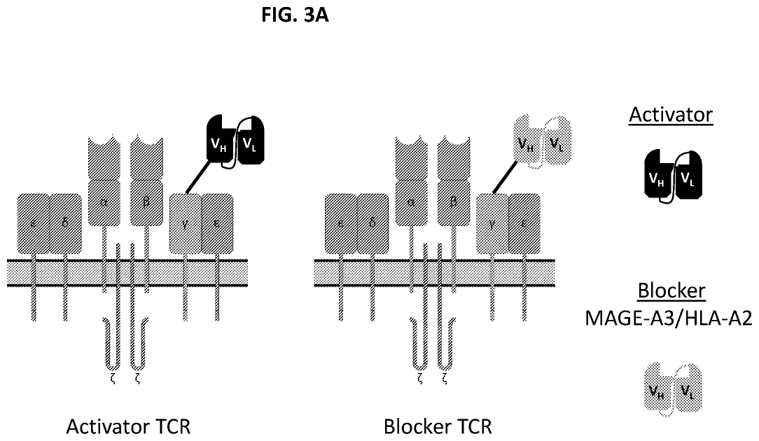

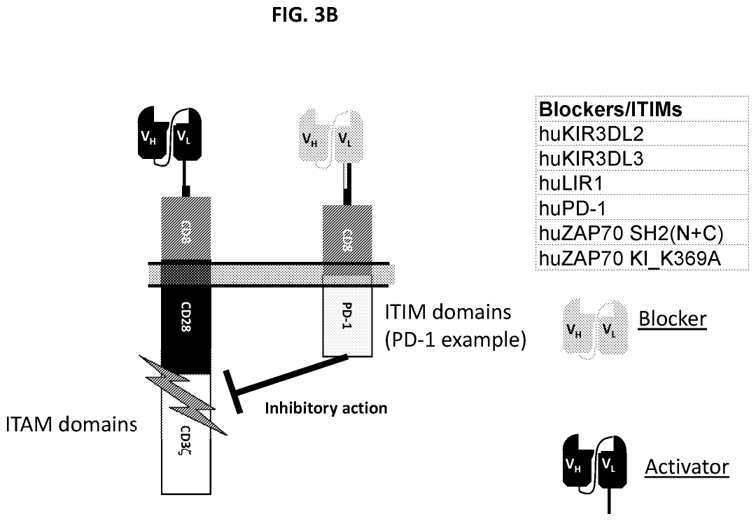

The inventors have developed a solution to the problems of identifying suitable markers and achieving cell selectivity in the treatment of diseases, particularly cancers, with cellular therapy. The primary object of the invention is to target cells based on loss of heterozygosity (FIG. 1). Using a two receptor system, in which activatory and inhibitory signals are integrated at the cellular level (FIGS. 2A, 2B, 3A and 3B), selective targeting of tumor but not non-tumor cells is achieved. Differences in expression of surface proteins that are absent or lost in target cells but present in normal cells are thereby converted to a targeted anti-tumor cell therapy. These differences improve targeting by cell therapies, and protect normal cells from the cytotoxic effects of effector cells used adoptive cell therapies.

This approach disclosed herein uses, in some embodiments, two engineered receptors, the first comprising a ligand binding domain for an activator ligand and the second comprising a ligand binding domain for an inhibitor ligand, which is selectively activated in target cells using an “AND NOT” Boolean logic (FIGS. 2A, 2B, 3A and 3B). Normal cells express both the activator and the inhibitor ligands, but activation of effector cells through the first receptor is blocked by binding of the second receptor comprising the inhibitor LBD to the inhibitor ligand, which exerts a protective effect and dominates the activity of the first, activator receptor. In contrast, in target cells that express the activator ligand but do not express the inhibitor ligand, binding of the activator ligand by the activator LBD leads to activation of the cell. Advantages of the dual activator/inhibitor receptor strategy of the instant disclosure include the ability to tune the activator and inhibitor combination to create a potent, but specific tumor-targeted adoptive cell therapy. Further, this approach can overcome the challenges of a variable effector to target cell ratio (E:T ratio) in the body, and the potentially massive excess of normal versus tumor cells seen when targeting tumor cells with adoptive cell therapies (e.g., 10¹³ normal cells versus 10⁹ tumor cells). Still further, the inventors have identified activators and inhibitors that cover large potential patient combinations, rendering this a commercially feasible approach.

Specificity of the adoptive cell therapy for a specific cell type can be achieved through the different activities of the first and second receptors, and the differential expression of the first and second ligands. Binding of the first ligand to the first receptor provides an activation signal, while binding of the second ligand to the second receptor prevents or reduces activation of effector cells even in the presence of the first ligand. The first ligand can be expressed more broadly than the second ligand, for example in both cells targeted by an adoptive cell therapy, and in healthy cells that are not target cells for an adoptive cell therapy (non-target cells). In contrast, the second ligand is expressed in the non-target cells, and is not expressed in the target cells. Only the target cells and not the non-target express the first and not the second ligand, thereby activating effector cells comprising the dual receptors of the disclosure in the presence of these cells.

The disclosure provides compositions and methods from targeting cells (e.g. tumor cells) based on loss of heterozygosity through use of two engineered receptors. The two engineered receptors, one an inhibitor and one activator, each comprise a different ligand binding domain that recognizes a different ligand. Differences in expression of the first and second ligands are used to selectively activate effector cells expressing the two receptors when only the first, activator ligand is present. Accordingly, in some embodiments, the first ligand binding domain and the second ligand binding domain are on different receptor molecules; i.e., separate receptors that are not part of a single genetic construct, fusion protein or protein complex. In some embodiments, one of the receptors activates the cell and other receptor inhibits the cell when each binds its cognate ligand. In some embodiments, the receptor comprising the second, inhibitor ligand binding domain dominates signaling so that if a target cell expresses both targets, the result is inhibition of the effector cell. Only when the inhibitory target is absent from the cell, does the first, activator ligand induce activation of the effector cell through the receptor comprising the first, activator ligand binding domain.

Any widely expressed cell surface molecule, for example a cell adhesion molecule, a cell-cell signaling molecule, an extracellular domain, a molecule involved in chemotaxis, a glycoprotein, a G protein-coupled receptor, a transmembrane, a receptor for a neurotransmitter or a voltage gated ion channel, or a peptide antigen of any of these, can be used as a first ligand. As a further example, the first ligand can be the transferrin receptor (TFRC). Any cell surface molecule not expressed on the surface of the target cell can be used as a second ligand. In those embodiments where an engineered receptor is used in the adoptive cell therapy to treat cancer, and the target cells are cancer cells, a second ligand may be chosen based on the loss of heterozygosity of the second ligand in cancer cells. Exemplary genes whose expression is frequently lost in cancer cells, for example due to mutations leading to loss of heterozygosity, include HLA class I alleles, minor histocompatibility antigens (MiHAs), and Y chromosome genes.

The disclosure further provides vectors and polynucleotides encoding the engineered receptors described herein.

The disclosure further provides methods of making immune cell populations comprising the engineered receptors described herein, and methods of treating disorders using the same.

### Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10% Mo, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “isolated” means material that is substantially or essentially free from components that normally accompany it in its native state. In particular embodiments, the term “obtained” or “derived” is used synonymously with isolated.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein “treatment” or “treating,” includes any beneficial or desirable effect, and may include even minimal improvement in symptoms. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of a symptom of disease. It also refers to delaying the onset or recurrence of a disease or condition or delaying the occurrence or recurrence of the symptoms of a disease. As used herein, “prevention” and similar words also includes reducing the intensity, effect, symptoms and/or burden of disease prior to onset or recurrence.

As used herein, the term “amount” refers to “an amount effective” or “an effective amount” of a virus to achieve a beneficial or desired prophylactic or therapeutic result, including clinical results.

A “prophylactically effective amount” refers to an amount of a virus effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus or cell may vary according to factors such as the disease state, age, sex, and weight of the individual, and the ability of the virus or cell to elicit a desired response in the individual. A therapeutically effective amount is also one in which any toxic or detrimental effects of the virus or cell are outweighed by the therapeutically beneficial effects. The term “therapeutically effective amount” includes an amount that is effective to “treat” a subject (e.g., a patient).

An “increased” or “enhanced” amount of a physiological response, e.g., electrophysiological activity or cellular activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

A “decrease” or “reduced” amount of a physiological response, e.g., electrophysiological activity or cellular activity, is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

By “maintain,” or “preserve,” or “maintenance,” or “no change,” or “no substantial change,” or “no substantial decrease” refers generally to a physiological response that is comparable to a response caused by either vehicle, or a control molecule/composition. A comparable response is one that is not significantly different or measurable different from the reference response.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

The term “MOI” is used herein to refer to multiplicity of infection, which is the ratio of agents (e.g. viral particles) to infection targets (e.g. cells).

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

As used herein, a “target cell” refers to cell that is targeted by an adoptive cell therapy. For example, a target cell can be cancer cell, which can be killed by the transplanted T cells of the adoptive cell therapy. Target cells of the disclosure express an activator ligand as described herein, and do not express an inhibitor ligand.

### Activators

The disclosure provides a first ligand, an activator, and a first engineered receptor comprising the first ligand binding domain that binds to the first activator ligand.

The disclosure provides a first engineered receptor comprising an extracellular region, the extracellular region comprising a first ligand binding domain capable of specifically binding a first ligand that activates or promotes activation of the receptor, which promotes activation of effector cells expressing the receptor. The disclosure further provides a second engineered receptor comprising a second ligand binding domain capable of binding a second ligand, wherein binding of the second ligand by the second ligand binding domain inhibits or reduces activation of effector cells even in the presence of the first receptor bound to the first ligand.

As used herein, an “activator” or “activator ligand” refers to a first ligand that binds to a first, activator ligand binding domain (LBD) of an engineered receptor of the disclosure, such as a CAR or TCR, thereby mediating activation of a T cell expressing the engineered receptor. The activator is expressed by target cells, for example cancer cells, and may also be expressed more broadly than just the target cells. For example the activator can be expressed on some, or all types of normal, non-target cells.

In some embodiments, the first ligand is a peptide ligand from any of the activator targets disclosed herein. In some embodiments, the first ligand is a peptide antigen complexed with a major histocompatibility (MHC) class I complex (peptide MHC, or pMHC), for example an MHC complex comprising human leukocyte antigen A*02 allele (HLA-A*02).

Target cell-specific first activator ligands comprising peptide antigens complexed with pMHC comprising any of human leukocyte antigen (HLA) HLA-A, HLA-B, HLA-C, HLA-E, HLA-F, and HLA-G are envisaged as within the scope of the disclosure. In some embodiments, the first ligand comprises a pMHC comprising HLA-A. HLA-A receptors are heterodimers comprising a heavy α chain and smaller β chain. The α chain is encoded by a variant of HLA-A, while the β chain (β2-microglobulin) is an invariant. There are several thousand HLA-A gene variants, all of which fall within the scope of the instant disclosure. In some embodiments, the MHC-1 comprises a human leukocyte antigen A*02 allele (HLA-A*02).

In some embodiments, the first activator ligand comprises a pMHC comprising HLA-B. Hundreds of versions (alleles) of the HLA-B gene are known, each of which is given a particular number (such as HLA-B*27).

In some embodiments, the first activator ligand comprises a pMHC comprising HLA-C. HLA-C belongs to the HLA class I heavy chain paralogues. This class 1 molecule is a heterodimer consisting of a heavy chain and a light chain (beta-2 microglobulin). Over one hundred HLA-C alleles are known in the art.

In some embodiments, the first activator ligand comprises a pMHC comprising HLA-A. In some embodiments, the first activator ligand comprises a pMHC comprising HLA-B. In some embodiments, the first activator ligand comprises a pMHC comprising HLA-C. In some embodiments, the first activator ligand comprises a pMHC comprising HLA-E. In some embodiments, the first activator ligand comprises a pMHC comprising HLA-F. In some embodiments, the first activator ligand comprises a pMHC comprising HLA-G.

In some embodiments, the first activator ligand comprises HLA-A. In some embodiments, the first activator ligand comprises HLA-B. In some embodiments, the first activator ligand comprises HLA-C. In some embodiments, the first activator ligand comprises HLA-E. In some embodiments, the first activator ligand comprises HLA-F. In some embodiments, the first activator ligand comprises HLA-G. In some embodiments, the first activator ligand comprises HLA-A, HLA-B, HLA-C, HLA-E, HLA-F or HLA-G.

In some embodiments, the first, activator ligand binding domain comprises an ScFv domain.

In some embodiments, the first, activator ligand binding domain comprises a Vβ-only ligand binding domain.

In some embodiments, the first, activator ligand binding domain comprises an antigen binding domain isolated or derived from a T cell receptor (TCR). For example, the first, activator ligand binding domain comprises TCR α and β chain variable domains.

In some embodiments, the first, activator ligand and the second, inhibitor ligand are not the same.

In some embodiments, the first, activator ligand is expressed by target cells and is not expressed by non-target cells (i.e. normal cells not targeted by the adoptive cell therapy). In some embodiments, the target cells are cancer cells and the non-target cells are non-cancerous cells.

In some embodiments, the activator ligand has high cell surface expression on the target cells. This high cell surface expression confers the ability to deliver large activation signals. Methods of measuring cell surface expression will be known to the person of ordinary skill in the art and include, but are not limited to, immunohistochemistry using an appropriate antibody against the activator ligand, followed by microscopy or fluorescence activated cell sorting (FACS).

In some embodiments, the activator ligand is encoded by a gene with an essential cellular function. Essential cellular functions are functions required for a cell to live, and include protein and lipid synthesis, cell division, replication, respiration, metabolism, ion transport, and providing structural support for tissues. Selecting activator ligands encoded by genes with essential cellular functions prevents loss of the activator ligand due to aneuploidy in cancer cells, and makes gene encoding the activator ligand less likely to undergo mutagenesis during the evolution of the cancer. In some embodiments, the activator ligand is encoded by a gene that is haploinsufficient, i.e. loss of copies of the gene encoding the activator ligand are not tolerated by the cell and lead to cell death or a disadvantageous mutant phenotype.

In some embodiments, the activator ligand is present on all target cells. In some embodiments, the target cells are cancer cells.

In some embodiments, the activator ligand is present on a plurality of target cells. In some embodiments, the target cells are cancer cells. In some embodiments, the activator ligand is present on at least 50%, at least 60%, at least 70%, at least 80%, at least 85%, at least 90%, at least 95%, at least 96%, at least 97%, at least 98%, at least 99%, at least 99.5% or at least 99.9% of target cells. In some embodiments, the activator ligand is present on at least 95% target cells. In some embodiments, the activator ligand is present on at least 99% target cells.

In some embodiments, the activator ligand is present on all cells (ubiquitous activator ligands). Activator ligands can be expressed on all cells, if, for example, the second inhibitor ligand is also expressed on all cells except the target cells.

In some embodiments, the first, activator ligand is expressed by a plurality of target cells and a plurality of non-target cells. In some embodiments, the plurality of non-target cells expresses both the first, activator ligand and the second inhibitor ligand.

In some embodiments, and the first, activator ligand and second, inhibitor ligand are present on the plurality of non-target target cells at a ratio of about 1:100 to about 100:1 of the first ligand to the second ligand. In some embodiments, and the first, activator ligand and second, inhibitor ligand are present on the plurality of non-target target cells at a ratio of about 1:50 to about 50:1 of the first ligand to the second ligand. In some embodiments, and the first, activator ligand and second, inhibitor ligand are present on the plurality of non-target target cells at a ratio of about 1:25 to about 25:1 of the first ligand to the second ligand. In some embodiments, and the first, activator ligand and second, inhibitor ligand are present on the plurality of non-target target cells at a ratio of about 1:10 to about 10:1 of the first ligand to the second ligand. In some embodiments, and the first, activator ligand and second, inhibitor ligand are present on the plurality of non-target target cells at a ratio of about 1:5 to about 5:1 of the first ligand to the second ligand. In some embodiments, and the first, activator ligand and second, inhibitor ligand are present on the plurality of non-target target cells at a ratio of about 1:3 to about 3:1 of the first ligand to the second ligand. In some embodiments, and the first, activator ligand and second, inhibitor ligand are present on the plurality of non-target target cells at a ratio of about 1:2 to about 2:1 of the first ligand to the second ligand. In some embodiments, and the first, activator ligand and second, inhibitor ligand are present on the plurality of non-target target cells at a ratio of about 1:1.

The first, activator ligand is recognized by a first ligand binding domain (sometimes referred to herein as the activator LBD).

Exemplary activator ligands include ligands selected from the group consisting of cell adhesion molecules, cell-cell signaling molecules, extracellular domains, molecule involved in chemotaxis, glycoproteins, G protein-coupled receptors, transmembrane proteins, receptors for neurotransmitters and voltage gated ion channels. In some embodiments, the first, activator ligand is transferrin receptor (TFRC) or a peptide antigen thereof. Human transferrin receptor is described in NCBI record No. AAA61153.1, the contents of which are incorporated herein by reference. In some embodiments, TFRC is encoded by a sequence of

| (SEQ ID NO: 18) | |
|---|---|
| 1 | MMDQARSAFS NLFGGEPLSY TRFSLARQVD GDNSHVEMKL AVDEEENADN NTKANVTKPK |
| 61 | RCSGSICYGT IAVIVFFLIG FMIGYLGYCK GVEPKTECER LAGTESPVRE EPGEDFPAAR |
| 121 | RLYWDDLKRK LSEKLDSTDF TSTIKLLNEN SYVPREAGSQ KDENLALYVE NQFREFKLSK |
| 181 | VWRDQHFVKI QVKDSAQNSV IIVDKNGRLV YLVENPGGYV AYSKAATVTG KLVHANFGTK |
| 241 | KDFEDLYTPV NGSIVIVRAG KITFAEKVAN AESLNAIGVL IYMDQTKFPI VNAELSFFGH |
| 301 | AHLGTGDPYT PGFPSFNHTQ FPPSRSSGLP NIPVQTISRA AAEKLFGNME GDCPSDWKTD |
| 361 | STCRMVTSES KNVKLTVSNV LKEIKILNIF GVIKGFVEPD HYVVVGAQRD AWGPGAAKSG |
| 421 | VGTALLLKLA QMFSDMVLKD GFQPSRSIIF ASWSAGDFGS VGATEWLEGY LSSLHLKAFT |
| 481 | YINLDKAVLG TSNFKVSASP LLYTLIEKTM QNVKHPVTGQ FLYQDSNWAS KVEKLTLDNA |
| 541 | AFPFLAYSGI PAVSFCFCED TDYPYLGTTM DTYKELIERI PELNKVARAA AEVAGQFVIK |
| 601 | LTHDVELNLD YERYNSQLLS FVRDLNQYRA DIKEMGLSLQ WLYSARGDFF RATSRLTTDF |
| 661 | GNAEKTDRFV MKKLNDRVMR VEYHFLSPYV SPKESPFRHV FWGSGSHTLP ALLENLKLRK |
| 721 | QNNGAFNETL FRNQLALATW TIQGAANALS GDVWDIDNEF. |

In some embodiments, the activator ligand is a tumor specific antigen (TSA). In some embodiments, the tumor specific antigen is mesothelin (MSLN), CEA cell adhesion molecule 5 (CEACAM5, or CEA), epidermal growth factor receptor (EGFR) or a peptide antigen thereof. In some embodiments, the TSA is MSLN, CEA, EGFR, delta like canonical Notch ligand 4 (DLL4), mucin 16, cell surface associated (MUC 16 also known as CA125), ganglioside GD2 (GD2), receptor tyrosine kinase like orphan receptor 1 (ROR1), erb-b2 receptor tyrosine kinase 2 (HER2/NEU) or a peptide antigen thereof. Exemplary mouse and humanized ScFv antigen binding domains targeting TSAs are shown in Table 1 below:

TABLE 1
Exemplary ScFv antigen binding domains that target tumor specific antigens
(TSAs)
MSLN binding domains

| | |
|---|---|
| C-002357 MSLN (M5): | C-002357 |
| QVQLVQSGAEVEKPGASVKVSCKASGYTFTDYYMHWVRQAPGQGLEWMGWINP | MSLN_(M5) |
| NSGGTNYAQKFQGRVTMTRDTSISTAYMELSRLRSDDTAVYYCASGWDFDYWGQG | DNA |
| TLVTVSSGGGGSGGGGSGGGGSGGDIVMTQSSSLSASVGDRVTITCRASQSIRYYLS | Sequence: SEQ |
| WYQQKPGKAPKLLIYTASILQNGVPSRFSGSGSGTDFTLTISSLQPEDFATYYCLQTYT | ID NO: 87 |
| TPDFGPGTKVEIK (SEQ ID NO: 86) | |
| C-002358 MSLN (M14): | C-002358 |
| QVQLVQSGAEVRAPGASVKISCKASGFTFRGYYIHWVRQAPGQGLEWMGIINPSGG | MSLN_(M14) |
| SRAYAQKFQGRVTMTRDTSTSTVYMELSSLRSDDTAMYYCARTASCGGDCYYLDYW | DNA |
| GQGTLVTVSSGGGGSGGGGSGGGGSGGDIQMTQSPPTLSASVGDRVTITCRASEN | Sequence: SEQ |
| VNIWLAWYQQKPGKAPKLLIYKSSSLASGVPSRFSGSGSGAEFTLTISSLQPDDFATYY | ID NO: 89 |
| CQQYQSYPLTFGGGTKVEIK (SEQ ID NO: 88) | |
| C-002359 MSLN (SSH): | C-002359 |
| QVQLVQSGAEVKKPGASVKVSCKASGYSFTGYTMNWVRQAPGQRLEWMGLITPY | MSLN_(S5H) |
| NGASSYNQKFRGRVTITRDTSASTAYMELSSLRSEDTAVYYCARGGYDGRGFDYWG | DNA |
| QGTTVTVSSGGGGSGGGGSGGGGSGGDIQMTQSPSSLSASVGDRVTITCSASSSVS | Sequence: SEQ |
| YMHWYQQKPGKAPKRLIYDTSKLASGVPSRFSGSGSGTEFTLTISSLQPEDFATYYCQ | ID NO: 91 |
| QWSGYPLTFGQGTKLEIK (SEQ ID NO: 90) | |
| C-002360 MSLN (S5M): | C-002360 |
| QVQLQQSGPELEKPGASVKISCKASGYSFTGYTMNWVKQSHGKSLEWIGLITPYNGA | MSLN_(S5M) |
| SSYNQKFRGKATLTVDKSSSTAYMDLLSLTSEDSAVYFCARGGYDGRGFDYWGQGT | DNA |
| TVTVSSGGGGSGGGGSGGGGSGGDIELTQSPAIMSASPGEKVTMTCSASSSVSYMH | Sequence: SEQ |
| WYQQKSGTSPKRWIYDTSKLASGVPGRFSGSGSGNSYSLTISSVEAEDDATYYCQQW | ID NO: 93 |
| SGYPLTFGAGTKLEIK (SEQ ID NO: 92) | |

CEACAMS binding domains

| | |
|---|---|
| C-002361 CEACAM5 (MFE23M): | C-002361 |
| QVQLQQSGAELVRSGTSVKLSCTASGFNIKDSYMHWLRQGPEQGLEWIGWIDPEN | CEACAM5_ |
| GDTEYAPKFQGKATFTTDTSSNTAYLQLSSLTSEDTAVYYCNEGTPTGPYYFDYWGQ | (MFE23M) DNA |
| GTTVTVSSGGGGSGGGGSGGGGSGGENVLTQSPAIMSASPGEKVTITCSASSSVSY | Sequence: SEQ |
| MHWFQQKPGTSPKLWIYSTSNLASGVPARFSGSGSGTSYSLTISRMEAEDAATYYCQ | ID NO: 95 |
| QRSSYPLTFGAGTKLELK (SEQ ID NO: 94) | |
| C-002362 CEACAM5 (MFE23H): | C-002362 |
| QVQLVQSGAEVKKPGASVKVSCKASGFNIKDSYMHWVRQAPGQGLEWMGWIDP | CEACAM5_ |
| ENGDTEYAPKFQGRVTMTTDTSTSTAYMELRSLRSDDTAVYYCNEGTPTGPYYFDY | (MFE23H) DNA |
| WGQGTTVTVSSGGGGSGGGGSGGGGSGGEIVLTQSPATLSLSPGERATLSCSASSSV | Sequence: SEQ |
| SYMHWYQQKPGLAPRLLIYSTSNLASGIPDRFSGSGSGTDFTLTISRLEPEDFAVYYCQ | ID NO: 97 |
| QRSSYPLTFGQGTKLEIK (SEQ ID NO: 96) | |
| C-002363 CEACAM5 (E8): | C-002363 |
| EVQLAESGGGLVQPGGSLRLSCAASGFTFSSDAMSWVRQAPGKGLEWVSAISGSGG | CEACAMS_(E8) |
| STYYADSVKGRFTISRDNSKNTLYLQMNSLRAEDTAVYYCAKSNEFLFDYWGQGTLV | DNA |
| TVSSGGGGSGGGGGGGGSGGSSELTQDPAVSVALGQTVRITCQGDSLRSSYASWY | Sequence: SEQ |
| RQRPGQAPVLVIYGKNNRPSGIPDRFSGSSSGNTASLTITGAQAEDEADYYWNSSYA | ID NO: 99 |
| WLPYVVFGGGTKLTVLG (SEQ ID NO: 98) | |
| C-002364 CEACAM5 (SM3E): | C-002364 |
| QVQLEQSGAGVVKPGASVKLSCKASGFNIKDSYMHWLRQGPGQRLEWIGWIDPEN | CEACAM5_ |
| GDTEYAPKFQGKATFTTDTSANTAYLGLSSLRPEDTAVYYCNEGTPTGPYYFDYWGQ | (SM3E) DNA |
| GTLVTVSSGGGGSGGGGSGGGGSGGENVLTQSPSSMSVSVGDRVNIACSASSSVPY | Sequence: SEQ |
| MHWLQQKPGKSPKLLIYLTSNLASGVPSRFSGSGSGTDYSLTISSVQPEDAATYYCQQ | ID NO: 101 |
| RSSYPLTFGGGTKLEIK (SEQ ID NO: 100) | |
| CT618 CEA ScFv: | CT618 CEA |
| QVQLVQSGSELKKPGASVKVSCKASGYTFTEFGMNWVRQAPGQGLEWMGWINTKTGEAT | ScFv DNA |
| YVEEFKGRFVFSLDTSVSTAYLQISSLKAEDTAVYYCARWDFAYYVEAMDYWGQGTTVTVSS | Sequence: SEQ |
| GGGGSGGGGSGGGGSGGDIQMTQSPSSLSASVGDRVTITCKASQNVGTNVAWYQQKPGK | ID NO: 283 |
| APKLLIYSASYRYSGVPSRFSGSGSGTDFTLTISSLQPEDFATYYCHQYYTYPLFTFGQGTKLEIK | |
| (SEQ ID NO: 282) | |
| CT619 CEA ScFv: | CT619 CEA ScFv |
| QVQLVQSGAEVKKPGASVKVSCKASGYTFTEFGMNWVRQAPGQGLEWMGWINTKTGEA | DNA Sequence: |
| TYVEEFKGRVTFTTDTSTSTAYMELRSLRSDDTAVYYCARWDFAYYVEAMDYWGQGTTVTV | |
| SSGGGGSGGGGSGGGGSGGDIQMTQSPSSLSASVGDRVTITCKASAAVGTYVAWYQQKPG | SEQ ID NO: 285 |
| KAPKLLIYSASYRKRGVPSRFSGSGSGTDFTLTISSLQPEDFATYYCHQYYTYPLFTFGQGTKLEI | |
| K (SEQ ID NO: 284) | |
| CT620 CEA ScFv: | CT620 CEA ScFv |
| QVQLVQSGSELKKPGASVKVSCKASGYTFTEFGMNWVRQAPGQGLEWMGWINTKTGEAT | DNA Sequence: |
| YVEEFKGRFVFSLDTSVSTAYLQISSLKAEDTAVYYCARWDFAHYFQTMDYWGQGTTVTVSS | SEQ ID NO: 287 |
| GGGGSGGGGSGGGGSGGDIQMTQSPSSLSASVGDRVTITCKASAAVGTYVAWYQQKPGK | |
| APKLLIYSASYRKRGVPSRFSGSGSGTDFTLTISSLQPEDFATYYCHQYYTYPLFTFGQGTKLEIK | |
| (SEQ ID NO: 286) | |

EGFR binding domains

| | |
|---|---|
| CT-478 EGFR (VH-VL ScFv Format): | CT-478 EGFR |
| QVQLVESGGGVVQPGRSLRLSCAASGFTFSTYGMHWVRQAPGKGLEWVAVIWDD | DNA |
| GSYKYYGDSVKGRFTISRDNSKNTLYLQMNSLRAEDTAVYYCARDGITMVRGVMKD | Sequence: SEQ |
| YFDYWGQGTLVTVSSGGGGSGGGGSGGGGSGGAIQLTQSPSSLSASVGDRVTITCR | ID NO: 103 |
| ASQDISSALVWYQQKPGKAPKLLIYDASSLESGVPSRFSGSESGTDFTLTISSLQPEDFA | |
| TYYCQQFNSYPLTFGGGTKVEIK (SEQ ID NO: 102) | |
| CT-479 EGFR (VL-VH ScFv Format): | CT-479 EGFR |
| AIQLTQSPSSLSASVGDRVTITCRASQDISSALVWYQQKPGKAPKLLIYDASSLESGVPSRFSGS | DNA |
| ESGTDFTLTISSLQPEDFATYYCQQFNSYPLTFGGGTKVEIKGGGGSGGGGSGGGGSGGQVQ | Sequence: SEQ |
| LVESGGGVVQPGRSLRLSCAASGFTFSTYGMHWVRQAPGKGLEWVAVIWDDGSYKYYGDS | ID NO: 105 |
| VKGRFTISRDNSKNTLYLQMNSLRAEDTAVYYCARDGITMVRGVMKDYFDYWGQGTLVTVS | |
| S (SEQ ID NO: 104) | |
| CT-480 EGFR (VH-VL ScFv format): | CT-480 EGFR |
| QIQLVQSGPELKKPGETVKISCKASGYTFTEYPIHWVKQAPGKGFKWMGMIYTDIGKPTYAE | DNA Sequence: |
| EFKGRFAFSLETSASTAYLQINNLKNEDTATYFCVRDRYDSLFDYWGQGTTLTVSSGGGGSGG | SEQ ID NO: 107 |
| GGSGGGGSGGDVVMTQTPLSLPVSLGDQASISCRSSQSLVHSNGNTYLHWYLQKPGQSPKL | |
| LIYKVSNRFSGVPDRFSGSGSGTDFTLKISRVEAEDLGVYFCSQSTHVPWTFGGGTKLEIK | |
| (SEQ ID NO: 106) | |
| CT-481 EGFR (VL-VH ScFv Format): | CT-481 EGFR |
| DVVMTQTPLSLPVSLGDQASISCRSSQSLVHSNGNTYLHWYLQKPGQSPKLLIYKVSNRFSGV | DNA Sequence: |
| PDRFSGSGSGTDFTLKISRVEAEDLGVYFCSQSTHVPWTFGGGTKLEIKGGGGSGGGGSGGG | SEQ ID NO: 109 |
| GSGGQIQLVQSGPELKKPGETVKISCKASGYTFTEYPIHWVKQAPGKGFKWMGMIYTDIGKP | |
| TYAEEFKGRFAFSLETSASTAYLQINNLKNEDTATYFCVRDRYDSLFDYWGQGTTLTVSS (SEQ | |
| ID NO: 108) | |
| CT-482 EGFR (VH-VL ScFv Format): | CT-482 EGFR |
| EMQLVESGGGFVKPGGSLKLSCAASGFAFSHYDMSWVRQTPKQRLEWVAYIASGGDITYYA | DNA Sequence: |
| DTVKGRFTISRDNAQNTLYLQMSSLKSEDTAMFYCSRSSYGNNGDALDFWGQGTSVTVSSG | SEQ ID NO: 111 |
| GGGSGGGGSGGGGSGGDVVMTQTPLSLPVSLGDQASISCRSSQSLVHSNGNTYLHWYLQK | |
| PGQSPKLLIYKVSNRFSGVPDRFSGSGSGTDFTLKISRVEAEDLGVYFCSQSTHVLTFGSGTKLEI | |
| K (SEQ ID NO: 110) | |
| CT-483 EGFR (VL-VH ScFv Format): | CT-483 EGFR |
| DVVMTQTPLSLPVSLGDQASISCRSSQSLVHSNGNTYLHWYLQKPGQSPKLLIYKVSNRFSGV | DNA Sequence: |
| PDRFSGSGSGTDFTLKISRVEAEDLGVYFCSQSTHVLTFGSGTKLEIKGGGGSGGGGSGGGGS | SEQ ID NO: 113 |
| GGEMQLVESGGGFVKPGGSLKLSCAASGFAFSHYDMSWVRQTPKQRLEWVAYIASGGDITY | |
| YADTVKGRFTISRDNAQNTLYLQMSSLKSEDTAMFYCSRSSYGNNGDALDFWGQGTSVTVS | |
| S (SEQ ID NO: 112) | |
| CT-486 EGFR (VH-VL ScFv Format): | CT-486 EGFR |
| QVQLKQSGPGLVQPSQSLSITCTVSGFSLTNYGVHWVRQSPGKGLEWLGVIWSGGNTDYNT | DNA Sequence: |
| PFTSRLSINKDNSKSQVFFKMNSLQSNDTAIYYCARALTYYDYEFAYWGQGTLVTVSAGGGG | SEQ ID NO: 115 |
| SGGGGSGGGGSGGDILLTQSPVILSVSPGERVSFSCRASQSIGTNIHWYQQRTNGSPRLLIKYA | |
| SESISGIPSRFSGSGSGTDFTLSINSVESEDIADYYCQQNNNWPTTFGAGTKLELK (SEQ ID | |
| NO: 114) | |
| CT-487 EGFR (VH-VL ScFv Format): | CT-487 EGFR |
| QVQLVQSGAEVKKPGASVKVSCKASGYTFTSHWMHWVRQAPGQGLEWIGEFNPSNGRTN | DNA Sequence: |
| YNEKFKSKATMTVDTSTNTAYMELSSLRSEDTAVYYCASRDYDYDGRYFDYWGQGTLVTVSS | SEQ ID NO: 117 |
| GGGGSGGGGSGGGGSGGDIQMTQSPSSLSASVGDRVTITCSASSSVTYMYWYQQKPGKAP | |
| KLLIYDTSNLASGVPSRFSGSGSGTDYTFTISSLQPEDIATYYCQQWSSHIFTFGQGTKVEIK | |
| (SEQ ID NO: 116) | |
| CT-488 EGFR (VH-VL ScFv Format): | CT-488 EGFR |
| QVQLQESGPGLVKPSETLSLTCTVSGGSVSSGDYYWTWIRQSPGKGLEWIGHIYYSGNTNYN | DNA Sequence: |
| PSLKSRLTISIDTSKTQFSLKLSSVTAADTAIYYCVRDRVTGAFDIWGQGTMVTVSSGGGGSGG | SEQ ID NO: 119 |
| GGSGGGGSGGDIQMTQSPSSLSASVGDRVTITCQASQDISNYLNWYQQKPGKAPKLLIYDAS | |
| NLETGVPSRFSGSGSGTDFTFTISSLQPEDIATYFCQHFDHLPLAFGGGTKVEIK (SEQ ID NO: | |
| 118) | |
| CT-489 EGFR ScFv: | ND |
| QVQLQESGPGLVKPSQTLSLTCTVSGGSISSGDYYWSWIRQPPGKGLEWIGYIYYSGS | |
| TDYNPSLKSRVTMSVDTSKNQFSLKVNSVTAADTAVYYCARVSIFGVGTFDYWGQG | |
| TLVTVSSGGGGSGGGGSGGGGSGGEIVMTQSPATLSLSPGERATLSCRASQSVSSYLAWYQ | |
| QKPGQAPRLLIYDASNRATGIPARFSGSGSGTDFTLTISSLEPEDFAVYYCHQYGSTPLTFGGG | |
| TKAEIK (SEQ ID NO: 391) | |

CD19 Binding Domains

| | |
|---|---|
| C-2096 CD19 ScFv: | C-2096 CD19 |
| DIQMTQTTSSLSASLGDRVTISCRASQDISKYLNWYQQKPDGTVKLLIYHTSRLHSGV | ScFv DNA |
| PSRFSGSGSGTDYSLTISNLEQEDIATYFCQQGNTLPYTFGGGTKLEITGGGGSGGGG | Sequence: SEQ |
| SGGGGSEVKLQESGPGLVAPSQSLSVTCTVSGVSLPDYGVSWIRQPPRKGLEWLGVI | ID NO: 276 |
| WGSETTYYNSALKSRLTIIKDNSKSQVFLKMNSLQTDDTAIYYCAKHYYYGGSYAMDY | |
| WGQGTSVTVSS (SEQ ID NO: 275) | |
| C-2815 CD19 ScFv: | C-2815 CD19 |
| DIQMTQTTSSLSASLGDRVTISCRASQDISKYLNWYQQKPDGTVKLLIYHTSRLHSGV | ScFv DNA |
| PSRFSGSGSGTDYSLTISNLEQEDIATYFCQQGNTLPYTFGGGTKLEITGGGGSGGGG | Sequence: SEQ |
| SGGGGSGGEVKLQESGPGLVAPSQSLSVTCTVSGVSLPDYGVSWIRQPPRKGLEWL | ID NO: 278 |
| GVIWGSETTYYNSALKSRLTIIKDNSKSQVFLKMNSLQTDDTAIYYCAKHYYYGGSYA | |
| MDYWGQGTSVTVSS (SEQ ID NO: 277) | |

In some embodiments, the activator ligand is MSLN or a peptide antigen thereof, and the activator ligand binding domain comprises a MSLN binding domain. In some embodiments, the MSLN ligand binding domain comprises an ScFv domain. In some embodiments, the MSLN ligand binding domain comprises a sequence of SEQ ID NO: 86, SEQ ID NO: 88, SEQ ID NO: 90 or SEQ ID NO: 92. In some embodiments, the MSLN ligand binding domain comprises a sequence at least 90%, at least 95% or at least 99% identical to SEQ ID NO: 86, SEQ ID NO: 88, SEQ ID NO: 90 or SEQ ID NO: 92. In some embodiments, the MSLN ligand binding domain is encoded by a sequence comprising SEQ ID NO: 87, SEQ ID NO: 89, SEQ ID NO: 91 or SEQ ID NO: 93. In some embodiments, the MSLN ligand binding domain is encoded by a sequence having at least 80% identity, at least 85% identity, at least 90% identity, at least 95% identity or at least 99% identity to a sequence of SEQ ID NO: 87, SEQ ID NO: 89, SEQ ID NO: 91 or SEQ ID NO: 93.

In some embodiments, the activator ligand is CEA or a peptide antigen thereof, and the activator ligand binding domain comprises a CEA binding domain. In some embodiments, the CEA ligand binding domain comprises an ScFv domain. In some embodiments, the CEA ligand binding domain comprises a sequence of SEQ ID NO: 94, SEQ ID NO: 96, SEQ ID NO: 98, SEQ ID NO: 100, SEQ ID NO: 282, SEQ ID NO: 284 or SEQ ID NO: 286. In some embodiments, the CEA ligand binding domain comprises a sequence at least 90%, at least 95% or at least 99% identical to SEQ ID NO: 94, SEQ ID NO: 96, SEQ ID NO: 98, SEQ ID NO: 100, SEQ ID NO: 282, SEQ ID NO: 284 or SEQ ID NO: 286. In some embodiments, the CEA ligand binding domain is encoded by a sequence comprising SEQ ID NO: 95, SEQ ID NO: 97, SEQ ID NO: 99, SEQ ID NO: 101, SEQ ID NO: 283, SEQ ID NO: 285 or SEQ ID NO: 287. In some embodiments, the CEA ligand binding domain is encoded by a sequence having at least 80% identity, at least 85% identity, at least 90% identity, at least 95% identity or at least 99% identity to a sequence of SEQ ID NO: 95, SEQ ID NO: 97, SEQ ID NO: 99, SEQ ID NO: 101, SEQ ID NO: 283, SEQ ID NO: 285 or SEQ ID NO: 287.

In some embodiments, the activator ligand is CEA or a peptide antigen thereof, and the activator ligand binding domain comprises a CEA binding domain. In some embodiments, the CEA ligand binding domain comprises a CDR-H1 of EFGMN (SEQ ID NO: 294), a CDR-H2 of WINTKTGEATYVEEFKG (SEQ ID NO: 295), a CDR-H3 of WDFAYYVEAMDY (SEQ ID NO: 2%) or WDFAHYFQTMDY (SEQ ID NO: 297), a CDR-L1 of KASQNVGTNVA (SEQ ID NO: 298) or KASAAVGTYVA (SEQ ID NO: 299), a CDR-L2 of SASYRYS (SEQ ID NO: 300) or SASYRKR (SEQ ID NO: 301), and a CDR-L3 of HQYYTYPLFT (SEQ ID NO: 302) or sequences having at least 85% or at least 95% identity thereto. In some embodiments, a CEA ScFv comprises a CDR-H1 of EFGMN (SEQ ID NO: 294), a CDR-H2 of WINTKTGEATYVEEFKG (SEQ ID NO: 295), a CDR-H3 of WDFAYYVEAMDY (SEQ ID NO: 2%) or WDFAHYFQTMDY (SEQ ID NO: 297), a CDR-L1 of KASQNVGTNVA (SEQ ID NO: 298) or KASAAVGTYVA (SEQ ID NO: 299), a CDR-L2 of SASYRYS (SEQ ID NO: 300) or SASYRKR (SEQ ID NO: 301) and a CDR-L3 of HQYYTYPLFT (SEQ ID NO: 302). In some embodiments, a CEA binding domain comprises a CDR-H1 of EFGMN (SEQ ID NO: 294), a CDR-H2 of WINTKTGEATYVEEFKG (SEQ ID NO: 295), a CDR-H3 of WDFAYYVEAMDY (SEQ ID NO: 2%), a CDR-L1 of KASQNVGTNVA (SEQ ID NO: 298), a CDR-L2 of SASYRYS (SEQ ID NO: 300) and a CDR-L3 of HQYYTYPLFT (SEQ ID NO: 302). In some embodiments, a CEA ScFv comprises a CDR-H1 of EFGMN (SEQ ID NO: 294), a CDR-H2 of WINTKTGEATYVEEFKG (SEQ ID NO: 295), a CDR-H3 of WDFAYYVEAMDY (SEQ ID NO: 2%), a CDR-L1 of KASAAVGTYVA (SEQ ID NO: 299), a CDR-L2 of SASYRKR, and a CDR-L3 of HQYYTYPLFT (SEQ ID NO: 302). In some embodiments, a CEA binding domain comprises a CDR-H1 of EFGMN (SEQ ID NO: 294), a CDR-H2 of WINTKTGEATYVEEFKG (SEQ ID NO: 295), a CDR-H3 of WDFAHYFQTMDY (SEQ ID NO: 297), a CDR-L1 of KASAAVGTYVA (SEQ ID NO: 299), a CDR-L2 of SASYRKR, and a CDR-L3 of HQYYTYPLFT (SEQ ID NO: 302).

In some embodiments, the activator ligand is CEA or a peptide antigen thereof, and the activator receptor is a CEA CAR. In some embodiments, the CEA CAR comprises sequence at least 90%, at least 95% or at least 99% identical to SEQ ID NO: 288, SEQ ID NO: 290 or SEQ ID NO: 292. In some embodiments, the CEA CAR comprises or consists essentially of SEQ ID NO: 288, SEQ ID NO: 290 or SEQ ID NO: 292. In some embodiments, the CEA CAR is encoded by a sequence comprising or consisting essentially of SEQ ID NO: 289, SEQ ID NO: 291 or SEQ ID NO: 293. In some embodiments, the CEA CAR is encoded by a sequence having at least 80% identity, at least 85% identity, at least 90% identity, at least 95% identity or at least 99% identity to SEQ ID NO: 289, SEQ ID NO: 291 or SEQ ID NO: 293.

In some embodiments, the activator ligand is EGFR or a peptide antigen thereof, and the activator ligand binding domain comprises an EGFR binding domain. In some embodiments, the EGFR ligand binding domain comprises an ScFv domain. In some embodiments, the EGFR ligand binding domain comprises a sequence of SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118 or SEQ ID NO: 391. In some embodiments, the EGFR ligand binding domain comprises a sequence at least 90%, at least 95% or at least 99% identical to SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118 or SEQ ID NO: 391. In some embodiments, the EGFR ligand binding domain is encoded by a sequence comprising SEQ ID NO: 103, SEQ ID NO: 104, SEQ ID NO: 107, SEQ ID NO: 109, SEQ ID NO: 111, SEQ ID NO: 113, SEQ ID NO: 115, SEQ ID NO: 117 or SEQ ID NO: 119. In some embodiments, the EGFR ligand binding domain is encoded by a sequence having at least 80% identity, at least 85% identity, at least 90% identity, at least 95% identity or at least 99% identity to a sequence of SEQ ID NO: 103, SEQ ID NO: 104, SEQ ID NO: 107, SEQ ID NO: 109, SEQ ID NO: 111, SEQ ID NO: 113, SEQ ID NO: 115, SEQ ID NO: 117 or SEQ ID NO: 119.

In some embodiments, the activator ligand is EGFR or a peptide antigen thereof, and the activator ligand binding domain comprises an EGFR ligand binding domain. In some embodiments, the EGFR binding domain comprises a VH and/or a VL domain selected from the group disclosed in Table 2 or a sequence having at least 90% identity thereto. In some embodiments, the EGFR ligand binding domain comprises a VH domain selected from the group consisting of SEQ ID NO: 120, SEQ ID NO: 122, SEQ ID NO: 124, SEQ ID NO: 126, SEQ ID NO: 128 and SEQ ID NO: 130. In some embodiments, the EGFR ligand binding domain comprises a VH selected from the group consisting of SEQ ID NO: 120, SEQ ID NO: 122, SEQ ID NO: 124, SEQ ID NO: 126 SEQ ID NO: 128 and SEQ ID NO: 130 or a sequence having at least 90, at least 95% or at least 99% identity thereto. In some embodiments, the EGFR ligand binding domain comprises a VL domain selected from the group consisting of SEQ ID NO: 121, SEQ ID NO: 123, SEQ ID NO: 125, SEQ ID NO: 127, SEQ ID NO: 129 and SEQ ID NO: 131. In some embodiments, the EGFR ligand binding domain comprises a VH selected from the group consisting of SEQ ID NO: 121, SEQ ID NO: 123, SEQ ID NO: 125, SEQ ID NO: 127, SEQ ID NO: 129 and SEQ ID NO: 131 or a sequence having at least 90%, at least 95% or at least 99% identity thereto.

TABLE 2
EGFR Variable Heavy (VH) and Variable Light (VL) domains

| EGFR VH | EGFR VL |
|---|---|
| CT478, CT479: | CT478, CT479; |
| QVQLVESGGGVVQPGRSLRLSCAASGFTFSTYGMHWVR | AIQLTQSPSSLSASVGDRVTITCRASQDISSALVWY |
| QAPGKGLEWVAVIWDDGSYKYYGDSVKGRFTISRDNSKN | QQKPGKAPKLLIYDASSLESGVPSRFSGSESGTDFT |
| TLYLQMNSLRAEDTAVYYCARDGITMVRGVMKDYFDYW | LTISSLQPEDFATYYCQQFNSYPLTFGGGTKVEIK |
| GQGTLVTVSS (SEQ ID NO: 120) | (SEQ ID NO: 121) |
| CT480, CT481: | CT480, CT481: |
| QIQLVQSGPELKKPGETVKISCKASGYTFTEYPIHWVKQAP | DVVMTQTPLSLPVSLGDQASISCRSSQSLVHSNG |
| GKGFKWMGMIYTDIGKPTYAEEFKGRFAFSLETSASTAYL | NTYLHWYLQKPGQSPKLLIYKVSNRFSGVPDRFSG |
| QINNLKNEDTATYFCVRDRYDSLFDYWGQGTTLTVSS | SGSGTDFTLKISRVEAEDLGVYFCSQSTHVPWTFG |
| (SEQ ID NO: 122) | GGTKLEIK (SEQ ID NO: 123) |
| CT482, CT483: | CT482, CT483: |
| EMQLVESGGGFVKPGGSLKLSCAASGFAFSHYDMSWVRQ | DVVMTQTPLSLPVSLGDQASISCRSSQSLVHSNG |
| TPKQRLEWVAYIASGGDITYYADTVKGRFTISRDNAQNTLY | NTYLHWYLQKPGQSPKLLIYKVSNRFSGVPDRFSG |
| LQMSSLKSEDTAMFYCSRSSYGNNGDALDFWGQGTSVTV | SGSGTDFTLKISRVEAEDLGVYFCSQSTHVLTFGSG |
| SS (SEQ ID NO: 124) | TKLEIK (SEQ ID NO: 125) |
| CT486: | CT486; |
| QVQLKQSGPGLVQPSQSLSITCTVSGFSLTNYGVHWVRQS | DILLTQSPVILSVSPGERVSFSCRASQSIGTNIHWY |
| PGKGLEWLGVIWSGGNTDYNTPFTSRLSINKDNSKSQVFF | QQRTNGSPRLLIKYASESISGIPSRFSGSGSGTDFTL |
| KMINSLQSNDTAIYYCARALTYYDYEFAYWGQGTLVTVSA | SINSVESEDIADYYCQQNNNWPTTFGAGTKLELK |
| (SEQ ID NO: 126) | (SEQ ID NO: 127) |
| CT487: | CT487: |
| QVQLVQSGAEVKKPGASVKVSCKASGYTFTSHWMHWVR | DIQMTQSPSSLSASVGDRVTITCSASSSVTYMYW |
| QAPGQGLEWIGEFNPSNGRTNYNEKFKSKATMTVDTSTN | YQQKPGKAPKLLIYDTSNLASGVPSRFSGSGSGTD |
| TAYMELSSLRSEDTAVYYCASRDYDYDGRYFDYWGQGTLV | YTFTISSLQPEDIATYYCQQWSSHIFTFGQGTKVEI |
| TVSS (SEQ ID NO: 128) | K (SEQ ID NO: 129) |
| CT488: | CT488: |
| QVQLQESGPGLVKPSETLSLTCTVSGGSVSSGDYYWTWIR | DIQMTQSPSSLSASVGDRVTITCQASQDISNYLN |
| QSPGKGLEWIGHIYYSGNTNYNPSLKSRLTISIDTSKTQFSLK | WYQQKPGKAPKLLIYDASNLETGVPSRFSGSGSG |
| LSSVTAADTAIYYCVRDRVTGAFDIWGQGTMVTVSS (SEQ | TDFTFTISSLQPEDIATYFCQHFDHLPLAFGGGTKV |
| ID NO: 130) | EIK (SEQ ID NO: 131) |

In some embodiments, the activator ligand is EGFR or a peptide antigen thereof, and the activator ligand binding domain is an EGFR ligand binding domain. In some embodiments, the EGFR binding domain comprises complementarity determining region (CDRs) selected from the group of CDRs disclosed in Table 3. In some embodiments, the EGFR ligand binding domain comprises CDRs having at least 95% sequence identity to CDRs disclosed in Table 3. In some embodiments, the EGFR ligand binding domain comprises CDRs selected from SEQ ID NOs: 131-166. In some embodiments, the EGFR ligand binding domain comprises a heavy chain CDR 1 (CDR H1) selected from the group consisting of SEQ ID NOs: 132-137. In some embodiments, the EGFR ligand binding domain comprises a heavy chain CDR 2 (CDR H2) selected from the group consisting of SEQ ID NOs: 138-143. In some embodiments, the EGFR ligand binding domain comprises a heavy chain CDR 3 (CDR H3) selected from the group consisting of SEQ ID NOs: 144-149. In some embodiments, the EGFR ligand binding domain comprises a light chain CDR 1 (CDR L1) selected from the group consisting of SEQ ID NOs: 150-155. In some embodiments, the EGFR ligand binding domain comprises a light chain CDR 2 (CDR L2) selected from the group consisting of SEQ ID NOs: 156-160. In some embodiments, the EGFR ligand binding domain comprises a light chain CDR 3 (CDR L3) selected from the group consisting of SEQ ID NOs: 161-166. In some embodiments, the EGFR ligand binding domain comprises a CDR H1 selected from SEQ ID NOs: 132-137, a CDR H2 selected from SEQ ID NOs: 138-143, a CDR H3 selected from SEQ ID NOs: 144-149, a CDR L1 selected from SEQ ID NOs: 150-155, a CDR L2 selected from SEQ ID NOs: 156-160, and a CDR L3 selected from SEQ ID NOs: 156-160.

TABLE 3
EGFR antigen binding domain CDRs.

| CDR H1 | CDR H2 | CDR H3 | CDR L1 | CDR L2 | CDR L3 |
|---|---|---|---|---|---|
| TYGMH | VIWDDGSYKYYG | DGITMVRGVMKDY | RASQDISSALV | DASSLES | QQFNSYPLT |
| (SEQ ID | DSVKG (SEQ ID | FDY (SEQ ID NO: | (SEQ ID NO: 150) | (SEQ ID | (SEQ ID NO: |
| NO: 132) | NO: 138) | 144) | | NO: 156) | 161) |
| EYPIH (SEQ | MIYTDIGKPTYAE | DRYDSLFDY (SEQ | RSSQSLVHSNGNT | KVSNRFS | SQSTHVPW |
| ID NO: 133) | EFKG (SEQ ID | ID NO: 145) | YLH (SEQ ID NO: | (SEQ ID | T (SEQ ID |
| | NO: 139) | | 151) | NO: 157) | NO: 162) |
| HYDMS | YIASGGDITYYAD | SSYGNNGDALDF | RSSQSLVHSNGNT | KVSNRFS | SQSTHVLT |
| (SEQ ID | TVKG (SEQ ID | (SEQ ID NO: 146) | YLH (SEQ ID NO: | (SEQ ID | (SEQ ID NO: |
| NO: 134) | NO: 140) | | 152) | NO: 157) | 163) |
| NYGVH | VIWSGGNTDYN | ALTYYDYEFAY (SEQ | RASQSIGTNIH | YASESIS | QQNNNWP |
| (SEQ ID | TPFTS (SEQ ID | ID NO: 147) | (SEQ ID NO: 153) | (SEQ ID | TT (SEQ ID |
| NO: 135) | NO: 141) | | | NO: 158) | NO: 164) |
| SHWMH | EFNPSNGRTNYN | RDYDYDGRYFDY | SASSSVTYMY | DTSNLAS | QQWSSHIFT |
| (SEQ ID | EKFKS (SEQ ID | (SEQ ID NO: 148) | (SEQ ID NO: 154) | (SEQ ID | (SEQ ID NO: |
| NO: 136) | NO: 142) | | | NO: 159) | 165) |
| SGDYYWT | HIYYSGNTNYNP | DRVTGAFDI (SEQ | QASQDISNYLN | DASNLET | QHFDHLPLA |
| (SEQ ID | SLKS (SEQ ID | ID NO: 149) | (SEQ ID NO: 155) | (SEQ ID | (SEQ ID NO: |
| NO: 137) | NO: 143) | | | NO: 160) | 166) |

In some embodiments, the activator ligand is a pan-HLA ligand, and the activator binding domain is a pan-HILA binding domain, i.e. a binding domain that binds to and recognizes an antigenic determinant shared among products of the HLA A, B and C loci. Various single variable domains known in the art or disclosed herein are suitable for use in embodiments. Such scFvs include, for example and without limitation, the following mouse and humanized pan-HILA scFv antibodies. An exemplary pan-HILA ligand is W6/32, which recognizes a conformational epitope, reacting with HILA class 1 alpha3 and alpha2 domains.

TABLE 4
pan-HLA ScFv binding domains derived from W6/32

| | Polynucleotide |
|---|---|
| Protein Sequence | Sequence |
| C-002170 W632 scFv (mouse): | C-002170 W632 |
| QVQLKQSGPGLVQPSQSLSLTCTVSGFSLTSYGVHWVRQPPGKGLEWLGVIWSG | scFv (mouse) SEQ |
| GSTDYNAAFISRLSIRKDNSKSQVFFKMNSLQADDTAIYYCARTFTTSTSAWFAYW | ID NO: 168 |
| GQGTLVTVSAGGGGSGGGGGGGGSGGSIVMTQTPKFLLVSAGDRVTITCKASQ | |
| SVSNDVAWYQQKPGQSPICLLIYYASNRYTGVPDRFTGSGYGTDFTFTISTVQAED | |
| LAVYFCQQDYSSPPWTFGGGTKLEIR (SEQ ID NO: 167) | |
| C-002171 W632.1 scFv (humanized): | C-002171 W632.1 |
| QVQLQESGPGLVKPSQTLSLTCTVSGFSLTSYGVHWIRQPPGKGLEWIGVIWSGG | scFv (humanized) |
| STDYNAAFISRVTISVDTSKNQFSLKLSSVTAADTAVYYCARTFTTSTSAWFAYWG | SEQ ID NO: 170 |
| QGTLVTVSSGGGGSGGGGSGGGGSGGDIVMTQSPDSLAVSLGERATINCKASQS | |
| VSNDVAWYQQKPGQPPKLLIYYASNRYTGVPDRFSGSGSGTDFTLTISSLQAEDV | |
| AVYYCQQDYSSPPWTFGGGTKVEIK (SEQ ID NO: 169) | |
| C-002172 W632.2 scFy (humanized): | C-002172 W632.2 |
| EVQLLESGGGLVQPGGSLRLSCAASGFSLTSYGVHWVRQAPGKGLEWVSVIWSG | scFv (humanized) |
| GSTDYNAAFISRFTISRDNSKNTLYLQMNSLRAEDTAVYYCARTFTTSTSAWFAY | SEQ ID NO: 172 |
| WGQGTLVTVSSGGGGSGGGGSGGGGSGGDIQMTQSPSSLSASVGDRVTITCKA | |
| SQSVSNDVAWYQQKPGKAPKLLIYYASNRYTGVPSRFSGSGSGTDFTFTISSLQPE | |
| DIATYYCQQDYSSPPWTFGGGTKVEIK (SEQ ID NO: 171) | |
| C-002173 W632.3 scFv (humanized): | C-002173 W632.3 |
| QVQLQESGPGLVKPSETLSLTCTVSGFSLTSYGVHWIRQPPGKGLEWIGVIWSGG | scFv (humanized) |
| STDYNAAFISRVTISRDTSKNQFSLKLSSVTAADTAVYYCARTFTTSTSAWFAYWG | SEQ ID NO: 174 |
| QGTLVTVSSGGGGSGGGGSGGGGSGGDIVMTQTPLSLSVTPGQPASISCKASQS | |
| VSNDVAWYLQKPGQSPQLLIYYASNRYTGVPDRFSGSGSGTDFTLKISRVEAEDV | |
| GVYYCQQDYSSPPWTFGGGTKVEIK (SEQ ID NO: 173) | |
| C-002174 W632.5 scFv (humanized): | C-002174 W632.5 |
| QVQLVESGGGVVQPGRSLRLSCAVSGFSLTSYGMHWVRQAPGKGLEWVAVIW | scFv (humanized) |
| SGGSTDYNAAFISRFTISRDNSKNTLYLQMNSLRAEDTAVYYCAKTFTTSTSAWFA | SEQ ID NO: 176 |
| YWGQGTLVTVSSGGGGSGGGGSGGGGSGGEIVLTQSPATLSLSPGERATLSCRA | |
| SQSVSNDLAWYQQKPGQAPRLLIYYASNRYTGVPDRFSGSGSGTDFTLTISSLEPE | |
| DFAVYYCQQDYSSPPWTFGQGTKVEIK (SEQ ID NO: 175) | |
| C-002175 W632.6 scFv (humanized): | C-002175 W632.6 |
| QVQLVESGGGVVQPGRSLRLSCAVSGFSLTSYGMHWVRQAPGKGLEWVAVIW | scFv (humanized) |
| SGGSTDYNAAFISRFTISRDNSKNTLYLQMNSLRAEDTAVYYCARTFTTSTSAWFA | SEQ ID NO: 178 |
| YWGQGTLVTVSSGGGGSGGGGSGGGGSGGDIQMTQSPSSLSASVGDRVTITCQ | |
| ASQSVSNDLNWYQQKPGKAPKLLIYYASNRYTGVPDRFSGSGSGTDFTFTISSLQP | |
| EDIATYYCQQDYSSPPWTFGGGTKVEIK (SEQ ID NO: 177) | |

In some embodiments, the activator ligand is pan-HLA ligand, and the activator ligand binding domain comprises a pan-HLA ligand binding domain. In some embodiments, the pan-HILA ligand binding domain comprises an ScFv domain. In some embodiments, the pan-HLA ligand binding domain comprises a sequence of SEQ ID NO: 167, SEQ ID NO: 169, SEQ ID NO: 171, SEQ ID NO: 173. SEQ ID NO: 175, or SEQ ID NO: 177. In some embodiments, the pan-LILA ligand binding domain comprises a sequence at least 90%, at least 95% or at least 99% identical to SEQ ID NO: 167, SEQ ID NO: 169, SEQ ID NO: 171, SEQ ID NO: 173, SEQ ID NO: 175, or SEQ ID NO: 177. In some embodiments, the pan-LILA ligand binding domain is encoded by a sequence comprising SEQ ID NO: 168, SEQ ID NO: 170, SEQ ID NO: 172, SEQ ID NO: 174. SEQ ID NO: 176, or SEQ ID NO: 178. In some embodiments, the pan-LILA ligand binding domain is encoded by a sequence having at least 80% identity, at least 85% identity, at least 90% identity, at least 95% identity or at least 99% identity to a sequence of SEQ ID NO: 168, SEQ ID NO: 170, SEQ ID NO: 172, SEQ ID NO: 174, SEQ ID NO: 176, or SEQ ID NO: 178.

In some embodiments, the activator ligand is CD19 molecule (CD19) or a peptide antigen thereof, and the activator ligand binding domain comprises a CD19 ligand binding domain. In some embodiments, the CD19 ligand binding domain comprises an ScFv domain. In some embodiments, the CD19 ligand binding domain comprises a sequence at least 90%, at least 95% or at least 99% identical to SEQ ID NO: 275 or SEQ ID NO: 277. In some embodiments, the CD-19 ligand binding domain comprises a sequence of SEQ ID NO: 275 or SEQ ID NO: 277. In some embodiments, the CD19 ligand binding domain is encoded by a sequence comprising SEQ ID NO: 276, or SEQ ID NO: 278. In some embodiments, the CD19 ligand binding domain is encoded by a sequence having at least 80% identity, at least 85% identity, at least 90% identity, at least 95% identity or at least 99% identity to a sequence of SEQ ID NO: 276 or SEQ ID NO: 278.

In some embodiments, activator ligand is CD19 molecule (CD19) or a peptide antigen thereof, and the activator receptor is a CAR. In some embodiments, the CD19 CAR comprises a sequence at least 90%, at least 95% or at least 99% identical to SEQ ID NO: 279 or SEQ ID NO: 281. In some embodiments, the CD19 CAR comprises or consists essentially of SEQ ID NO: 279 or SEQ ID NO: 281. In some embodiments, the CD19 CAR is encoded by a sequence having at least 80% identity, at least 85% identity, at least 90% identity, at least 95% identity or at least 99% identity to a sequence of SEQ ID NO: 280 or SEQ ID NO: 390. In some embodiments, the CD19 CAR is encoded by a sequence comprising or consisting essentially of SEQ ID NO: 280 or SEQ ID NO: 390. It will be appreciated by the person of ordinary skill that first, activator ligand binding domains for the first receptor may be isolated or derived from any source known in the art, including, but not limited to, art recognized T cell receptors, chimeric antigen receptors and antibody binding domains. For example, the first ligand binding domain may be derived from any of the antibodies disclosed in Table 5, and bind to a first ligand selected from the antigens described in Table 5. Accordingly, the immune cells comprising the two receptor system described can be used to treat any of the diseases or disorders described in Table 5. Selection of an appropriate first, activator receptor ligand binding domain to treat any the cancers described herein will be apparent to those of skill in the art.

TABLE 5
Exemplary Antibodies

| Antigen | Antibody | Exemplary Diseases and Disorders |
|---|---|---|
| TNF receptor superfamily | Urelumab, Utomilumab | cancer, diffuse large B-cell lymphoma |
| member 9 (4-1BB, CD137) | | |
| 5′-nucleotidase | Oleclumab | pancreatic and colorectal cancer |
| trophoblast glycoprotein | Naptumomab | non-small cell lung carcinoma, renal |
| (5T4) | | cell carcinoma |
| activin receptor-like | Ascrinvacumab | cancer |
| kinase 1 | | |
| alpha-fetoprotein | Tacatuzumab | cancer |
| angiopoietin 2 | Nesvacumab, Vanucizumab | cancer |
| TNF superfamily member | Belimumab, Tabalumab, | cancers and autoimmune disorders |
| 13b (BAFF) | Tibulizumab | |
| TNF receptor superfamily | Belantamab | multiple myeloma |
| member 17 (BCMA) | | |
| mucin 16, cell surface | Igovomab, Oregovomab, | ovarian cancer |
| associated (CA-125) | Sofituzumab | |
| C-C motif chemokine | Mogamulizumab | adult T-cell leukemia/lymphoma |
| receptor 4 (CCR4) | | |
| interleukin 3 receptor | Talacotuzumab | leukemia |
| subunit alpha (CD123) | | |
| TNF receptor superfamily | Tavolimab, Vonlerolizumab | cancer |
| member 4 (CD134) | | |
| cytotoxic T-lymphocyte | Ipilimumab | melanoma |
| associated protein 4 | | |
| (CD152) | | |
| CD19 molecule (CD19) | Duvortuxizumab, Blinatumomab, | cancer |
| | Coltuximab, Denintuzumab, | |
| | Inebilizumab, Loncastuximab, | |
| | Taplitumomab | |
| membrane spanning 4- | Ibritumomab, Obinutuzumab, | cancers, multiple sclerosis, |
| domains A1 (CD20) | Ocaratuzumab, Ocrelizumab, | autoimmune disorders |
| | Ofatumumab, Rituximab, | |
| | Tositumomab, Veltuzumab | |
| CD200 molecule (CD200) | Samalizumab | cancer |
| CD22 molecule (CD22) | Bectumomab, Epratuzumab, | cancer |
| | Inotuzumab, Moxetumomab, | |
| | Pinatuzumab | |
| Fc fragment of IgE | Gomiliximab, Lumiliximab | chronic lymphocytic leukemia |
| receptor II (CD23, IgE | | |
| receptor) | | |
| interleukin 2 receptor | Camidanlumab, Basiliximab, | leukemias and lymphomas |
| subunit alpha (CD25) | Inolimomab, Daclizumab | |
| CD27 molecule (CD27) | Varlilumab | solid tumors and hematologic |
| | | malignancies |
| CD276 molecule (CD276) | Enoblituzumab, Omburtamab | cancer |
| TNF receptor superfamily | Brentuximab, Iratumumab | Hodgkin's lymphoma |
| member 8 (CD30, | | |
| TNFRSF8) | | |
| CD33 molecule (CD33) | Gemtuzumab, Lintuzumab, | acute myelogenous leukemia |
| | Vadastuximab | |
| CD37 molecule (CD37) | Lilotomab, Otlertuzumab, | cancer |
| | Tetulomab | |
| CD38 molecule (CD38) | Daratumumab, Isatuximab | multiple myeloma |
| CD44 molecule v6 (CD44 | Bivatuzumab | squamous cell carcinoma |
| v6) | | |
| integrin subunit alpha V | Abituzumab, Intetumumab | cancer |
| (CD51) | | |
| neural cell adhesion | Lorvotuzumab | cancer |
| molecule 1 (CD56) | | |
| CD6 molecule (CD6) | Itolizumab | psoriasis |
| CD70 molecule (CD70) | Cusatuzumab, Vorsetuzumab | cancer |
| CD74 molecule (CD74) | Milatuzumab | hematological malignancies |
| CD79B molecule (CD79B) | Polatuzumab, Iladatuzumab | Hematological cancers |
| CD80 molecule (CD80) | Galiximab | B-cell lymphoma |
| CEA cell adhesion | Altumomab, Arcitumomab, | cancer, colorectal cancer |
| molecule 5 (CEA) | Labetuzumab, Cibisatamab | |
| Claudin 18 Isoform 2 | Zolbetuximab | gastric cancer |
| Colony stimulating factor | Lacnotuzumab | cancer |
| 1 (CSF1) | | |
| colony stimulating factor 1 | Cabiralizumab, Emactuzumab | cancer |
| receptor (CSF1R) | | |
| Colony stimulating factor | Gimsilumab, Lenzilumab, | leukemias |
| 2 (CSF2) | Otilimab, Mavrilimumab | |
| cytotoxic T-lymphocyte | Tremelimumab | non-small cell lung, head & neck, |
| associated protein 4 | | urothelial cancer |
| (CTLA-4) | | |
| CXCR4 (CD184) | Ulocuplumab | hematologic malignancies |
| dendritic cell-associated | Tepoditamab | cancer |
| lectin 2 | | |
| delta like canonical Notch | Rovalpituzumab | small cell lung cancer |
| ligand 3 (DLL3) | | |
| delta like canonical Notch | Demcizumab | cancer |
| ligand 4 (DLL4) | | |
| TNF receptor superfamily | Drozitumab | cancer |
| member 10b (DR5) | | |
| EGF like domain multiple 7 | Parsatuzumab | cancer |
| (EGFL7) | | |
| epidermal growth factor | Cetuximab, Depatuxizumab, | cancer |
| receptor (EGFR) | Futuximab, Imgatuzumab, | |
| | Laprituximab, Matuzumab, | |
| | Necitumumab, Nimotuzumab, | |
| | Panitumumab, Zalutumumab, | |
| | Modotuximab, Amivantamab, | |
| | Tomuzotuximab, Losatuxizumab | |
| epithelial cell adhesion | Adecatumumab, Citatuzumab, | cancer |
| molecule (EpCAM) | Edrecolomab, Oportuzumab, | |
| | Solitomab, Tucotuzumab, | |
| | Catumaxomab | |
| EPH receptor A3 (EPHA3) | Ifabotuzumab | glioblastoma multiforme |
| erb-b2 receptor tyrosine | Duligotuzumab, Elgemtumab, | cancer |
| kinase 3 (ERBB3, HER3) | Lumretuzumab, Patritumab, | |
| | Seribantumab, Zenocutuzumab | |
| fibroblast growth factor | Aprutumab, Bemarituzumab | cancer |
| receptor (FGFR2) | | |
| Frizzled receptor | Vantictumab | cancer |
| GD2 ganglioside | Dinutuximab | neuroblastoma |
| GD3 ganglioside | Ecromeximab | malignant melanoma |
| GD3 ganglioside | Mitumomab | small cell lung carcinoma |
| glypican 3 | Codrituzumab | cancer |
| glycoprotein nmb | Glembatumumab | melanoma, breast cancer |
| (GPNMB) | | |
| epidermal growth factor | Zatuximab | cancer |
| receptor (HER1) | | |
| erb-b2 receptor tyrosine | Ertumaxomab, Margetuximab, | cancer, breast cancer |
| kinase 2 (HER2) | Timigutuzumab, Gancotamab, | |
| | Pertuzumab, Trastuzumab | |
| hepatocyte growth factor | Ficlatuzumab, Rilotumumab | cancer |
| (HGF) | | |
| MET proto-oncogene, | Telisotuzumab, Emibetuzumab | cancer |
| receptor tyrosine kinase | | |
| (HGFR) | | |
| IGF-1 receptor (CD221) | Cixutumumab, Dalotuzumab, | cancer |
| | Figitumumab, Ganitumab, | |
| | Robatumumab, Teprotumumab | |
| Interleukin 3 receptor | Flotetuzumab | hematological malignancies |
| Interleukin 1 alpha (IL1A) | Bermekimab | colorectal cancer |
| Interleukin 2 (IL2) | Cergutuzumab | cancer |
| integrin α5β1 | Volociximab | solid tumors |
| integrin αᵥβ₃ | Etaracizumab | melanoma, prostate cancer, ovarian |
| | | cancer |
| lymphocyte activating 3 | Relatlimab | melanoma |
| (LAG3) | | |
| C-C motif chemokine | Carlumab | cancer |
| ligand 2 (MCP-1) | | |
| mesothelin | Amatuximab | cancer |
| Mucin 1 | Clivatuzumab, Gatipotuzumab, | cancer |
| | Pemtumomab, Cantuzumab, | |
| | Pankomab | |
| NGNA ganglioside | Racotumomab | non-small cell lung cancer |
| Notch 1 | Brontictuzumab | cancer |
| Notch receptor | Tarextumab | cancer |
| neuropilin 1 (NRP1) | Vesencumab | cancer |
| programmed cell death 1 | Camrelizumab, Cetrelimab, | cancer |
| (PD-1) | Nivolumab, Pembrolizumab, | |
| | Pidilizumab, Cemiplimab, | |
| | Spartalizumab | |
| CD274 molecule (PD-L1) | Atezolizumab, Avelumab, | cancer |
| | Durvalumab | |
| receptor tyrosine kinase | Cirmtuzumab | leukemia |
| like orphan receptor 1 | | |
| (ROR1) | | |
| tenascin C | Tenatumomab | cancer |
| transforming growth | Fresolimumab | cancer |
| factor beta 1 (TGF-β) | | |
| VEGF-A | Brolucizumab, Bevacizumab, | cancer |
| | Ranibizumab, Varisacumab, | |
| | Faricimab | |
| VEGFR-1 | Icrucumab | cancer |
| VEGFR2 | Alacizumab, Ramucirumab | cancer |

### Inhibitors

The disclosure provides a second ligand, an inhibitor, and a second engineered receptor comprising a second ligand binding domain that binds to the inhibitor ligand.

The disclosure provides a second engineered receptor comprising an extracellular region, the extracellular region comprising a second ligand binding domain capable of specifically binding to a second ligand that inhibits activation of effector cells expressing the first and second receptors, wherein the effector cells are activated by binding of the first ligand to the first engineered receptor.

As used herein an “inhibitor” or “inhibitor ligand,” sometimes called a “blocker,” refers to a second ligand that binds to a second, ligand binding domain (inhibitor LBD) of an engineered receptor of the disclosure, but inhibits activation of an immune cell expressing the engineered receptor. The inhibitor is not expressed by the target cells. The inhibitor ligand is also expressed in a plurality of normal, non-target cells, including normal, non-target cells that express the activator ligand, thereby protecting these cells from the cytotoxic effects of the adoptive cell therapy. Without wishing to be bound by theory, inhibitor ligands can block activation of the effector cells through a variety of mechanisms. For example, binding of the inhibitor ligand to the inhibitor LBD can block transmission of a signal that occurs upon binding of the activator ligand to the activator LBD that would, in the absence of the inhibitor, lead to activation of the immune cell expressing the engineered receptors described herein.

Alternatively, or in addition, binding of the inhibitor ligand to the second engineered receptor can cause loss of cell surface expression the first, activator receptor from the surface of the immune cells comprising the two receptor system described herein. Without wishing to be bound by theory, it is thought that immune cell engagement of activator and inhibitor ligands on normal cells causes the inhibitor receptor to cause removal of nearby activator receptor molecules from the immune cell surface. This process locally desensitizes the immune cell, reversibly raising its activation threshold. Immune cells that engage only the activator ligand on a target cell cause local activation signals which are unimpeded by signals from the second, inhibitory receptor. This local activation increases until release of cytotoxic granules leads to target cell selective cell death. However, modulation of surface receptor expression levels may not be the only mechanism by which blocker receptors inhibit activation of immune cells by the first activator receptor. Without wishing to be bound by theory, other mechanisms may come into play, including, but not limited to, cross-talk between activator and blocker receptor signaling pathways.

In some embodiments, the second ligand is not expressed by the target cells, and is expressed by the non-target cells. In some embodiments, the target cells are cancer cells and the non-target cells are non-cancerous cells.

In some embodiments, the second, inhibitor ligand binding domain comprises an ScFv domain.

In some embodiments, the second, inhibitor ligand binding domain comprises a Vβ-only ligand binding domain.

In some embodiments, the second, inhibitor ligand binding domain comprises an antigen binding domain isolated or derived from a T cell receptor (TCR). For example, the second, inhibitor ligand binding domain comprises TCR α and β chain variable domains.

### Inhibitor Targets

In some embodiments, the inhibitor ligand comprises a gene with high, homogeneous surface expression across tissues, or a peptide antigen thereof. Without wishing to be bound by theory, high, homogeneous surface expression across tissues allows the inhibitor ligand to deliver a large, even inhibitory signal. Alternatively, or in addition, expression of activator and inhibitor targets may be correlated, i.e. the two are expressed at similar levels on non-target cells.

In some embodiments, the second, inhibitor ligand is a peptide ligand. In some embodiments, the second, inhibitor ligand is a peptide antigen complexed with a major histocompatibility (MHC) class I complex (peptide MHC, or pMHC). Inhibitor ligands comprising peptide antigens complexed with pMHC comprising any of HLA-A, HLA-B or HLA-C are envisaged as within the scope of the disclosure.

In some embodiment, the inhibitor ligand is encoded by a gene that is absent or polymorphic in many tumors.

Methods of distinguishing the differential expression of inhibitor ligands between target and non-target cells will be readily apparent to the person or ordinary skill in the art. For example, the presence or absence of inhibitor ligands in non-target and target cells can be assayed by immunohistochemistry with an antibody that binds to the inhibitor ligand, followed by microscopy or FACS, RNA expression profiling of target cells and non-target cells, or DNA sequencing of non-target and target cells to determine if the genomic locus of the inhibitor ligand comprises mutations in either the target or non-target cells.

### Alleles Lost Due to Loss of Heterozygosity (LOH)

Homozygous deletions in primary tumors are rare and small, and therefore unlikely to yield target B candidates. For example, in an analysis of 2218 primary tumors across 21 human cancer types, the top four candidates were cyclin dependent kinase inhibitor 2A (CDKN2A), RB transcriptional corepressor 1 (RB1), phosphatase and tensin homolog (PTEN) and N3PB2. However, CDKN2A (P16) was deleted in only 5% homozygous deletion across all cancers. Homozygous HLA-A deletions were found in less than 0.2% of cancers (Cheng et al., Nature Comm. 8:1221 (2017)). In contrast, deletion of a single copy of a gene in cancer cells due to loss of hemizygosity occurs far more frequently.

In some embodiments, the second, inhibitor ligand comprises an allele of a gene that is lost in target cells due to loss of heterozygosity. In some embodiments, the target cells comprises cancer cells. Cancer cells undergo frequent genome rearrangements, including duplication and deletions. These deletions can lead to the deletion of one copy of one or more genes in the cancer cells.

As used herein, “loss of heterozygosity (LOH)” refers to a genetic change that occurs at high frequency in cancers, whereby one of the two alleles is deleted, leaving a single mono-allelic (hemizygous) locus.

### HLA Class I Alleles

In some embodiments, the second, inhibitor ligand comprises an HLA class I allele. The major histocompatibility complex (MHC) class I is a protein complex that displays antigens to cells of the immune system, triggering immune response. The Human Leukocyte Antigens (HLAs) corresponding to MHC class I are HLA-A, HLA-B and HLA-C.

In some embodiments, the second, inhibitor ligand comprises an HLA class I allele. In some embodiments, the second, inhibitor ligand comprises an allele of HLA class I that is lost in a target cell through LOH. HLA-A is a group of human leukocyte antigens (HLA) of the major histocompatibility complex (MHC) that are encoded by the HLA-A locus. HLA-A is one of three major types of human MHC class I cell surface receptors. The receptor is a heterodimer comprising a heavy α chain and smaller β chain. The α chain is encoded by a variant of HLA-A, while the p chain (β2-microglobulin) is invariant. There are several thousand HLA-A variants, all of which fall within the scope of the instant disclosure.

In some embodiments, the second, inhibitor ligand comprises an HLA-B allele. The HLA-B gene has many possible variations (alleles). Hundreds of versions (alleles) of the HLA-B gene are known, each of which is given a particular number (such as HLA-B27).

In some embodiments, the second, inhibitor ligand comprises an HLA-C allele. HLA-C belongs to the HLA class I heavy chain paralogues. This class I molecule is a heterodimer consisting of a heavy chain and a light chain (beta-2 microglobulin). Over one hundred HLA-C alleles have been described.

In some embodiments, the HLA class I allele has broad or ubiquitous RNA expression.

In some embodiments, the HLA class I allele has a known, or generally high minor allele frequency.

In some embodiments, the HLA class I allele does not require a peptide-MHC antigen, for example when the HLA class I allele is recognized by a pan-HLA ligand binding domain.

In some embodiments, the second inhibitor ligand comprises an HLA-A allele. In some embodiments the HLA-A allele comprises HLA-A*02. Various single variable domains known in the art or disclosed herein that bind to and recognize HLA-A*02 are suitable for use in embodiments. Such scFvs include, for example and without limitation, the following mouse and humanized scFv antibodies that bind HLA-A*02 in a peptide-independent way shown in Table 6 below (complementarity determining regions underlined):

TABLE 6
HLA-A*02 ScFv binding domains
HLA-A*02 antigen binding domains derived from PA2.1 mAb

| | |
|---|---|
| C-001765 PA2.1 scFv (mouse): | C-001765 PA2.1 |
| DVLMTQTPLSLPVSLGDQASISCRSSQSIVHSNGNTYLEWYLQKPGQSPKLLIYKVS | scFv (mouse) |
| NRFSGVPDRFSGSGSGTDFTLKISRVEAEDLGVYYCFQGSHVPRTSGGGTKLEIKGG | DNA Sequence: |
| GGSGGGGSGGGGSGGQVQLQQSGPELVKPGASVRISCKASGYTFTSYHIHWVKQ | SEQ ID NO: 179 |
| RPGQGLEWIGWIYPGNVNTEYNEKFKGKATLTADKSSSTAYMHLSSLTSEDSAVYF | |
| CAREEITYAMDYWGQGTSVTVSS (SEQ ID NO: 53) | |
| C-002159 PA2.1.8 scFv (humanized): | C-002159 |
| QVQLVQSGAEVKKPGSSVKVSCKASGYTFTSYHIHWVRQAPGQGLEWMGWIYP | PA2.1.8 scFv |
| GNVNTEYNEKFKGKATITADKSTSTAYMELSSLRSEDTAVYYCAREEITYAMDYWG | (humanized) |
| QGTTVTVSSGGGGSGGGGSGGGGSGGEIVLTQSPGTLSLSPGERATLSCRSSQSIV | DNA Sequence: |
| HSNGNTYLEWYQQKPGQAPRLLIYKVSNRFSGIPDRFSGSGSGTDFTLTISRLEPED | SEQ ID NO: 180 |
| FAVYYCFQGSHVPRTFGGGTKVEIK (SEQ ID NO: 54) | |
| C-002160 PA2.1.9 scFv (humanized): | C-002160 |
| QVQLVQSGAEVKKPGSSVKVSCKASGYTFTSYHIHWVRQAPGQGLEWMGWIYP | PA2.1.9 scFv |
| GNVNTEYNEKFKGKATITADKSTSTAYMELSSLRSEDTAVYYCAREEITYAMDYWG | (humanized) |
| QGTTVTVSSGGGGSGGGGSGGGGSGGDIVMTQTPLSLPVTPGEPASISCRSSQSIV | DNA Sequence: |
| HSNGNTYLEWYLQKPGQSPQLLIYKVSNRFSGVPDRFSGSGSGTDFTLKISRVEAED | SEQ ID NO: 181 |
| VGVYYCFQGSHVPRTFGGGTKVEIK | |
| (SEQ ID NO: 55) | |
| C-002161 PA2.1.10 scFv (humanized): | C-002161 |
| EVQLVESGGGLVKPGGSLRLSCAASGYTFTSYHIHWVRQAPGKGLEWVGWIYPG | PA2.1.10 scFv |
| NVNTEYNEKFKGRFTISRDDSKNTLYLQMNSLKTEDTAVYYCAREEITYAMDYWG | (humanized) |
| QGTTVTVSSGGGGSGGGGSGGGGSGGDIQMTQSPSSLSASVGDRVTITCRSSQSI | DNA Sequence: |
| VHSNGNTYLEWYQQKPGKAPKLLIYKVSNRFSGVPSRFSGSGSGTDFTLTISSLQPE | SEQ ID NO: 182 |
| DFATYYCFQGSHVPRTFGGGTKVEIK (SEQ ID NO: 56) | |
| C-002162 PA2.1.14 scFv (humanized): | C-002162 |
| QVQLVQSGAEVKKPGSSVKVSCKASGYTFTSYHIHWVRQAPGQGLEWIGWIYPG | PA2.1.14 scFv |
| NVNTEYNEKFKGKATITADESTNTAYMELSSLRSEDTAVYYCAREEITYAMDYWGQ | (humanized) |
| GTLVTVSSGGGGSGGGGSGGGGSGGDIQMTQSPSTLSASVGDRVTITCRSSQSIV | DNA Sequence: |
| HSNGNTYLEWYQQKPGKAPKLLIYKVSNRFSGVPARFSGSGSGTEFTLTISSLQPDD | SEQ ID NO: 183 |
| FATYYCFQGSHVPRTFGQGTKVEVK (SEQ ID NO: 57) | |
| C-002163 PA2.1.18 scFv (humanized): | C-002163 |
| QVQLVQSGAEVKKPGSSVKVSCKASGYTFTSYHMHWVRQAPGQGLEWIGYIYPG | PA2.1.18 scFv |
| NVNTEYNEKFKGKATLTADKSTNTAYMELSSLRSEDTAVYFCAREEITYAMDYWG | (humanized) |
| QGTLVTVSSGGGGGGGGSGGGGSGGDVQMTQSPSTLSASVGDRVTITCSSSQSI | DNA Sequence: |
| VHSNGNTYMEWYQQKPGKAPKLLIYKVSNRFSGVPDRFSGSGSGTEFTLTISSLQP | SEQ ID NO: 184 |
| DDFATYYCHQGSHVPRTFGQGTKVEVK (SEQ ID NO: 58) | |

HLA-A*02 antigen binding domains derived from BB7.2 mAb

| | |
|---|---|
| C-002164 BB7.2 scFv (mouse): | C-002164 BB7.2 |
| QVQLQQSGPELVKPGASVKMSCKASGYTFTSYHIQWVKQRPGQGLEWIGWIYPG | scFv (mouse) |
| DGSTQYNEKFKGKTTLTADKSSSTAYMLLSSLTSEDSAIYFCAREGTYYAMDYWGQ | DNA Sequence: |
| GTSVTVSSGGGGSGGGGSGGGGSGGDVLMTQTPLSLPVSLGDQVSISCRSSQSIV | SEQ ID NO: 185 |
| HSNGNTYLEWYLQKPGQSPKLLIYKVSNRFSGVPDRFSGSGSGTDFTLKISRVEAED | |
| LGVYYCFQGSHVPRTFGGGTKLEIK (SEQ ID NO: 59) | |
| C-002165 BB7.2.1 scFv (humanized): | C-002165 |
| QLQLQESGPGLVKPSETLSLTCTVSGYTFTSYHIQWIRQPPGKGLEWIGWIYPGDG | BB7.2.1 scFv |
| STQYNEKFKGRATISVDTSKNQFSLNLDSVSAADTAIYYCAREGTYYAMDYWGKGS | (humanized) |
| TVTVSSGGGGSGGGGSGGGGSGGDIQMTQSPSSLSASVGDRVTITCRSSQSIVHS | DNA Sequence: |
| NGNTYLEWYQQKPGKAPKLLIYKVSNRFSGVPSRFSGSGSGTDFTFTISSLQPEDIA | SEQ ID NO: 186 |
| TYYCFQGSHVPRTFGPGTKVDIK (SEQ ID NO: 60) | |
| C-002166 BB7.2.2 scFv (humanized): | C-002166 |
| EVQLVQSGAELKKPGSSVKVSCKASGYTFTSYHIQWVKQAPGQGLEWIGWIYPGD | BB7.2.2 scFv |
| GSTQYNEKFKGKATLTVDKSTNTAYMELSSLRSEDTAVYYCAREGTYYAMDYWGQ | (humanized) |
| GTLVTVSSGGGGSGGGGSGGGGSGGDIQMTQSPSTLSASVGDRVTITCRSSQSIV | DNA Sequence: |
| HSNGNTYLEWYQQKPGKAPKLLIYKVSNRFSGVPSRFSGSGSGTDFTLTISSLQPDD | SEQ ID NO: 187 |
| FATYYCFQGSHVPRTFGQGTKVEVK (SEQ ID NO: 61) | |
| C-002167 BB7.2.3 scFv (humanized): | C-002167 |
| QVQLVQSGAEVKKPGSSVKVSCKASGYTFTSYHIQWVRQAPGQGLEWMGWIYP | BB7.2.3 scFv |
| GDGSTQYNEKFKGRVTITADKSTSTAYMELSSLRSEDTAVYYCAREGTYYAMDYW | (humanized) |
| GQGTTVTVSSGGGGSGGGGSGGGGSGGEIVLTQSPGTLSLSPGERATLSCRSSQSI | DNA Sequence: |
| VHSNGNTYLEWYQQKPGQAPRLLIYKVSNRFSGIPDRFSGSGSGTDFTLTISRLEPE | SEQ ID NO: 188 |
| DFAVYYCFQGSHVPRTFGGGTKVEIK (SEQ ID NO: 62) | |
| C-002168 BB7.2.5 scFv (humanized): | C-002168 |
| QVTLKQSGAEVKKPGSSVKVSCTASGYTFTSYHVSWVRQAPGQGLEWLGRIYPGD | BB7.2.5 scFv |
| GSTQYNEKFKGKVTITADKSMDTSFMELTSLTSEDTAVYYCAREGTYYAMDLWGQ | (humanized) |
| GTLVTVSSGGGGSGGGGSGGGGSGGEIVLTQSPGTLSLSPGERATLSCRSSQSIVHS | DNA Sequence: |
| NGNTYLAWYQQKPGQAPRLLISKVSNRFSGVPDRFSGSGSGTDFTLTISRLEPEDFA | SEQ ID NO: 189 |
| VYYCQQGSHVPRTFGGGTKVEIK (SEQ ID NO: 63) | |
| C-002169 BB7.2.6 scFv (humanized): | C-002169 |
| QVQLVQSGAEVKKPGASVKVSCKASGYTFTSYHMHWVRQAPGQRLEWMGWIY | BB7.2.6 scFv |
| PGDGSTQYNEKFKGKVTITRDTSASTAYMELSSLRSEDTAVYYCAREGTYYAMDY | (humanized) |
| WGQGTLVTVSSGGGGSGGGGSGGGGSGGDIVMTQTPLSLPVTPGEPASISCRSS | DNA Sequence: |
| QSIVHSNGNTYLDWYLQKPGQSPQLLIYKVSNRFSGVPDRFSGSGSGTDFTLKISRV | SEQ ID NO: 190 |
| EAEDVGVYYCMQGSHVPRTFGGGTKVEIK (SEQ ID NO: 64) | |

Exemplary heavy chain and light chain CDRs (CDR-H1, CDR-H2 and CDR-H3, or CDR-L1, CDR-L2 and CDR-L3, respectively) for HLA-A*02 ligand binding domains are shown in table 7 below.

TABLE 7
CDRs corresponding to HLA-A*02 antigen binding domains

| CDR-L1 | CDR-L2 | CDR-L3 | CDR-H1 | CDR-H2 | CDR-H3 |
|---|---|---|---|---|---|
| RSSQSIVHSN | KVSNRFSGVP | FQGSHVPRT | ASGYTFTSYHI | WIYPGNVNT | EEITYAMDY |
| GNTYLE (SEQ | DR (SEQ ID | (SEQ ID NO: | H (SEQ ID NO: | EYNEKFKGK | (SEQ ID NO: |
| ID NO: 41) | NO: 42) | 43) | 44) | (SEQ ID NO: | 46) |
| | | | | 45) | |
| RSSQSIVHSN | KVSNRFSGVP | MQGSHVPRT | SGYTFTSYHM | WIYPGDGST | EGTYYAMDY |
| GNTYLD (SEQ | DR (SEQ ID | (SEQ ID NO: | H (SEQ ID NO: | QYNEKFKG | (SEQ ID NO: |
| ID NO: 47) | NO: 48) | 49) | 50) | (SEQ ID NO: | 52) |
| | | | | 51) | |

In some embodiments, the scFv comprises the complementarity determined regions (CDRs) of any one of SEQ ID NOS: 41-52. In some embodiments, the scFv comprises a sequence at least 95% identical to any one of SEQ ID NOS: 41-52. In some embodiments, the scFv comprises a sequence identical to any one of SEQ ID NOS: 41-52. In some embodiments, the heavy chain of the antibody comprises the heavy chain CDRs of any one of SEQ ID NOS: 53-64, and wherein the light chain of the antibody comprises the light chain CDRs of any one of SEQ ID NOS: 53-64. In some embodiments, the heavy chain of the antibody comprises a sequence at least 95% identical to the heavy chain portion of any one of SEQ ID NOS: 53-64, and wherein the light chain of the antibody comprises a sequence at least 95% identical to the light chain portion of any one of SEQ ID NOS: 53-64.

In some embodiments, the heavy chain of the antibody comprises a sequence identical to the heavy chain portion of any one of SEQ ID NOS: 53-64, and wherein the light chain of the antibody comprises a sequence identical to the light chain portion of any one of SEQ ID NOS: 53-64.

In some embodiments, the ScFv comprises a sequence at least 95% identical, at least 96% identical, at least 97% identical, at least 98% identical, at least 99% identical or identical to any one of SEQ ID NOS: 53-64.

In some embodiments, the second, inhibitory ligand is HLA-A*02, and the inhibitory ligand binding domain comprises an HLA-A*02 ligand binding domain. In some embodiments, the second ligand binding domain binds HLA-A*02 independent of the peptide in a pMHC complex comprising HLA-A*02. In some embodiments, the HLA-A*02 ligand binding domain comprises an ScFv domain. In some embodiments, the HLA-A*02 ligand binding domain comprises a sequence of any one of SEQ ID NOs: 53-64. In some embodiments, the HLA-A*02 ligand binding domain comprises a sequence at least 90%, at least 95% or at least 99% identical to a sequence of any one of SEQ ID NOs: 53-64. In some embodiments, the HLA-A*02 ligand binding domain is encoded by a sequence comprising any one of SEQ ID NOs: 179-190. In some embodiments, the HLA-A*02 ligand binding domain is encoded by a sequence having at least 80% identity, at least 85% identity, at least 90°/% identity, at least 95% identity or at least 99% identity to a sequence of any one of SEQ ID NOs: 179-190.

### Minor Histocompatibility Antigens

In some embodiments, the second, inhibitor ligand comprises a minor histocompatibility antigen (MiHA). In some embodiments, the second, inhibitor ligand comprises an allele of a MiHA that is lost in a target cell through LOH.

MiHAs are peptides derived from proteins that contain nonsynonymous differences between alleles and are displayed by common HLA alleles. The non-synonymous differences can arise from SNPs, deletions, frameshift mutations or insertions in the coding sequence of the gene encoding the MiHA. Exemplary MiHAs can be about 9-12 amino acids in length and can bind to MHC class 1 and MHC class II proteins. Binding of the TCR to the MHC complex displaying the MiHA can activate T cells. The genetic and immunological properties of MiHAs will be known to the person of ordinary skill in the art. Candidate MiHAs are known peptides presented by known HLA class I alleles, are known to elicit T cell responses in the clinic (for example, in graft versus host disease, or transplant rejection, and allow for patient selection by simple SNP genotyping.

In some embodiments, the MiHA has broad or ubiquitous RNA expression.

In some embodiments, the MHA has high minor allele frequency.

In some embodiments, the MiHA comprises a peptide derived from a Y chromosome gene.

In some embodiments, the second inhibitor ligand comprises a MiHA selected from the group of MiHAs disclosed in Tables 8 and 9.

Exemplary, but non-limiting, examples of MiHAs that are envisaged as within the scope of the instant invention are disclosed in Table 8 below. Columns in Table 8 indicate, from left to right, the name of the MiHA, the gene which from which it is derived, MHC class I variant which can display the MiHA and the sequences of the peptide variants [A/B variants indicated in brackets).

TABLE 8
HLA Class I Autosomal MiHAs.

| MIHA | Gene | HLA | Peptide A/B |
|---|---|---|---|
| LB-CYBA-1Y | cytochrome b-245 alpha chain | A*01:01 | STMERWGQK[Y/H] (SEQ ID NO: |
| | (CYBA) | | 303) |
| LB-OAS1-1R | 2′-5′-oligoadenylate synthetase 1 | A*01:01 | ETDDPR[R/T]YQKY (SEQ ID NO: 304) |
| | (OAS1) | | |
| HA-1/A2 | Rho GTPase activating protein 45 | A*02:01 | VL[H/R]DDLLEA (SEQ ID NO: 273) |
| | (HMHA1) | | |
| HA-2 | myosin IG (MYO1G) | A*02:01 | YIGEVLVS[V/M] (SEQ ID NO: 305) |
| HA-8 | pumilio RNA binding family | A*02:01 | [R/P]TLDKVLEV (SEQ ID NO: 306) |
| | member 3 (KIAA0020, PUM3) | | |
| HA-3 | A-kinase anchoring protein 13 | A*01:01 | V[T/M]EPGTAQY (SEQ ID NO: 307) |
| | (AKAP13) | | |
| HwA11-S | chromosome 19 open reading | A*02:01 | CIPPD[S/T]LLFPA (SEQ ID NO: 308) |
| | frame 48 (C19ORF48) | | |
| LB-ADIR-1F | torsin family 3 member A (TOR3A) | A*02:01 | SVAPALAL[F/S]PA (SEQ ID NO: 309) |
| LB-HIVEP1-1S | HIVEP zinc finger 1 (HIVEP1) | A*02:01 | SLPKH[S/N]VTI (SEQ ID NO: 310) |
| LB-NISCH-1A | nischarin (NISCH) | A*02:01 | ALAPAP[A/V]EV (SEQ ID NO: 311) |
| LB-SSR1-1S | signal sequence receptor subunit 1 | A*02:01 | [S/L]LAVAQDLT (SEQ ID NO:312) |
| | (SSR1) | | |
| LB-WNK1-11 | WNK lysine deficient protein kinase | A*02:01 | RTLSPE[I/M]ITV (SEQ ID NO: 313) |
| | 1 (WNK1) | | |
| T4A | tripartite motif containing 4 | A*02:01 | GLYTYWSAG[A/E] (SEQ ID NO: 314) |
| | (TRIM42) | | |
| UTA2-1 | retroelement silencing factor 1 | A*02:01 | QL[L/P]NSVLTL (SEQ ID NO: 315) |
| | (KIAA1551) | | |
| LB-CLYBL-1Y | citramalyl-CoA lyase (CLYBL) | A*02:01 | SLAA(Y/D)IPRL (SEQ ID NO: 316) |
| TRIM22 | tripartite motif containing 22 | A*02:01 | MAVPPC[C/R]IGV (SEQ ID NO: 317) |
| | (TRIM22) | | |
| PARP10-1L | poly(ADP-ribose) polymerase | A*02:01 | GL[L/P]GQEGLVEI (SEQ ID NO: 318) |
| | family member 10 (PARP10) | | |
| FAM119A-1T | methyltransferase like 21A | A*02:01 | AMLERQF[T/I]V (SEQ ID NO: 319) |
| | FAM119A) | | |
| GLRX3-1S | glutaredoxin 3 (GLRX3) | A*02:01 | FL[S/P]SANEHL (SEQ ID NO: 320) |
| HNF4G-1M | hepatocyte nuclear factor 4 gamma | A*02:01 | M[M/I]YKDILLL (SEQ ID NO: 321) |
| | (HNF4G) | | |
| HMMR-1V | hyaluronan mediated motility | A*02:01 | SLQEK[V/A]AKA (SEQ ID NO: 322) |
| | receptor (HMMR) | | |
| BCL2A1 | BCL2 related protein A1 (BCL2A1) | A*02:01 | VLQ[N/K]VAFSV (SEQ ID NO: 323) |
| CDC26-1F | cell division cycle 26 (CDC26) | A*02:01 | [F/S]VAGTQEVFV (SEQ ID NO: 324) |
| APOBEC3F- | apolipoprotein B mRNA editing | A*02:01 | FL[S/A]EHPNVTL (SEQ ID NO: 325) |
| 1S/A | enzyme catalytic subunit 3F | | |
| | (APOBEC3F) | | |
| LB-PRCP-1D | prolylcarboxypeptidase (PRCP) | A*02:01 | FMWDVAE[D/E]L (9 mer) (SEQ ID |
| | | | NO: 326), FMWDVAE[D/E]LKA |
| | | | (11 mer) (SEQ ID NO: 327) |
| LB-CCL4-1T | C-C motif chemokine ligand 4 | A*02:01 | CADPSE[T/S]WV (SEQ ID NO: 328) |
| | (CCL4) | | |
| LB-NCAPD3- | non-SMC condensin Il complex | A*02:01 | WL[Q/R]GVVPVV (SEQ ID NO: 329) |
| 1Q | subunit D (NCAPD3) | | |
| LB-NDC80-1P | NDC80 kinetochore complex | A*02:01 | HLEEQI[P/A]KV (SEQ ID NO: 330) |
| | component (NDC80) | | |
| LB-TTK-1D | TTK protein kinase (TTK) | A*02:01 | RLH[D/E]GRVFV (SEQ ID NO: 331) |
| WDR27-1L | WD repeat domain 27 (WDR27) | A*02:01 | S[L/P]DDHVVAV (SEQ ID NO: 332) |
| MIIP | migration and invasion inhibitory | A*02:01 | SEESAVP[K/E]RSW (11 mer) (SEQ ID |
| | protein (MIIP) | | NO: 333), EESAVP[K/E]RSW (10 mer) |
| | | | (SEQ ID NO: 334) |
| HER-2/NEU | E erb-b2 receptor tyrosine kinase 2 | A*02:01 | not reported |
| | (RBB2) | | |
| LB-DHX33-1C | DEAH-box helicase 33 (DHX33) | A*02:01, | YLYEGGIS[C/R] (SEQ ID NO: 335) |
| | | C*03:03 | |
| PANE1 | centromere protein M (CENPM) | A*03:01 | RVWDLPGVLK (SEQ ID NO: 336) |
| SP110 | SP110 nuclear body protein (SP110) | A*03:01 | SLP[R/G]GTSTPK (SEQ ID NO: 337) |
| ACC-1C/Y | BCL2 related protein A1 (BCL2A1) | A*24:02 | DYLQ[Y/C]VLQI (SEQ ID NO: 338) |
| P2RX7 | purinergic receptor P2X 7 (P2RX7) | A*29:02 | WFHHC[H/R]PKY (SEQ ID NO: 339) |
| ACC-4 | cathepsin H (CTSH) | A*31:01 | ATLPLLCA[R/G] (SEQ ID NO: 340) |
| ACC-5 | CTSH | A*33:03 | WATLPLLCA[R/G] (SEQ ID NO: 341) |
| AKAP13 | A-kinase anchoring protein 13 | B*07:02 | APAGVREV[M/T] (SEQ ID NO: 342) |
| | (AKAP13) | | |
| LB- | apolipoprotein B mRNA editing | B*07:02, | [K/E]PQYHAEMCF (SEQ ID NO: 343) |
| APOBEC3B- | enzyme catalytic subunit 3B | B*08:01 | |
| 1K | (APOBEC3B) | | |
| APOBEC3H | apolipoprotein B mRNA editing | B*07:02 | KPQQ[K/E]GLRL (SEQ ID NO: 344) |
| | enzyme catalytic subunit 3H | | |
| | (APOBEC3H) | | |
| LB-ARHGDIB- | Rho GDP dissociation inhibitor beta | B*07:02 | LPRACW[R/P]EA (SEQ ID NO: 345) |
| 1R | (ARHGDIB) | | |
| LB-BCAT2-1R | BCAT2-branched chain amino | B*07:02 | QP[R/T]RALLFVIL (SEQ ID NO: 346) |
| | acid transaminase 2 (BCAT2) | | |
| BFAR | bifunctional apoptosis regulator | B*07:02 | APNTGRANQQ[M/R] (SEQ ID NO: |
| | (BFAR) | | 347) |
| C14orf169 | ribosomal oxygenase 1 (C14orf169 | B*07:02 | RPR[A/V]PTEELAL (SEQ ID NO: 348) |
| | or RIOX1) | | |
| LB-C16ORF- | C16ORF | B*07:02 | [R/W]PCPSVGLSFL (SEQ ID NO: 349) |
| 1R | | | |
| C18orf21 | chromosome 18 open reading | B*07:02 | NPATP[A/T]SKL (SEQ ID NO: 350) |
| | frame 21 (C18orf21) | | |
| LB-EB13-11 | Epstein-Barr virus induced 3 (EBI3) | B*07:02 | RPRARYY[I/V]QV (SEQ ID NO: 351) |
| POP1 | POP1 homolog, ribonuclease | B*07:02 | LPQKKSIN/K]AL (SEQ ID NO: 352) |
| | P/MRP subunit (POP1) | | |
| SCRIB | scribble planar cell polarity protein | B*07:02 | LPQQPP[L/P]SL (SEQ ID NO: 353) |
| | (SCRIB) | | |
| MTRR | 5-methyltetrahydrofolate- | B*07:02 | SPAS[S/L]RTDL (SEQ ID NO: 354) |
| | homocysteine methyltransferase | | |
| | reductase (MTRR) | | |
| LLGL2 | LLGL scribble cell polarity complex | B*07:02 | SPSL[R/H]ILAI (SEQ ID NO: 355) |
| | component 2 (LLGL2) | | |
| LB-ECGF-1H | thymidine phosphorylase (TYMP) | B*07:02 | RP[H/R]AIRRPLAL (SEQ ID NO: 356) |
| LB-ERAP1-1R | endoplasmic reticulum | B*07:02 | HP[R/P]QEQIALLA (11 mer) (SEQ ID |
| | aminopeptidase 1 (ERAP1) | | NO: 357), HP[R/P]QEQIAL (9 mer) |
| | | | (SEQ ID NO: 358) |
| LB-FUCA2-1V | alpha-L-fucosidase 2 (FUCA2) | B*07:02 | RLRQ[V/M]GSWL (SEQ ID NO: 359) |
| LB-GEMIN4- | gem nuclear organelle associated | B*07:02, | FPALRFVE[V/E] (SEQ ID NO: 360) |
| 1V | protein 4 (GEMIN4) | B*08:01 | |
| HDGF | heparin binding growth factor | B*07:02 | LPMEVEKNST[L/P] (SEQ ID NO: 361) |
| | (HDGF) | | |
| LB-PDCD11- | programmed cell death 11 | B*07:02 | GPDSSKT[F/L]LCL (SEQ ID NO: 362) |
| 1F | (PDCD11) | | |
| LB-PFAS-1P | phosphoribosylformylglycinamidine | B*07:02 | A[P/S]GHTRRKL (SEQ ID NO: 363) |
| | synthase (PFAS) | | |
| LB-TEP1-1S | telomerase associated protein 1 | B*07:02 | APDGAKVA[S/P]L (SEQ ID NO: 364) |
| | (TEP1) | | |
| LB-TMEM8A- | post-glycosylphosphatidylinositol | B*07:02 | RPRSVT[I/V]QPLL (SEQ ID NO: 365) |
| 1 | attachment to proteins 6 (TMEM8A | | |
| | or PGAP6) | | |
| LB-USP15-11 | ubiquitin specific peptidase 15 | B*07:02 | MPSHLRN[I/T]LL (SEQ ID NO: 366) |
| | (USP15) | | |
| LRH-1 | purinergic receptor P2X 5 (P2RX5) | B*07:02 | TPNQRQNVC (SEQ ID NO: 367) |
| LB-MOB3A- | MOB kinase activator 3A (MOB3A) | B*07:02 | [C/S]PRPGTWTC (SEQ ID NO: 368) |
| 1C | | | |
| LB-ZDHHC6- | zinc finger DHHC-type | B*07:02 | RPR[Y/H]WILLVKI (SEQ ID NO: 369) |
| 1Y | palmitoyltransferase 6 (ZDHHC6) | | |
| ZAPHIR | zinc finger protein 419 (ZNF419) | B*07:02 | IPRDSWWVEL (SEQ ID NO: 370) |
| HEATR1 | HEAT repeat containing 1 (HEATR1) | B*08:01 | ISKERA[E/G]AL (SEQ ID NO: 371) |
| LB-GSTP1-1V | glutathione S-transferase pi 1 | B*08:01 | DLRCKY[V/I]SL (SEQ ID NO: 372) |
| | (GSTP1) | | |
| HA-1/B60 | Rho GTPase activating protein 45 | B*40:01 | KECVL[H/R]DDL (SEQ ID NO: 373) |
| | (HMHA1) | | |
| LB-SON-1R | SON DNA and RNA binding protein | B*40:01 | SETKQ[R/C]TVL (SEQ ID NO: 374) |
| | (SON) | | |
| LB-SWAP70- | switching B cell complex subunit | B*40:01 | MEQLE[Q/E]LEL (SEQ ID NO: 375) |
| 1Q | SWAP70 (SWAP70) | | |
| LB-TRIP10- | thyroid hormone receptor | B*40:01 | G[E/G][P/S]QDL[C/G]TL (SEQ ID |
| 1EPC | interactor 10 (TRIP10) | | NO: 376) |
| LB-NUP133- | nucleoporin 133 (NUP133) | B*40:01 | SEDLILC[R/Q]L (SEQ ID NO: 377) |
| 1R | | | |
| LB-ZNFX1-1Q | zinc finger NFX1-type containing 1 | B*40:01 | NEIEDVW[Q/H]LDL (SEQ ID NO: |
| | (ZNFX1) | | 378) |
| SLC1A5 | solute carrier family 1 member 5 | B*40:02 | AE[A/P]TANGGLAL (SEQ ID NO: 379) |
| | (SLC1A5) | | |
| ACC-2 | BCL2A1 | B*44:02, | KEFED[D/G]IINW (SEQ ID NO: 380) |
| | | B*44:03 | |
| ACC-6 | histocompatibility minor serpin | B*44:03 | MEIFIEVFSHF (SEQ ID NO: 381) |
| | domain containing (HMSD) | | |
| HB-1H/Y | histocompatibility minor HB-1 | B*44:03 | EEKRGSL[H/Y]VW (SEQ ID NO: 382) |
| | (HMHB1) | | |
| DPH1 | diphthamide biosynthesis 1 (DPH1) | B*57:01 | S[V/L]LPEVDVW (SEQ ID NO: 383) |
| UGT2B17/A02 | UDP glucuronosyltransferase family | A*02:06 | CVATMIFMI (SEQ ID NO: 384) |
| | 2 member B17 (UGT2B17) | | |
| UGT2B17/A29 | UGT2B17 | A*29:02 | AELLNIPFLY (SEQ ID NO: 385) |
| UGT2B17/844 | UGT2817 | B*44:03 | AELLNIPFLY (SEQ ID NO: 386) |

Exemplary, but non-limiting, examples of MiHAs that are envisaged as within the scope of the instant invention are disclosed in Table 9 below. Columns in Table 9 indicate, from left to right, the name of the MiHA, the gene which from which it is derived, MH-C class 1 variant which can display the MiHA and the sequences of the peptide variants [A/B variants indicated in brackets).

TABLE 9
HLA Class I Y linked MiHAs.

| MIHA | Gene | HLA | Peptide A/B |
|---|---|---|---|
| DFFRY | ubiquitin specific peptidase 9 Y- | A*01:01 | IVD[C/S]LTEMY (SEQ ID NO: 387) |
| | linked (DFFRY) | | |
| SMCY | lysine demethylase 5 (SMCY) | A*02:01 | FIDSYICQV (SEQ ID NO: 388) |
| TMSB4Y | thymosin beta 4 Y-linked (TMSB4Y) | A*33:03 | EVLLRPGLHFR (SEQ ID NO: 389) |
| SMCY | SMCY | B*07:02 | SP[S/A]VDKA[R/Q]AEL (SEQ ID NO: |
| | | | 34) |
| UTY | ubiquitously transcribed | B*08:01 | LPHN[H/R]T[D/N]L (SEQ ID NO: 25) |
| | tetratricopeptide repeat | | |
| | containing, Y-linked (UTY) | | |
| RPS4Y | ribosomal protein $4 Y-linked 1 | B*52:01 | TIRYPDP[V/L]I (SEQ ID NO: 24) |
| | (RPS4Y) | | |
| UTY | UTY | B*60:01 | [R/G]ESEE[E/A]S[V/P]SL (SEQ ID |
| | | | NO: 23) |

In some embodiments, the MiHA comprises HA-1. HA-1 is a peptide antigen having a sequence of VL[H/R]DDLLEA (SEQ ID NO: 273), and is derived from the Rho GTPase activating protein 45 (HA-1) gene.

Exemplary ligand binding domains that selectively bind to HA-1 variant H peptide (VLHDDLLEA (SEQ ID NO: 191)) are shown in Table 10 below. TCR alpha and TCR beta sequences in SEQ ID NO: 193 are separated by a P2A self-cleaving polypeptide of sequence ATNFSLLKQAGDVEENPGP (SEQ ID NO: 192) with an N terminal GSG linker.

TABLE 10
Ftcr HA-1(H) Inhibitory Receptor Sequences

| C-003754 KP7 HA-1H TCRalpha T48C P2A KP7 HA-1H TCRbeta S57C: | C-003754 |
|---|---|
| MVKIRQFLLAILWLQLSCVSAAKNEVEQSPQNLTAQEGEFITINCSYSVGISALHWLQQHP | KP7 HA-1H |
| GGGIVSLFMLSSGKKKHGRLIATINIQEKHSSLHITASHPRDSAVYICAVRSVSGAGSYQLTF | TCRalpha |
| GKGTKLSVIPNIQNPDPAVYQLRDSKSSDKSVCLFTDFDSQTNVSQSKDSDVYITDKCVLD | T48C P2A |
| MRSMDFKSNSAVAWSNKSDFACANAFNNSIIPEDTFFPSPESSCDVKLVEKSFETDTNLN | KP7 HA-1H |
| FQNLSVIGFRILLLKVAGFNLLMTLRLWSSGSGATNFSLLKQAGDVEENPGPMGTSLLCW | TCRbeta |
| MALCLLGADHADTGVSQNPRHKITKRGQNVTFRCDPISEHNRLYWYRQTLGQGPEFLTY | S57C DNA |
| FQNEAQLEKSRLLSDRFSÅERPKGSFSTLEIQRTEQGDSAMYLCASSIDSFNEQFFGPGTRL | Sequence: |
| TVLEDLKNVFPPEVAVFEPSEAEISHTQKATLVCLATGFYPDHVELSWWVNGKEVHSGVC | SEQ ID NO: |
| TDPQPLKEQPALNDSRYCLSSRLRVSATFWQNPRNHFRCQVQFYGLSENDEWTQDRAK | 194 |
| PVTQIVSAEAWGRADCGFTSESYQQGVLSATILYEILLGKATLYAVLVSALVLMAMVKRKD | |
| SRG (SEQ ID NO: 193) | |
| HA-1H TCR alpha: | HA-1H TCR |
| MVKIRQFLLAILWLQLSCVSAAKNEVEQSPQNLTAQEGEFITINCSYSVGISALHWLQQHP | alpha DNA |
| GGGIVSLFMLSSGKKKHGRLIATINIQEKHSSLHITASHPRDSAVYICAVRSVSGAGSYQLTF | Sequence: |
| GKGTKLSVIPNIQNPDPAVYQLRDSKSSDKSVCLFTDFDSQTNVSQSKDSDVYITDKCVLD | SEQ ID NO: |
| MRSMDFKSNSAVAWSNKSDFACANAFNNSIIPEDTFFPSPESSCDVKLVEKSFETDTNLN | 201 |
| FQNLS (SEQ ID: 199) | |
| HA-1(H) TCRbeta: | HA-1H |
| MGTSLLCWMALCLLGADHADTGVSQNPRHKITKRGQNVTFRCDPISEHNRLYWYRQTL | TCRbeta |
| GQGPEFLTYFQNEAQLEKSRLLSDRFSAERPKGSFSTLEIQRTEQGDSAMYLCASSIDSFNE | DNA |
| QFFGPGTRLTVLEDLKNVFPPEVAVFEPSEAEISHTQKATLVCLATGFYPDHVELSWWVN | Sequence: |
| GKEVHSGVCTDPQPLKEQPALNDSRYCLSSRLRVSATFWQNPRNHFRCQVQFYGLSEND | SEQ ID NO: |
| EWTQDRAKPVTQIVSAEAWGRADCGFTSESYQQGVLS (SEQ ID NO: 200) | 202 |
| C-003755 KP7 HA-1H FTCRalpha LIR1 TICD: | C-003755 |
| MVKIRQFLLAILWLQLSCVSAAKNEVEQSPQNLTAQEGEFITINCSYSVGISALHWLQQHP | KP7 HA-1H |
| GGGIVSLFMLSSGKKKHGRLIATINIQEKHSSLHITASHPRDSAVYICAVRSVSGAGSYQLTF | FTCRalpha |
| GKGTKLSVIPNIQNPDPAVYQLRDSKSSDKSVCLFTDFDSQTNVSQSKDSDVYITDKCVLD | LIR1 TICD |
| MRSMDFKSNSAVAWSNKSDFACANAFNNSIIPEDTFFPSPESSCDVKLVEKSFETDTNLN | DNA |
| FQNLSVVIGILVAVILLLLLLLLLFLILRHRRQGKHWTSTQRKADFQHPAGAVGPEPTDRGL | Sequence: |
| QWRSSPAADAQEENLYAAVKHTQPEDGVEMDTRSPHDEDPQAVTYAEVKHSRPRREM | SEQ ID NO: |
| ASPPSPLSGEFLDTKDRQAEEDRQMDTEAAASEAPQDVTYAQLHSLTLRREATEPPPSQE | 196 |
| GPSPAVPSIYATLAIH (SEQ ID NO: 195) | |
| C-003756 KP7 HA-1H FTCRbeta LIR1 TICD: | C-003756 |
| MGTSLLCWMALCLLGADHADTGVSQNPRHKITKRGQNVTFRCDPISEHNRLYWYRQTL | KP7 HA-1H |
| GQGPEFLTYFQNEAQLEKSRLLSDRFSAERPKGSFSTLEIQRTEQGDSAMYLCASSIDSFNE | FTCRbeta |
| QFFGPGTRLTVLEDLKNVFPPEVAVFEPSEAEISHTQKATLVCLATGFYPDHVELSWWVN | LIR1 TICD |
| GKEVHSGVCTDPQPLKEQPALNDSRYCLSSRLRVSATFWQNPRNHFRCQVQFYGLSEND | DNA |
| EWTQDRAKPVTQIVSAEAWGRADCGFTSESYQQGVLSVVIGILVAVILLLLLLLLLFLILRHR | Sequence: |
| RQGKHWTSTQRKADFQHPAGAVGPEPTDRGLQWRSSPAADAQEENLYAAVKHTQPE | SEQ ID NO: |
| DGVEMDTRSPHDEDPQAVTYAEVKHSRPRREMASPPSPLSGEFLDTKDRQAEEDRQMD | 198 |
| TEAAASEAPQDVTYAQLHSLTLRREATEPPPSQEGPSPAVPSIYATLAIH (SEQ ID NO: | |
| 197) | |

In some embodiments, the second, inhibitory ligand comprises HA-1 (H). In some embodiments, the second, inhibitory ligand binding is isolated or derived from a TCR. In some embodiments, the second, inhibitory ligand binding domain comprises TCR alpha and TCR beta variable domains. In some embodiments, the TCR alpha and TCR beta variable domains are separated by a self cleaving polypeptide sequence. In some embodiments, the TCR alpha and TCR beta variable domains separated by a self cleaving polypeptide sequence comprise SEQ ID NO; 193. In some embodiments, the TCR alpha and TCR beta variable domains separated by a self cleaving polypeptide sequence comprise SEQ ID NO: 193, or a sequence having at least 90%, at least 95%, or at least 99% identity thereto. In some embodiments, the TCR alpha and TCR beta variable domains are encoded by a sequence of SEQ ID NO; 194, or a sequence having at least 80% identity, at least 90%, at least 95%, or at least 99% identity thereto. In some embodiments, the TCR alpha variable domain comprises SEQ ID NO: 199 or a sequence having at least 90%, at least 95%, or at least 99% identity thereto. In some embodiments, the TCR beta variable domain comprises SEQ ID NO: 200 or a sequence having at least 90%, at least 95%, or at least 99% identity thereto.

### Loss of Y Chromosome Antigens

In some embodiments, the second, inhibitor ligand comprises a Y chromosome gene, i.e. peptide encoded by a gene on the Y chromosome. In some embodiments, the second, inhibitor ligand comprises a peptide encoded by a Y chromosome gene that is lost in target cells through loss of Y chromosome (LoY). For example, about a third of the characterized MiHAs come from the Y chromosome. The Y chromosome contains over 200 protein coding genes, all of which are envisaged as within the scope of the instant disclosure.

As used herein, “loss of Y”, or “LoY” refers a genetic change that occurs at high frequency in tumors whereby one copy of part or all of the Y chromosome is deleted, leading to a loss of Y chromosome encoded gene(s).

Loss of Y chromosome is known to occur in certain cancers. For example, there is a reported 40% somatic loss of Y chromosome in renal clear cell cancers (Arseneault et al., Sci. Rep. 7: 44876 (2017)). Similarly, clonal loss of the Y chromosome was reported in 5 out of 31 in male breast cancer subjects (Wong et al., Oncotarget 6(42):44927-40 (2015)). Loss of the Y chromosome in tumors from male patients has been described as a “consistent feature” of head and neck cancer patients (el-Naggar et al., Am J Clin Pathol 105(1):102-8 (1996)). Further, Y chromosome loss was associated with X chromosome disomy in four of seven male patients with gastric cancer (Saal et al., Virchows Arch B Cell Pathol (1993)). Thus, Y chromosome genes can be lost in a variety of cancers, and can be used as inhibitor ligands with the engineered receptors of the instant disclosure targeting cancer cells.

### Antigen Binding Domains

The disclosure provides a first ligand binding domain that activates a first engineered receptor, thereby activating immune cells expressing the first engineered receptor, and a second ligand binding domain that activates a second engineered receptor that inhibits activation of immune cells expressing the second engineered receptor, even in the presence of the first engineered receptor bound to the first ligand.

Any type of ligand binding domain that can regulate the activity of a receptor in a ligand dependent manner is envisaged as within the scope of the instant disclosure. In some embodiments, the ligand binding domain is an antigen binding domain. Exemplary antigen binding domains include, inter alia, ScFv, SdAb, Vβ-only domains, and TCR antigen binding domains derived from the TCR α and β chain variable domains.

In some embodiments, the first, activator LBD comprises an antigen binding domain. In some embodiments, the second, inhibitor LBD comprises an antigen binding domain. Any type of antigen binding domain is envisaged as within the scope of the instant disclosure.

For example, the first, activator LBD and/or the second, inhibitor LBD can comprise an antigen binding domain that can be expressed as part of a contiguous polypeptide chain including, for example, a single domain antibody fragment (sdAb) or heavy chain antibodies HCAb, a single chain antibody (scFv) derived from a murine, humanized or human antibodies (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some aspects, the first, activator LBD and/or the second, inhibitor LBD comprises an antigen binding domain that comprises an antibody fragment. In further aspects, the activator LBD comprises an antibody fragment that comprises a scFv or an sdAb. In further aspects, the inhibitor LBD comprises an antibody fragment that comprises a scFv or an sdAb.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)2, and Fv fragments, single-chain (sc)Fv (“scFv”) antibody fragments, linear antibodies, single domain antibodies (abbreviated “sdAb”) (either VL or VH), camelid VHH domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “VH” (or, in the case of single domain antibodies, e.g., nanobodies, “VHH”) with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein a scFv may have the VL and VH variable regions in either order, e.g., with respect to the N-terminal and C-terminal ends of the polypeptide, the scFv may comprise VL-linker-VH or may comprise VH-linker-VL.

The term “antibody light chain,” refers to the smaller of the two types of polypeptide chains present in antibody molecules in their naturally occurring conformations. Kappa (“K”) and lambda (“ ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “Vβ domain”, “Vβ-only domain”, “β chain variable domain” or “single variable domain TCR (svd-TCR)” refers to an antigen binding domain that consists essentially of a single T Cell Receptor (TCR) beta variable domain that specifically binds to an antigen in the absence of a second TCR variable domain. In some embodiments, the first, activator LBD comprises or consists essentially of a Vβ-only domain. In some embodiments, the second, inhibitor LBD comprises or consists essentially of a Vβ-only domain.

In some embodiments, the Vβ-only domain may include additional elements besides the TCR variable domain, including additional amino acid sequences, additional protein domains (covalently associated, non-covalently associated or covalently and non-covalently associated with the TCR variable domain), fusion or non-covalent association of the TCR variable domain with other types of macromolecules (for example polynucleotides, polysaccharides, lipids, or a combination thereof), fusion or non-covalent association of the TCR variable domain with one or more small molecules, compounds, or ligands, or a combination thereof. Any additional element, as described, may be combined provided that the TCR variable domain is configured to specifically bind the epitope in the absence of a second TCR variable domain.

In other embodiments, the Vβ-only domain as described herein functions independently of an α chain that lacks a Vα segment. For example, in some embodiments the one or more Vβ-only domains are fused to transmembrane (e.g., CD3ζ and CD28) and intracellular domain proteins (e.g., CD3ζ, CD28, and/or 4-1BB) that are capable of activating T cells in response to antigen.

In some embodiments, the Vβ-only domain engages antigen using complementarity-determining regions (CDRs). Each s Vβ-only domain contains three complement determining regions (CDR1, CDR2, and CDR3).

In some embodiments, the first Vβ-only domain comprises a TCR Vβ domain or an antigen-binding fragment thereof.

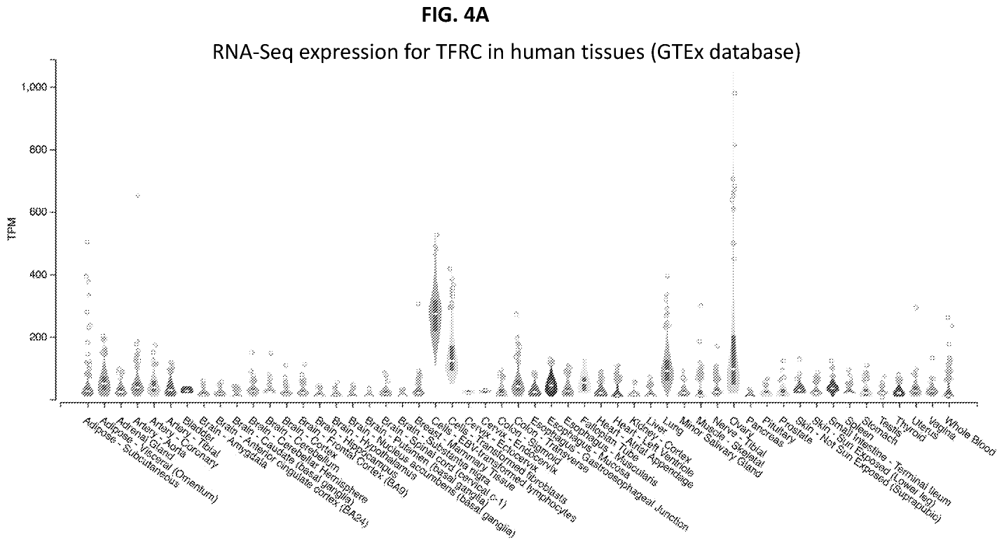

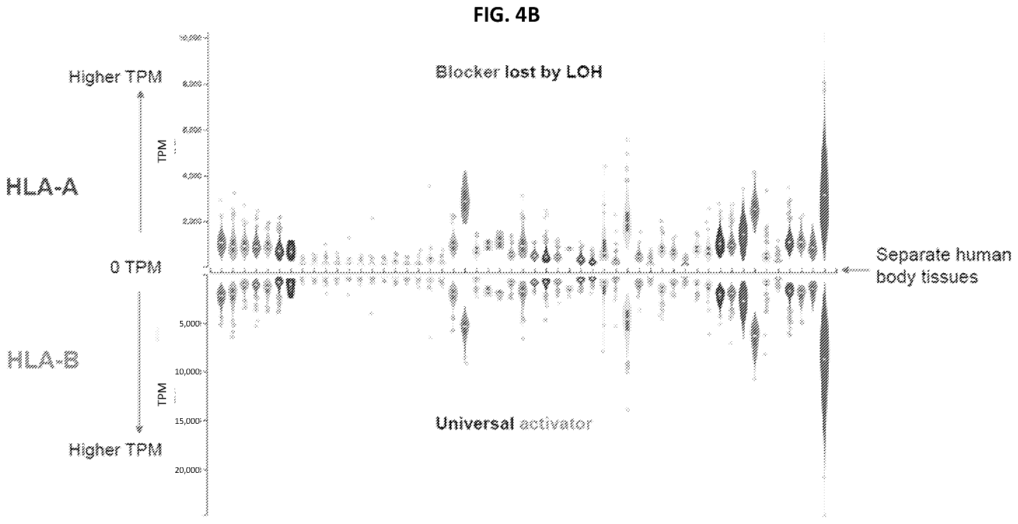

In humans, the TCR variable regions of the α and γ chains are each encoded by a V and a J segment, whereas the variable region of β and δ chains are each additionally encoded by a D segment. There are multiple Variable (V), Diversity (D) and Joining (J) gene segments (e.g. 52 Vβ gene segments, 2 Dβ gene segments and 13 Jβ gene segments) (Janeway et al. (eds.), 2001, Immunobiology: The Immune System in Health and Disease. 5th Edition, New York, FIG. 4.13) which can be recombined in different V(D)J arrangements using the enzymes RAG-1 and RAG-2, which recognize recombination signal sequences (RSSs) adjacent to the coding sequences of the V, D and J gene segments. The RSSs consist of conserved heptamers and nonamers separated by spacers of 12 or 23 bp. The RSSs are found at the 3′ side of each V segment, on both the 5′ and 3′ sides of each D segment, and at the 5′ of each J segment. During recombination, RAG-1 and RAG-2 cause the formation of DNA hairpins at the coding ends of the joint (the coding joint) and removal of the RSSs and intervening sequence between them (the signal joint). The variable regions are further diversified at the junctions by deletion of a variable number of coding end nucleotides, the random addition of nucleotides by terminal deoxynucleotidyl transferase (TdT), and palindromic nucleotides that arise due to template-mediated fill-in of the asymmetrically cleaved coding hairpins.

Patent applications WO 2009/129247 (herein incorporated by reference in its entirety) discloses an in vitro system, referred to as the HuTarg system, which utilizes V(D)J recombination to generate de novo antibodies in vitro. This same system was used to generate the variable regions of the Vβ-only domain as in patent application WO 2017/091905 (herein incorporated by reference in its entirety) by using TCR-specific V, D and J elements. In natural in vivo systems, the nucleic acid sequences which encode CDR1 and CDR2 are contained within the V (α, β, γ or δ) gene segment and the sequence encoding CDR3 is made up from portions of V and J segments (for Vα or Vγ) or a portion of the V segment, the entire D segment and a portion of the J segment (for Vβ or Vδ), but with random insertions and deletions of nucleotides at the V-J and V-D-J recombination junctions due to action of TdT and other recombination and DNA repair enzymes. The recombined T-cell receptor gene comprises alternating framework (FR) and CDR sequences, as does the resulting T-cell receptor expressed therefrom (i.e. FR1-CDR1-FR2-CDR2-FR3-CDR3-FR4). Using in vitro V(D)J recombination (i.e. V-J or V-D-J recombination), randomized insertions and deletions may be added in or adjacent to CDR1, CDR2 and/or CDR3 (i.e. not just CDR3), additional insertions may be added using flanking sequences in recombination substrates before and/or after CDR1, CDR2 and/or CDR3, and additional deletions may be made by deleting sequences in recombination substrates in or adjacent to CDR1, CDR2 and/or CDR3.

In some embodiments, TCR Vβ chains were identified that specifically bind epitopes in the absence of TCR Vα chains. Exemplary CDR3 amino acid sequences that bind epitopes in the absence of TCR Vα chains are listed in Table 11 below.

TABLE 11
CDR3 amino acid sequences of identified VB domains

| Epitope | TRBV gene | TRBD gene | TRBJ gene | CDR3 amino acid sequence |
|---|---|---|---|---|
| NY-ESO | TRBV5- | N/A | TRBJ2- | CASSIGLGYEQYF (SEQ ID NO: |
| | 8*01 | | 7*01 | 203) |
| NY-ESO | TRBV5- | TRBD2*02 | TRBJ2- | CASSLGGPRGLAGLRGDEQF (SEQ ID |
| | 8*01 | | 1*01 | NO: 204) |
| NY-ESO | TRBV5- | TRBD2*01 | TRBJ2- | CASSLRRDNEQF (SEQ ID NO: |
| | 8*01 | | 1*01 | 205) |
| MAGE-A3 | TRBV5- | TRBD1*01 | TRBJ2- | CASSLEVLLGADFPDTOYF (SEQ ID |
| | 8*01 | | 3*01 | NO: 206) |
| MAGE-A3 | TRBV5- | TRBD2*02 | TRBJ2- | CASSFPAGHGADLDNEQF (SEQ ID |
| | 8*01 | | 1*01 | NO: 207) |
| MAGE-A3 | TRBV5- | TRBD1*01 | TRBJ2- | CASSEITGRIGEQF (SEQ ID NO: |
| | 8*01 | | 1*01 | 208) |
| MAGE-A3 | TRBV5- | TRBD1*01 | TRBJ2- | CASSLGGDELGADGNEQF (SEQ ID |
| | 8*01 | | 1*01 | NO: 209) |

In some embodiments, the Vβ-only domain specifically binds to an epitope in the absence of a second TCR variable domain, and consists of optional N-terminal and/or C-terminal amino acid sequences (of any length or sequence) flanking a variable domain defined by FR1-CDR1-FR2-CDR2-FR3-CDR3-FR4 regions. FR1, FR2, FR3 and FR4 may be obtained from a natural Vα, Vβ, Vγ or Vδ domain or encoded by natural Vα, Vβ, Vγ or Vδ gene segments, but optionally include deletions or insertions of (e.g. 0, 1, 2, 3, 4, 5 or more than 5 amino acids) amino acids independently at one or more of the C-terminus of FR1, the N-terminus of FR2, the C-terminus of FR2, the N-terminus of FR3, the C-terminus of FR3 and the N-terminus of FR4. CDR1, CDR2 and CDR3 may be obtained from a natural Vα, Vβ, Vγ or Vδ domain, or encoded by natural Vα, Vβ, Vγ or Vδ gene segments, but wherein one or more of CDR1, CDR2 and CDR3 independently contains an insertion (e.g. 0, 1, 2, 3, 4, 5, 6, 7, 8, 9, 10 or more than 10 amino acids) and/or a deletion (e.g. 0, 1, 2, 3, 4, 5 or more than 5 amino acids) at the C-terminus, the N-terminus or anywhere within the CDR sequence. In some embodiments, the CDR1 contains an insertion or deletion of amino acids N-terminally, C-terminally or internally, wherein at least 50% (or optionally 60%, 70% or 80%) of natural CDR amino acid residues are retained. In some embodiments, the CDR2 contains an insertion or deletion of amino acids N-terminally, C-terminally or internally, wherein at least 50% (or optionally 60%, 70% or 80%) of natural CDR amino acid residues are retained. In some embodiments, the CDR3 contains an insertion or deletion of amino acids N-terminally, C-terminally or internally, wherein at least 50% (or optionally 60%, 70% or 80%) of natural CDR amino acid residues are retained. Insertions and/or deletions may be produced as a result of in vitro V(D)J recombination methods or from the in-vitro action of TdT and recombination and DNA repair enzymes (e.g. one or more of Artemis nuclease, NDA-dependent protein kinase (DNA-PK), X-ray repair cross-complementing protein 4 (XRCC4), DNA ligase IV, non-homologous end-joining factor 1 (NHEJ1), PAXX, and DNA polymerases λ and μ). Insertion and/or deletion (which includes substitution) may further result from insertions and/or deletions to CDR nucleic acid sequences of the in vitro V(D)J recombination substrates. The Vβ-only domain may further comprise a TCR constant region or portion thereof. The Vβ-only domain may be fused to and/or complexed with additional protein domains. A double stranded break in DNA may be introduced prior to in vitro use of the above recombination and DNA repair enzymes. The Vβ-only domain may be (or may be incorporated into) a fusion protein. As used herein, the term “fusion protein” means a protein encoded by at least one nucleic acid coding sequence that is comprised of a fusion of two or more coding sequences from separate genes, regardless of whether the organism source of those genes is the same or different.

In some embodiments, the first, activator LBD comprises an ScFv domain and the second, inhibitor LBD comprises a Vβ-only domain. In some embodiments, the first, activator LBD comprises a Vβ-only domain and the second, inhibitor LBD comprises an ScFv domain. In some embodiments, both the first, activator LBD and the second, inhibitor LBD are ScFv domains. In some embodiments, both the first, activator LBD and the second, inhibitor LBD are Vβ-only domains.

Additional antigen binding domains used with the activator and/or inhibitor receptors of the disclosure are described in Table 12 below. In table 12, the name of the construct is described as ScFv Inhibitor name [B]/ScFv Activator name [A]. In some embodiments, the first or second ligand binding domain comprises a sequence of any one of SEQ ID NO: 210, SEQ ID NO:212, SEQ ID NO: 214, SEQ ID NO:216, SEQ ID NO: 218, SEQ ID NO:220, SEQ ID NO: 222 Or SEQ ID NO:224, or a sequence having at least 90%, at least 95% or at least 99% identity thereto.

TABLE 12
Additional antigen binding domain sequences

| | |
|---|---|
| C-1761/C-266 (NY-ESO −1 scFv): | C-1761/C-266 |
| DIQMTQSPSSLSASVGDRVTITCRASQSISSYLNWYQQKPGKAPKLLIYAASSLQSGVPSRFSGS | (NY-ESO −1 |
| GSGTDFTLTISSLQPEDFATYYCQQSYSTPLTFGGGTKVEIKGGGGGGGGSGGGGSGGEVQL | scFv) DNA |
| VESGGGLVQPGGSLRLSCAASGFTVYDYMSWVRQAPGKGLEWVSVIYSGGSTYYADSVKGRF | Sequence: SEQ |
| TISRDNSKNTLYLQMNSLRAEDTAVYYCARYSYYYYYMDVWGKGTTVTVSS (SEQ ID NO: | ID NO: 211 |
| 210) | |
| C-3393/C-563 (MAGE-A3 pep1): | C-3393/C-563 |
| QVQLQESGPGLVKPSDTLSLTCAVSGYSISSSNWWGWIRQPPGKGLEWIGYIYYSGSTYYNPSL | (MAGE-A3 |
| KSRVTMSVDTSKNQFSLKLSSVTAVDTAVYYCARIPFGDWWYFDLWGRGTLVTVSSGGGGSG | pep1) DNA |
| GGGSGGGGSGGDIQMTQSPSSLSASVGDRVTITCRASQSISSYLNWYQQKPGKAPKLLIYAAS | Sequence: SEQ |
| SLQSGVPSRFSGSGSGTDFTLTISSLQPEDFATYYCQQSYSFVLTFGGGTKVEIK (SEQ ID NO: | ID NO: 213 |
| 212) | |
| C-3394/C-582 (MAGE-A3 pep1): | C-3394/C-582 |
| QVQLVQSGAEVKKPGASVKVSCKVSGYTLTELSMHWVRQAPGKGLEWMGGFDPEDGETIYA | (MAGE-A3 |
| QKFQGRVTMTEDTSTDTAYMELSSLRSEDTAVYYCATDLYSSSWYCDAFDIWGQGTMVTVSS | pep1) DNA |
| GGGGSGGGGSGGGGSGGDIQMTQSPSSLSASVGDRVTITCRASQSISSYLNWYQQKPGKAP | Sequence: SEQ |
| KLLIYAASSLQSGVPSRFSGSGSGTDFTLTISSLOPEDFATYYCQQSWASTPLTFGGGTKVEIK | ID NO: 215 |
| (SEQ ID NO: 214) | |
| C-3390/C-2387 (HPV E6 pep1): | C-3390/C-2387 |
| EVQLVESGGGLVQPGGSLRLSCAASGFTFSSYWMHWVRQAPGKGLVWVSRINSDGSSTSYA | (HPV E6 pep1) |
| DSVKGRFTISRDNAKNTLYLQMNSLRAEDTAVYYCARENGVVKWYFDLWGRGTLVTVSSGG | DNA Sequence: |
| GGSGGGGSGGGGSGGDIQMTQSPSSLSASVGDRVTITCRASQSISSYLNWYQQKPGKAPKLLI | SEQ ID NO: 217 |
| YAASSLQSGVPSRFSGSGSGTDFTLTISSLQPEDFATYYCQQSYSTPLFPFGGGTKVEIK (SEQ ID | |
| NO: 216) | |
| C-3391/C-1043 (HPV E6 pep1): | C-3391/C-1043 |
| EVQLVESGGGLVQPGRSLRLSCAASGFTFDDYAMHWVRQAPGKGLEWVSGISWNSGSIGYA | (HPV E6 pep1) |
| DSVKGRFTISRDNAKNSLYLQMNSLRAEDTALYYCAKDGRGSPFYGGAFDIWGQGTMVTVSS | DNA Sequence: |
| GGGGSGGGGSGGGGSGGDIQMTQSPSSLSASVGDRVTITCRASQSISSYLNWYQQKPGKAP | SEQ ID NO: 219 |
| KLLIYAASSLQSGVPSRFSGSGSGTDFTLTISSLQPEDFATYYCQQSYSTPLTFGGGTKVEIK (SEQ | |
| ID NO: 218) | |
| C-2753/C-782 (HPV E7 pep2): | C-2753/C-782 |
| EVQLVESGGGLVKPGGSLRLSCAASGFTFSNAWMSWVRQAPGKGLEWVGRIKSKTDGGTTD | (HPV E7 pep2) |
| YAAPVKGRFTISRDDSKNTLYLQMNSLKTEDTAVYYCTTSYDYLLNPYRWNWFDPWGQGTLV | DNA Sequence: |
| TVSSGGGGSGGGGSGGGGSGGDIQMTQSPSSLSASVGDRVTITCRASQSISSYLNWYQQKPG | SEQ ID NO: 221 |
| KAPKLLIYAASSLQSGVPSRFSGSGSGTDFTLTISSLQPEDFATYYCQQSYSTPLTFGGGTKVEIK | |
| (SEQ ID NO: 220) | |
| C-2752/C-1511 (MAGE-A3 pep2): | C-2752/C-1511 |
| QVQLVQSGAEVKKPGSSVKVSCKASGGTFSSYAISWVRQAPGQGLEWMGGIIPIFGTANYAQ | (MAGE-A3 |
| KFQGRVTITADESTSTAYMELSSLRSEDTAVYYCARDMDTFSMVTLFDYWGQGTLVTVSSGG | pep2) DNA |
| GGSGGGGSGGGGSGGDIQMTQSPSSLSASVGDRVTITCRASQSISSYLNWYQQKPGKAPKLLI | |
| YAASSLQSGVPSRFSGSGSGTDFTLTISSLQPEDFATYYCQQSYSWPLTFGGGTKVEIK | Sequence: SEQ |
| (SEQ ID NO: 222) | ID NO: 223 |
| C-2300/C-2195 (KRAS G12V pep14): | C-2300/ |
| QVTLRESGPALVKPTQTLTLTCTFSGFSLSTSGMCVSWIRQPPGKALEWLALIDWDDDKYYSTS | C-2195 (KRAS |
| LKTRLTISKDTSKNQVVLTMTNMDPVDTATYYCARSYDELYYFDYWGQGTLVTVSSGGGGSG | G12V pep14) |
| GGGSGGGGSGGDIQMTQSPSSLSASVGDRVTITCRASQSIWTSYLNWYQQKPGKAPKLLIYA | DNA |
| ASSLQSGVPSRFSGSGSGTDFTLTISSLOPEDFATYYCQQSYSTPLTFGGGTKVEIK (SEQ ID | Sequence: |
| NO: 224) | SEQ ID NO: 225 |

### Engineered Receptors

The disclosure provides a first engineered receptor comprising a first activator ligand binding domain and a second engineered receptor comprising a second inhibitor ligand binding domain described herein.

### Chimeric Antigen Receptors (CARs)

In some embodiments, the either the first or the second engineered receptor is a chimeric antigen receptor (CAR). In some embodiments, the first and second engineered receptors are chimeric antigen receptors. All CAR architectures are envisaged as within the scope of the instant disclosure.

### Extracellular Domains

In some embodiments, the first or second ligand binding domain is fused to the extracellular domain of the CAR.

### Hinge Region

In some embodiments, the CARs of the present disclosure comprise an extracellular hinge region. Incorporation of a hinge region can affect cytokine production from CAR-T cells and improve expansion of CAR-T cells in vivo. Exemplary hinges can be isolated or derived from IgD and CD8 domains, for example IgG1.

In some embodiments, the hinge is isolated or derived from CD8α or CD28. In some embodiments, the CD8α hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD (SEQ ID NO: 1). In some embodiments, the CD8α hinge comprises SEQ ID NO: 1. In some embodiments, the CD8α hinge consists essentially of SEQ ID NO: 1. In some embodiments, the CD8α hinge is encoded by a nucleotide sequence having at least 80% identity, at least 90% identity, at least 95% identity, at least 99% identity or is identical to a sequence of

(SEQ ID NO: 2)
ACCACGACGCCAGCGCCGCGACCACCAACACCGGCGCCCACCATCGCGT
CGCAGCCCCTGTCCCTGCGCCCAGAGGCGTGCCGGCCAGCGGCGGGGGG
CGCAGTGCACACGAGGGGGCTGGACTTCGCCTGTGAT.

In some embodiments, the CD8α hinge is encoded by SEQ ID NO: 2.

In some embodiments, the CD28 hinge comprises an amino acid sequence having at least 80% identity, at least 90°/% identity, at least 95% identity, at least 99% identity or is identical to a sequence of CTIEVMYPPPYLDNEKSNGTHIHVKGKHLCPSPLFPGPSKP (SEQ ID NO: 3). In some embodiments, the CD28 hinge comprises or consists essentially of SEQ ID NO: 3. In some embodiments, the CD28 hinge is encoded by a nucleotide sequence having at least 80% identity, at least 90% identity, at least 95% identity, at least 99% identity or is identical to a sequence of

(SEQ ID NO: 4)
TGTACCATTGAAGTTATGTATCCTCCTCCTTACCTAGACAATGAGAAGA
GCAATGGAACCATTATCCATGTGAAAGGGAAACACCTTTGTCCAAGTCC
CCTATTTCCCGGACCTTCTAAGCCC.

In some embodiments, the CD28 hinge is encoded by SEQ ID NO: 4.

### Transmembrane Domain

The CARs of the present disclosure can be designed to comprise a transmembrane domain that is fused to the extracellular domain of the CAR. In some embodiments, the transmembrane domain that naturally is associated with one of the domains in the CAR is used. For example, a CAR comprising a CD28 co-stimulatory domain might also use a CD28 transmembrane domain. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In some embodiments of the CARs of the disclosure, the CARs comprise a CD28 transmembrane domain. In some embodiments, the CD28 transmembrane domain comprises an amino acid sequence having at least 80% identity, at least 90% identity, at least 95% identity, at least 99% identity or is identical to a sequence of FWVLVVVGGVLACYSLLVTVAFIIFWV (SEQ ID NO: 5). In some embodiments, the CD28 transmembrane domain comprises or consists essentially of SEQ ID NO: 5. In some embodiments, the CD28 transmembrane domain is encoded by a nucleotide sequence having at least 80% identity, at least 90% identity, at least 95% identity, at least 99% identity or is identical to a sequence of

(SEQ ID NO: 6)
TTCTGGGTGCTGGTCGTTGTGGGCGGCGTGCTGGCCTGCTACAGCCTGC
TGGTGACAGTGGCCTTCATCATCTTTTGGGTG.

In some embodiments, the CD28 transmembrane domain is encoded by SEQ ID NO: 6.

In some embodiments of the CARs of the disclosure, the CARs comprise an IL-2Rbeta transmembrane domain. In some embodiments, the IL-2Rbeta transmembrane domain comprises an amino acid sequence having at least 80% identity, at least 90°/% identity, at least 95% identity, at least 99% identity or is identical to a sequence of IPWLGHLLVGLSGAFGFIILVYLLI (SEQ ID NO: 7). In some embodiments, the IL-2Rbeta transmembrane domain comprises or consists essentially of SEQ ID NO: 7. In some embodiments, the IL-2Rbeta transmembrane domain is encoded by a nucleotide sequence having at least 80% identity, at least 90° %1 identity, at least 95% identity, at least 99% identity or is identical to a sequence of

(SEQ ID NO: 8)
ATTCCGTGGC TCGGCCACCT CCTCGTGGGC CTCAGCGGGG
CTTTTGGCTT CATCATCTTA GTGTACTTGC TGATC.

In some embodiments, the IL-2Rbeta transmembrane domain is encoded by SEQ ID NO: 8.

### Cytoplasmic Domain

The cytoplasmic domain or otherwise the intracellular signaling domain of the CARs of the instant invention is responsible for activation of at least one of the normal effector functions of the immune cell in which the CAR has been placed. The term “effector function” refers to a specialized function of a cell. Effector functions of a regulatory T cell, for example, include the suppression or downregulation of induction or proliferation of effector T cells. Thus the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact chain as long as it transduces the effector function signal. In some cases, multiple intracellular domains can be combined to achieve the desired functions of the CAR-T cells of the instant disclosure. The term intracellular signaling domain is thus meant to include any truncated portion of one or more intracellular signaling domains sufficient to transduce the effector function signal.

Examples of intracellular signaling domains for use in the CARs of the instant disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability. Accordingly, the intracellular domain of CARs of the instant disclosure comprises at least one cytoplasmic activation domain. In some embodiments, the intracellular activation domain ensures that there is T-cell receptor (TCR) signaling necessary to activate the effector functions of the CAR T-cell. In some embodiments, the at least one cytoplasmic activation is a CD247 molecule (CD3ζ) activation domain, a stimulatory killer immunoglobulin-like receptor (KIR) KIR2DS2 activation domain, or a DNAX-activating protein of 12 kDa (DAP12) activation domain. In some embodiments, the CD3ζ activation domain comprises an amino acid sequence having at least 80% identity, at least 90% identity, at least 95% identity, at least 99% identity or is identical to a sequence of

(SEQ ID NO: 9)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the CD3 activation domain comprises or consists essentially of SEQ ID NO: 9. In some embodiments, the CD3, activation domain is encoded by a nucleotide sequence having at least 80% identity, at least 90% identity, at least 95% identity, at least 99% identity or is identical to a sequence of

(SEQ ID NO: 10)
AGAGTGAAGTTCAGCAGGAGCGCAGACGCCCCCGCGTACAAGCAGGGCC
AGAACCAGCTCTATAACGAGCTCAATCTAGGACGAAGAGAGGAGTACGA
TGTTTTGGACAAGCGTAGAGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGACTCAGTACAGCCACCAAG
GACACCTACGACGCCCTTCACATGCAGGCCCTGCCCCCTCGC.

In some embodiments, the CD3ζ activation domain is encoded by SEQ ID NO: 10.

It is known that signals generated through the TCR alone are often insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. In some embodiments, the ITAM contains a tyrosine separated from a leucine or an isoleucine by any two other amino acids (YxxL) (SEQ ID NO: 21).

In some embodiments, the cytoplasmic domain contains 1, 2, or 3 ITAMs. In some embodiments, the cytoplasmic domain contains 1 ITAM. In some embodiments, the cytoplasmic domain contains 2 ITAMs. In some embodiments, the cytoplasmic domain contains 3 ITAMs. In some embodiments, the cytoplasmic domain contains 4 ITAMs. In some embodiments, the cytoplasmic domain contains 5 ITAMs.

In some embodiments, the cytoplasmic domain is a CD3ζ activation domain. In some embodiments, CD3ζ activation domain comprises a single ITAM. In some embodiments, CD3ζ activation domain comprises two ITAMs. In some embodiments, CD3ζ activation domain comprises three ITAMs.

In some embodiments, the CD3ζ activation domain comprising a single ITAM comprises an amino acid sequence having at least 80% identity, at least 90% identity, at least 95% identity, at least 99% identity or is identical to a sequence of RVKFSRSADAPAYQQGQNQLYNELNLGRREEYDVLHMQALPPR (SEQ ID NO: 11). In some embodiments, the CD3ζ activation domain comprises SEQ ID NO: 11. In some embodiments, the CD3ζ activation domain comprising a single ITAM consists essentially of an amino acid sequence of RVKFSRSADAPAYQQGQNQLYNELNLGRREEYDVLHMQALPPR (SEQ ID NO: 11). In some embodiments, the CD3ζ activation domain comprising a single ITAM is encoded by a nucleotide sequence having at least 80% identity, at least 90% identity, at least 95% identity, at least 99% identity or is identical to a sequence of

(SEQ ID NO: 12)
AGAGTGAAGT TCAGCAGGAG CGCAGACGCC CCCGCGTACC
AGCAGGGCCA GAACCAGCTC TATAACGAGC TCAATCTAGG
ACGAAGAGAG GAGTACGATG TTTTGCACAT GCAGGCCCTG
CCCCCTCGC.

In some embodiments, the CD3ζ activation domain is encoded by SEQ ID NO: 12.

Further examples of ITAM containing primary cytoplasmic signaling sequences that can be used in the CARs of the instant disclosure include those derived from TCRζ, FcRγ, FcRβ, CD3γ, CD3δ, CD3ε, CD3ζ, CD5, CD22, CD79a, CD79b, and CD66d. It is particularly preferred that cytoplasmic signaling molecule in the CAR of the instant invention comprises a cytoplasmic signaling sequence derived from CD3ζ.

### Co-Stimulatory Domain

In some embodiments, the cytoplasmic domain of the CAR can be designed to comprise the CD3ζ signaling domain by itself or combined with any other desired cytoplasmic domain(s) useful in the context of the CAR of the instant disclosure. For example, the cytoplasmic domain of the CAR can comprise a CD3ζ chain portion and a co-stimulatory domain. The co-stimulatory domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include the co-stimulatory domain is selected from the group consisting of IL-2Rβ, Fc Receptor gamma (FcRγ), Fc Receptor beta (FcRβ), CD3g molecule gamma (CD3γ), CD3δ, CD3ε, CD5 molecule (CD5), CD22 molecule (CD22), CD79a molecule (CD79a), CD79b molecule (CD79b), carcinoembryonic antigen related cell adhesion molecule 3 (CD66d), CD27 molecule (CD27), CD28 molecule (CD28), TNF receptor superfamily member 9 (4-1BB), TNF receptor superfamily member 4 (OX40), TNF receptor superfamily member 8 (CD30), CD40 molecule (CD40), programmed cell death 1 (PD-1), inducible T cell costimulatory (ICOS), lymphocyte function-associated antigen-1 (LFA-1), CD2 molecule (CD2), CD7 molecule (CD7), TNF superfamily member 14 (LIGHT), killer cell lectin like receptor C2 (NKG2C) and CD276 molecule (B7-H3) c-stimulatory domains, or functional fragments thereof.

The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker.

In some embodiments, the intracellular domains of CARs of the instant disclosure comprise at least one co-stimulatory domain. In some embodiments, the co-stimulatory domain is isolated or derived from CD28. In some embodiments, the CD28 co-stimulatory domain comprises an amino acid sequence having at least 80% identity, at least 90% identity, at least 95% identity, at least 99% identity or is identical to a sequence of

(SEQ ID NO: 13)
RSKRSRLLHSDYMNMTPRRPGPTRKHYQPYAPPRDFAAYRS.

In some embodiments, the CD28 co-stimulatory domain comprises or consists essentially of SEQ ID NO: 13. In some embodiments, the CD28 co-stimulatory domain is encoded by a nucleotide sequence having at least 80% identity, at least 90% identity, at least 95% identity, at least 99% identity or is identical to a sequence of

(SEQ ID NO: 14)
AGGAGCAAGCGGAGCAGACTGCTGCACAGCGACTACATGAACATGACCC
CCCGGAGGCCTGGCCCCACCCGGAAGCACTACCAGCCCTACGCCCCTCC
CAGGGATTTCGCCGCCTACCGGAGC.

In some embodiments, the CD28 co-stimulatory domain is encoded by SEQ ID NO: 14.

In some embodiments, the intracellular domain of the CARs of the instant disclosure comprises an interleukin-2 receptor beta-chain (IL-2Rbeta or IL-2R-beta) cytoplasmic domain. In some embodiments, the IL-2Rbeta domain is truncated. In some embodiments, the IL-2Rbeta cytoplasmic domain comprises one or more STAT5-recruitment motifs. In some embodiments, the CAR comprises one or more STAT5-recruitment motifs outside the IL-2Rbeta cytoplasmic domain.

In some embodiments, the IL-2-Rbeta intracellular domain comprises an amino acid sequence having at least 80% identity, at least 90% identity, at least 95% identity, at least 99% identity or is identical to a sequence of

(SEQ ID NO: 15)
NCRNTGPWLKKVLKCNTPDPSKFFSQLSSEHGGDVQKWLSSPFPSSSFS
PGGLAPEISPLEVLERDKVTQLLPLNTDAYLSLQELQGQDPTHLV.

In some embodiments, the IL2R-beta intracellular domain comprises or consists essentially of SEQ ID NO: 15. In some embodiments, the IL-2R-beta intracellular domain is encoded by a nucleotide sequence having at least 80% identity, at least 90% identity, at least 95% identity, at least 99% identity or is identical to a sequence of

| (SEQ ID NO: 16) | |
|---|---|
| 1 | AACTGCAGGA ACACCGGGCC ATGGCTGAAG AAGGTCCTGA AGTGTAACAC CCCAGACCCC |
| 61 | TCGAAGTTCT TTTCCCAGCT GAGCTCAGAG CATGGAGGCG ACGTCCAGAA GTGGCTCTCT |
| 121 | TCGCCCTTCC CCTCATCGTC CTTCAGCCCT GGCGGCCTGG CACCTGAGAT CTCGCCACTA |
| 181 | GAAGTGCTGG AGAGGGACAA GGTGACGCAG CTGCTCCCCC TGAACACTGA TGCCTACTTG |
| 241 | TCTCTCCAAG AACTCCAGGG TCAGGACCCA ACTCACTTGG TG. |

In some embodiments, the IL-2R-beta intracellular domain is encoded by SEQ ID NO: 16.

In an embodiment, the IL-2R-beta cytoplasmic domain comprises one or more STAT5-recruitment motifs. Exemplary STAT5-recruitment motifs are provided by Passerini et al. (2008) STAT5-signaling cytokines regulate the expression of FOXP3 in CD4+CD25+ regulatory T cells and CD4+CD25+ effector T cells. International Immunology, Vol. 20, No. 3, pp. 421-431, and by Kagoya et al. (2018) A novel chimeric antigen receptor containing a JAK-STAT signaling domain mediates superior antitumor effects. Nature Medicine doi:10.1038/nm.4478.

In some embodiments, the STAT5-recruitment motif(s) consists of the sequence Tyr-Leu-Ser-Leu (SEQ ID NO: 17).

### Inhibitory Domains

In some embodiments, for example in the second engineered receptors of the disclosure which provide an inhibitory signal, the inhibitory signal is transmitted through the intracellular domain of the receptor. In some embodiments, the engineered receptor comprises an inhibitory intracellular domain. In some embodiments, the second engineered receptor is a CAR comprising an inhibitory intracellular domain (an inhibitory CAR).

In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells, and play a pivotal role in attenuating or terminating T cell responses.

Inhibitory domains can be isolated from human tumor necrosis factor related apoptosis inducing ligand (TRAIL) receptor and CD200 receptor 1.

In some embodiments, the inhibitory domain comprises an intracellular domain, a transmembrane or a combination thereof. In some embodiments, the inhibitory domain comprises an intracellular domain, a transmembrane domain, a hinge region or a combination thereof. In some embodiments, the inhibitory domain comprises an immunoreceptor tyrosine-based inhibitory motif (ITIM). In some embodiments, the inhibitory domain comprising an ITIM can be isolated or derived from an immune checkpoint inhibitor such as CTLA-4 and PD-1.

Inhibitory domains can be isolated from human tumor necrosis factor related apoptosis inducing ligand (TRAIL) receptor and CD200 receptor 1. In some embodiments, the inhibitory domain is isolated or derived from a human protein, for example a human TRAIL receptor, CTLA-4, or PD-1 protein. In some embodiments, the TRAIL receptor comprises TR10A, TR10B or TR10D.

Endogenous TRAIL is expressed as a 281-amino acid type II trans-membrane protein, which is anchored to the plasma membrane and presented on the cell surface. TRAIL is expressed by natural killer cells, which, following the establishment of cell-cell contacts, can induce TRAIL-dependent apoptosis in target cells. Physiologically, the TRAIL-signaling system was shown to be essential for immune surveillance, for shaping the immune system through regulating T-helper cell 1 versus T-helper cell 2 as well as “helpless” CD8+ T-cell numbers, and for the suppression of spontaneous tumor formation.

In some embodiments, the inhibitory domain comprises an intracellular domain isolated or derived from a CD200 receptor. The cell surface glycoprotein CD200 receptor 1 (Uniprot ref: Q8TD46) represents another example of an inhibitory intracellular domain of the present invention. This inhibitory receptor for the CD200/OX2 cell surface glycoprotein limits inflammation by inhibiting the expression of proinflammatory molecules including TNF-alpha, interferons, and inducible nitric oxide synthase (iNOS) in response to selected stimuli.

In some embodiments, the engineered receptor comprises an inhibitory domain isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1, also called LIR-1 and LILRB1), programmed cell death 1 (PD-1), Fc gamma receptor IIB (FcgRIIB), killer cell lectin like receptor K1 (NKG2D), CTLA-4, a domain containing a synthetic consensus ITIM, a ZAP70 SH2 domain (e.g., one or both of the N and C terminal SH2 domains), or ZAP70 KI_K369A (kinase inactive ZAP70).

In some embodiments, the inhibitory domain is isolated or derived from a human protein.

In some embodiments, the second, inhibitory receptor comprises a cytoplasmic domain and transmembrane domain isolated or derived from the same protein, for example an ITIM containing protein. In some embodiments, the second, inhibitory receptor comprises a cytoplasmic domain, a transmembrane domain, and an extracellular domain or a portion thereof isolated or derived isolated or derived from the same protein, for example an ITIM containing protein. In some embodiments, the second, inhibitory receptor comprises a hinge region isolated or derived from isolated or derived from the same protein as the intracellular domain and/or transmembrane domain, for example an ITIM containing protein.

In some embodiments, the second, inhibitory engineered receptor comprises an inhibitory domain. In some embodiments, the second, inhibitory engineered receptor comprises an inhibitory intracellular domain and/or an inhibitory transmembrane domain. In some embodiments, the second engineered receptor is a CAR comprising an inhibitory domain (an inhibitory CAR). In some embodiments, the inhibitory intracellular domain is fused to the intracellular domain of a CAR. In some embodiments, the inhibitory intracellular domain is fused to the transmembrane domain of a CAR.

### T Cell Receptors (TCRs)

In some embodiments, the first or second engineered receptor is a T Cell Receptor (TCR). In some embodiments, the first and second engineered receptors are a T Cell Receptors (TCR).

As used herein, a “TCR”, sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-MHC complexes. Once the TCR alpha/beta heterodimer engages peptide-MHC, conformational changes in the TCR complex in the associated invariant CD3 subunits are induced, which leads to their phosphorylation and association with downstream proteins, thereby transducing a primary stimulatory signal. In an exemplary TCR complex, the TCR alpha and TCR beta polypeptides form a heterodimer, CD3 epsilon and CD3 delta form a heterodimer, CD3 epsilon and CD3 gamma for a heterodimer, and two CD3 zeta form a homodimer.

### Extracellular Domains

The disclosure provides a first engineered receptor comprising a first extracellular ligand binding domain and a second engineered receptor comprising a second extracellular ligand binding domain. Either the first engineered receptor, the second engineered receptor, or both, may be a TCR Any suitable ligand binding domain may be fused to an extracellular domain, hinge domain or transmembrane of the engineered TCRs described herein.

In some embodiments, the first and/or second ligand binding domain is fused to an extracellular domain of a TCR subunit. The TCR subunit can be TCR alpha, TCR beta, CD3 delta, CD3 epsilon or CD3 gamma. In some embodiments, both the first and second ligand binding domains are fused to the same TCR subunit in different TCR receptors. In some embodiments, the first and second ligand binding domains are fused to different TCR subunits in different TCR receptors. In some embodiments, the first, activator ligand binding domain is fused to a first TCR subunit in a first engineered receptor and the second, inhibitor ligand binding domain is fused to a second TCR subunit in a second engineered receptor. In some embodiments, the first and second TCR subunits are not the same subunit. In some embodiments, the first and second TCR subunits are the same subunit. For example, the first ligand binding domain can be fused to TCR alpha, and the second ligand binding domain can be fused to TCR beta. As a further example, the first ligand binding is fused to TCR beta and the second ligand binding domain used fused to TCR alpha.

In some embodiments, the first, activator LBD comprises an ScFv domain and the second, inhibitor LBD comprises a Vβ-only domain. In some embodiments, the first, activator LBD comprises a Vβ-only domain and the second, inhibitor LBD comprises an ScFv domain. In some embodiments, both the first, activator LBD and the second, inhibitor LBD are ScFv domains. In some embodiments, both the first, activator LBD and the second, inhibitor LBD are Vβ-only domains.

In some embodiments, the first engineered TCR of the disclosure comprises an extracellular domain comprising a Vβ-only domain, a transmembrane domain and an intracellular domain. In some embodiments, the intracellular domain comprises one or more exogenous domains.

In some embodiments, the first engineered TCR of the disclosure comprises an extracellular domain comprising an ScFv domain, a transmembrane domain and an intracellular domain. In some embodiments, the intracellular domain comprises one or more exogenous domains.

In some embodiments, the second engineered TCR of the disclosure comprises an extracellular domain comprising a Vβ-only domain, a transmembrane domain and an inhibitory intracellular domain.

In some embodiments, the second engineered TCR of the disclosure comprises an extracellular domain comprising an ScFv domain, a transmembrane domain and an inhibitory intracellular domain.

TCR subunits include TCR alpha, TCR beta, CD3 zeta, CD3 delta, CD3 gamma and CD3 epsilon. Any one or more of TCR alpha, TCR beta chain, CD3 gamma, CD3 delta or CD3 epsilon, or fragments or derivative thereof, can be fused to one or more domains capable of providing a stimulatory signal of the disclosure, thereby enhancing TCR function and activity. Any one or more of TCR alpha, TCR beta chain, CD3 gamma, CD3 delta or CD3 epsilon, or fragments or derivative thereof, can be fused to an inhibitory intracellular domain of the disclosure.

In some embodiments, for example those embodiments wherein the first engineered receptor or second engineered receptor comprises a first and a second polypeptide, the antigen binding domain is isolated or derived from a T cell receptor (TCR) extracellular domain or an antibody.

In some embodiments, the first engineered receptor and second engineered receptor comprise a first antigen binding domain and a second antigen binding domain. The antigen-binding domain or domains of the engineered receptor may be provided on the same or a different polypeptide as the intracellular domain.

In some embodiments, the antigen-binding domain of the first and/or second engineered receptor comprises a single chain variable fragment (scFv).

In some embodiments, the first and/or second engineered receptor comprises a second polypeptide. The disclosure provides receptors having two polypeptides each having a part of a ligand-binding domain (e.g. cognates of a heterodimeric LDB, such as a TCRα/β- or Fab-based LBD). The disclosure further provides receptors having two polypeptides, each having a part of a ligand-binding domain (e.g. cognates of a heterodimeric LDB, such as a TCRα/β- or Fab-based LBD) and one part of the ligand binding domain is fused to a hinge or transmembrane domain, while the other part of the ligand binding domain has no intracellular domain. Further variations include receptors where each polypeptide has a hinge domain, and where each polypeptide has a hinge and transmembrane domain. In some embodiments, the hinge domain is absent. In other embodiments, the hinge domain is a membrane proximal extracellular region (MPER), such as the LILRB1 D3D4 domain.

In some embodiments, for example those embodiments where the first and/or second engineered receptor comprises at least two polypeptides, the first polypeptide comprises a first chain of an antibody and the second polypeptide comprise a second chain of said antibody.

In some embodiments, the receptor comprises a Fab fragment of an antibody. In embodiments, a first polypeptide comprises an antigen-binding fragment of the heavy chain of the antibody and an intracellular domain, and a second polypeptide comprises an antigen-binding fragment of the light chain of the antibody. In some embodiments, the first polypeptide comprises an antigen-binding fragment of the light chain of the antibody and the intracellular domain, and the second polypeptide comprises an antigen-binding fragment of the heavy chain of the antibody.

In some embodiments, the first and/or second engineered receptor comprises an extracellular fragment of a T cell receptor (TCR). In some embodiments, a first polypeptide comprises an antigen-binding fragment of the alpha chain of the TCR and the intracellular domain, and a second polypeptide comprises an antigen-binding fragment of the beta chain of the TCR. In some embodiments, a first polypeptide comprises an antigen-binding fragment of the beta chain of the TCR and the intracellular domain, and the second polypeptide comprises an antigen-binding fragment of the alpha chain of the TCR.

### TCRs Comprising Vβ-Only Domains

Certain embodiments of present disclosure relate to engineered TCRs comprising a TCR variable domain, the TCR variable domain specifically binding to an antigen in the absence of a second TCR variable domain (a Vβ-only domain).

In some embodiments, the engineered TCR comprises additional elements besides the TCR variable domain, including additional amino acid sequences, additional protein domains (covalently associated, non-covalently associated or covalently and non-covalently associated with the TCR variable domain), fusion or non-covalent association of the TCR variable domain with other types of macromolecules (for example polynucleotides, polysaccharides, lipids, or a combination thereof), fusion or non-covalent association of the TCR variable domain with one or more small molecules, compounds, or ligands, or a combination thereof. Any additional element, as described, may be combined provided that the TCR variable domain is configured to specifically bind the epitope in the absence of a second TCR variable domain.

An engineered TCR comprising a Vβ-only domain as described herein may comprise a single TCR chain (e.g. α, β, γ, or δ chain), or it may comprise a single TCR variable domain (e.g. of α, β, γ, or δ chain). If the engineered TCR is a single TCR chain, then the TCR chain comprises a transmembrane domain, a constant (or C domain) and a variable (or V domain), and does not comprise a second TCR variable domain. The engineered TCR may therefore comprise or consist of a TCR α chain, a TCR β chain, a TCR γ chain or a TCR δ chain. The engineered TCR may be a membrane bound protein. The engineered TCR may alternatively be a membrane-associated protein.

In some embodiments, the engineered TCR as described herein utilizes a surrogate α chain that lacks a Vα segment, which forms activation-competent TCRs complexed with the six CD3 subunits.

In other embodiments, the engineered TCR as described herein functions independently of a surrogate α chain that lacks a Vα segment. For example, in some embodiments the one or more engineered TCRs are fused to transmembrane (e.g., CD3ζ and CD28) and intracellular domain proteins (e.g., CD3ζ, CD28, and/or 4-1BB) that are capable of activating T cells in response to antigen.

In some embodiments, the engineered TCR comprises one or more single TCR chains fused to the Vβ-only domain described herein. For example, the engineered TCR may comprise, or consist essentially of single α TCR chain, a single β TCR chain, a single γ TCR chain, or a single δ TCR chain fused to one or more Vβ-only domains.

In some embodiments, the engineered TCR engages antigen using complementarity-determining regions (CDRs). Each engineered TCR contains three complement determining regions (CDR1, CDR2, and CDR3).

The first and/or second ligand binding Vβ-only domain may be a human TCR variable domain. Alternatively, the first and/or second Vβ-only domain may be a non-human TCR variable domain. The first and/or second Vβ-only domain may be a mammalian TCR variable domain. The first and/or second Vβ-only domain may be a vertebrate TCR variable domain.

In embodiments where Vβ-only domain is incorporated into a fusion protein, for example a fusion protein comprising a TCR subunit, and optionally, an additional stimulatory intracellular domain. The fusion protein may comprise a Vβ-only domain and any other protein domain or domains.

### Transmembrane Domains

The disclosure provide a first fusion protein comprising a first, activator LBD and a second fusion protein comprising a second, inhibitor LBD and an inhibitor intracellular domain. In some embodiments, the first and second fusion proteins comprise transmembrane domains.

The disclosure provides polypeptides comprising a transmembrane domain, and an intracellular domain capable of providing a stimulatory signal or an inhibitory signal. In some embodiments, the engineered TCR comprises multiple intracellular domains capable of providing a stimulatory signal.

A “transmembrane domain”, as used herein, refers to a domain of a protein that spans membrane of the cell. Transmembrane domains typically consist predominantly of non-polar amino acids, and may traverse the lipid bilayer once or several times. Transmembrane domains usually comprise alpha helices, a configuration which maximizes internal hydrogen bonding.

Transmembrane domains isolated or derived from any source are envisaged as within the scope of the fusion proteins of the disclosure.

In some embodiments, the transmembrane domain is one that is associated with one of the other domains of the fusion protein, or isolated or derived from the same protein as one of the other domains of the fusion protein. In some embodiments, the transmembrane domain and the second intracellular domain are from the same protein, for example a TCR complex subunit such as TCR alpha, TCR beta, CD3 delta, CD3 epsilon or CD3 gamma. In some embodiments, the extracellular domain (svd-TCR), the transmembrane domain and the second intracellular domain are from the same protein, for example a TCR complex subunit such as TCR alpha, TCR beta, CD3 delta, CD3 epsilon or CD3 gamma. In other embodiments, the extracellular domain (comprising one or more ligand binding domains, such as Vβ-only domain and ScFv domains), the transmembrane domain and the intracellular domain(s) are from different proteins. For example, in some embodiments the engineered svd-TCR comprises a CD28 transmembrane domain with a CD28, 4-1BB and CD3ζ intracellular domain.

The transmembrane domain may be derived either from a natural or from a recombinant source. Where the source is natural, the domain may be derived from any membrane-bound or transmembrane protein.

In some embodiments, the transmembrane domain is capable of signaling to the intracellular domain(s) whenever the TCR complex has bound to a target. A transmembrane domain of particular use in this invention may include at least the transmembrane region(s) of e.g., the alpha, beta or zeta chain of the TCR, CD3 delta, CD3 epsilon or CD3 gamma, CD28, CD3 epsilon, CD45, CD4, CD5, CD8, CD9, CD16, CD22, CD33, CD37, CD64, CD80, CD86, CD134, CD137, CD154.

In some embodiments, the transmembrane domain can be attached to the extracellular region of the fusion protein, e.g., the antigen binding domain of the TCR alpha or beta chain, via a hinge, e.g., a hinge from a human protein. For example, in one embodiment, the hinge can be a human immunoglobulin (Ig) hinge, e.g., an IgG4 hinge, or a CD8α hinge.

In some embodiments, the hinge is isolated or derived from CD8α or CD28. In some embodiments, the CD8α hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD (SEQ ID NO: 1). In some embodiments, the CD8α hinge comprises SEQ ID NO: 1. In some embodiments, the CD8α hinge consists essentially of SEQ ID NO: 1. In some embodiments, the CD8α hinge is encoded by a nucleotide sequence having at least 80% identity, at least 90% identity, at least 95% identity, at least 99% identity or is identical to a sequence of:

(SEQ ID NO: 2)
ACCACGACGCCAGCGCCGCGACCACCAACACCGGCGCCCACCATCGCGT
CGCAGCCCCTGTCCCTGCGCCCAGAGGCGTGCCGGCCAGCGGCGGGGGG
CGCAGTGCACACGAGGGGGCTGGACTTCGCCTGTGAT.

In some embodiments, the CD8α hinge is encoded by SEQ ID NO: 2.

In some embodiments, the CD28 hinge comprises an amino acid sequence having at least 80% identity, at least 90% identity, at least 95% identity, at least 99% identity or is identical to a sequence of CTIEVMYPPPYLDNEKSNGTHIHVKGKHLCPSPLFPGPSKP (SEQ ID NO: 3. In some embodiments, the CD28 hinge comprises or consists essentially of SEQ ID NO: 3. In some embodiments, the CD28 hinge is encoded by a nucleotide sequence having at least 80% identity, at least 90% identity, at least 95% identity, at least 99% identity or is identical to a sequence of

(SEQ ID NO: 4)
TGTACCATTGAAGTTATGTATCCTCCTCCTTACCTAGACAATGAGAAGA
GCAATGGAACCATTATCCATGTGAAAGGGAAACACCTTTGTCCAAGTCC
CCTATTTCCCGGACCTTCTAAGCCC.

In some embodiments, the CD28 hinge is encoded by SEQ ID NO: 4.

In some embodiments, the transmembrane domain comprises a TCR alpha transmembrane domain. In some embodiments, the TCR alpha transmembrane domain comprises an amino acid sequence having at least 85% identity, at least 90% identity, at least 95% identity, at least 96% identity, at least 97% identity, at least 98% identity, at least 99% identity or is identical to a sequence of; VIGFRILLLKVAGFNLLMTLRLW (SEQ ID NO: 26). In some embodiments, the TCR alpha transmembrane domain comprises, or consists essentially of, SEQ ID NO: 26. In some embodiments, the TCR alpha transmembrane domain is encoded by a sequence of

(SEQ ID NO: 27)
GTGATTGGGTTCCGAATCCTCCTCCTGAAAGTGGCCGGGTTTAATCTGC
TCATGACGCTGCGGCTGTGG.

In some embodiments, the transmembrane domain comprises a TCR beta transmembrane domain. In some embodiments, the TCR beta transmembrane domain comprises an amino acid sequence having at least 85% identity, at least 90% identity, at least 95% identity, at least 96% identity, at least 97% identity, at least 98% identity, at least 99% identity or is identical to a sequence of: TILYEILLGKATLYAVLVSALVL (SEQ ID NO: 28). In some embodiments, the TCR beta transmembrane domain comprises, or consists essentially of, SEQ ID NO: 28. In some embodiments, the TCR beta transmembrane domain is encoded by a sequence of

(SEQ ID NO: 20)
ACCATCCTCTATGAGATCTTGCTAGGGAAGGCCACCTTGTATGCCGTGC
TGGTCAGTGCCCTCGTGCTG.

In some embodiments, the transmembrane comprises a CD3 zeta transmembrane domain. In some embodiments, the CD3 zeta transmembrane domain comprises an amino acid sequence having at least 85% identity, at least 90% identity, at least 95% identity, at least 96% identity, at least 97% identity, at least 98% identity, at least 99% identity or is identical to a sequence of: LCYLLDGILFIYGVILTALFL (SEQ ID NO: 29). In some embodiments, the CD3 zeta transmembrane domain comprises, or consists essentially of, SEQ ID NO: 29.

A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region).

In some embodiments, the transmembrane domain can be selected or modified by amino acid substitution to avoid binding of such domains to the transmembrane domains of the same or different surface membrane proteins, e.g., to minimize interactions with other members of the receptor complex.

When present, the transmembrane domain may be a natural TCR transmembrane domain, a natural transmembrane domain from a heterologous membrane protein, or an artificial transmembrane domain. The transmembrane domain may be a membrane anchor domain. Without limitation, a natural or artificial transmembrane domain may comprise a hydrophobic a-helix of about 20 amino acids, often with positive charges flanking the transmembrane segment. The transmembrane domain may have one transmembrane segment or more than one transmembrane segment. Prediction of transmembrane domains/segments may be made using publicly available prediction tools (e.g. TMHMM, Krogh et al. Journal of Molecular Biology 2001; 305(3):567-580; or TMpred, Hofmann & Stoffel Biol. Chem. Hoppe-Seyler 1993; 347: 166). Non-limiting examples of membrane anchor systems include platelet derived growth factor receptor (PDGFR) transmembrane domain, glycosylphosphatidylinositol (GPI) anchor (added post-translationally to a signal sequence) and the like.

### Intracellular Domain

The disclosure provides fusion proteins comprising an intracellular domain. An “intracellular domain,” as the term is used herein, refers to an intracellular portion of a protein.

In some embodiments, the intracellular domain comprises one or more domains capable of providing a stimulatory signal to a transmembrane domain. In some embodiments, the intracellular domain comprises a first intracellular domain capable of providing a stimulatory signal and a second intracellular domain capable of providing a stimulatory signal. In other embodiments, the intracellular domain comprises a first, second and third intracellular domain capable of providing a stimulatory signal. The intracellular domains capable of providing a stimulatory signal are selected from the group consisting of a CD28 molecule (CD28) domain, a LCK proto-oncogene, Src family tyrosine kinase (Lck) domain, a TNF receptor superfamily member 9 (4-1BB) domain, a TNF receptor superfamily member 18 (GITR) domain, a CD4 molecule (CD4) domain, a CD8α molecule (CD8a) domain, a FYN proto-oncogene, Src family tyrosine kinase (Fyn) domain, a zeta chain of T cell receptor associated protein kinase 70 (ZAP70) domain, a linker for activation of T cells (LAT) domain, lymphocyte cytosolic protein 2 (SLP76) domain, (TCR) alpha, TCR beta, CD3 delta, CD3 gamma and CD3 epsilon intracellular domains.

In some embodiments, an intracellular domain comprises at least one intracellular signaling domain. An intracellular signaling domain generates a signal that promotes a function a cell, for example an immune effector function of a TCR containing cell, e.g., a TCR-expressing T-cell. In some embodiments, the intracellular domain of the fusion proteins of the disclosure includes at least one intracellular signaling domain. For example, the intracellular domains of CD3 gamma, delta or epsilon comprise signaling domains.

In some embodiments, the extracellular domain, transmembrane domain and intracellular domain are isolated or derived from the same protein, for example T-cell receptor (TCR) alpha, TCR beta, CD3 delta, CD3 gamma or CD3 epsilon.

Examples of intracellular domains for use in the fusion proteins of the disclosure include the cytoplasmic sequences of the TCR alpha, TCR beta, CD3 zeta, and 4-1BB, and the intracellular signaling co-receptors that act in concert to initiate signal transduction following antigen receptor engagement, as well as any derivative or variant of these sequences and any recombinant sequence that has the same functional capability.

In some embodiments, the intracellular signaling domain comprises a primary intracellular signaling domain. Exemplary primary intracellular signaling domains include those derived from the proteins responsible for primary stimulation, or antigen dependent stimulation.

An intracellular signaling domain is generally responsible for activation of at least one of the normal effector functions of the immune cell in which the fusion protein has been introduced. The term “effector function” refers to a specialized function of a cell. Effector function of a T-cell, for example, may be cytolytic activity or helper activity including the secretion of cytokines. Thus the term “intracellular signaling domain” refers to the portion of a protein which transduces the effector function signal and directs the cell to perform a specialized function.

While in some cases the entire intracellular signaling domain can be employed, in many cases it is not necessary to use the entire intracellular signaling domain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular domain comprises a CD3 delta intracellular domain. In some embodiments, the CD3 delta intracellular domain comprises an amino acid sequence having at least 85% identity, at least 90% identity, at least 95% identity, at least 96% identity, at least 97% identity, at least 98% identity, at least 99% identity or is identical to a sequence of

(SEQ ID NO: 30)
GHETGRLSGAADTQALLRNDQVYQPLRDRDDAQYSHLGGNWARNKGGSR
SKRSRLLHSDYMNMTPRRPGPTRKHYQPYAPPRDFAAYRS.

In some embodiments, the CD3 delta intracellular domain comprises or consists essentially of, SEQ ID NO: 30. In some embodiments, the CD3 delta intracellular domain is encoded by a sequence of

| (SEQ ID NO: 31) | |
|---|---|
| 1 | GGACATGAGA CTGGAAGGCT GTCTGGGGCT GCCGACACAC AAGCTCTGTT GAGGAATGAC |
| 61 | CAGGTCTATC AGCCCCTCCG AGATCGAGAT GATGCTCAGT ACAGCCACCT TGGAGGAAAC |
| 121 | TGGGCTCGGA ACAAGGGCGG AAGCAGGAGC AAGCGGAGCA GACTGCTGCA CAGCGACTAC |
| 181 | ATGAACATGA CCCCCCGGAG GCCTGGCCCC ACCCGGAAGC ACTACCAGCC CTACGCCCCT |
| 241 | CCCAGGGATT TCGCCGCCTA CCGGAGCTA. |

In some embodiments, the intracellular domain comprises a CD3 epsilon intracellular domain. In some embodiments, the CD3 epsilon intracellular domain comprises an amino acid sequence having at least 85% identity, at least 90% identity, at least 95% identity, at least 96% identity, at least 97% identity, at least 98% identity, at least 99% identity or is identical to a sequence of: KNRKAKAKPVTRGAGAGGRQRGQNKERPPPVPNPDYEPIRKGQRDLYSGLNQRRIGGS RSKRSRLLHSDYMNMTPRRPGPTRKHYQPYAPPRDFAAYRS (SEQ ID NO: 32). In some embodiments, the CD3 epsilon intracellular domain comprises or consists essentially of, SEQ ID NO: 32. In some embodiments, the CD3 epsilon intracellular domain is encoded by a sequence of

| (SEQ ID NO: 19) | |
|---|---|
| 1 | AAGAATAGAA AGGCCAAGGC CAAGCCTGTG ACACGAGGAG CGGGTGCTGG CGGCAGGCAA |
| 61 | AGGGGACAAA ACAAGGAGAG GCCACCACCT GTTCCCAACC CAGACTATGA GCCCATCCGG |
| 121 | AAAGGCCAGC GGGACCTGTA TTCTGGCCTG AATCAGCGCA GAATCGGCGG AAGCAGGAGC |
| 181 | AAGCGGAGCA GACTGCTGCA CAGCGACTAC ATGAACATGA CCCCCCGGAG GCCTGGCCCC |
| 241 | ACCCGGAAGC ACTACCAGCC CTACGCCCCT CCCAGGGATT TCGCCGCCTA CCGGAGCTAG. |

In some embodiments, the intracellular domain comprises a CD3 gamma intracellular domain. In some embodiments, the CD3 gamma intracellular domain comprises an amino acid sequence having at least 85% identity, at least 90% identity, at least 95% identity, at least 96% identity, at least 97% identity, at least 98% identity, at least 99% identity or is identical to a sequence of

(SEQ ID NO: 33)
GQDGVRQSRASDKQTLLPNDQLYQPLKDREDDQYSHLQGNQLRRNGGSR
SKRSRLLHSDYMNMTPRRPGPTRKHYQPYAPPRDFAAYRS.

In some embodiments, the CD3 gamma intracellular domain comprises, or consists essentially of, SEQ ID NO: 33. In some embodiments, the CD3 gamma intracellular domain is encoded by a sequence of

| (SEQ ID NO: 22) | |
|---|---|
| 1 | GGACAGGATG GAGTTCGCCA GTCGAGAGCT TCAGACAAGC AGACTCTGTT GCCCAATGAC |
| 61 | CAGCTCTACC AGCCCCTCAA GGATCGAGAA GATGACCAGT ACAGCCACCT TCAAGGAAAC |
| 121 | CAGTTGAGGA GGAATGGCGG AAGCAGGAGC AAGCGGAGCA GACTGCTGCA CAGCGACTAC |
| 181 | ATGAACATGA CCCCCCGGAG GCCTGGCCCC ACCCGGAAGC ACTACCAGCC CTACGCCCCT |
| 241 | CCCAGGGATT TCGCCGCCTA CCGGAGCTAG. |

In some embodiments, the intracellular domain comprises a CD3 zeta intracellular domain. In some embodiments, the CD3 zeta intracellular domain comprises an amino acid sequence having at least 85% identity, at least 90% identity, at least 95% identity, at least 96% identity, at least 97% identity, at least 98% identity, at least 99% identity or is identical to a sequence of RVKFSRSADAPAYKQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 9) or a subsequence thereof.

In some embodiments, the CD3 zeta intracellular domain comprises, or consists essentially of, SEQ ID NO: 9.

In some embodiments, the intracellular domain comprises a TCR alpha intracellular domain. In some embodiments, a TCR alpha intracellular domain comprises Ser-Ser. In some embodiments, a TCR alpha intracellular domain is encoded by a sequence of TCCAGC.

In some embodiments, the intracellular domain comprises a TCR beta intracellular domain. In some embodiments, the TCR beta intracellular domain comprises an amino acid sequence having at least 80% identity, at least 90% identity, or is identical to a sequence of: MAMVKRKDSR (SEQ ID NO: 35). In some embodiments, the TCR beta intracellular domain comprises, or consists essentially of SEQ ID NO: 35. In some embodiments, the TCR beta intracellular domain is encoded by a sequence of

(SEQ ID NO: 36)
ATGGCCATGGTCAAGAGAAAGGATTCCAGA.

In some embodiments, the intracellular signaling domain comprises at least one stimulatory intracellular domain. In some embodiments, the intracellular signaling domain comprises a primary intracellular signaling domain, such as a CD3 delta, CD3 gamma and CD3 epsilon intracellular domain, and one additional stimulatory intracellular domain, for example a co-stimulatory domain. In some embodiments, the intracellular signaling domain comprises a primary intracellular signaling domain, such as a CD3 delta, CD3 gamma and CD3 epsilon intracellular domain, and two additional stimulatory intracellular domains.

Exemplary co-stimulatory intracellular signaling domains include those derived from proteins responsible for co-stimulatory signals, or antigen independent stimulation.

The term “co-stimulatory molecule” refers to the cognate binding partner on a T-cell that specifically binds with a co-stimulatory ligand, thereby mediating a co-stimulatory response by the T-cell, such as, but not limited to, proliferation. Co-stimulatory molecules are cell surface molecules other than antigen receptors. Co-stimulatory molecules and their ligands are required for an efficient immune response. Co-stimulatory molecules include, but are not limited to an MHC class I molecule, BTLA, a Toll ligand receptor, as well as DAP10, DAP12, CD30, LIGHT, OX40, CD2, CD27, CDS, ICAM-1, LFA-1 (CD11a/CD18) 4-1BB (CD137, TNF receptor superfamily member 9), and CD28 molecule (CD28).

A “co-stimulatory domain”, sometimes referred to as “a co-stimulatory intracellular signaling domain” can be the intracellular portion of a co-stimulatory protein. A co-stimulatory domain can be a domain of a co-stimulatory protein that transduces the co-stimulatory signal. A co-stimulatory protein can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, a ligand that specifically binds with CD83, CD4, and the like. The co-stimulatory domain can comprise the entire intracellular portion, or the entire native intracellular signaling domain, of the molecule from which it is derived, or a functional fragment thereof.

In some embodiments, the stimulatory domain comprises a co-stimulatory domain. In some embodiments, the co-stimulatory domain comprises a CD28 or 4-1BB co-stimulatory domain. CD28 and 4-1BB are well characterized co-stimulatory molecules required for full T cell activation and known to enhance T cell effector function. For example, CD28 and 4-1BB have been utilized in chimeric antigen receptors (CARs) to boost cytokine release, cytolytic function, and persistence over the first-generation CAR containing only the CD3 zeta signaling domain. Likewise, inclusion of co-stimulatory domains, for example CD28 and 4-1BB domains, in engineered TCR can increase T cell effector function and specifically allow co-stimulation in the absence of co-stimulatory ligand, which is typically down-regulated on the surface of tumor cells.

In some embodiments, the stimulatory domain comprises a CD28 intracellular domain. In some embodiments, the CD28 intracellular domain comprises an amino acid sequence having at least 85% identity, at least 90% identity, at least 95% identity, at least 96% identity, at least 97% identity, at least 98% identity, at least 99% identity or is identical to a sequence of: RSKRSRLLHSDYMNMTPRRPGPTRKHYQPYAPPRDFAAYRS (SEQ ID NO: 37). In some embodiments, the CD28 intracellular domain comprises, or consists essentially of, RSKRSRLLHSDYMNMTPRRPGPTRKHYQPYAPPRDFAAYRS (SEQ ID NO: 37). In some embodiments, a CD28 intracellular domain is encoded by a nucleotide sequence comprising:

(SEQ ID NO: 38)
AGGAGCAAGCGGAGCAGACTGCTGCACAGCGACTACATGAACATGACCC
CCCGGAGGCCTGGCCCCACCCGGAAGCACTACCAGCCCTACGCCCCTCC
CAGGGATTTCGCCGCCTACCGGAGC.

In some embodiments, the stimulatory domain comprises a 4-1BB intracellular domain. In some embodiments, the 4-1BB intracellular domain comprises an amino acid sequence having at least 85% identity, at least 90% identity, at least 95% identity, at least 96% identity, at least 97% identity, at least 98% identity, at least 99% identity or is identical to a sequence of: KRGRKKLLYIFKQPFMRPVQTTQEEDGCSCRFPEEEEGGCEL (SEQ ID NO: 39). In some embodiments, the 4-1BB intracellular domain comprises, or consists essentially of, KRGRKKLLYIFKQPFMRPVQTTQEEDGCSCRFPEEEEGGCEL (SEQ ID NO: 39). In some embodiments, a 4-1BB intracellular domain is encoded by a nucleotide sequence comprising:

(SEQ ID NO: 40)
AAACGGGGCAGAAAGAAACTCCTGTATATATTCAAACAACCATTTATGA
GGCCAGTACAAACTACTCAAGAGGAAGATGGCTGTAGCTGCCGATTTCC
AGAAGAAGAAGAAGGAGGATGTGAACTG.

### Inhibitory Domains

The disclosure provides inhibitory intracellular domains which can be fused to the transmembrane or intracellular domain of any of the TCR subunits to generate an inhibitory TCR

In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells, and play a pivotal role in attenuating or terminating T cell responses.

Inhibitory domains can be isolated from human tumor necrosis factor related apoptosis inducing ligand (TRAIL) receptor and CD200 receptor 1.

In some embodiments, the inhibitory domain comprises an intracellular domain, a transmembrane or a combination thereof. In some embodiments, the inhibitory domain comprises an intracellular domain, a transmembrane domain, a hinge region or a combination thereof. In some embodiments, the inhibitory domain comprises an immunoreceptor tyrosine-based inhibitory motif (ITIM). In some embodiments, the inhibitory domain comprising an ITIM can be isolated or derived from an immune checkpoint inhibitor such as CTLA-4 and PD-1.

Inhibitory domains can be isolated from human tumor necrosis factor related apoptosis inducing ligand (TRAIL) receptor and CD200 receptor 1. In some embodiments, the inhibitory domain is isolated or derived from a human protein, for example a human TRAIL receptor, CTLA-4, or PD-1 protein. In some embodiments, the TRAIL receptor comprises TR10A, TR10B or TR10D.

Endogenous TRAIL is expressed as a 281-amino acid type II trans-membrane protein, which is anchored to the plasma membrane and presented on the cell surface. TRAIL is expressed by natural killer cells, which, following the establishment of cell-cell contacts, can induce TRAIL-dependent apoptosis in target cells. Physiologically, the TRAIL-signaling system was shown to be essential for immune surveillance, for shaping the immune system through regulating T-helper cell 1 versus T-helper cell 2 as well as “helpless” CD8+ T-cell numbers, and for the suppression of spontaneous tumor formation.

In some embodiments, the inhibitory domain comprises an intracellular domain isolated or derived from a CD200 receptor. The cell surface glycoprotein CD200 receptor 1 (Uniprot ref: Q8TD46) represents another example of an inhibitory intracellular domain of the present invention. This inhibitory receptor for the CD200/OX2 cell surface glycoprotein limits inflammation by inhibiting the expression of proinflammatory molecules including TNF-alpha, interferons, and inducible nitric oxide synthase (iNOS) in response to selected stimuli.

In some embodiments, the engineered receptor comprises an inhibitory domain isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1), programmed cell death 1 (PD-1), Fc gamma receptor IIB (FcgRIIB), killer cell lectin like receptor K1 (NKG2D), CTLA-4, a domain containing a synthetic consensus ITIM, a ZAP70 SH2 domain (e.g., one or both of the N and C terminal SH2 domains), or ZAP70 KI_K369A (kinase inactive ZAP70).

In some embodiments, the inhibitory domain is isolated or derived from a human protein.

In some embodiments, the second, inhibitory receptor comprises a cytoplasmic domain and transmembrane domain isolated or derived from the same protein, for example an ITIM containing protein. In some embodiments, the second, inhibitory receptor comprises a cytoplasmic domain, a transmembrane domain, and an extracellular domain or a portion thereof isolated or derived isolated or derived from the same protein, for example an ITIM containing protein. In some embodiments, the second, inhibitory receptor comprises a hinge region isolated or derived from isolated or derived from the same protein as the intracellular domain and/or transmembrane domain, for example an ITIM containing protein.

In some embodiments, the second engineered receptor is a TCR comprising an inhibitory domain (an inhibitory TCR). In some embodiments, the inhibitory TCR comprises an inhibitory intracellular domain and/or an inhibitory transmembrane domain. In some embodiments, the inhibitory intracellular domain is fused to the intracellular domain of TCR alpha, TCR beta, CD3 delta, CD3 gamma or CD3 epsilon or a portion thereof a TCR. In some embodiments, the inhibitory intracellular domain is fused to the transmembrane domain of TCR alpha, TCR beta, CD3 delta, CD3 gamma or CD3 epsilon.

In some embodiments, the second engineered receptor is a TCR comprising an inhibitory domain (an inhibitory TCR). In some embodiments, the inhibitory domain is isolated or derived from LILRB1.

### LILRB1 Inhibitory Receptors

The disclosure provides a second, inhibitory receptor comprising a LILRB1 inhibitory domain, and optionally, a LILRB1 transmembrane and/or hinge domain, or functional variants thereof. The inclusion of the LILRB1 transmembrane domain and/or the LILRB1 hinge domain in the inhibitory receptor may increase the inhibitory signal generated by the inhibitory receptor compared to a reference inhibitory receptor having another transmembrane domain or another hinge domains. The second, inhibitory receptor comprising the LILRB1 inhibitory domain may be a CAR or TCR, as described herein. Any suitable ligand binding domain, as described herein, may be fused to the LILRB1-based second, inhibitory receptors.

Leukocyte immunoglobulin-like receptor subfamily B member 1 (LILRB1), also known as Leukocyte immunoglobulin-like receptor B1, as well as ILT2, LIR1, MIR7, PIRB, CD85J, ILT-2 LIR-1, MIR-7 and PIR-B, is a member of the leukocyte immunoglobulin-like receptor (LIR) family. The LILRB1 protein belongs to the subfamily B class of LIR receptors. These receptors contain two to four extracellular immunoglobulin domains, a transmembrane domain, and two to four cytoplasmic immunoreceptor tyrosine-based inhibitory motifs (ITIMs). The LILRB1 receptor is expressed on immune cells, where it binds to MHC class I molecules on antigen-presenting cells and transduces a negative signal that inhibits stimulation of an immune response. LILRB1 is thought to regulate inflammatory responses, as well as cytotoxicity, and to play a role in limiting auto-reactivity. Multiple transcript variants encoding different isoforms of LILRB1 exist, all of which are contemplated as within the scope of the instant disclosure.

In some embodiments of the inhibitory receptors described herein, the inhibitory receptor comprises one or more domains isolated or derived from LILRB1. In some embodiments of the receptors having one or more domains isolated or derived from LILRB1, the one or more domains of LILRB1 comprise an amino acid sequence that is at least 80%, at least 90%, at least 95%, at least 96%, at least 97%, at least 98%, at least 99% or is identical to a sequence or subsequence of SEQ ID NO: 65. In some embodiments, the one or more domains of LILRB1 comprise an amino acid sequence that is identical to a sequence or subsequence of SEQ ID NO: 65. In some embodiments, the one or more domains of LILRB1 consist of an amino acid sequence that is at least 80%, at least 90%, at least 95%, at least 96%, at least 97%, at least 98%, at least 99% or is identical to a sequence or subsequence of SEQ ID NO: 65. In some embodiments, the one or more domains of LILRB1 consist of an amino acid sequence that is identical to a sequence or subsequence of SEQ ID NO: 65.

In some embodiments of the receptors having one or more domains isolated or derived from LILRB1, the one or more domains of LILRB1 are encoded by a polynucleotide sequence that is at least 80%, at least 90%, at least 95%, at least 96%, at least 97%, at least 98%, at least 99% or is identical to a sequence or subsequence of SEQ ID NO: 66.

In some embodiments of the receptors having one or more domains of LILRB1, the one or more domains of LILRB1 are encoded by a polynucleotide sequence that is identical to a sequence or subsequence of SEQ ID NO: 66.

In various embodiments, an inhibitory receptor is provided, comprising a polypeptide, wherein the polypeptide comprises one or more of: an LILRB1 hinge domain or functional fragment or variant thereof; an LILRB1 transmembrane domain or a functional variant thereof; and an LILRB1 intracellular domain or an intracellular domain comprising at least one, or at least two immunoreceptor tyrosine-based inhibitory motifs (ITIMs), wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

As used herein an “immunoreceptor tyrosine-based inhibitory motif” or “ITIM” refers to a conserved sequence of amino acids with a consensus sequence of S/I/V/LxYxxI/V/L (SEQ ID NO: 274), or the like, that is found in the cytoplasmic tails of many inhibitory receptors of the immune system. After ITIM-possessing inhibitory receptors interact with their ligand, the ITIM motif is phosphorylated, allowing the inhibitory receptor to recruit other enzymes, such as the phosphotyrosine phosphatases SHP-1 and SHP-2, or the inositol-phosphatase called SHIP.

In some embodiments, the polypeptide comprises an intracellular domain comprising at least one immunoreceptor tyrosine-based inhibitory motif (ITIM), at least two ITIMs, at least 3 ITIMs, at least 4 ITIMs, at least 5 ITIMs or at least 6 ITIMs. In some embodiments, the intracellular domain has 1, 2, 3, 4, 5, or 6 ITIMs.

In some embodiments, the polypeptide comprises an intracellular domain comprising at least one ITIM selected from the group of ITIMs consisting of NLYAAV (SEQ ID NO: 67), VTYAEV (SEQ ID NO: 68), VTYAQL (SEQ ID NO: 69), and SIYATL (SEQ ID NO: 70).

In further particular embodiments, the polypeptide comprises an intracellular domain comprising at least two immunoreceptor tyrosine-based inhibitory motifs (ITIMs), wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

In some embodiments, the intracellular domain comprises both ITIMs NLYAAV (SEQ ID NO: 67) and VTYAEV (SEQ ID NO: 68). In some embodiments, the intracellular domain comprises a sequence at least 95% identical to SEQ ID NO: 71. In some embodiments, the intracellular domain comprises or consists essentially of a sequence identical to SEQ ID NO: 71.

In some embodiments, the intracellular domain comprises both ITIMs VTYAEV (SEQ ID NO: 68) and VTYAQL (SEQ ID NO: 69). In some embodiments, the intracellular domain comprises a sequence at least 95% identical to SEQ ID NO: 72. In some embodiments, the intracellular domain comprises or consists essentially of a sequence identical to SEQ ID NO: 72.

In some embodiments, the intracellular domain comprises both ITIMs VTYAQL (SEQ ID NO: 69) and SIYATL (SEQ ID NO: 70). In some embodiments, the intracellular domain comprises a sequence at least 95% identical to SEQ ID NO: 73. In some embodiments, the intracellular domain comprises or consists essentially of a sequence identical to SEQ ID NO: 73.

In some embodiments, the intracellular domain comprises the ITIMs NLYAAV (SEQ ID NO: 67), VTYAEV (SEQ ID NO: 68), and VTYAQL (SEQ ID NO: 69). In some embodiments, the intracellular domain comprises a sequence at least 95% identical to SEQ ID NO: 74. In some embodiments, the intracellular domain comprises or consists essentially of a sequence identical to SEQ ID NO: 74.

In some embodiments, the intracellular domain comprises the ITIMs VTYAEV (SEQ ID NO: 68), VTYAQL (SEQ ID NO: 69), and SIYATL (SEQ ID NO: 70). In some embodiments, the intracellular domain comprises a sequence at least 95% identical to SEQ ID NO: 75. In some embodiments, the intracellular domain comprises or consists essentially of a sequence identical to SEQ ID NO: 75.

In some embodiments, the intracellular domain comprises the ITIMs NLYAAV (SEQ ID NO: 67), VTYAEV (SEQ ID NO: 68), VTYAQL (SEQ ID NO: 69), and SIYATL (SEQ ID NO: 70). In embodiments, the intracellular domain comprises a sequence at least 95% identical to SEQ ID NO: 76. In some embodiments, the intracellular domain comprises or consists essentially of a sequence identical to SEQ ID NO: 76.

In some embodiments, the intracellular domain comprises a sequence at least 95% identical to the LILRB1 intracellular domain (SEQ ID NO: 81). In some embodiments, the intracellular domain comprises or consists essentially of a sequence identical to the LILRB1 intracellular domain (SEQ ID NO: 81).

LILRB1 intracellular domains or functional variants thereof of the disclosure can have at least 1, at least 2, at least 4, at least 4, at least 5, at least 6, at least 7, or at least 8 ITIMs. In some embodiments, the LILRB1 intracellular domain or functional variant thereof has 2, 3, 4, 5, or 6 ITIMs.

In particular embodiments, the intracellular domain comprises two, three, four, five, or six immunoreceptor tyrosine-based inhibitory motifs (ITIMs), wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

In particular embodiments, the intracellular domain comprises at least three immunoreceptor tyrosine-based inhibitory motifs (ITIMs), wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

In particular embodiments, the intracellular domain comprises three immunoreceptor tyrosine-based inhibitory motifs (ITIMs), wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

In particular embodiments, the intracellular domain comprises four immunoreceptor tyrosine-based inhibitory motifs (ITIMs), wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

In particular embodiments, the intracellular domain comprises five immunoreceptor tyrosine-based inhibitory motifs (ITIMs), wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

In particular embodiments, the intracellular domain comprises six immunoreceptor tyrosine-based inhibitory motifs (ITIMs), wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

In particular embodiments, the intracellular domain comprises at least seven immunoreceptor tyrosine-based inhibitory motifs (ITIMs), wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

The LILRB1 protein has four immunoglobulin (Ig) like domains termed D1, D2, D3 and D4. In some embodiments, the LILRB1 hinge domain comprises an LILRB1 D3D4 domain or a functional variant thereof. In some embodiments, the LILRB1 D3D4 domain comprises a sequence at least 95%, at least 96%, at least 97%, at least 98%, at least 99% or identical to SEQ ID NO: 77. In some embodiments, the LILRB1 D3D4 domain comprises or consists essentially of SEQ ID NO: 77.

In some embodiments, the polypeptide comprises the LILRB1 hinge domain or functional fragment or variant thereof. In embodiments, the LILRB1 hinge domain or functional fragment or variant thereof comprises a sequence at least 95%, at least 96%, at least 97%, at least 98%, at least 99% identical or identical to SEQ ID NO: 84, SEQ ID NO: 77, or SEQ ID NO: 78. In embodiments, the LILRB1 hinge domain or functional fragment or variant thereof comprises a sequence at least 95% identical to SEQ ID NO: 84, SEQ ID NO: 77, or SEQ ID NO: 78.

In some embodiments, the LILRB1 hinge domain comprises a sequence identical to SEQ ID NO: 84, SEQ ID NO: 77, or SEQ ID NO: 78.

In some embodiments, the LILRB1 hinge domain consists essentially of a sequence identical to SEQ ID NO: 84, SEQ ID NO: 77, or SEQ ID NO: 78.

In some embodiments, the transmembrane domain is a LILRB1 transmembrane domain or a functional variant thereof. In some embodiments, the LILRB1 transmembrane domain or a functional variant thereof comprises a sequence at least 95% identical, at least 96% identical, at least 97% identical, at least 98% identical or at least 99% to SEQ ID NO: 85. In some embodiments, the LILRB1 transmembrane domain or a functional variant thereof comprises a sequence at least 95% identical to SEQ ID NO: 85. In some embodiments, the LILRB1 transmembrane domain comprises a sequence identical to SEQ ID NO: 85. In embodiments, the LILRB1 transmembrane domain consists essentially of a sequence identical to SEQ ID NO: 85.

In some embodiments, the transmembrane domain can be attached to the extracellular region of the second, inhibitory receptor, e.g., the antigen binding domain or ligand binding domain, via a hinge, e.g., a hinge from a human protein. For example, in some embodiments, the hinge can be a human immunoglobulin (Ig) hinge, e.g., an IgG4 hinge, a CD8α hinge or an LILRB1 hinge.

In some embodiments, the second, inhibitory receptor comprises an inhibitory domain. In some embodiments, the second, inhibitory receptor comprises an inhibitory intracellular domain and/or an inhibitory transmembrane domain. In some embodiments, the inhibitory domain is isolated or derived from LILR1B.

### Inhibitory Receptors Comprising Combinations of LILRB1 Domains

In some embodiments, the LILRB1-based inhibitory receptors of the disclosure comprise more than one LILRB1 domain or functional equivalent thereof. For example, in some embodiments, the inhibitory receptor comprises an LILRB1 transmembrane domain and intracellular domain, or an LILRB1 hinge domain, transmembrane domain and intracellular domain.

In particular embodiments, the inhibitory receptor comprises an LILRB1 hinge domain or functional fragment or variant thereof, and the LILRB1 transmembrane domain or a functional variant thereof. In some embodiments, the polypeptide comprises a sequence at least 95% identical, at least 96% identical, at least 97% identical, at least 98% identical, at least 99% identical or identical to SEQ ID NO: 79. In some embodiments, the polypeptide comprises a sequence at least 95% identical to SEQ ID NO: 79. In some embodiments, the polypeptide comprises a sequence identical to SEQ ID NO: 79.

In further embodiments, the inhibitory receptor comprises: the LILRB1 transmembrane domain or a functional variant thereof, and an LILRB1 intracellular domain and/or an intracellular domain comprising at least one immunoreceptor tyrosine-based inhibitory motif (ITIM), wherein the ITIM is selected from NLYAAV (SEQ ID NO: 67), VTYAEV (SEQ ID NO: 68), VTYAQL (SEQ ID NO: 69), and SIYATL (SEQ ID NO: 70). In some embodiments, the polypeptide comprises the LILRB1 transmembrane domain or a functional variant thereof, and an LILRB1 intracellular domain and/or an intracellular domain comprising at least two ITIM, wherein each ITIM is independently selected from NLYAAV (SEQ ID NO: 67), VTYAEV (SEQ ID NO: 68), VTYAQL (SEQ ID NO: 69), and SIYATL (SEQ ID NO: 70).

In some embodiments, the inhibitory receptor comprises a LILRB1 transmembrane domain and intracellular domain. In some embodiments, the polypeptide comprises a sequence at least 95% identical, at least 96% identical, at least 97% identical, at least 98% identical, at least 99% identical or identical to SEQ ID NO: 80. In some embodiments, the polypeptide comprises a sequence at least 95% identical to SEQ ID NO: 80. In some embodiments, the polypeptide comprises a sequence identical to SEQ ID NO: 80. In some embodiments, the inhibitory receptor comprises the LILRB1 transmembrane domain and intracellular domain of SEQ ID NO: 80 fused to an extracellular ligand binding domain. In some embodiments, the inhibitory receptor comprises a first polypeptide comprising SEQ ID NO: 80 fused to a TCR alpha variable domain, and a second polypeptide comprising SEQ ID NO: 80 fused to a TCR beta variable domain.

In preferred embodiments, the inhibitory receptor comprises: an LILRB1 hinge domain or functional fragment or variant thereof; an LILRB1 transmembrane domain or a functional variant thereof; and an LILRB1 intracellular domain and/or an intracellular domain comprising at least two immunoreceptor tyrosine-based inhibitory motifs (ITIMs), wherein each ITIM is independently selected from LYAAV (SEQ ID NO: 67), VTYAE (SEQ ID NO: 68), VTYAQL (SEQ ID NO: 69), and SIYATL (SEQ ID NO: 11).

In some embodiments, the inhibitory receptor comprises a sequence at least 95% identical to SEQ ID NO: 82 or SEQ ID NO: 83, or at least 99% identical to SEQ ID NO: 82 or SEQ ID NO: 83, or identical to SEQ ID NO: 82 or SEQ ID NO: 83.

In some embodiments, the polypeptide comprises a sequence at least 99% identical to SEQ ID NO: 79, or at least 99% identical to SEQ ID NO: 79, or identical to SEQ ID NO: 79.

In some embodiments, the polypeptide comprises a sequence at least 99% identical to SEQ ID NO: 80, or at least 99% identical to SEQ ID NO: 80, or identical to SEQ ID NO: 80.

TABLE 13
Polypeptide sequences for illustrative LILRB1-based inhibitory receptors

| Name | Sequence |
|---|---|
| LILRB1 | MTPILTVLICLGLSLGPRTHVQAGHLPKPTLWAEPGSVITQGSPVTLRCQ |
| | GGQETQEYRLYREKKTALWITRIPQELVKKGQFPIPSITWEHAGRYRCYY |
| | GSDTAGRSESSDPLELVVTGAYIKPTLSAQPSPVVNSGGNVILQCDSQV |
| | AFDGFSLCKEGEDEHPQCLNSQPHARGSSRAIFSVGPVSPSRRWWYRC |
| | YAYDSNSPYEWSLPSDLLELLVLGVSKKPSLSVQPGPIVAPEETLTLQCGS |
| | DAGYNRFVLYKDGERDFLQLAGAQPQAGLSQANFTLGPVSRSYGGQY |
| | RCYGAHNLSSEWSAPSDPLDILIAGQFYDRVSLSVQPGPTVASGENVTLL |
| | CQSQGWMQTFLLTKEGAADDPWRLRSTYQSQKYQAEFPMGPVTSAH |
| | AGTYRCYGSQSSKPYLLTHPSDPLELVVSGPSGGPSSPTTGPTSTSGPED |
| | QPLTPTGSDPQSGLGRHLGVVIGILVAVILLLLLLLLLFLILRHRRQGKHW |
| | TSTQRKADFQHPAGAVGPEPTDRGLQWRSSPAADAQEENLYAAVKHT |
| | QPEDGVEMDTRSPHDEDPQAVTYAEVKHSRPRREMASPPSPLSGEFL |
| | DTKDRQAEEDRQMDTEAAASEAPQDVTYAQLHSLTLRREATEPPPSQE |
| | GPSPAVPSIYATLAIHPSQEGPSPAVPSIYATLAIH |
| | SEQ ID NO: 65 |
| LILRB1 hinge- | YGSQSSKPYLLTHPSDPLELVVSGPSGGPSSPTTGPTSTSGPEDQPLTPT |
| transmembrane- | GSDPQSGLGRHLGVVIGILVAVILLLLLLLLLFLILRHRRQGKHWTSTQRK |
| intracellular domain | ADFQHPAGAVGPEPTDRGLQWRSSPAADAQEENLYAAVKHTQPEDG |
| | VEMDTRSPHDEDPQAVTYAEVKHSRPRREMASPPSPLSGEFLDTKDRQ |
| | AEEDRQMDTEAAASEAPQDVTYAQLHSLTLRREATEPPPSQEGPSPAV |
| | PSIYATLAIH |
| | SEQ ID NO: 82 |
| LILRB1 hinge- | VVSGPSGGPSSPTTGPTSTSGPEDQPLTPTGSDPQSGLGRHLGVVIGILV |
| transmembrane- | AVILLLLLLLLLFLILRHRRQGKHWTSTQRKADFQHPAGAVGPEPTDRGL |
| intracellular domain (w/o | QWRSSPAADAQEENLYAAVKHTQPEDGVEMDTRSPHDEDPQAVTYA |
| YGSQSSKPYLLTHPSDPLEL) | EVKHSRPRREMASPPSPLSGEFLDTKDRQAEEDRQMDTEAAASEAPQ |
| | DVTYAQLHSLTLRREATEPPPSQEGPSPAVPSIYATLAIH |
| | SEQ ID NO: 83 |
| LILRB1 hinge domain | YGSQSSKPYLLTHPSDPLELVVSGPSGGPSSPTTGPTSTSGPEDQPLTPT |
| | GSDPQSGLGRHLG |
| | SEQ ID NO: 84 |
| LILRB1 transmembrane | VVIGILVAVILLLLLLLLLFLIL |
| domain | SEQ ID NO: 85 |
| LILRB1 intracellular | RHRRQGKHWTSTQRKADFQHPAGAVGPEPTDRGLQWRSSPAADAQ |
| domain | EENLYAAVKHTQPEDGVEMDTRSPHDEDPQAVTYAEVKHSRPRREMA |
| | SPPSPLSGEFLDTKDRQAEEDRQMDTEAAASEAPQDVTYAQLHSLTLRR |
| | EATEPPPSQEGPSPAVPSIYATLAIH |
| | SEQ ID NO: 81 |
| ITIM1 | NLYAAV |
| | SEQ ID NO: 67 |
| ITIM2 | VTYAEV |
| | SEQ ID NO: 68 |
| ITIM3 | VTYAQL |
| | SEQ ID NO: 69 |
| ITIM4 | SIYATL |
| | SEQ ID NO: 70 |
| ITIM1-2 | NLYAAVKHTQPEDGVEMDTRSPHDEDPQAVTYAEV |
| | SEQ ID NO: 71 |
| ITIM2-3 | VTYAEVKHSRPRREMASPPSPLSGEFLDTKDRQAEEDRQMDTEAAASE |
| | APQDVTYAQL |
| | SEQ ID NO: 72 |
| ITIM3-4 | VTYAQLHSLTLRREATEPPPSQEGPSPAVPSIYATL |
| | SEQ ID NO: 73 |
| ITIM1-3 | NLYAAVKHTQPEDGVEMDTRSPHDEDPQAVTYAEVKHSRPRREMAS |
| | PPSPLSGEFLDTKDRQAEEDRQMDTEAAASEAPQDVTYAQL |
| | SEQ ID NO: 74 |
| ITIM2-4 | VTYAEVKHSRPRREMASPPSPLSGEFLDTKDRQAEEDRQMDTEAAASE |
| | APQDVTYAQLHSLTLRREATEPPPSQEGPSPAVPSIYATL |
| | SEQ ID NO: 75 |
| ITIM1-4 | NLYAAVKHTQPEDGVEMDTRSPHDEDPQAVTYAEVKHSRPRREMAS |
| | PPSPLSGEFLDTKDRQAEEDRQMDTEAAASEAPQDVTYAQLHSLTLRR |
| | EATEPPPSQEGPSPAVPSIYATL |
| | SEQ ID NO: 76 |
| D3D4 domain | YGSQSSKPYLLTHPSDPLEL |
| | SEQ ID NO: 77 |
| Short hinge | VVSGPSGGPSSPTTGPTSTSGPEDQPLTPTGSDPQSGLGRHLG |
| | SEQ ID NO: 78 |
| Hinge-transmembrane | YGSQSSKPYLLTHPSDPLELVVSGPSGGPSSPTTGPTSTSGPEDQPLTPT |
| | GSDPQSGLGRHLGVVIGILVAVILLLLLLLLLFLIL |
| | SEQ ID NO: 79 |
| Transmembrane- | VVIGILVAVILLLLLLLLLFLILRHRRQGKHWTSTQRKADFQHPAGAVGP |
| intracellular domain. | EPTDRGLQWRSSPAADAQEENLYAAVKHTQPEDGVEMDTRSPHDED |
| | PQAVTYAEVKHSRPRREMASPPSPLSGEFLDTKDRQAEEDRQMDTEAA |
| | ASEAPQDVTYAQLHSLTLRREATEPPPSQEGPSPAVPSIYATLAIH |
| | SEQ ID NO: 80 |

### Linkers

In some embodiments, the engineered receptors comprise a linker linking two domains of the engineered receptor. Provided herein are linkers that, in some embodiments, can be used to link domains of the engineered receptors described herein.

The terms “linker” and “flexible polypeptide linker” as used in the context of linking protein domains, for example intracellular domains or domains within an scFv, refers to a peptide linker that consists of amino acids such as glycine and/or serine residues used alone or in combination, to link two domains together.

Any linker may be used and many fusion protein linker formats are known. For example, the linker may be flexible or rigid. Non-limiting examples of rigid and flexible linkers are provided in Chen et al. (Adv Drug Deliv Rev. 2013; 65(10):1357-1369).

The antigen-binding domains described herein may be linked to each other in a random or specified order.

The antigen-binding domains described herein may be linked to each other in any orientation of N to C terminus.

Optionally, a short oligo- or polypeptide linker, for example, between 2 and 40 amino acids (e.g., 2, 3, 4, 5, 6, 7, 8, 9, or 10 amino acids) in length may form the linkage between the domains.

In some embodiments, the linker is a peptide of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 amino acid residues. Non-limiting examples of amino acids found in linkers include Gly, Ser, Glu, Gin, Ala, Leu, Iso, Lys, Arg, Pro, and the like. In some embodiments, the linker is [(Gly)n1Ser]n2, where n1 and n2 may be any number (e.g. n1 and n2 may independently be 1, 2, 4, 5, 6, 7, 8, 9, 10 or more than 10). In some embodiments, n1 is 4.

In some embodiments, the flexible polypeptide linker is a Gly/Ser linker and comprises the amino acid sequence (Gly-Gly-Ser), (Gly-Gly-Gly-Ser, SEQ ID NO: 231), or (Gly-Gly-Gly-Gly-Ser, SEQ ID NO: 226) which can be repeated n times, where n is a positive integer equal to or greater than 1. For example, n=1, n=2, n=3, n=4, n=5, n=6, n=7, n=8, n=9 and n=10. In some embodiments, the flexible polypeptide linkers include, but are not limited to, GGS, GGGGS (SEQ ID NO: 226), GGGGS GGGGS (SEQ ID NO: 227), GGGGS GGGGS GGGGS (SEQ ID NO: 228), GGGGS GGGGS GGGGS GG (SEQ ID NO: 229) or GGGGS GGGGS GGGGS GGGGS (SEQ ID NO: 230).

In some embodiments, the linkers include multiple repeats of (Gly Gly Ser), (Gly Ser) or (Gly Gly Gly Ser (SEQ ID NO: 231)). Also included within the scope of the invention are linkers described in WO2012/138475 (incorporated herein by reference).

In some embodiments, the linker sequence comprises a long linker (LL) sequence. In some embodiments, the long linker sequence comprises GGGGS (SEQ ID NO: 226), repeated four times. In some embodiments, a GGGGS GGGGS GGGGS GGGGS (SEQ ID NO: 230) is used to link intracellular domains in a TCR alpha fusion protein of the disclosure.

In some embodiments, the long linker sequence comprises GGGGS (SEQ ID NO: 226), repeated three times. In some embodiments, a GGGGS GGGGS GGGGS (SEQ ID NO: 228) is used to link intracellular domains in a TCR beta fusion protein of the disclosure.

In some embodiments, the linker sequence comprises a short linker (SL) sequence. In some embodiments, the short linker sequence comprises GGGGS (SEQ ID NO: 226).

In some embodiments, a glycine-serine doublet can be used as a suitable linker.

In some embodiments, domains are fused directly to each other via peptide bonds without use of a linker.

### Assays

Provided herein are assays that can be used to measure the activity of the engineered receptors of the disclosure.

The activity of engineered receptors can be assayed using a cell line engineered to express a reporter of receptor activity such as a luciferase reporter. Exemplary cell lines include Jurkat T cells, although any suitable cell line known in the art may be used. For example, Jurkat cells expressing a luciferase reporter under the control of an NFAT promoter can be used as effector cells. Expression of luciferase by this cell line reflects TCR-mediated signaling.

The reporter cells can be transfected with each of the various fusion protein constructs, combinations of fusion protein constructs or controls described herein.

Expression of the fusion proteins in reporter cells can be confirmed by using fluorescently labeled MHC tetramers, for example Alexa Fluor 647-labeled NY-ESO-1-MHC tetramer, to detect expression of the fusion protein.

To assay the activity of engineered receptors, target cells are loaded with antigen prior to exposure to the effector cells comprising the reporter and the engineered receptor. For example, target cells can be loaded with antigen at least 12, 14, 16, 18, 20, 22 or 24 hours prior to exposure to effector cells. Exemplary target cells include A375 cells, although any suitable cells known in the art may be used. In some cases, target cells can be loaded with serially diluted concentrations of an antigen, such as NY-ESO-1 peptide. The effector cells can then be co-cultured with target cells for a suitable period of time, for example 6 hours. Luciferase is then measured by luminescence reading after co-culture. Luciferase luminescence can be normalized to maximum and minimum intensity to allow comparison of activating peptide concentrations for each engineered receptor construct.

Provided herein are methods of determining the relative EC50 of engineered receptors of the disclosure. As used herein, “EC50” refers to the concentration of an inhibitor or agent where the response (or binding) is reduced by half. EC50s of engineered receptors of the disclosure refer to concentration of antigen where binding of the engineered receptor to the antigen is reduced by half. Binding of the antigen, or probe to the engineered receptor can be measured by staining with labeled peptide or labeled peptide-MHC complex, for example MHC:NY-ESO-1 pMHC complex conjugated with fluorophore. EC50 can be obtained by nonlinear regression curve fitting of reporter signal with peptide titration. Probe binding and EC50 can be normalized to the levels of benchmark TCR without a fusion protein, e.g. NY-ESO-1 (clone 1G4).

### Polynucleotides

The disclosure provides polynucleotides encoding the sequence(s) of the engineered receptors described herein.

In some embodiments, the sequence of the first and/or second fusion protein is operably linked to a promoter. In some embodiments, the sequence encoding the first fusion protein is operably linked to a first promoter, and the sequence encoding a second fusion protein is operably linked to a second promoter.

The disclosure provides vectors comprising the polynucleotides described herein.

The disclosure provides vectors encoding the coding sequence or sequences of any of the engineered receptors described herein. In some embodiments, the sequence of the first and/or second fusion protein is operably linked to a promoter. In some embodiments, the sequence encoding the first fusion protein is operably linked to a first promoter, and the sequence encoding a second fusion protein is operably linked to a second promoter.

In some embodiments, the first engineered receptor is encoded by a first vector and the second engineered receptor is encoded by second vector. In some embodiments, both engineered receptors are encoded by a single vector.

In some embodiments, the first and second receptors are encoded by a single vector. Methods of encoding multiple polypeptides using a single vector will be known to persons of ordinary skill in the art, and include, inter alia, encoding multiple polypeptides under control of different promoters, or, if a single promoter is used to control transcription of multiple polypeptides, use of sequences encoding internal ribosome entry sites (IRES) and/or self-cleaving peptides. Exemplary self-cleaving peptides include T2A, P2A, E2A and F2A self-cleaving peptides. In some embodiments, the T2A self-cleaving peptide comprises a sequence of EGRGSLLTCGDVEENPGP (SEQ ID NO: 271). In some embodiments, the P2A self-cleaving peptide comprises a sequence of ATNFSLLKQAGDVEENPGP (SEQ ID NO: 192). In some embodiments, the E2A self-cleaving peptide comprises a sequence of QCTNYALLKLAGDVESNPGP (SEQ ID NO: 272). In some embodiments, the F2A self-cleaving peptide comprises a sequence of VKQTLNFDLLKLAGDVESNPGP (SEQ ID NO: 273).

In some embodiments, the vector is an expression vector, i.e. for the expression of the fusion protein in a suitable cell.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

The expression of natural or synthetic nucleic acids encoding fusion proteins is typically achieved by operably linking a nucleic acid encoding the fusion protein or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The polynucleotides encoding the fusion proteins can be cloned into a number of types of vectors. For example, the polynucleotides can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a fusion protein, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected or transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Immune Cells

Provided herein are immune cells comprising the polynucleotides, vectors, fusion proteins and engineered receptors described herein.

As used herein, the term “immune cell” refers to a cell involved in the innate or adaptive (acquired) immune systems. Exemplary innate immune cells include phagocytic cells such as neutrophils, monocytes and macrophages, Natural Killer (NK) cells, polymophonuclear leukocytes such as neutrophils eosinophils and basophils and mononuclear cells such as monocytes, macrophages and mast cells. Immune cells with roles in acquired immunity include lymphocytes such as T-cells and B-cells.

As used herein, a “T-cell” refers to a type of lymphocyte that originates from a bone marrow precursor that develops in the thymus gland. There are several distinct types of T-cells which develop upon migration to the thymus, which include, helper CD4+ T-cells, cytotoxic CD8+ T cells, memory T cells, regulatory CD4+ T-cells and stem memory T-cells. Different types of T-cells can be distinguished by the ordinarily skilled artisan based on their expression of markers. Methods of distinguishing between T-cell types will be readily apparent to the ordinarily skilled artisan.

In some embodiments, the engineered immune cell expresses the first and second receptors at a ratio of about 100:1 to 1:100 of first receptor to second receptor. In some embodiments, the engineered immune cell expresses the first and second receptors at a ratio of about 50:1 to 1:50 of first receptor to second receptor. In some embodiments, the engineered immune cell expresses the first and second receptors at a ratio of about 10:1 to 1:10 of first receptor to second receptor. In some embodiments, the engineered immune cell expresses the first and second receptors at a ratio of about 5:1 to 1:5 of first receptor to second receptor. In some embodiments, the engineered immune cell expresses the first and second receptors at a ratio of about 3:1 to 1:3 of first receptor to second receptor. In some embodiments, the engineered immune cell expresses the first and second receptors at a ratio of about 2:1 to 1:2 of first receptor to second receptor. In some embodiments, the engineered immune cell expresses the first and second receptors at a ratio of about 1:1.

In some embodiments, the engineered immune cell comprising the engineered receptors of the disclosure is a T cell. In some embodiments, the T cell is an effector T cell or a regulatory T cell.

Methods transforming populations of immune cells, such as T cells, with the vectors of the instant disclosure will be readily apparent to the person of ordinary skill in the art. For example, CD3+ T cells can be isolated from PBMCs using a CD3+ T cell negative isolation kit (Miltenyi), according to manufacturer's instructions. T cells can be cultured at a density of 1×10\hat{ }6 cells/mL in X-Vivo 15 media supplemented with 5% human A/B serum and 1% Pen/strep in the presence of CD3/28 Dynabeads (1:1 cell to bead ratio) and 300 Units/mL of IL-2 (Miltenyi). After 2 days, T cells can be transduced with viral vectors, such as lentiviral vectors using methods known in the art. In some embodiments, the viral vector is transduced at a multiplicity of infection (MOI) of 5. Cells can then be cultured in IL-2 or other cytokines such as combinations of IL-7/15/21 for an additional 5 days prior to enrichment. Methods of isolating and culturing other populations of immune cells, such as B cells, or other populations of T cells, will be readily apparent to the person of ordinary skill in the art. Although this method outlines a potential approach it should be noted that these methodologies are rapidly evolving. For example excellent viral transduction of peripheral blood mononuclear cells can be achieved after 5 days of growth to generate a >99% CD3+ highly transduced cell population.

Methods of activating and culturing populations of T cells comprising the engineered TCRs, CARs, fusion proteins or vectors encoding the fusion proteins of the instant disclosure, will be readily apparent to the person of ordinary skill in the art.

Whether prior to or after genetic modification of T cells to express an engineered TCR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, 10,040,846; and U.S. Pat. Appl. Pub. No. 2006/0121005.

In some embodiments, T cells of the instant disclosure are expanded and activated in vitro. Generally, the T cells of the instant disclosure are expanded in vitro by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In some embodiments, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. In some embodiments, a ratio of 1:1 cells to beads is used. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached to contact the T cells. In one embodiment the cells (for example, CD4+ T cells) and beads (for example, DYNABEADS CD3/CD28 T paramagnetic beads at a ratio of 1:1) are combined in a buffer. Again, those of ordinary skill in the art can readily appreciate any cell concentration may be used. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. In some embodiments, cells that are cultured at a density of 1×10⁶ cells/mL are used.

In some embodiments, the mixture may be cultured for several hours (about 3 hours) to about 14 days or any hourly integer value in between. In another embodiment, the beads and T cells are cultured together for 2-3 days. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some embodiments, the media comprises X-VIVO-15 media supplemented with 5% human A/B serum, 1% penicillin/streptomycin (pen/strep) and 300 Units/ml of IL-2 (Miltenyi).

The T cells are maintained under conditions necessary to support growth, for example, an appropriate temperature (e.g., 37° C.) and atmosphere (e.g., air plus 5% C02).

In some embodiments, the T cells comprising engineered TCRs of the disclosure are autologous. Prior to expansion and genetic modification, a source of T cells is obtained from a subject. Immune cells such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation.

In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In alternative embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed and the cells directly resuspended in culture media.

In some embodiments, immune cells such as T cells are isolated from peripheral blood lymphocytes by lysing the red blood cells and depleting the monocytes, for example, by centrifugation through a PERCOLL™ gradient or by counterflow centrifugal elutriation. Specific subpopulations of immune cells, such as T cells, B cells, or CD4+ T cells can be further isolated by positive or negative selection techniques. For example, in one embodiment, T cells are isolated by incubation with anti-CD4-conjugated beads, for a time period sufficient for positive selection of the desired T cells.

Enrichment of an immune cell population, such as a T cell population, by negative selection can be accomplished with a combination of antibodies directed to surface markers unique to the negatively selected cells. One method is cell sorting and/or selection via negative magnetic immune-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD 11 b, CD 16, HLA-DR, and CD8.

For isolation of a desired population of immune cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads.

In some embodiments, the cells may be incubated on a rotator for varying lengths of time at varying speeds at either 2-10° C. or at room temperature.

T cells for stimulation, or PBMCs from which immune cells such as T cells are isolated, can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

### Pharmaceutical Compositions

The disclosure provides pharmaceutical compositions comprising immune cells comprising the engineered receptors of the disclosure and a pharmaceutically acceptable diluent, carrier or excipient.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; and preservatives.

### Methods of Treating Disease

Provided herein are methods of treating a subject in need thereof, comprising administering to the subject a therapeutically effective amount of a composition comprising immune cells comprising the engineered receptors of the disclosure. The immune cells express both engineered receptors in the same cell.

In some embodiments, the subject in need thereof has cancer. Cancer is a disease in which abnormal cells divide without control and spread to nearby tissue. In some embodiments, the cancer comprises a liquid tumor or a solid tumor. Exemplary liquid tumors include leukemias and lymphomas. Further cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Exemplary solid tumors include sarcomas and carcinomas. Cancers can arise in virtually an organ in the body, including blood, bone marrow, lung, breast, colon, bone, central nervous system, pancreas, prostate and ovary. Further cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

Any cancer wherein a plurality of the cancer cells express the first, activator ligand and do not express the second, inhibitor ligand is envisaged as within the scope of the instant disclosure. For example, CEA positive cancers that can be treated using the methods described herein include colorectal cancer, pancreatic cancer, esophageal cancer, gastric cancer, lung adenocarcinoma, head and neck cancer, diffuse large B cell cancer or acute myeloid leukemia cancer.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 100% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5% more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20/o; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

### Kits and Articles of Manufacture

The disclosure provides kits and articles of manufacture comprising the polynucleotides and vectors encoding the engineered receptors described herein, and immune cells comprising the engineered receptors described herein. In some embodiments, the kit comprises articles such as vials, syringes and instructions for use.

In some embodiments, the kit comprises a polynucleotide or vector comprising a sequence encoding one or more engineered receptors of the disclosure.

In some embodiments, the kit comprises a plurality of immune cells comprising an engineered receptor as described herein. In some embodiments, the plurality of immune cells comprises a plurality of T cells.

### EXAMPLES

### Example 1: Selection of Activator Target Ligands

The inventors surveyed the GTex gene expression database (gtexportal.org/home/) for activator ligands. Activator ligands should have the following properties: first, One type of activator ligand should have high surface expression, which confers the potential to deliver large activation signals. Alternatively, activators such as MiHAs can have low density on the cell surface. Second, activator ligands can have essential cellular functions, which prevents alleles of the activator ligands being lost due to aneuploidy in tumor cells, and makes them less likely to undergo mutagenesis during the evolution of the tumor. Lastly, activator ligands should be present on all tumor cells. Activator ligands can be expressed on all cells, if the inhibitor ligand is also expressed on all cells except the target cells. Activators should also be expressed on cancer cells. Activators, when used in combination with inhibitors can be widely expressed, for example on all cells.

FIG. 4A shows the RNA expression profile of an exemplary activator ligand, the transferrin receptor (TFRC). As seen in FIG. 4A, expression of TFRC at the RNA level is ubiquitous and relatively even. Further, TFRC is an essential gene: loss of function homozygous TFRC mutations are embryonic lethal in mice.

FIG. 4B shows the expression profiles of a candidate blocker, HLA-A, and candidate activator, HLA-B. As can be seen in FIG. 4B, candidate activator and blocker HLA class I expression tracks together, easing the challenging of optimizing activator and blocker pairs.

### Example 2: Selection of Inhibitor Target Ligands that are Lost in Cancer Cells

### Loss of Heterozygosity

One potential pool of inhibitor ligands are ligands that are lost in tumor cells due to loss of heterozygosity. In an analysis of 3131 tumor samples across 26 histological types, Beroukhim et al. found that in a typical tumor, 25% of the genome is affected by arm number single copy number alterations (duplications and deletions), and 10% of the genome is affected by focal single copy number alterations, with 2% overlap. (Beroukhim et al, Nature 463:899-905 (2010)). Further, many of the LOH regions overlap between tumor types, and only 22% of regions are unique to one tumor type. For example, Beroukhim et al. found that 80% of amplification peaks and 78% of deletion peaks were common to the 17 most represented tumor types. Thus, alleles that are lost to LOH that can be selectively bound by an inhibitor LBD are potential inhibitor targets that are not expressed by target cells.

The inventors surveyed the Cancer Genome Atlas Program (http://portals.broadinstitute.org/tcga/home) for potential inhibitor ligands that were lost in cancers through loss of heterozygosity. The dataset all_cancers dataset (all_cancers) consisted of 10,844 cancer samples from 33 cancer types. One type of inhibitor ligands should have the following properties: first, inhibitor ligands should have high, homogeneous surface expression across tissues. This confers the ability to deliver a large, even-handed inhibitory signal. Inhibitor ligands should be absent or polymorphic in many tumors. Further, it should be easy to distinguish loss of the inhibitor ligand in tumor cells via conventional methods such as antibody stains or genetic analysis. Other types of inhibitor ligands, such as MiHAs, can have low surface expression.

One pool of inhibitor ligands are major histocompatibility complex (MHC) alleles that are lost through LOH in cancer cells. Use of these alleles as inhibitor ligands does not require a peptide MHC target (pMHC), for example, a pan HLA-A*02 allele can be used.

### Loss of Y Chromosome

Adult male-expressed Y chromosome genes are potential inhibitor ligands through loss of Y chromosome. There are at least 60 protein coding genes on the Y chromosome. Several Y chromosome genes are expressed broadly in adult males and may be lost in cancers via loss of Y chromosome. Several other broadly expressed cytoplasmic proteins are pMHC inhibitor candidates (e.g., TMSB4Y, EIF1AY). NLGN4Y is a Type I integral membrane protein expressed broadly in males, and also a candidate.

### Example 3: Targeting Cells Lacking Surface Antigen with Paired A and B Receptors

We show that the targeting system for loss of heterozygosity works in vitro and in a mouse cancer model.

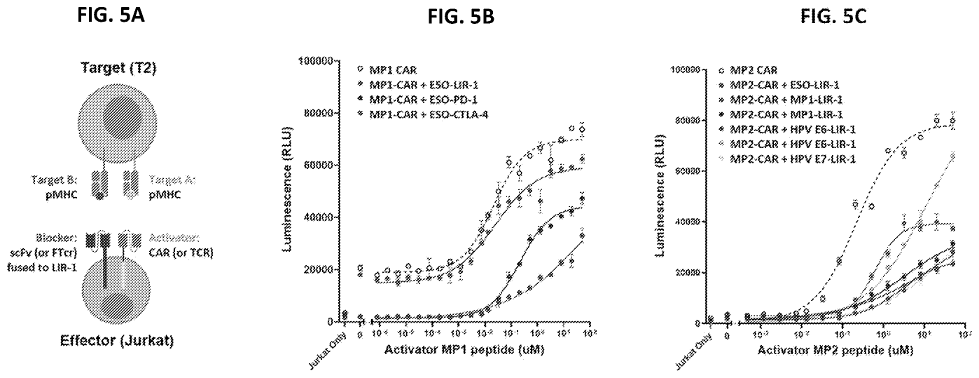

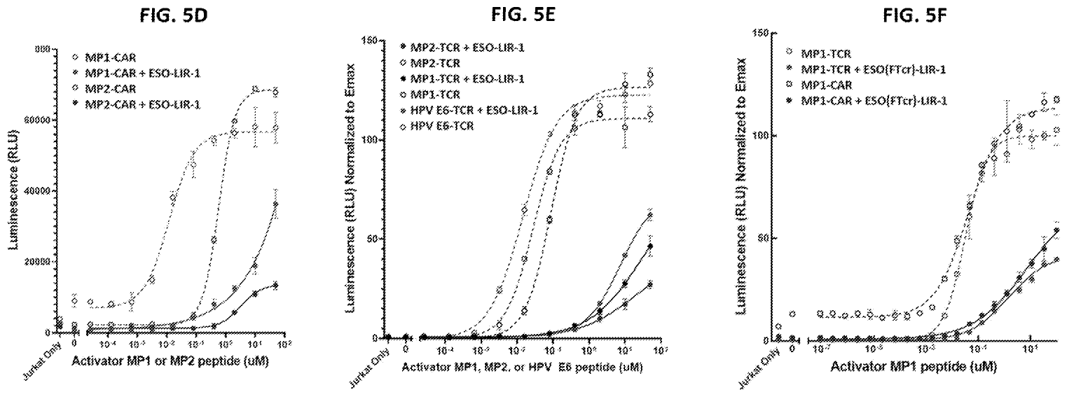

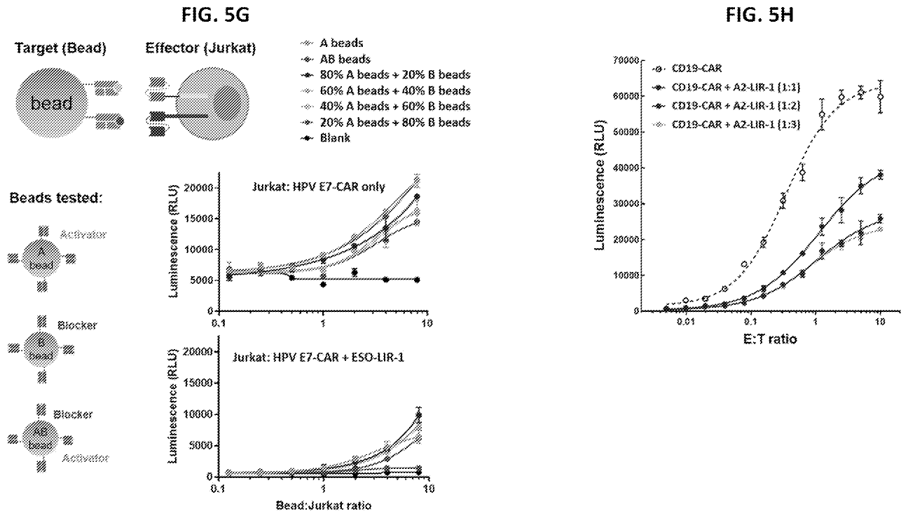

Discrimination between normal and tumor cells depends on two functions: (i) an activator (“A”) receptor that recognizes an epitope on the surface of normal cells that is also retained on the tumor; and, (ii) a blocker (“B”) receptor that recognizes a second surface epitope on an allelic product lost from the tumor cell. In this Example, we used peptide-MHC (pMHC) targets for both the A and B (see FIG. 5A):
a chimeric antigen receptor comprising an scFv against HLA-A*02-MAGE-A3 (FLWGPRALV) pMHC as the A receptor; and
a chimeric antigen receptor comprising an scFv that binds HLA-A*02-NY-ESO-1 (SLLMWITQC/V) as the B receptor and comprising a PD-1 intracellular domain (ICD), a CTLA-4 intracellular domain (ICD) or a LILRB1 (LIR1) intracellular domain (ICD).

Each blocker (B) receptor, with a PD-1 ICD or a CTLA-4-ICD, mediated a shift in EC50 of activation in Jurkat cells of <10×, measured by titration of peptides loaded on T2 cells as stimulus (FIG. 5B). Surprisingly, B receptors comprising a NY-ESO-1 LBD and the intracellular, transmembrane and hinge domains of the LIR-1 (LILRB1) receptor mediated an EC50 shift of >5,000× (also FIG. 5B). Titration of unrelated control HLA-A*02-binding peptides other than SLLMWITQC/V provided an estimate of the shift caused by competition of loaded peptides on T2 cells for available HLA molecules, a contribution to the total shift typically <10× (FIG. 8). For the EC50 shift values reported here, we typically compare to EC50s of activator-only constructs.

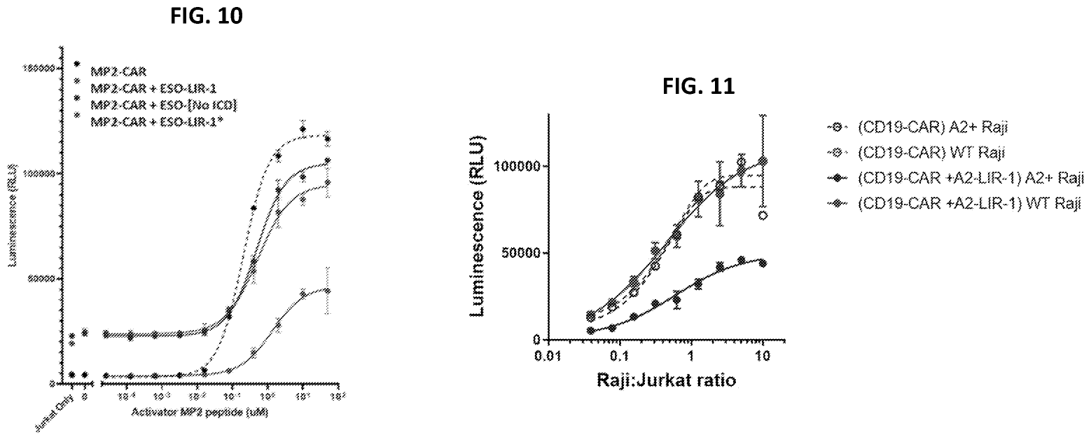

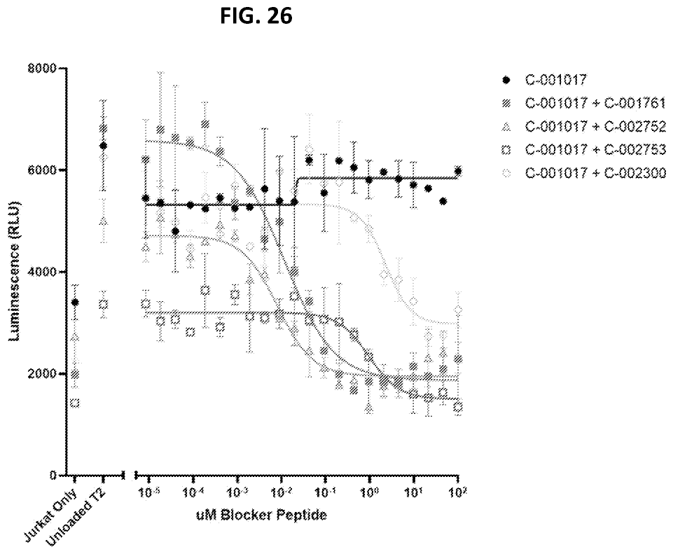

For four different pMHC targets, a total of six different scFvs grafted onto LIR-1 mediated dramatic shifts in EC50, ranging from 10 to 1,000× (FIG. 5C). The degree of EC50 shift (i.e., blocking strength) correlated with the EC50 of the scFv when fused to a standard CAR (data not shown). LIR-1 B signaling blocked A signaling from multiple A targets and scFvs (FIG. 5D, FIG. 26). The blockade was ligand-dependent (FIG. 9). Control B receptors with a LBD, but which lack an ICD or contain mutations in key elements of the ICD do not block activation by A receptor signal. (FIG. 10). Engineered T cells with A receptor and B receptor function across multiple target antigens and antigen binding domains (i.e., LBD sequences).

The LIR-1 ICD also functions when fused to a T cell receptor (TCR) extracellular domain with three different pMHC targets (see Methods). TCRs against three different pMHC targets, two from MAGE-A3 and one form HPV, were assayed. In every case, a LIR-1 based B receptor shifted the activation EC50 by large amounts, ranging from 1,000 to 10,000×. LIR-1 based B receptors with an NY-ESO-1 TCR variable domain LBD “ESO (Ftcr)” fused to it were also able to block activation by a CAR or TCR. This included the following receptor pairs
1. An activator (A) TCR comprising a TCR LBD (“MP1-TCR”) that binds MAGE-A3_{FLWGPRALV} peptide:MHC complexes was blocked by a B receptor comprising an scFv NY-ESO-1 scFv LBD (“ESO”) and a LIR-1 ICD, which shifted the activation EC50 by a large amount (FIG. 5E);
2. An A TCR comprising a second TCR LBD (“MP2-TCR”) that binds the MAGE-A3 _{MPKVAELVHFL} peptide:MHC complexes was blocked by a B receptor comprising an scFv NY-ESO-1 scFv LBD (“ESO”) and a LIR-1 ICD, which shifted the activation EC50 by a large amount (FIG. 5E);
3. An A TCR comprising a TCR LBD (“HPV E6-TCR”) that binds an HPV_{TIHDNLECV} peptide:MHC complex was blocked by a B receptor comprising an scFv NY-ESO-1 scFv LBD (“ESO”) and a LIR-1 ICD, which shifted the activation EC50 by large amounts (FIG. 5E);
4. An A TCR comprising a TCR LBD (“MP1-TCR”) that binds a MAGE-A3_{FLWGPRALV} peptide:MHC complexes was blocked by a B receptor comprising an NY-ESO-1 TCR LBD (“ESO(Ftcr)”), and a LIR-1 ICD, This blocker shifted the activation EC50 by large amounts (FIG. 5F);
5. An A CAR comprising a scFv LBD (“MP1-CAR”) that binds the MAGE-A3 _{FLWGPRALV} peptide:MHC complexes was blocked by a B receptor comprising an TCR NY-ESO-1 TCR variable domain LBD (“ESO(Ftcr)”), and a LIR-1 ICD. This blocker shifted the activation EC50 by large amounts (FIG. 5F).

### Confirming Cis Effect

Engineered effector cells should discriminate potential target cells that are A+ only, i.e. display only the activator, from those that are dual A+ and B+. To affirm our receptor system works as intended, target-loaded beads roughly the size of cells (d˜2.8 μm) were tested with engineered effector cells (Jurkat cells) having A receptor and B receptor (FIG. 5G). Effector cells were indeed activated by a mixture of A+ and B+ beads, even when the A+ beads comprised only 20% of the total beads. This confirms effector cells are able to recognize targets having loss of heterozygosity (represented by A+ beads) in a mixed population comprising normal cells (represented by B+ beads).

### Confirming Target Concentration Independence

In patients, target density will vary depending on the expression levels of the A target and B target. We confirmed the system works with both high density and low density targets, both when A target density is varied (data not shown) and when B target density is varied (FIG. 5H). In FIG. 5H, scFvs that bound either the B-cell marker CD19 or HLA-A*02 in a peptide-independent fashion were tested. These non-pMHC targets represent surface antigens that can extend into the realm of 100,000 epitopes/cell. In this case, the ratio of A to B module expression was varied using different DNA concentrations in transient transfection assays. Emax shifts of over 10× were observed. These experiments showed that the properties of the dual receptor system observed for pMHC targets were generally the same for high-density targets.

### B Receptor Function in Primary T Cells

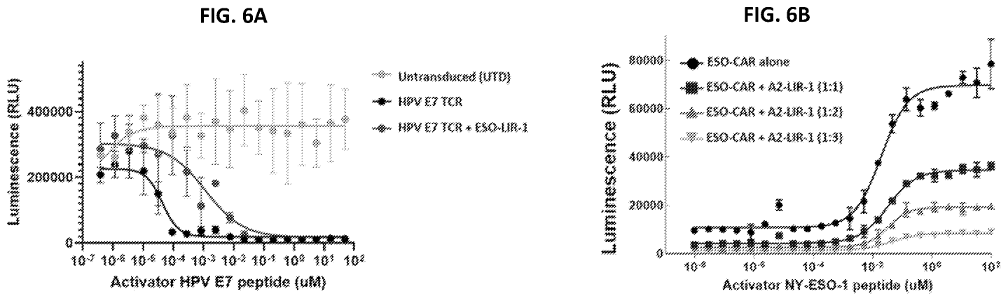

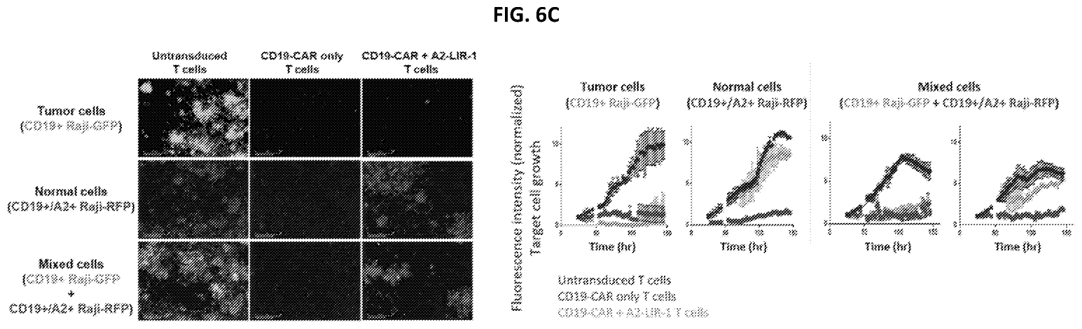

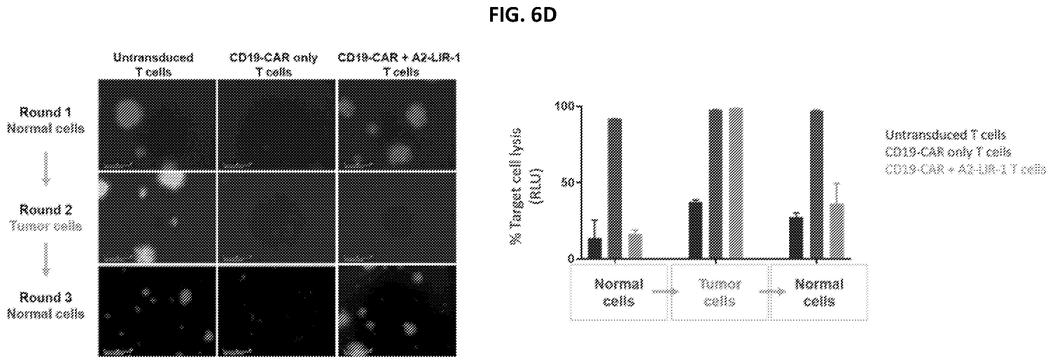

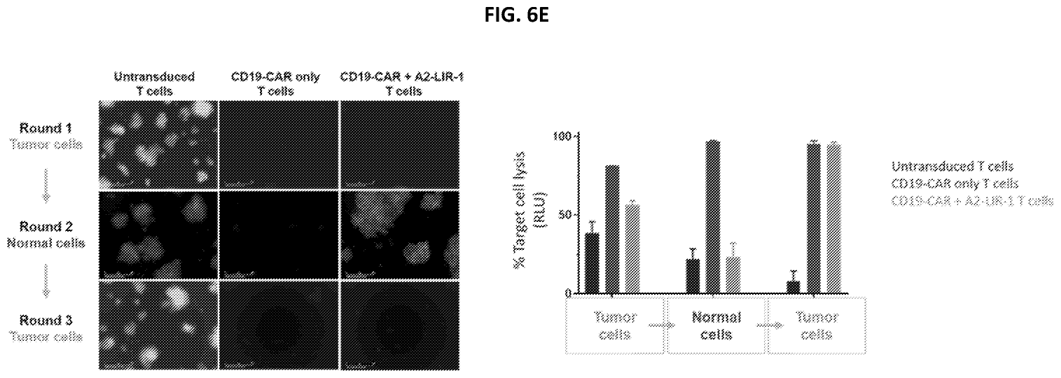

MCF7 tumor cells expressing Renilla luciferase (Biosettia) loaded with a titration of target peptide were used as target cells, with the luciferase as the readout for cell viability. Primary T cells were transduced with an HPV TCR as the A receptor (“HPV E7 TCR”) and a B receptor comprising an anti-NY-ESO-1 scFv fused to a LIR-1 hinge, transmembrane domain and ICD (“ESO-LIR-1”), or not transduced (“Untransduced”). Transduced T cells were enriched via physical selection using beads coupled to HLA-A*02 tetramers that bind to the B receptor LBD. To vary target concentration, the target cells were loaded with varying amounts of HPV peptide. Primary T cells were activated in a dose dependent matter. Expression of the B receptor shifted the EC50 curve by ˜100× (FIG. 6A). A similar result was obtained for an anti-NY-ESO-1 CAR A receptor paired with B receptor comprising an anti-HLA-A*02 LBD and an LIR-1 hinge, transmembrane domain and ICD at various ratios of A receptor to B receptor (achieved by transfecting various activator:blocker DNA ratios) in Jurkat cells (FIG. 6B). This result was confirmed with a CD19 CAR activator paired with an HLA-A*02 blocker in T cells (FIG. 6C). Thus, the basic function of the activator and blocker receptor pair was reproduced in primary T cells, despite their complexity, heterogeneity and donor-to-donor variability.

### Example 4: Targeting Loss of Heterozygosity with Paired A and B Receptors

The HLA locus is polymorphic with only a subset of the population having the HLA-A*02 allele. A ligand-binding domain that binds MHC of the HLA*A02 allele independent of loaded peptide (a “pan HLA-A*02” LBD) may be used to target tumors in subjects heterozygous for HLA and having LOH of the HLA-A*02 allele in tumor cells.

An HLA-A*02-specific scFv was fused to the LIR-1 module and shown to function as a blocker in the presence of pMHC-dependent activators (ESO-CAR, FIG. 6B) in Jurkat cells. Furthermore, in primary T cells expressing both an A receptor comprising an anti-CD19 scFv and a B receptor comprising an HLA-A*02-specific scFv and a LIR-1 LBD, the B receptor blocked the A receptor as desired (FIG. 6C).

Raji target cells that are CD19+ and negative for HLA-A*02 can be used to model tumor cells that have lost HLA-A*02 through LOH. The same cell line stably expressing HLA-A*02 can be used as a model of normal cells. Raji cell lines activated Jurkat effector cells expressing a CD19 CAR and the HLA-A*02 LIR-1 blocker if the Raji target cells expressed CD19 only. When the Raji target cells were transfected with a polynucleotide encoding HLA-A*02, activation of the Jurkat effector cells was blocked (FIG. 11).

As described above, the A receptor binding CD19 and B receptor binding HLA-A*02 worked in primary T cells as well as Jurkat cells. Engineered T cells killed CD19-expressing Raji cells in the absence of HLA-A*02 expression (FIG. 6C, upper panels). Raji cells that expressed both CD19 and HLA-A*02 were killed by T cells expressing only the activating module, but blocked from both gamma-interferon (IFNg) secretion (data not shown) and cytotoxicity when co-cultured with T cells expressing both activator and blocker modules (FIG. 6C, middle panels). Primary T cells bearing the activator and blocker modules distinguished CD19+“tumor” (FIG. 6C, lower panels) from CD19+/HLA-A*02+“normal” cells (FIG. 6C, right panels) in a mixed culture.

A T cell therapeutic based on the activator and blocker mechanism should be able to function reversibly, i.e. be able to cycle from a state of blockade to activation and back to blockade. Effector cells were co-cultured with multiple rounds of Raji cells that were either CD19+ or D19+/HLA-A2*02+, which were removed from culture between rounds. As desired, effector cells exposed to normal cells were not activated when exposed to Raji target cells for both block-kill-block (FIG. 6D) and kill-block-kill programs of target cell exposure (FIG. 6E). Effector T cells were able to cycle from a state of block to cytotoxicity and back, depending on the target cells to which they were exposed.

### Example 5: In Vivo Targeting of Loss of Heterozygosity with Paired A and B Receptors

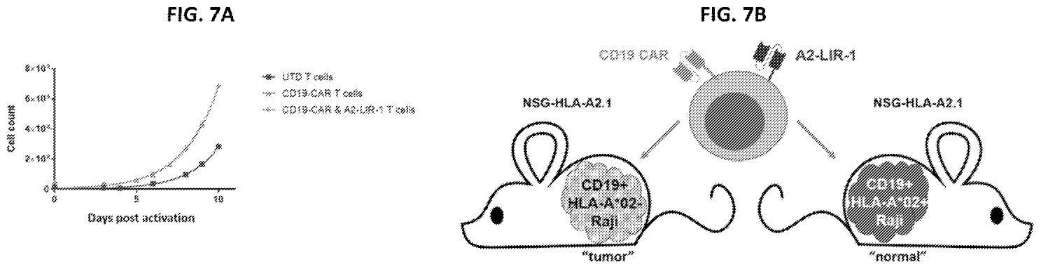

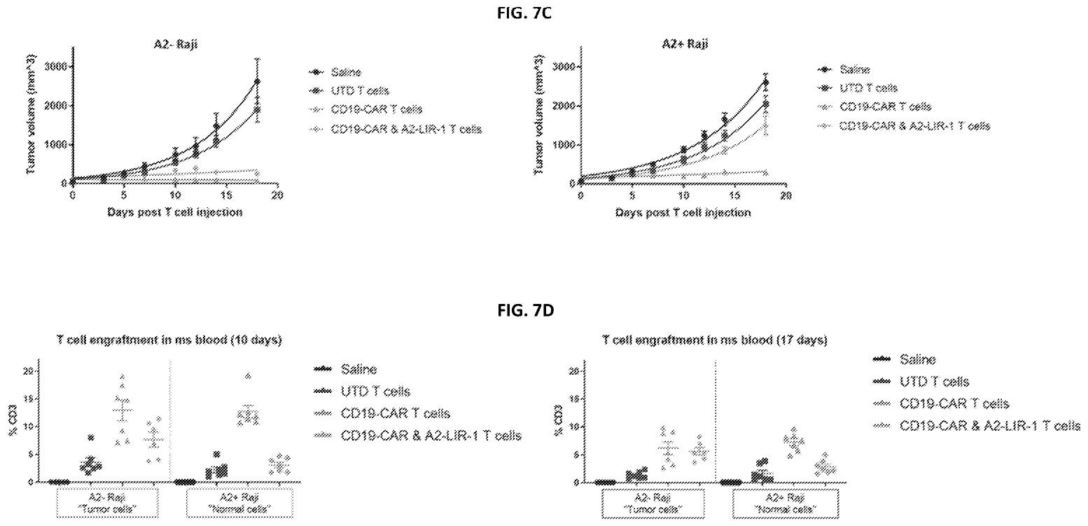

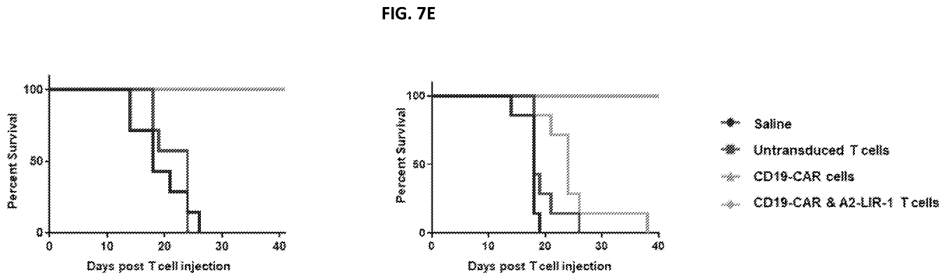

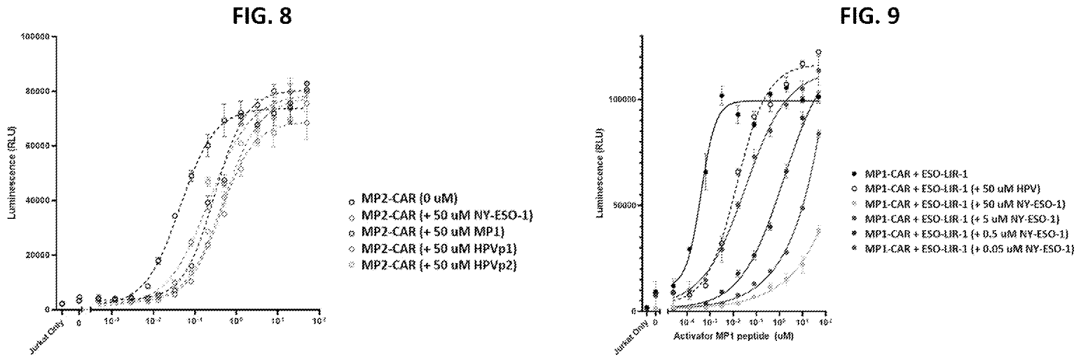

To prepare for in vivo experiments, we showed that the CD19/HLA-A*02 activator/blocker pair engineered in primary T cells allowed expansion in vitro to large numbers using standard CD3/CD28 stimulation (FIG. 7A). Thus, a cell product can be produced in sufficient quantity for use in patients as a therapeutic.

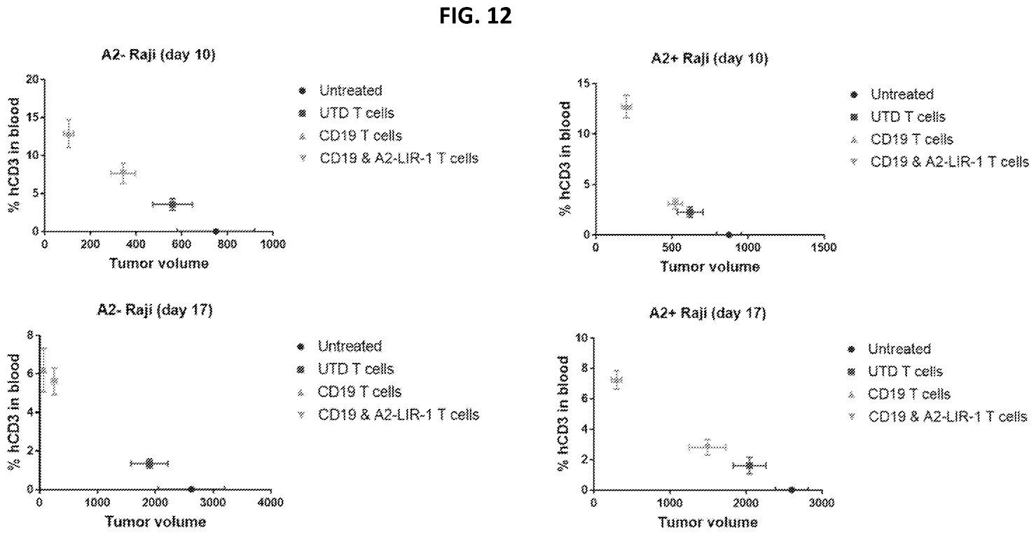

A CD19+/HLA-A*02+ or CD19+/HLA-A*02− tumor cell mouse xenograft was generated by injecting Raji target cells into the flanks of immunocompromised (NGS-HLA-A2.1) mice (FIG. 7B). Raji cells were injected at two doses, 2e6 or 1e7 T cells, and the growth of the tumor and the persistence of the implanted T cells were analyzed over time. Only CD19+/HLA-A*02− tumor cells were killed in the mouse and the tumor control tracked with transferred T cell numbers, promoting survival of the host mice (FIG. 7C-7E and FIG. 12). Normal CD19+/HLA-A*02+ cells designed to model normal cells were unaffected by treatment.

### SUMMARY

We have developed a synthetic signal integration system that can take advantage of a large, new class of cancer targets derived from LOH. The system, without undue experimentation, meets requirements of a cell therapy for patients with LOH. The system works robustly in Jurkat cells, primary T cells, and in vivo. This system is also (i) modular and flexible, and works across CAR and TCR modalities with different target densities: (ii) silenced when the blocker and activator targets are present on one surface in cis, but not when a minority of cells expresses only the activator; and, (iii) switches states reversibly, consistent with the need to hunt tumor cells throughout the body.

### Example 6: Methods for Examples 3-5

### Cell Culture

Jurkat cells encoding an NFAT Luciferase reporter were obtained from BPS Bioscience. All other cell lines used in this study were obtained from ATCC. In culture, Jurkat cells were maintained in RPMI media supplemented with 10% FBS, 1% Pen/Strep and 0.4 mg/mL G418/Geneticin. T2, MCF7, and Raji cells were maintained as suggested by ATCC. “Normal” Raji cells were made by transducing Raji cells with HLA-A*02 lentivirus (custom lentivirus, Alstem) at a MOI of 5. HLA-A*02-positive Raji cells were sorted using a FACSMelody Cell Sorter (BD).

### Plasmid Construction

The NY-ESO-1-responsive inhibitory construct was created by fusing the NY-ESO-1 scFv LBD to domains of receptors including hinge, transmembrane region, and/or intracellular domain of leukocyte immunoglobulin-like receptor subfamily B member 1, LILRB1 (LIR-1), programmed cell death protein 1, PDCD1 (PD-1), or cytotoxic T-lymphocyte protein 4, CTLA4 (CTLA-4). Gene segments were combined using Golden Gate cloning and inserted downstream of a human EF1α promoter contained in a lentiviral expression plasmid.

### Jurkat Cell Transfection

Jurkat cells were transiently transfected via 100 uL format Neon electroporation system (Thermo Fisher Scientific) according to manufacturer's protocol using the following settings: 3 pulses, 1500V, 10 msec. Cotransfection was performed with 1-3 ug of activator CAR or TCR construct and 1-3 ug of either scFv or Ftcr blocker constructs or empty vector per 1e6 cells and recovered in RPMI media supplemented with 20% heat-inactivated FBS and 0.1% Pen/Strep.

### Jurkat-NFAT-Luciferase Activation Studies

Peptides, MAGE-A3 (MP1; FLWGPRALV), MAGE-A3 (MP2; MPKVAELVHFL), HPV E6 (TIHDIILECV), HPV E7 (YMLDLQPET) and modified NY-ESO-1 ESO (ESO; SLLMWITQV), were synthesized by Genscript. Activating peptide was serially diluted starting at 50 uM. Blocker peptide, NY-ESO-1, was diluted to 50 uM (unless otherwise indicated) which was added to the activating peptide serial dilutions and subsequently loaded onto 1e4 T2 cells in 15 uL of RPMI supplemented with 1% BSA and 0.1% Pen/Strep and incubated in Corning® 384-well Low Flange White Flat Bottom Polystyrene TC-treated Microplates. The following day, 1e4 Jurkat cells were resuspended in 15 uL of RPMI supplemented with 10% heat-inactivated FBS and 0.1% Pen/Strep, added to the peptide-loaded T2 cells and cocultured for 6 hours. ONE-Step Luciferase Assay System (BPS Bioscience) was used to evaluate Jurkat luminescence. Assays were performed in technical duplicates.

### Primary T Cell Transduction, Expansion, and Enrichment

Frozen PBMCs were thawed in 37° C. water bath and cultured at 1e6 cells/mL in LymphoONE (Takara) with 1% human serum and activated using 1:100 of T cell TransAct (Miltenyi) supplemented with IL-15 (10 ng/mL) and IL-21 (10 ng/mL). After 24 hours, lentivirus was added to PBMCs at a MOI of 5. PBMCs were cultured for 2-3 additional days to allow cells to expand under TransAct stimulation. Post expansion, activator and blocker transduced primary T cells were enriched using anti-PE microbeads (Miltenyi) according to manufacturer's instructions. Briefly, primary T cells were incubated with CD19-Fc (R&D Systems) at 1:100 dilution for 30 minutes at 4° C. in MACS buffer (0.5% BSA+2 mM EDTA in PBS). Cells were washed 3 times in MACS buffer and incubated in secondary antibody (1:200) for 30 minutes at 4° C. in MACS buffer. Cells were then incubated in anti-PE microbeads and passed through the LS column (Miltenyi).

### Primary T Cell In Vitro Cytotoxicity Studies

For cytotoxicity studies with pMHC targets, enriched primary T cells were incubated with 2e3 MCF7 cells expressing Renilla luciferase (Biosettia) loaded with a titration of target peptide as described above at an effector:target ratio of 3:1 for 48 hours. Live luciferase-expressing MCF7 cells were quantified using a Renilla Luciferase Reporter Assay System (Promega). For cytotoxicity studies with non-pMHC targets, enriched primary T cells were incubated with 2e3 WT Raji cells (“tumor” cells) or HLA-A*02 transduced Raji cells (“normal” cells) at an effector:target ratio of 3:1 for up to 6 days. WT “tumor” Raji cells stably expressing GFP and Renilla luciferase (Biosettia) or HLA-A*02 “normal” Raji cells were stably expressing RFP and firefly luciferase (Biosettia) were imaged together with unlabeled primary T cells using an IncuCyte live cell imager. Fluorescence intensity of live Raji cells over time was quantified using IncuCyte imaging software. For reversibility studies, enriched primary T cells were similarly cocultured with “normal” or “tumor” Raji cells for 3 days and imaged. After 3 days, T cells were separated from remaining Raji cells using CD19 negative selection and reseeded with fresh “normal” or “tumor” Raji cells as described. In separate wells, live luciferase-expressing Raji cells were quantified using a Dual-Luciferase Reporter Assay System (Promega).

### Mouse Xenograft Study

Frozen PBMCs were thawed in 37° C. water bath and rested overnight in serum-free TexMACS Medium (Miltenyi) prior to activation. PBMCs were activated in 1.5e6 cells/mL using T cell TransAct (Miltenyi) and TexMACS Medium supplemented with IL-15 (20 ng/mL) and IL-21 (20 ng/mL). After 24 hours, lentivirus was added to PBMCs at a MOI of 5. PBMCs were cultured for 8-9 additional days to allow cells to expand under TransAct stimulation. Post expansion, T cells were enriched on A2-LIR-1 (pMHC HLA-A*02 ScFv fused to a LIR-1 hinge, TM and ICD) using anti-PE microbeads (Miltenyi) against streptavidin-PE-HLA-A*02-pMHC prior to in vivo injection.

5-6 week old female NOD.Cg-Prkdcscid Il2rgtm1Wjl Tg(HLA-A/H2-D/B2M)1Dvs/SzJ (NSG-HLA-A2/HHD) mice were purchased from The Jackson Labs. Animals were acclimated to the housing environment for at least 3 days prior to the initiation of the study. Animals were injected with 2e6 WT Raji cells or HLA-A*02 transduced Raji cells in 100 uL volume subcutaneously in the right flank. When tumors reached an average of 70 mm³ (V=L×W×W/2), animals were randomized into 5 groups (n=7) and 2e6 (data not shown) or 1e7 T cells were administered via the tail vein. Post T cell injection, tumor measurements were performed 3 times per week and blood was collected 10 days and 17 days after for flow analysis. Post RBC lysis, cells were stained with anti-hCD3 antibody, anti-hCD4 antibody, anti-hCD8 antibody, anti-msCD45 antibody (Biolegend).

### Example 7

The ability of a blocker receptor with an HLA-A-A*02 antigen binding domain and a LIR-1 ICD (C1765) to block activation of Jurkat cells expressing an activator CAR with an EGFR antigen binding domain (CT479) was assayed using the NFAT-luciferase reporter system as previously described. Wild type HeLa tumor cells, which were EGFR+ and HLA-A*02−, were used as target cells. EGFR+/HLA-A*02− HeLa cells were also transduced with a polynucleotide encoding HLA-A*02+ to generate EGFR+/HLA-A*02+ HeLa cells to use as target cells expressing both activator and blocker antigens.

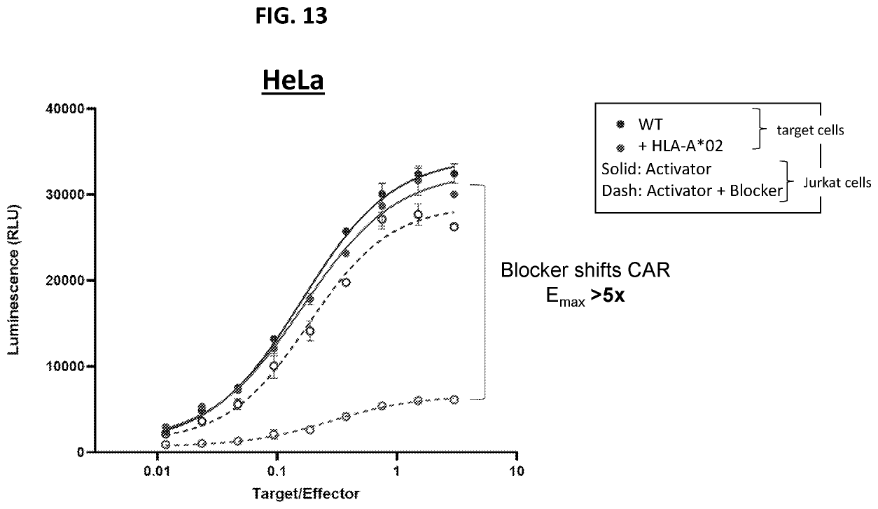

As shown in FIG. 13, expression of the HLA-A*02 LIR-1 blocker in Jurkat cells expressing the EGFR CAR shifts the CAR E_{MAX} by >5 fold compared to the CAR Emax of Jurkat cells that do not express the blocker.

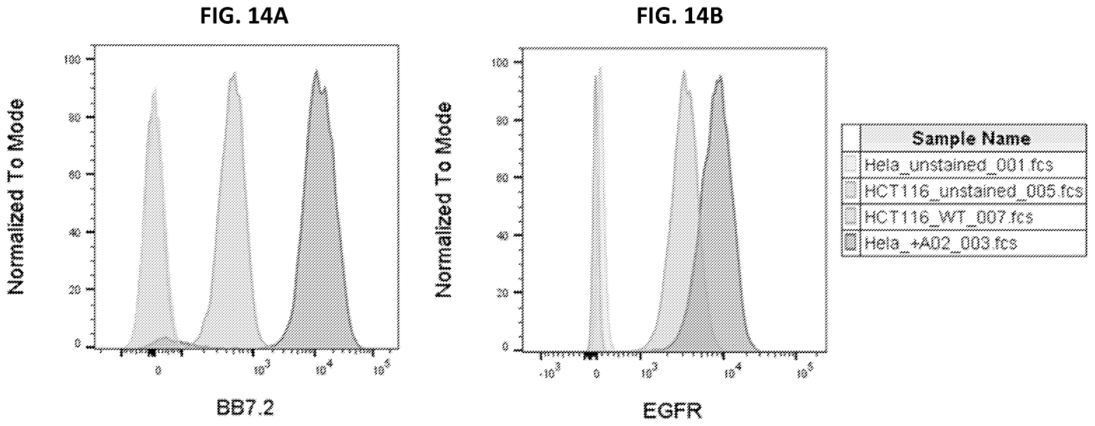

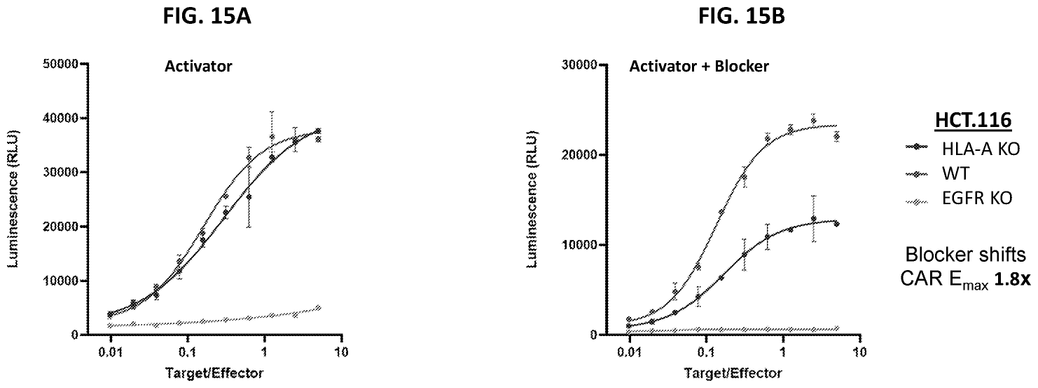

Furthermore, lower blocking was observed with lower HLA-A2 expression levels on target cells. Wild type HCT116 cells are EGFR+ and HLA-A*02. The levels of EGFR and HLA-A*02 were assayed in HCT116 cells and HeLa cells transduced with polynucleotides encoding the HLA-A*02 polynucleotides using an anti-EGFR antibody and an anti-HLA-A*02 antibody (BB7.2) followed by FACs sorting. As shown in FIGS. 14A & 14B, HCT116 cells have lower levels of blocker HLA-A*02 antigen than transduced HeLa cells. When Jurkat cells expressing the EGFR CAR and HLA-A*02 LIR-1 blocker were presented with HCT116 target cells expressing EGFR and HLA-A*02 antigens, presence of the HLA-A*02 LIR-1 blocker shifted the E_{MAX} of the EGFR CAR 1.8 fold (FIG. 15B). In contrast, transduced HeLa cells, which expressed a higher level of HLA-A*02 antigen, were able to mediate an EGFR CAR E_{MAX} shift of >5 fold (FIG. 13). As a control, there was minimal activation by EGFR knockout HCT116 cells (FIG. 15A).

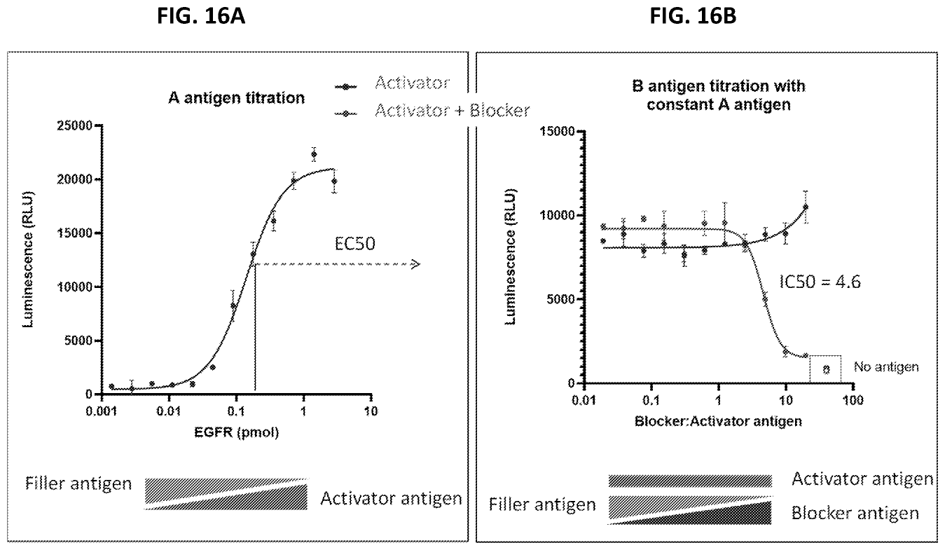

The ratio of blocker to activator necessary to achieve 50% blocking using the EGFR CAR and HLA-A*02 LIR-1 blocker was assayed using a bead based system, which is shown in FIGS. 16A and 16B.

To determine the EC50 of the activator antigen, activator beads were coated with activator antigen at different concentrations. An irrelevant protein was added to each concentration so that the total protein concentration was the same, and a constant amount of beads was added to Jurkat effector cells expressing the EGFR CAR (FIG. 16A).

To determine blocker antigen IC50, beads were coated with activator antigen at the EC50 concentration (determined in FIG. 16A), and coated with blocker antigen at different concentrations. An irrelevant protein was added at each concentration so that the total protein concentration remained the same, and a constant amount of beads was added to Jurkat effector cells expressing either the EGFR CAR or the EGFR CAR and the HLA-A*02 LIR-1 blocker (FIG. 16B).

### Example 8: LIR-1 Based Blockers can Inhibit TCR Signaling Using a Solid Tumor Cell Line

Jurkat effector cells expressing a MAGE-A3 activator TCR and a NY-ESO-1 scFv LIR-1 based inhibitory receptor (comprising a LIR-1 hinge, TM and ICD), were assayed using A375 target cells loaded with different concentrations of activator and blocker peptides. Jurkat cell activation was assayed using an NFAT luciferase assay (see Example 6).

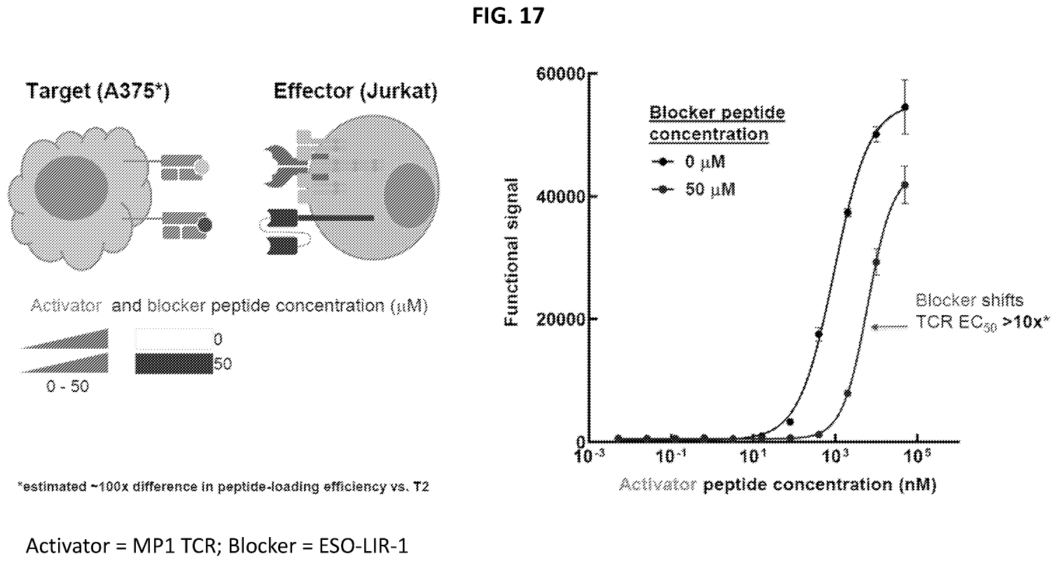

As shown in FIG. 17, loading A375 cells with 50 μM NY-ESO-1 peptide shifted the activator TCR E_{MAX} greater than 10 fold. There is an estimated ˜100× difference in peptide loading efficiency in A375 cells versus T2 target cells. Peptide loading may account for the apparent therapeutic window.

### Example 9: HLA-A*02 LIR-1 Based Blockers can Inhibit CAR Signaling Using a B Cell Leukemia Cell Line

Jurkat effector cells expressing the non-pMHC, high density CD19 specific activator (CD19 scFv CAR activator), with or without co-expression of a pMHC HLA-A*02 scFv LIR-1 based inhibitory receptor (comprising a LIR hinge, TIM and ICD), were assayed using NALM6 target cells. Jurkat cell activation was assayed using an NFAT luciferase assay (see Example 6), and the effector to target cell (E:T) ratio was varied.

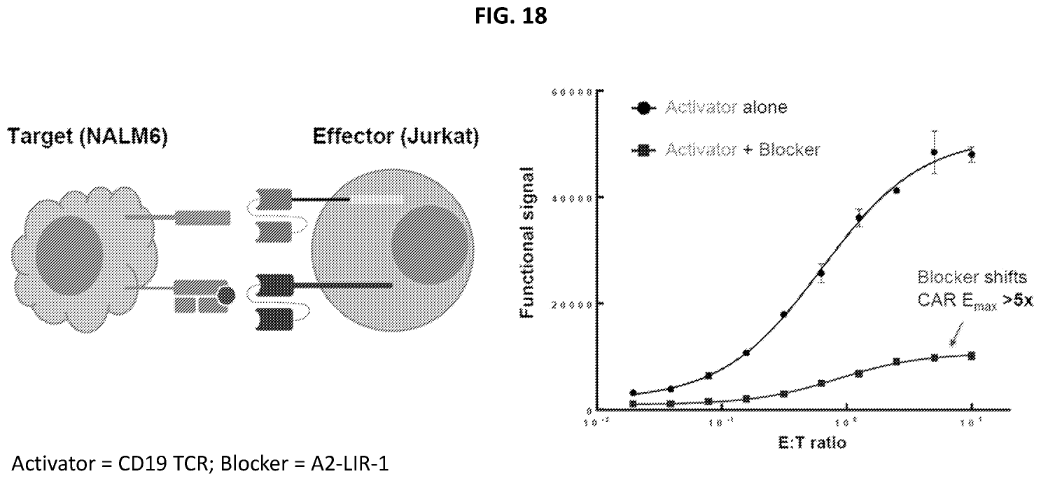

As shown in FIG. 18, expression of the blocker by Jurkat cells was able to shift the E_{MAX} of the CAR by greater than 5 fold.

### Example 10: HLA-A*02 LIR-1 Based Blockers can Inhibit CAR Signaling in a Dose Dependent Manner

Jurkat effector cells expressing an NY-ESO-1 scFv CAR, and a pMHC HLA-A*02 scFv LIR-1 based inhibitory receptor, were assayed using T2 target cells loaded with varying amounts of peptide (note, in this case the same peptide is recognized by both the activator and blocker ScFv). Jurkat cell activation was assayed using an NFAT luciferase assay (see Example 6). Jurkat cells were transfected with varying ratios of activator to blocker DNA, i.e. 1:1, 1:2 and 1:3 activator to blocker, to vary the ratios of the receptors expressed by the Jurkat cells.

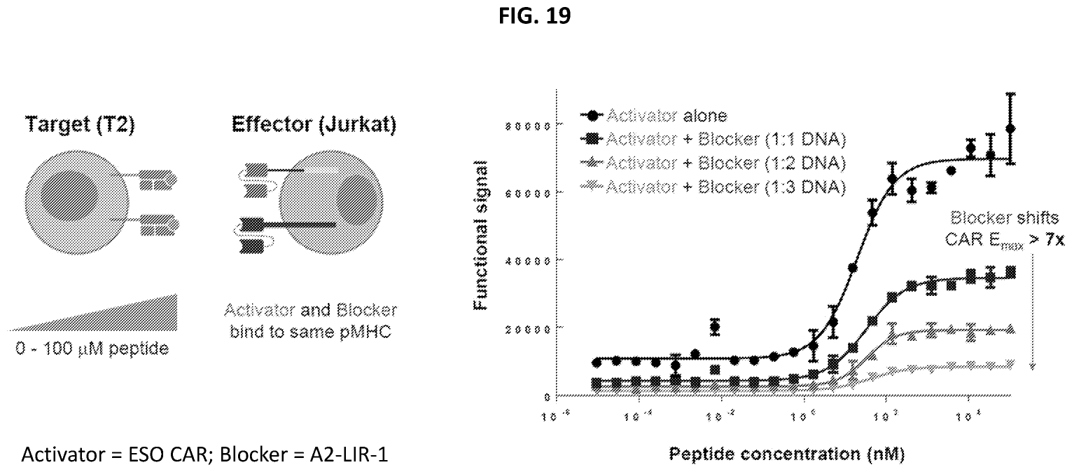

As can be seen in FIG. 19, even with Jurkat cells transfected with activator and blocker receptor DNA at a ratio of 1:1, the MHC HLA-A*02 scFv LIR-1 based inhibitory receptor (blocker) was able to inhibit activation of Jurkat cells by the activator CAR Furthermore, the degree to which the inhibitory receptor blocked activation increased with an increased amount of inhibitory receptor DNA compared to activator receptor DNA used in Jurkat cell transfection.

### Example 11: HLA-A*02 LIR-1 Based Blockers can Inhibit a Universal (Pan HLA Class I) Activator with Tunable Strengths

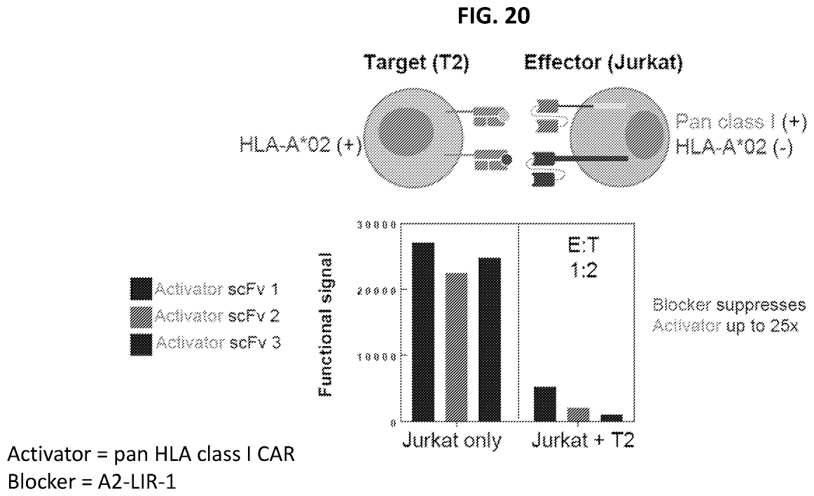

Activation of Jurkat effector cells expressing pan HLA scFv CARs with three different scFv binding domains based on the pan HLA antibody W6/32, and a pMHC HLA-A*02 scFv LIR-1 based inhibitory receptor were assayed using HLA-A*02 positive T2 cells. As can be seen from FIG. 20, each activator scFv supported a different functional signal in HLA-A*02-negative Jurkat cells. The pMHC HLA-A*02 scFv LIR-1 based inhibitory receptor was able to block functional signal from all three pan HLA scFv CARs when Jurkat cells were contacted with HLA-A*02− positive T2 target cells at an E:T ratio of 1:2. Moreover, the pMHC HLA-A*02 scFv LIR-1 based inhibitory receptor was able to suppress the activator up to 25 fold.

### Example 12: HLA-A*02 LIR-1 Based Inhibitory Receptors can Block Activation by an MSLN CAR Activator

Activation of Jurkat effector cells expressing an MSLN CAR activator and a pMHC HLA-A*02 ScFv LIR-1 based inhibitory receptor was assayed using the NFAT Luciferase assay described in Example 6.

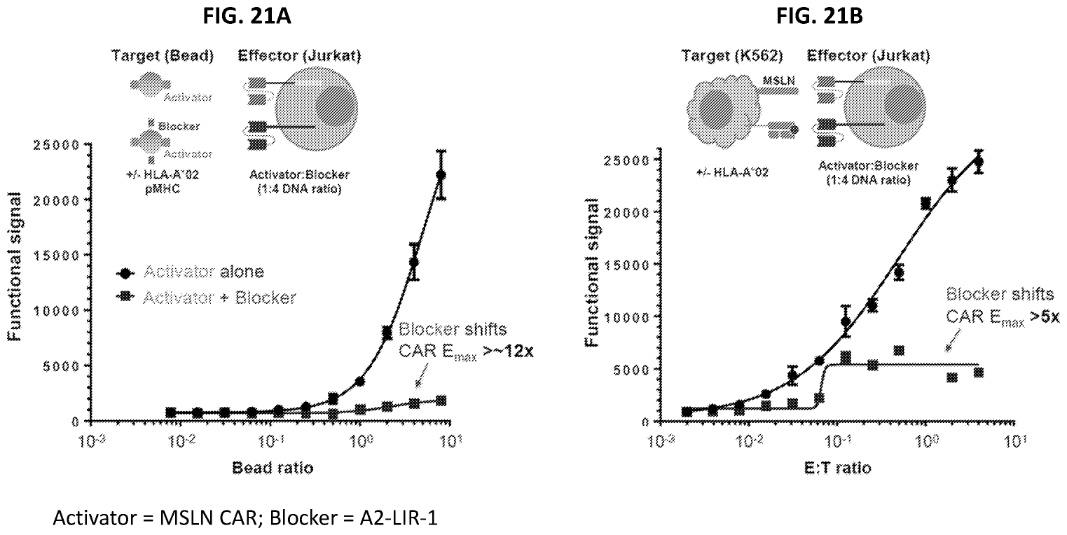

Jurkat cells were transfected with activator:blocker DNA at a ratio of 1:4, and activation was assayed in a cell-free bead based assay (FIG. 21A). Beads were loaded with either activator antigen, or activator and blocker antigens, and the ratio of beads to Jurkat cells was varied. In the cell-free bead based assay, the pMHC HLA-A*02 ScFv LIR-1 based inhibitory receptor was able to block activation of the Jurkat cells when cells were contacted with beads carrying the pMHC HLA-A*02 blocker and MSLN activator in cis. Presence of the pMHC HLA-A*02 blocker on the beads was able to shift E_{MAX} of MSLN CAR by greater than or equal to 12× (FIG. 21A).

Activation Jurkat cells transfected with the same activator and blocker at a 1:4 DNA ratio were assayed for activation using the chronic myelogenous leukemia cell line K562. K562 expresses MLSN, the activator antigen. The response of Jurkat effector cells to K562 cells transduced with HLA-A*02 to express both activator and blocker antigens (MSLN+ HLA-A*02+) and untransduced K562 (MSLN+ HLA-A*02−) that expressed the activator but not the blocker antigen was assayed. As can be seen in FIG. 21B, expression of HLA-A*02+ by the K562 cells was able to shift the MSLN CAR E_{MAX} by greater than 5×.

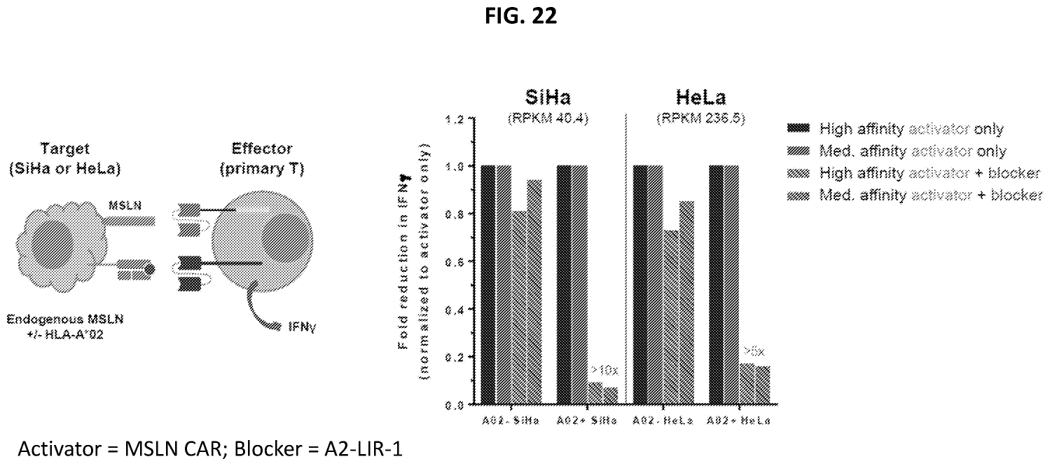

The ability of the pMHC HLA-A*02 inhibitory receptor to block activation via the MSNL ScFv CAR was also assayed using effector primary T cells and SiHa or HeLa target cells as described for Raji in Example 6. SiHa and HeLa cells endogenously express MSLN, and were transduced to express the HLA-A*02 inhibitory receptor target. Activation of primary effector T cells was assayed by looking at fold induction of IFNγ. As shown in FIG. 22, the pMHC HLA-A*02 LIR-1 inhibitory receptor was able to block activation of primary T cells when the primary T cells were presented with SiHa or HeLa target cells expressing HLA-A*02 (greater than 10× and 5× inhibition, respectively).

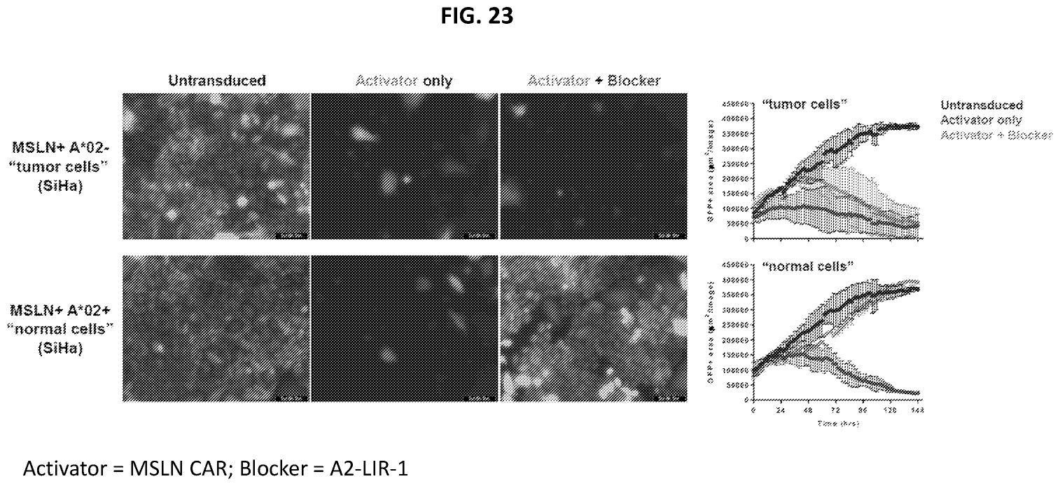

The pMHC HLA-A*02 inhibitory receptor was also able to inhibit killing by T cells expressing both the MSLN ScFv CAR and the pMHC HLA-A*02 LIR-1 inhibitory receptor, when the T cells were presented with SiHa cells that expressed MSLN but not HLA-A*02 (FIG. 23).

### Example 13: HLA-A*02 LIR-1 Based Inhibitory Receptors can Block Activation by an EGFR CAR Activator

Activation of Jurkat effector cells expressing an EGFR CAR activator and a pMHC HLA-A*02 ScFv LIR-1 based inhibitory receptor (comprising a LIR-1 hinge, transmembrane and ICD) was assayed using the NFAT Luciferase assay described in Example 6.

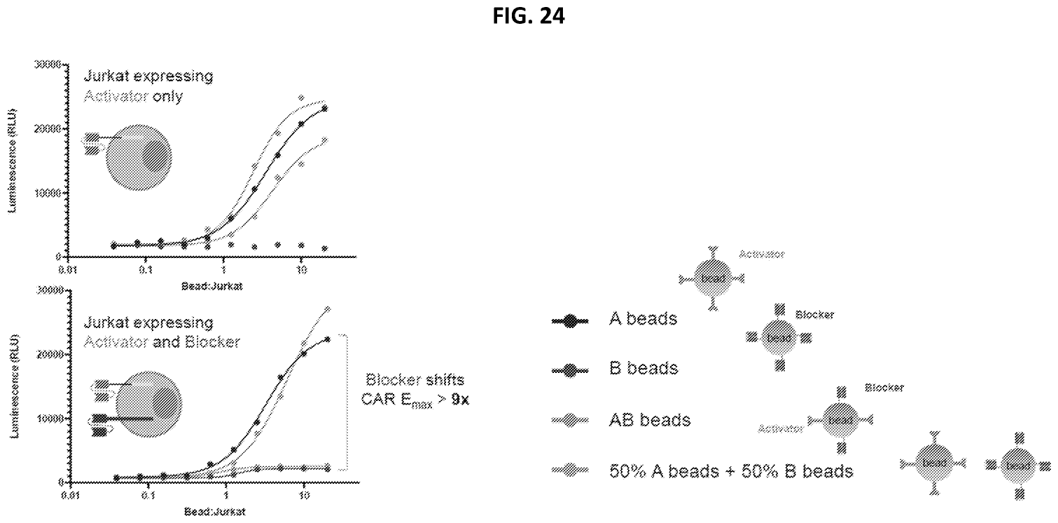

Jurkat cells were transfected with activator and blocker receptor DNA, and activation was assayed in a cell-free bead based assay (FIG. 24). Beads were loaded with either activator antigen, blocker antigen, or activator and inhibitor antigens, and the ratio of beads to Jurkat cells was varied. In the cell-free bead based assay, the HLA-A*02 ScFv LIR-1 based inhibitory receptor was able to block activation of the Jurkat cells when cells were contacted with beads carrying the HLA-A*02 blocker and EGFR activator in cis, but not when the HLA-A*02 blocker and EGFR activator were in trans (on different beads). Presence of the HLA-A*02 blocker on the beads was able to shift E_{MAX} of EGFR CAR by greater than or equal to 9× (FIG. 24).

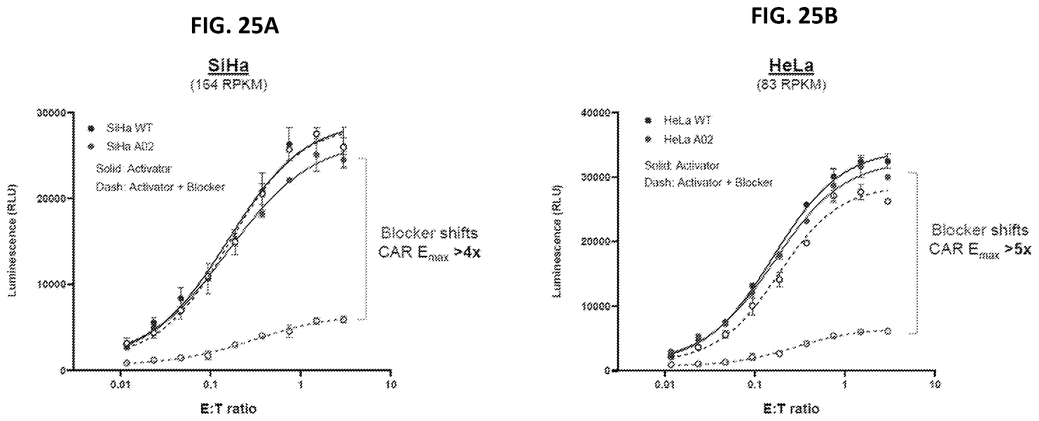

Activation of Jurkat cells expressing the EGFR CAR activator and a HLA-A*02 ScFv LIR-1 based inhibitory receptor was also assayed using HeLa and SiHa cells as target cells. Wild type HeLa and SiHa cell lines express EGFR but not HLA-A*02 (SiHa WT and HeLa WT), but were transduced to express the HLA-A*02 inhibitory receptor target (SiHa A02 and HeLa A02). As can be seen in FIGS. 25A-25B, the HLA-A*02 ScFv LIR-1 based inhibitory receptor was able to shift the EGFR E_{MAX} by greater than 4× using SiHa target cells (FIG. 25A) and greater than 5× using HeLa target cells (FIG. 25B).

### Example 14: Activator and Blocker Pairs can Discriminate Between KRAS Alleles

MiHAs are peptides derived from proteins that contain nonsynonymous differences between alleles. Using KRAS as a model for MiHAs, the activator and blocker pairs were able to discriminate and respond to different KRAS variants using antigen binding domains specific to the KRAS G12V and KRAS G12D mutations.

Using the Jurkat-NFAT-luciferase activation studies described in Example 6 and T2 target cells, the ability of KRAS ScFv or Ftcr inhibitory LIR-1 based receptors to inhibit activation mediated by an activator KRAS CAR or TCR was assayed.

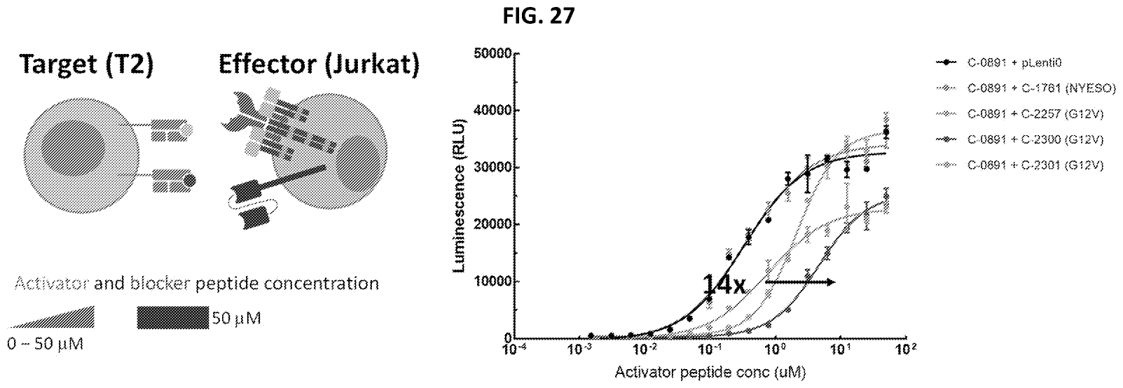

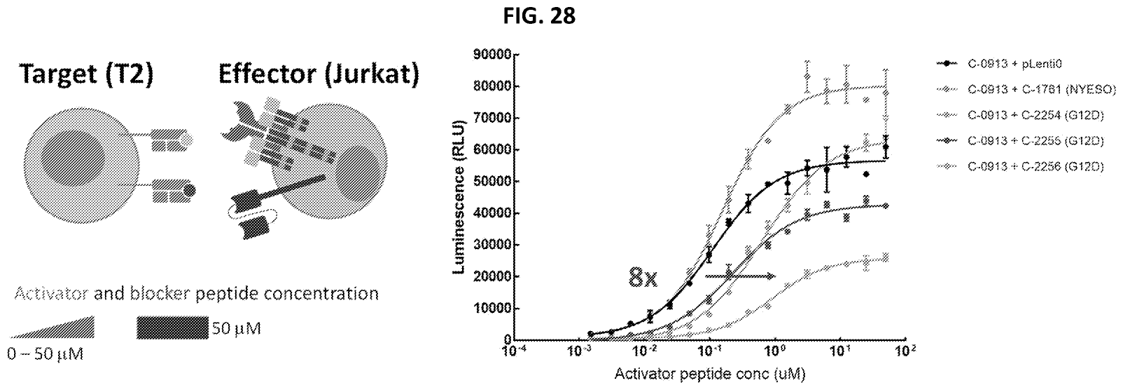

FIG. 27 shows that a KRAS G12V ScFV blocker was able to inhibit activation of Jurkat cells by a KRAS G12D TCR (C-891) and shift the KRAS G12D E_{MAX} by 14×. FIG. 28 shows a similar result with a reciprocal pair, a KRAS G12D ScFv blocker and a KRAS G12V TCR activator (C-913), where the inhibitor was able to shift the KRAS G12V E_{MAX} by 8×.

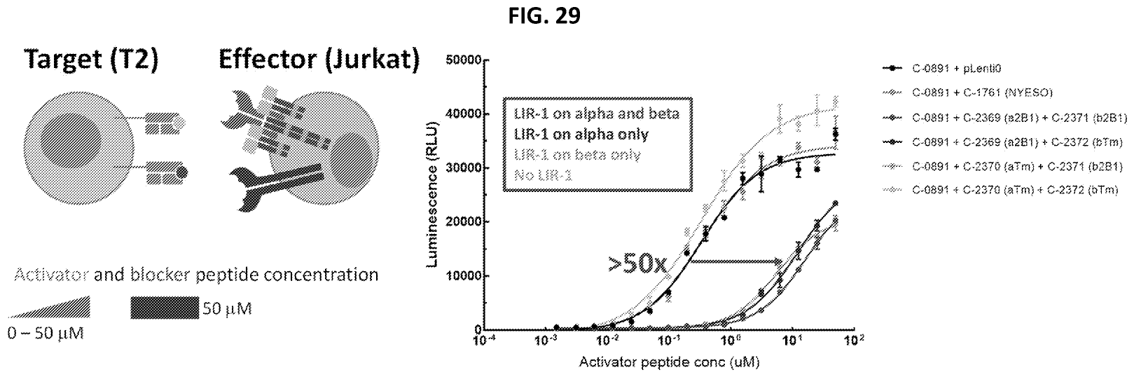

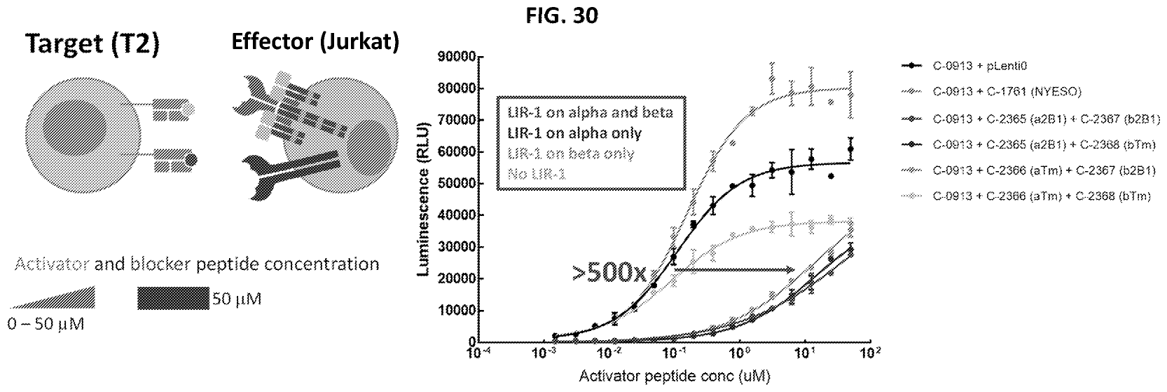

FIG. 29 shows that a KRAS G12V Ftcr blocker was able to inhibit a KRAS G12D TCR. The inhibitor was able to shift the KRAS G12D E_{MAX} by greater than 50×. In this case, constructs with a LIR-1 transmembrane domain and intracellular domain were included on both the alpha and beta chains of the inhibitory TCR (LIR-1 on alpha and beta), on the TCR alpha chain only (LIR-1 on alpha only), on the TCR beta chain only (LIR-1 on beta only), and a version with no LIR-1 ICD was included as a control (no LIR-1). In a reciprocal experiment, a KRAS G12D Ftcr blocker was able to inhibit a KRAS G12V activator TCR, shifting the KRAS G12V E_{MAX} by greater than 500×.

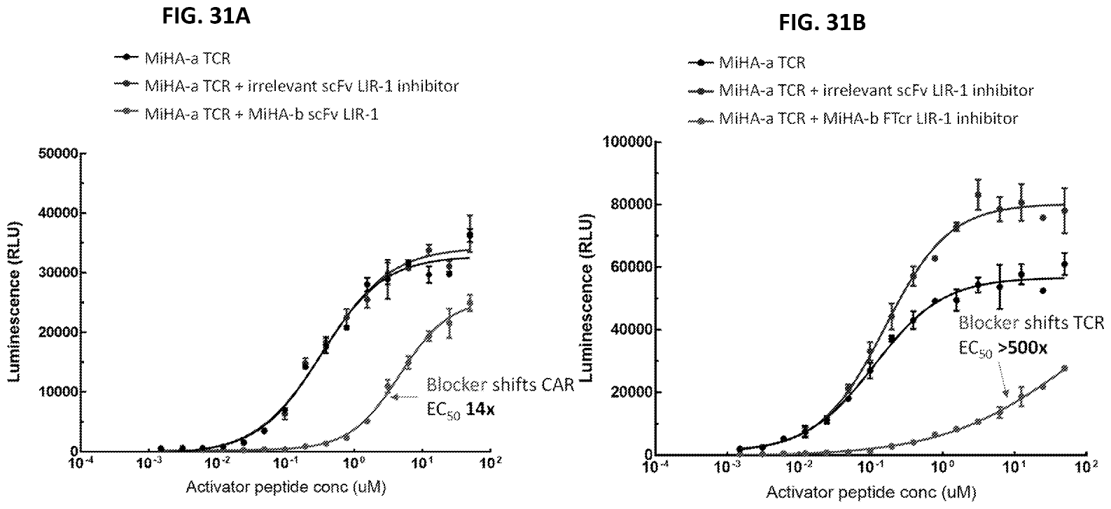

Finally, this effect was dependent on the specific ligand binding domains, as pairs with an inhibitory receptor that had an irrelevant ScFv domain had little effect on activator E_{MAX} (FIGS. 31A-31B).

Table 14 lists the KRAS ScFv and Ftcr sequences. All ScFvs were fused to a LIR-1 binge, TM and ICD. All Ftcrs were fused to a LIR-1 TM and ICD.

TABLE 14
KRAS ScFv and Ftcr Sequences.

| | |
|---|---|
| C-02256 (Kp33A1101 H125 scFv): | C-02256 |
| QVQLVESGGGLVKPGGSLRLSCAASGFTFSDYYMSWIRQAPGKGLEWVSYISSSGSTIYYAD | (Kp33A1101_H125 |
| SVKGRFTISRDNAKNSLYLQMNSLRAEDTAVYYCARDFTRDYYYYYYMDVWGKGTTVTVSS | scFv) DNA |
| GGGGSGGGGSGGGGSGGDIQMTQSPSSLSASVGDRVTITCRASQSISSYLNWYQQKPGKA | Sequence: SEQ ID |
| PKLLIYAASSLQSGVPSRFSGSGSGTDFTLTISSLQPEDFATYYCQQSYSTPLTFGGGTKVEIK | NO: 233 |
| (SEQ ID NO: 232) | |
| C-02257 (K14A11:01 V001 scFv): | C-02257 |
| QVTLRESGPALVKPTQTLTLTCTFSGFSLSTSGMCVSWIRQPPGKALEWLALIDWDDDKYYS | (K14A11:01_V001_ |
| TSLKTRLTISKDTSKNQVVLTMTNMDPVDTATYYCARSYDELYYFDYWGQGTLVTVSSGGG | scFv) DNA |
| GSGGGGGGGGSGGDIQMTQSPSSLSASVGDRVTITCRASQSISSYLNWYQQKPGKAPKLL | Sequence: SEQ ID |
| IYAASSLQSGVPSRFSGSGSGTDFTLTISSLQPEDFATYYCQQSYSTPLTFGGGTKVEIK (SEQ | NO: 235 |
| ID NO: 234) | |
| C-002300 (K14A11:01 H001/L004 scFv): | C-002300 |
| QVTLRESGPALVKPTQTLTLTCTFSGFSLSTSGMCVSWIRQPPGKALEWLALIDWDDDKYYS | (K14A11:01 |
| TSLKTRLTISKDTSKNQVVLTMTNMDPVDTATYYCARSYDELYYFDYWGQGTLVTVSSGGG | H001/L004_scFv) |
| GSGGGGSGGGGSGGDIQMTQSPSSLSASVGDRVTITCRASQSIWTSYLNWYQQKPGKAPK | DNA Sequence: |
| LLIYAASSLQSGVPSRFSGSGSGTDFTLTISSLQPEDFATYYCQQSYSTPLTFGGGTKVEIK | SEQ ID NO: 237 |
| (SEQ ID NO: 236) | |
| C-002301 (K14A11:01 H001/L010 scFv): | C-002301 |
| QVTLRESGPALVKPTQTLTLTCTFSGFSLSTSGMCVSWIRQPPGKALEWLALIDWDDDKYYS | (K14A11:01 |
| TSLKTRLTISKDTSKNQVVLTMTNMDPVDTATYYCARSYDELYYFDYWGQGTLVTVSSGGG | H001/L010 scFv) |
| GSGGGGSGGGGSGGDIQMTQSPSSLSASVGDRVTITCRASQSISSYLNWYQQKPGKAPKLL | DNA Sequence: |
| IYAASSLQSGVPSRFSGSGSGTDFTLTISSLQPEDFATYYCQQSYSTRLTFGGGTKVEIK (SEQ | SEQ ID NO: 239 |
| ID NO: 238) | |
| C-002365 [pLenti 1 K33A1101 V002 TCRa T48C (G12D TRAV4-4/DV10*01)]: | C-002365 |
| MQRNLGAVLGILWVQICWVRGDQVEQSPSALSLHEGTDSALRCNFTTTMRSVQWFRQNS | [plenti 1 |
| RGSLISLFYLASGTKENGRLKSAFDSKERRYSTLHIRDAQLEDSGTYFCAADSSNTGYQNFYFG | K33A1101_V002 |
| KGTSLTVIPNIQNPEPAVYQLKDPRSQDSTLCLFTDFDSQINVPKTMESGTFITDKCVLDMKA | TCRa T48C (G12D |
| MDSKSNGAIAWSNQTSFTCQDIFKETNATYPSSDVPCDATLTEKSFETDMNLNFQNLS | TRAV4- |
| (SEQ ID NO: 240) | 4/DV10*01)] |
| | DNA Sequence: |
| | SEQ ID NO: 241 |
| C-002367 [pLenti 1 K33A1101 V002 TCRb S51C (TRBV12-2*01)]: | C-002367 |
| MSNTAFPDPAWNTTLLSWVALFLLGTSSANSGVVQSPRYIIKGKGERSILKCIPISGHLSVAW | [pLenti 1 |
| YQQTQGQELKFFIQHYDKMERDKGNLPSRFSVQQFDDYHSEMNMSALELEDSAVYFCASS | K33A1101_V002 |
| LTDPLDSDYTFGSGTRLLVIEDLRNVTPPKVSLFEPSKAEIANKQKATLVCLARGFFPDHVELS | TCRb S51C |
| WWVNGKEVHSGVCTDPQAYKESNYSYCLSSRLRVSATFWHNPRNHFRCQVQFHGLSEED | (TRBV12-2*01)] |
| KWPEGSPKPVTQNISAEAWGRADCGITSASYQQGVLS | DNA Sequence: |
| (SEQ ID NO: 242) | SEQ ID NO: 243 |
| C-002368 [pLenti 1 K33A1101 V002 TCRb S51C (TRBV12-2*01)]: | C-002368 |
| MSNTAFPDPAWNTTLLSWVALFLLGTSSANSGVVQSPRYIIKGKGERSILKCIPISGHLSVAW | [pLenti 1 |
| YQQTQGQELKFFIQHYDKMERDKGNLPSRFSVQQFDDYHSEMNMSALELEDSAVYFCASS | K33A1101_V002 |
| LTDPLDSDYTFGSGTRLLVIEDLRNVTPPKVSLFEPSKAEIANKQKATLVCLARGFFPDHVELS | TCRb S51C |
| WWVNGKEVHSGVCTDPQAYKESNYSYCLSSRLRVSATFWHNPRNHFRCQVQFHGLSEED | (TRBV12-2*01)] |
| KWPEGSPKPVTQNISAEAWGRADCGITSASYQQGVLS (SEQ ID NO: 244) | DNA Sequence: |
| | SEQ ID NO: 245 |
| C-002369 [pLenti 1 Kp514A1101 V001 TCRa T48C (G12 V TRAV3-3*01)1: | C-002369 |
| MKTVTGPLFLCFWLQLNCVSRGEQVEQRPPHLSVREGDSAVITCTYTDPNSYYFFWYKQEP | [pLenti 1 |
| GASLQLLMKVFSSTEINEGQGFTVLLNKKDKRLSLNLTAAHPGDSAAYFCAVSGGTNSAGNK | Kp514A1101_ |
| LTFGIGTRVLVRPDIQNPEPAVYQLKDPRSQDSTLCLFTDFDSQINVPKTMESGTFITDKCVL | V001 TCRa T48C |
| DMKAMDSKSNGAIAWSNQTSFTCQDIFKETNATYPSSDVPCDATLTEKSFETDMNLNFQN | (G12 V TRAV3- |
| LS | 3*01)] DNA |
| (SEQ ID NO: 246) | Sequence: SEQ |
| | ID NO: 247 |
| C-002371 [pLenti 1 Kp514A1101 V001 TCRb S51C (TRBV4*01)]: | C-002371 |
| MGCRLLSCVAFCLLGIGPLETAVFQTPNYHVTQVGNEVSFNCKQTLGHDTMYWYKQDSKK | [plenti 1 |
| LLKIMFSYNNKQLIVNETVPRRFSPQSSDKAHLNLRIKSVEPEDSAVYLCASSRDWGPAEQFF | Kp514A1101_V001 |
| GPGTRLTVLEDLRNVTPPKVSLFEPSKAEIANKQKATLVCLARGFFPDHVELSWWVNGKEV | TCRb S51C |
| HSGVCTDPQAYKESNYSYCLSSRLRVSATFWHNPRNHFRCQVQFHGLSEEDKWPEGSPKP | (TRBV4*01)] DNA |
| VTQNISAEAWGRADCGITSASYHQGVLS (SEQ ID NO: 248) | Sequence: SEQ ID |
| | NO: 249 |
| C-002372 [pLenti 1 Kp514A1101 V001 TCRb S51C (TRBV4*01)]: | C-002372 |
| MGCRLLSCVAFCLLGIGPLETAVFQTPNYHVTQVGNEVSFNCKQTLGHDTMYWYKQDSKK | [plenti 1 |
| LLKIMFSYNNKQLIVNETVPRRFSPQSSDKAHLNLRIKSVEPEDSAVYLCASSRDWGPAEQFF | Kp514A1101_V001 |
| GPGTRLTVLEDLRNVTPPKVSLFEPSKAEIANKQKATLVCLARGFFPDHVELSWWVNGKEV | TCRb S51C |
| HSGVCTDPQAYKESNYSYCLSSRLRVSATFWHNPRNHFRCQVQFHGLSEEDKWPEGSPKP | (TRBV4*01)] DNA |
| VTQNISAEAWGRADCGITSASYHQGVLS (SEQ ID NO: 250) | Sequence: SEQ ID |
| | NO: 251 |

TABLE 15
Ftcr sequences fused to LIR-1 TM and no ICD as controls.

| | |
|---|---|
| C-002366 [pLenti 1 K33A1101 V002 TCRa T48C (G12D TRAV4-4/DV10*01)]: | C-002366 |
| MQRNLGAVLGILWVQICWVRGDQVEQSPSALSLHEGTDSALRCNFTTTMRSVQWFRQNS | [pLenti 1 |
| RGSLISLFYLASGTKENGRLKSAFDSKERRYSTLHIRDAQLEDSGTYFCAADSSNTGYQNFYFG | K33A1101 V002 |
| KGTSLTVIPNIQNPEPAVYQLKDPRSQDSTLCLFTDFDSQINVPKTMESGTFITDKCVLDMKA | TCRa T48C (G12D |
| MDSKSNGAIAWSNQTSFTCQDIFKETNATYPSSDVPCDATLTEKSFETDMNLNFQNLS | TRAV4- |
| (SEQ ID NO: 252) | 4/DV10*01)] |
| | DNA Sequence: |
| | SEQ ID NO: 253 |
| C-002370 [pLenti 1 Kp514A1101 V001 TCRa T48C (G12 V TRAV3-3*01)]: | C-002370 |
| MKTVTGPLFLCFWLQLNCVSRGEQVEQRPPHLSVREGDSAVITCTYTDPNSYYFFWYKQEP | [pLenti 1 |
| GASLQLLMKVFSSTEINEGQGFTVLLNKKDKRLSLNLTAAHPGDSAAYFCAVSGGTNSAGNK | Kp514A1101 V001 |
| LTFGIGTRVLVRPDIQNPEPAVYQLKDPRSQDSTLCLFTDFDSQINVPKTMESGTFITDKCVL | TCRa T48C |
| DMKAMDSKSNGAIAWSNQTSFTCQDIFKETNATYPSSDVPCDATLTEKSFETDMNLNFQN | (G12V TRAV3- |
| LS (SEQ ID NO: 254) | 3*01)] DNA |
| | Sequence: SEQ ID |
| | NO: 255 |

### Example 15: Characterization of TCRs Recognizing a MiHA-Y

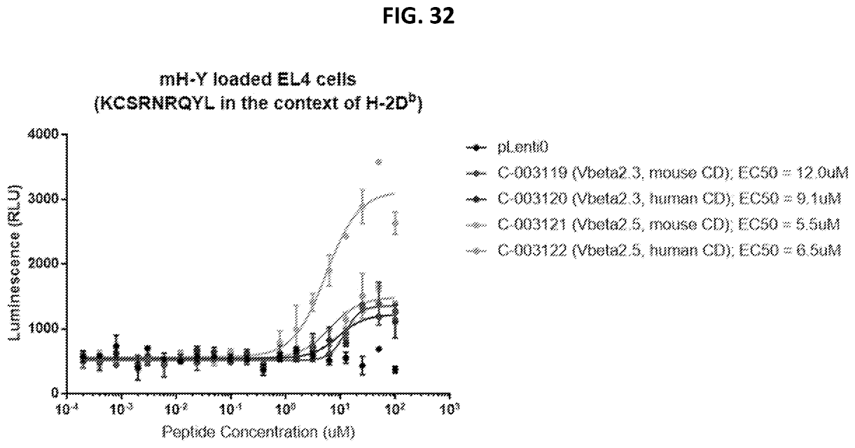

TCR alpha and beta extracellular domains from 2.3 and P2A mouse TCRs were cloned into activator TCR constructs. Either the native mouse or human constant regions were used. EL5 cells loaded with mH-Y H-2D^{b} peptide KCSRNRQYL (SEQ ID) NO: 256) were used as target cells to assay the activation of Jurkat cells transfected with the MiHA-Y TCRs (FIG. 32). As can be seen from FIG. 32, C-003121 supports robust Jurkat cell activation.

TABLE 16
Mouse miHA-Y TCR sequences with mouse or human constant regions

| | |
|---|---|
| C-003119 (H-Y TCRalpha P2A TCRbeta Jb2.3 mouse): | C-003119 (H- |
| MFPVTILLLSAFFSLRGNSAQSVDQPDAHVTLSEGASLELRCSYSYSAAPYLFWYVQYPGQSLQF | Y TCRalpha |
| LLKYITGDTVVKGTKGFEAEFRKSNSSFNLKKSPAHWSDSAKYFCALEGQDQGGSAKLIFGEGTK | P2A TCRbeta |
| LTVSSPDIQNPEPAVYQLKDPRSQDSTLCLFTDFDSQINVPKTMESGTFITDKTVLDMKAMDSKS | Jb2.3 mouse) |
| NGAIAWSNQTSFTCQDIFKETNATYPSSDVPCDATLTEKSFETDMNLNFQNLSVMGLRILLLKV | DNA |
| AGFNLLMTLRLWSSRAKRSGSGATNFSLLKQAGDVEENPGPMSNTAFPDPAWNTTLLSWVAL | Sequence: |
| FLLGTKHMEAAVTQSPRNKVAVTGGKVTLSCNQTNNHNNMYWYRQDTGHGLRLIHYSYGAG | SEQ ID NO: |
| STEKGDIPDGYKASRPSQENFSLILELATPSQTSVYFCASGDNSAETLYFGPGTRLTVLEDLRNVTP | 258 |
| PKVSLFEPSKAEIANKQKATLVCLARGFFPDHVELSWWVNGKEVHSGVSTDPQAYKESNYSYCL | |
| SSRLRVSATFWHNPRNHFRCQVQFHGLSEEDKWPEGSPKPVTQNISAEAWGRADCGITSASY | |
| HQGVLSATILYEILLGKATLYAVLVSGLVLMAMVKKKNS | |
| (SEQ ID NO: 257) | |
| C-003120 (H-Y TCRalpha T48C P2A H-Y TCRbeta Jb2.3 S57C human): | C-003120 (H- |
| MFPVTILLLSAFFSLRGNSAQSVDQPDAHVTLSEGASLELRCSYSYSAAPYLFWYVQYPGQSLQF | YTCRalpha |
| LLKYITGDTVVKGTKGFEAEFRKSNSSFNLKKSPAHWSDSAKYFCALEGQDQGGSAKLIFGEGTK | T48C P2A H-Y |
| LTVSSPYIQNPDPAVYQLRDSKSSDKSVCLFTDFDSQTNVSQSKDSDVYITDKCVLDMRSMDFK | TCRbeta Jb2.3 |
| SNSAVAWSNKSDFACANAFNNSIIPEDTFFPSPESSCDVKLVEKSFETDTNLNFQNLSVIGFRILLL | S57C human) |
| KVAGFNLLMTLRLWSSGSGATNFSLLKQAGDVEENPGPMSNTAFPDPAWNTTLLSWVALFLL | DNA |
| GTKHMEAAVTQSPRNKVAVTGGKVTLSCNQTNNHNNMYWYRQDTGHGLRLIHYSYGAGSTE | Sequence: |
| KGDIPDGYKASRPSQENFSLILELATPSQTSVYFCASGDNSAETLYFGPGTRLTVLEDLKNVFPPE | SEQ ID NO: |
| VAVFEPSEAEISHTQKATLVCLATGFYPDHVELSWWVNGKEVHSGVCTDPQPLKEQPALNDSR | 260 |
| YCLSSRLRVSATFWQNPRNHFRCQVQFYGLSENDEWTQDRAKPVTQIVSAEAWGRADCGFTS | |
| ESYQQGVLSATILYEILLGKATLYAVLVSALVLMAMVKRKDSRG (SEQ ID NO: 259) | |
| C-003121 (H-Y TCRalpha P2A TCRbeta Jb2.3L mouse): | C-003121 (H- |
| MFPVTILLLSAFFSLRGNSAQSVDQPDAHVTLSEGASLELRCSYSYSAAPYLFWYVQYPGQSLQF | Y TCRalpha |
| LLKYITGDTVVKGTKGFEAEFRKSNSSFNLKKSPAHWSDSAKYFCALEGQDQGGSAKLIFGEGTK | P2A TCRbeta |
| LTVSSPDIQNPEPAVYQLKDPRSQDSTLCLFTDFDSQINVPKTMESGTFITDKTVLDMKAMDSKS | Jb2.3L mouse) |
| NGAIAWSNQTSFTCQDIFKETNATYPSSDVPCDATLTEKSFETDMNLNFQNLSVMGLRILLLKV | DNA |
| AGFNLLMTLRLWSSRAKRSGSGATNFSLLKQAGDVEENPGPMSNTAFPDPAWNTTLLSWVAL | Sequence: |
| FLLGTKHMEAAVTQSPRNKVAVTGGKVTLSCNQTNNHNNMYWYRQDTGHGLRLIHYSYGAG | SEQ ID NO: |
| STEKGDIPDGYKASRPSQENFSLILELATPSQTSVYFCASGDNSAETLYFGPGTRLLVLEDLRNVTP | 262 |
| PKVSLFEPSKAEIANKQKATLVCLARGFFPDHVELSWWVNGKEVHSGVSTDPQAYKESNYSYCL | |
| SSRLRVSATFWHNPRNHFRCQVQFHGLSEEDKWPEGSPKPVTQNISAEAWGRADCGITSASY | |
| HQGVLSATILYEILLGKATLYAVLVSGLVLMAMVKKKNS (SEQ ID NO: 261) | |
| C-003122 (H-Y TCRalpha T48C P2A H-Y TCRbeta Jb2.3L S57C human): | C-003122 (H- |
| MFPVTILLLSAFFSLRGNSAQSVDQPDAHVTLSEGASLELRCSYSYSAAPYLFWYVQYPGQSLQF | Y TCRalpha |
| LLKYITGDTVVKGTKGFEAEFRKSNSSFNLKKSPAHWSDSAKYFCALEGQDQGGSAKLIFGEGTK | T48C P2A H-Y |
| LTVSSPYIQNPDPAVYQLRDSKSSDKSVCLFTDFDSQTNVSQSKDSDVYITDKCVLDMRSMDFK | TCRbeta |
| SNSAVAWSNKSDFACANAFNNSIIPEDTFFPSPESSCDVKLVEKSFETDTNLNFQNLSVIGFRILLL | Jb2.3L S57C |
| KVAGFNLLMTLRLWSSGSGATNFSLLKQAGDVEENPGPMSNTAFPDPAWNTTLLSWVALFLL | human) DNA |
| GTKHMEAAVTQSPRNKVAVTGGKVTLSCNQTNNHNNMYWYRQDTGHGLRLIHYSYGAGSTE | Sequence: |
| KGDIPDGYKASRPSQENFSLILELATPSQTSVYFCASGDNSAETLYFGPGTRLLVLEDLKNVFPPEV | SEQ ID NO: |
| AVFEPSEAEISHTQKATLVCLATGFYPDHVELSWWVNGKEVHSGVCTDPQPLKEQPALNDSRY | 264 |
| CLSSRLRVSATFWQNPRNHFRCQVQFYGLSENDEWTQDRAKPVTQIVSAEAWGRADCGFTSE | |
| SYQQGVLSATILYEILLGKATLYAVLVSALVLMAMVKRKDSRG (SEQ ID NO: 263) | |

### Example 16: Minor Histocompatibility Antigen HA-1 Inhibitory Receptors

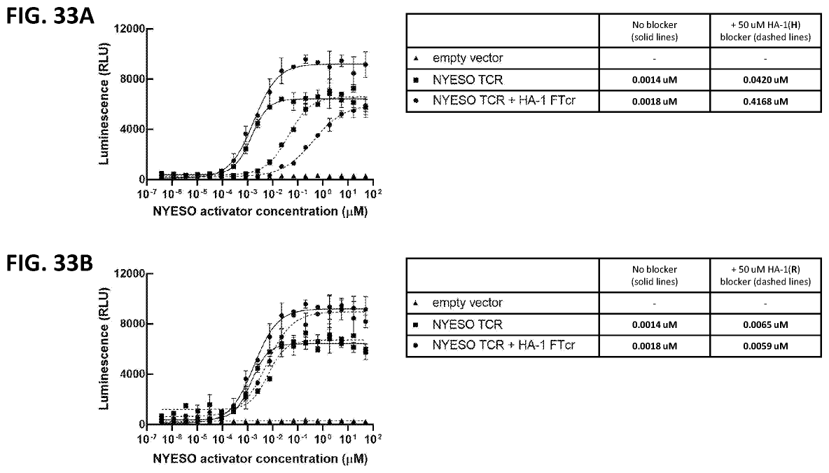

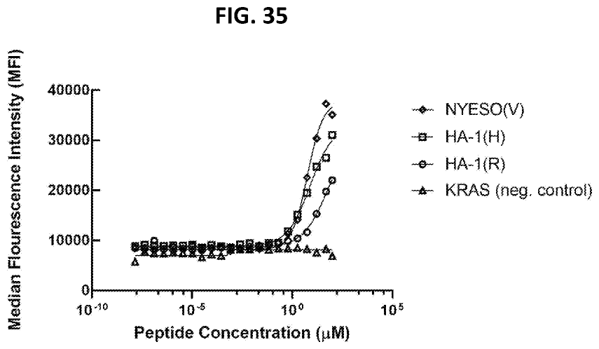

T2 cells carrying HLA-A*02 and A* 11 class I alleles were loaded with a titration of A*02-specific NY-ESO-1 peptide in the absence or presence of 50 uM A*02-specific HA-1 blocker peptides. Activation of Jurkat effector cells expressing either an NY-ESO-1 TCR or an NY-ESO-1 TCR and HA-1 Ftcr was assayed as described above (see Example 6). FIG. 33A shows that in the absence of blocker peptide, sensitivity of the NY-ESO-1 TCR is not affected by the presence of HA-1 Ftcr. In the presence of HA-1(H) blocker peptide, NY-ESO-1 and HA-1(H) peptides compete for the same HLA-A*02 allele causing an EC50 right shift of ˜30× (solid squares to dashed squares). Yet, an additional right shift in activity of ˜10× is observed in the presence of HA-1 Ftcr blocker (dashed squares to dashed circles). In addition, Emax is shifted downward 1.5× (solid circles to dashed circles). FIG. 33B shows that in the presence of the non-specific, allelic variant HA-1(R) blocker peptide, essentially no blocking is observed, suggesting blocking is specific to a single amino acid. In general, since NY-ESO-1 loads into HLA-A*02 more efficiently than HA-1(R) (see FIG. 35), there is also only a 3-5× right shift observed in the presence of HA-1(R) blocker peptide (solid to dashed lines).

Peptide sequences were as follows:

NY-ESO-1 =
(SEQ ID NO: 265)
SLLMWITQV,
HA-1(H) =
(SEQ ID NO: 191)
VLHDDLLEA,
HA-1(R) =
(SEQ ID NO: 266)
VLRDDLLEA.

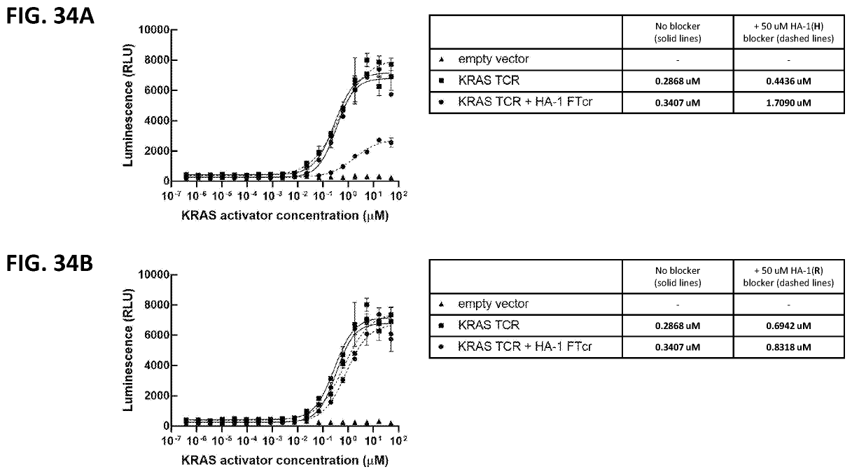

HA-1 Ftcr can also block a KRAS TCR specifically in the presence of HA-1(H) peptide. T2 cells carrying HLA-A*02 and A*11 class I alleles were loaded with a titration of A*11-specific KRAS peptide in the absence or presence of 50 uM A*02-specific HA-1 blocker peptides. Activation of Jurkat effector cells expressing either an NY-ESO-1 TCR or an NY-ESO-1 TCR and HA-1 Ftcr was assayed as described above (see Example 6). FIG. 34A shows that in the absence of blocker peptide, sensitivity of the KRAS TCR is not affected by the presence of HA-1 Ftcr. In the presence of HA-1(H) blocker peptide, HA-1 Ftcr blocks KRAS TCR by ˜5× in activity (solid circles to dashed circles). In addition, Emax is shifted downward 2.7× (solid circles to dashed circles). FIG. 34B shows that in the presence of the non-specific, allelic variant HA-1(R) blocker peptide, essentially no blocking is observed, suggesting blocking is specific to a single amino acid. In general, since KRAS and HA-1(H) or HA-1(R) do not load into the same alleles, there is no significant right shift observed in the presence of blocker peptide (solid to dashed lines).

Loading of the NY-ESO, HA-1(H) and HA-1(R) peptides by T2 cells was compared using BB7.2 staining, which specifically recognizes peptide-loaded HLA-A*02 class I allelic products, and quantified using flow cytometry. FIG. 35 shows that loading of HLA-A*02-specific NY-ESO-1 and HA-1(H) peptides is very similar in T2 cells. The allelic variant, HA-1(R), loads slightly less efficiently than HA-1(H) and NY-ESO-1 peptides.

HA-1(H) Ftcr sequences are described in Table 10, NY-ESO-1 and KRAS TCR are shown in Table 17 below.

TABLE 17
NY-ESO-1 and KRAS TCR sequences

| C-000063 pLenti 1 NY-ESO1 1G4 TCRalpha T95L, S96Y, T48C P2A TCRbeta S57C: | C-000063 |
|---|---|
| METLLGLLILWLQLQWVSSKQEVTQIPAALSVPEGENLVLNCSFTDSAIYNLQWFRQDP | plenti 1 NY- |
| GKGLTSLLLIQSSQREQTSGRLNASLDKSSGRSTLYIAASQPGDSATYLCAVRPLYGGSYIP | ESO1 1G4 |
| TFGRGTSLIVHPYIQNPDPAVYQLRDSKSSDKSVCLFTDFDSQTNVSQSKDSDVYITDKCV | TCRalpha |
| LDMRSMDFKSNSAVAWSNKSDFACANAFNNSIIPEDTFFPSPESSCDVKLVEKSFETDT | T95L, S96Y, |
| NLNFQNLSVIGFRILLLKVAGFNLLMTLRLWSSGSGATNFSLLKQAGDVEENPGPMSIGL | T48C P2A |
| LCCAALSLLWAGPVNAGVTQTPKFQVLKTGQSMTLQCAQDMNHEYMSWYRQDPGM | TCRbeta |
| GLRLIHYSVGAGITDQGEVPNGYNVSRSTTEDFPLRLLSAAPSQTSVYFCASSYVGNTGEL | S57C DNA |
| FFGEGSRLTVLEDLKNVFPPEVAVFEPSEAEISHTQKATLVCLATGFYPDHVELSWWVNG | Sequence: |
| KEVHSGVCTDPQPLKEQPALNDSRYCLSSRLRVSATFWQNPRNHFRCQVQFYGLSEND | SEQ ID NO: |
| EWTQDRAKPVTQIVSAEAWGRADCGFTSESYQQGVLSATILYEILLGKATLYAVLVSALV | 268 |
| LMAMVKRKDSRG (SEQ ID NO: 267) | |
| C-000891 plenti 1 K33A1101_V002 TCR (G12D TRAV4-4/DV10*01/BV12-2*01): | C-000891 |
| MQRNLGAVLGILWVQICWVRGDQVEQSPSALSLHEGTDSALRCNFTTTMRSVQWFR | plenti 1 |
| QNSRGSLISLFYLASGTKENGRLKSAFDSKERRYSTLHIRDAQLEDSGTYFCAADSSNTGY | K33A1101_ |
| QNFYFGKGTSLTVIPNIQNPEPAVYQLKDPRSQDSTLCLFTDFDSQINVPKTMESGTFITD | V002 TCR |
| KTVLDMKAMDSKSNGAIAWSNQTSFTCQDIFKETNATYPSSDVPCDATLTEKSFETDM | (G12D |
| NLNFQNLSVMGLRILLLKVAGFNLLMTLRLWSSRAKRSGSGATNFSLLKQAGDVEENPG | TRAV4- |
| PMSNTAFPDPAWNTTLLSWVALFLLGTSSANSGVVQSPRYIIKGKGERSILKCIPISGHLS | 4/DV10*01/ |
| VAWYQQTQGQELKFFIQHYDKMERDKGNLPSRFSVQQFDDYHSEMNMSALELEDSA | BV12-2*01) |
| VYFCASSLTDPLDSDYTFGSGTRLLVIEDLRNVTPPKVSLFEPSKAEIANKQKATLVCLARG | DNA |
| FFPDHVELSWWVNGKEVHSGVSTDPQAYKESNYSYCLSSRLRVSATFWHNPRNHFRCQ | Sequence: |
| VQFHGLSEEDKWPEGSPKPVTQNISAEAWGRADCGITSASYQQGVLSATILYEILLGKAT | SEQ ID NO: |
| LYAVLVSTLVVMAMVKRKNS (SEQ ID NO: 269) | 270 |

### Example 17: Ratios of Activator and Inhibitor Peptides

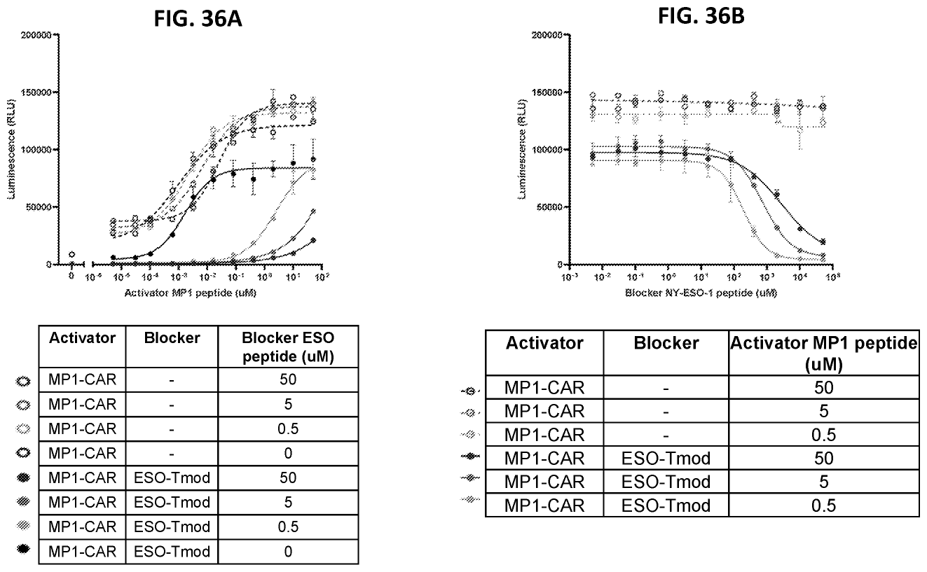

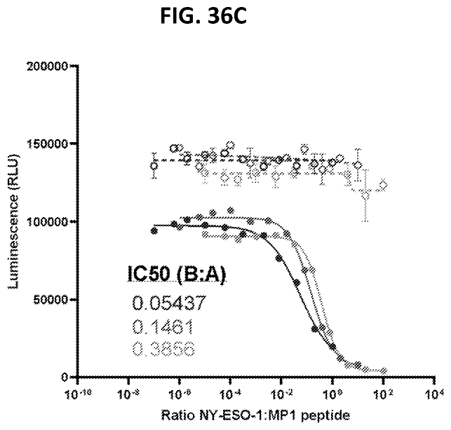

The NFAT-luciferase signal of Jurkat cells transfected with either activator MAGE-A3 CAR alone or in combination with NY-ESO-1 ScFv LIR1 blocker was measured after 6 hours of co-culture with activator and blocker peptide-loaded T2 cells. FIG. 36A shows the response of Jurkat cells co-cultured with T2 cells that were loaded with titrated amounts of activator MAGE-A3 peptide and a fixed concentration of blocker NY-ESO-1 peptide. FIG. 36B shows the response of Jurkat to T2 cells that were loaded with titrated amounts of blocker NY-ESO-1 peptide and a fixed concentration of activator MAGE-A3 peptide that was above the Emax concentration (˜0.1 μM). FIG. 36C shows x-value blocker NY-ESO-1 peptide concentrations from FIG. 36B that were normalized to the constant activator MAGE peptide concentrations used for each curve and plotted on the x-axis. The ratio of blocker peptide to activator peptide required for 50% blocking (IC50) are indicated for each curve. The B:A peptide ratio required is less than 1 indicating that, for this pair of activator CAR and blocker, similar (or fewer) blocker pMH-C antigens are required on target cells to block activator pMHC antigens. Blocking is possible at pMHC antigen densities that are similar to those that generate responses in activating pMHC CARs.

### Example 18: Optimization of Specific Receptor Pairs

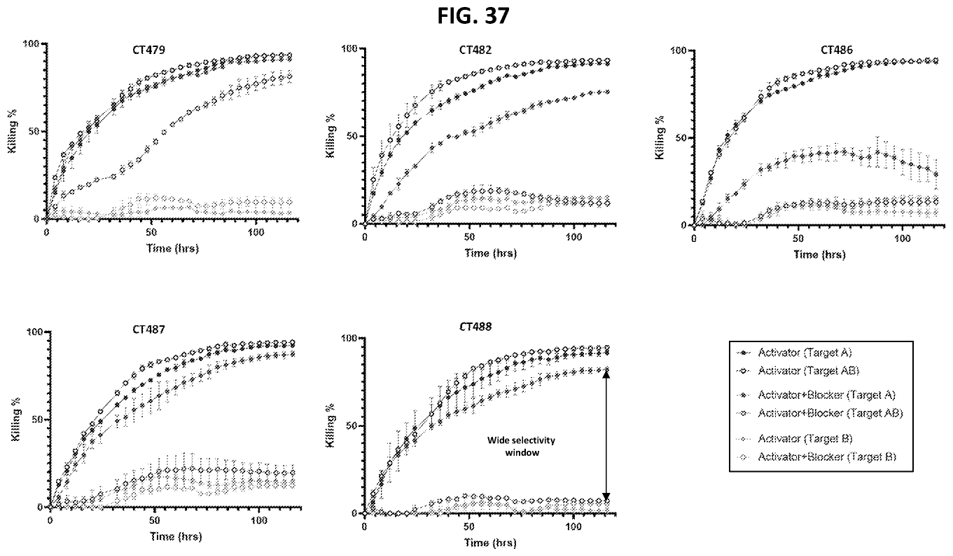

T cells transfected with either an EGFR ScFv CAR activator (CT-479, CT-482, CT-486, CT-487 or CT-488, as indicated in FIG. 37), or with EGFR ScFv CAR activator and an HLA-A*02 PA2.1 ScFv LIR1 inhibitor (C1765) were co-cultured with HeLa target cells. Wild type HeLa cell lines express EGFR but not HLA-A*02, but were transduced to express the HLA-A*02 inhibitory receptor target. Cells were co-cultured at a 1:1 ratio of effector to target (E:T). In the lower right of FIG. 37, effector cell receptor expression is indicated first, while HeLa cell expression is in parentheses. As can be seen from FIG. 37, a different degree of blocking is observed when the same HLA-A*02 PA2.1 ScFv LIR1 inhibitor was used with different EGFR activator receptors.

### Example 19: Inhibitory Receptors Reversibly Decrease Surface Level of Activator Receptors in T Cells

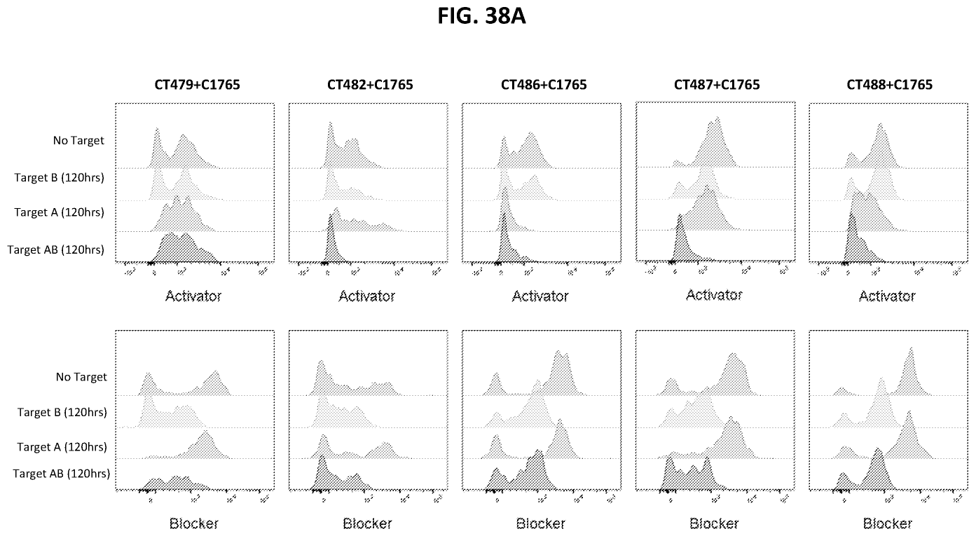

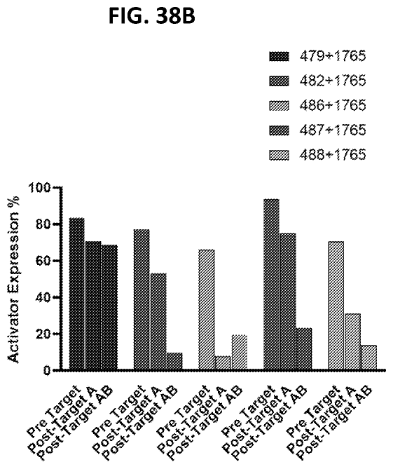

Primary T cells from two HLA-A*02 negative donors were transduced with an EGFR ScFv CAR activator (CT-479, CT-482, CT-486, CT-487 or CT-488) and an HLA-A*02 PA2.1 ScFv LIR1 inhibitor (C1765). Transduced cells were enriched by FACS sorting on the blocker and activator receptors, or by double column purification on the blocker and activator receptors. Transduced T cells were co-cultured with HeLa target cells. Wild type HeLa cell lines express EGFR but not HLA-A*02, but were transduced to express the HLA-A*02 inhibitory receptor target. Cells were co-cultured at a 1:1 ratio of effector to target (E:T). Surface expression of the EGFR CAR activator was assayed after 120 hours using labeled peptides that bound the activator and blocker receptors, and fluorescence activated cell sorting. The change in activator surface level following co-culture with HeLa cells expressing both activator and blocker ligands corresponded to the T cells' ability to kill target cells (compare FIG. 37 and FIG. 38).

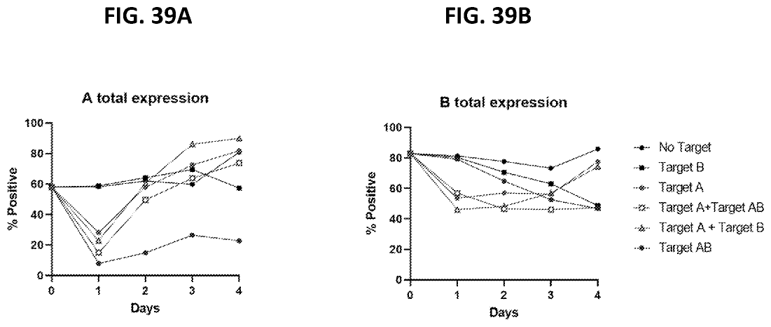

T cells expressing the CT-482 EGFR ScFv CAR activator and HLA-A*02 PA2.1 ScFv LIR1 inhibitor (C1765) combination, were co-cultured with HeLa cells expressing EGFR (Target A), HLA-A*02 (Target B), a combination of EGFR and HLA-A*02 on the same cell (Target AB), a mixed population of HeLa cells expressing Target A and Target AB on different cells, or a mixed population of HeLa cells expressing Target B and Target AB on different cells (FIGS. 39A-39B). T cells were cultured with HeLa target cells at a ratio of 1:1 effector cell to target cell. When T cells were co-cultured with a Target A plus Target AB population of HeLa cells, levels of activator decreased, then recovered (FIG. 39A). Furthermore, the activator and blocker antigens must be present together on the same cell to trigger activator surface expression loss on effector T cells. In contrast to the activator, blocker expression was largely unchanged (FIG. 39B).

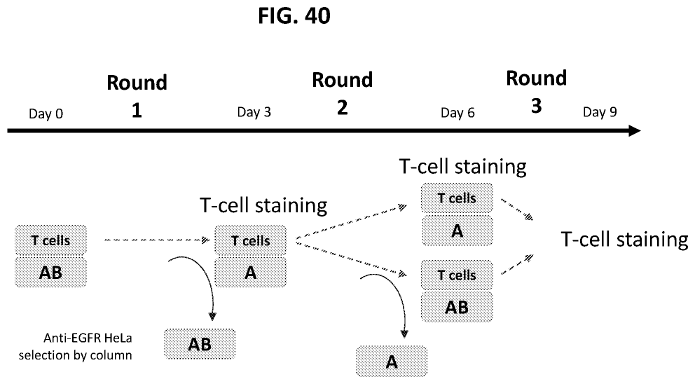

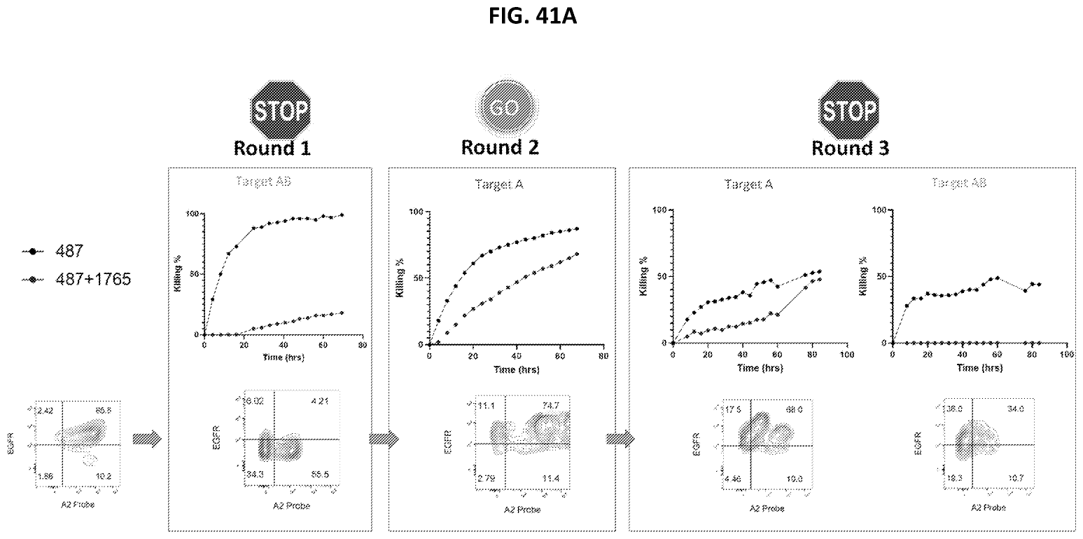

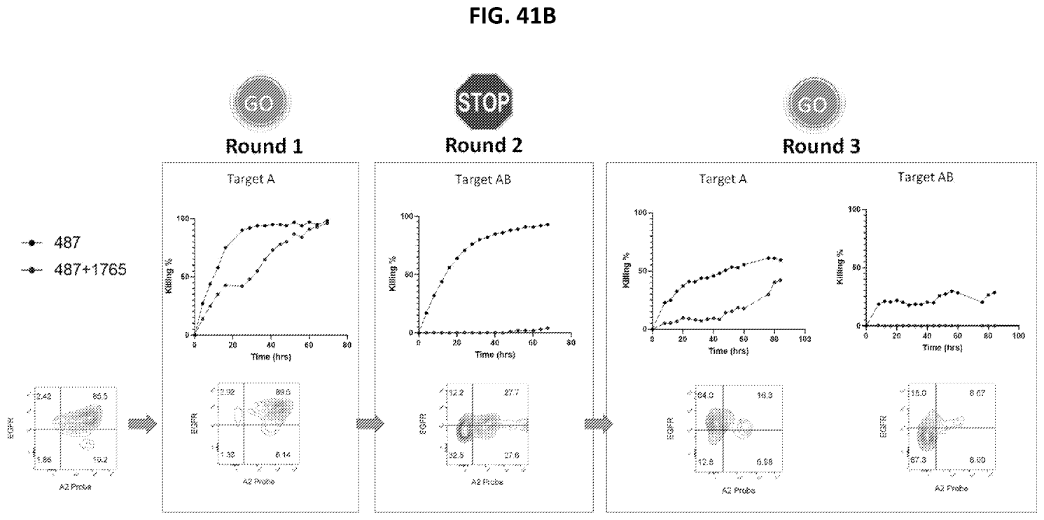

FIG. 40 shows a schematic for an experiment to determine if loss of expression of activator receptor by T cells was reversible. T cells expressing EGFR ScFv CAR activator receptor (CT-487) and HLA-A*02 PA2.1 ScFv LIR1 (C1765) inhibitor receptor were co-cultured with HeLa target cells expressing both the activator and blocker receptor targets (AB). Following 3 days co-culture, HeLa cells were removed using an anti-EGFR column, and the T cells were either stained for the activator and inhibitor receptors, or co-cultured with HeLa cells expressing EGFR activator target only for an additional 3 days. After the additional 3 days co-culture, HeLa cells were again removed using an anti-EGFR column, and the T cells were either stained for the activator and inhibitor receptors, or were co-cultured for an additional 3 days with HeLa cells expressing EGFR activator target only, or both the activator and blocker targets (AB), before staining. T cells were assayed for the presence of activator and inhibitor receptors (stained) using labeled EGFR and A2 probes, and the levels of receptor expression were quantified using fluorescence activated cell sorting. Results of the experiment are shown in FIGS. 41A-41B. As shown in FIGS. 41A-41B, co-culture of T cells with HeLa cells expressing both activator and inhibitor targets reduces EGFR activator staining (FIGS. 41A-41B, left panel). When T cells are co-cultured with HeLa cells expressing activator (Target A only) at round 2, expression of EGFR activator increases. Thus, activator surface loss is reversible and tracks with T cell cytotoxicity.

1. An immune cell, comprising:
a. a first engineered receptor, the first engineered receptor comprising a transmembrane region and an extracellular region, the extracellular region comprising a first ligand binding domain capable of specifically binding a first ligand; and
b. a second engineered receptor, the second engineered receptor comprising a transmembrane region and an extracellular region, the extracellular region comprising a second ligand binding domain capable of specifically binding a second ligand,
wherein binding of the first ligand binding domain to the first ligand activates or promotes activation of the immune cell by the first receptor, and
wherein binding of the second ligand binding domain to the second ligand inhibits activation of the immune cell by the first receptor.

2. The immune cell of claim 1, wherein the second ligand not expressed in a target cell due to loss of heterozygosity of a gene encoding the second ligand.

3. The immune cell of claim 1, wherein the second ligand is an HLA class I allele.

4. The immune cell of claim 1, wherein the second ligand is not expressed in the target cell due to loss of Y chromosome.

5.-6. (canceled)

7. The immune cell of claim 3, wherein the HLA class I allele comprises HLA-A, HLA-B or HLA-C.

8. The immune cell of claim 7, wherein the HLA class I allele is an HLA-A*02 allele.

9. The immune cell of claim 4, wherein the second ligand is encoded by a Y chromosome gene.

10.-11. (canceled)

12. The immune cell of claim 1, wherein the first ligand is expressed by target cells and a plurality of non-target cells.

13. (canceled)

14. The immune cell of claim 1, wherein the second ligand is not expressed by the target cells, and is expressed by the plurality of non-target cells.

15. The immune cell of claim 12, wherein the target cells are cancer cells and the non-target cells are non-cancerous cells.

16.-17. (canceled)

18. The immune cell of claim 1, wherein the first ligand is selected from the group of antigens in Table 5.

19. The immune cell of claim 18, wherein the first ligand binding domain is isolated or derived from the antigen binding domain of an antibody in Table 5.

20. (canceled)

21. The immune cell of claim 1, wherein the first ligand is a pan-HLA ligand.

22. The immune cell of claim 1, wherein the first ligand comprises HLA-A, HLA-B, HLA-C, HLA-E, HLA-F, of HLA-G.

23.-30. (canceled)

31. The immune cell of claim 1, wherein the first ligand is CD19 or a peptide antigen thereof, and the first ligand binding domain comprises SEQ ID NO: 275 or SEQ ID NO: 277, or a sequence having at least 90%, at least 95% or at least 99% identity thereto.

32. The immune cell of claim 1, wherein the first ligand comprises a pan-HLA ligand, and the first ligand binding domain comprises a sequence of SEQ ID NO: 167, SEQ ID NO: 169, SEQ ID NO: 171, SEQ ID NO: 173, SEQ ID NO: 175, or SEQ ID NO: 177, or a sequence having at least 90%, at least 95% or at least 99% identity thereto.

33.-57. (canceled)

58. A pharmaceutical composition, comprising a plurality of the immune cells of any one of claim 1.

59.-60. (canceled)

61. A method of increasing the specificity of an adoptive cell therapy in a subject, comprising administering to the subject a plurality of the immune cell of claim 1.

62. A method of treating cancer with an adoptive cell therapy, comprising administering to the subject a plurality of the immune cell of any one of claim 1.

63.-65. (canceled)

66. A method of making the immune cell of any one of claim 1, comprising
a. providing a plurality of immune cells; and
b. transforming the immune cells with a vector encoding a first engineered receptor comprising a transmembrane region and an extracellular region, the extracellular region comprising a first ligand binding domain capable of specifically binding a first ligand, and a vector encoding a second engineered receptor comprising a transmembrane region and an extracellular region, the extracellular region comprising a second ligand binding domain capable of specifically binding a second ligand;
wherein binding of the first ligand binding domain to the first ligand activates or promotes activation of the immune cell, and
wherein binding of the second ligand binding domain to a second ligand inhibits activation of the immune cell by the first ligand.

67.-102. (canceled)


SEQUENCE LISTING (391 sequences)

SEQ ID NO: 1 - protein, 45 aa, Artificial Sequence
  CD8alpha hinge
TTTPAPRPPTPAPTIASQPLSLRPEACRPAAGGAVHTRGLDFACD

SEQ ID NO: 2 - DNA, 135 bp, Artificial Sequence
  CD8alpha hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 3 - protein, 41 aa, Artificial Sequence
  CD28 hinge
CTIEVMYPPPYLDNEKSNGTIIHVKGKHLCPSPLFPGPSKP

SEQ ID NO: 4 - DNA, 123 bp, Artificial Sequence
  CD28 hinge
tgtaccattgaagttatgtatcctcctccttacctagacaatgagaagagcaatggaaccattatccatgtgaaagggaaacacctttgtccaagtcccctatttcccggaccttctaagccc

SEQ ID NO: 5 - protein, 27 aa, Artificial Sequence
  CD28 transmembrane domain
FWVLVVVGGVLACYSLLVTVAFIIFWV

SEQ ID NO: 6 - DNA, 81 bp, Artificial Sequence
  CD28 transmembrane domain
ttctgggtgctggtcgttgtgggcggcgtgctggcctgctacagcctgctggtgacagtggccttcatcatcttttgggtg

SEQ ID NO: 7 - protein, 25 aa, Artificial Sequence
  IL-2R beta transmembrane domain
IPWLGHLLVGLSGAFGFIILVYLLI

SEQ ID NO: 8 - DNA, 75 bp, Artificial Sequence
  IL-2R beta transmembrane domain
attccgtggctcggccacctcctcgtgggcctcagcggggcttttggcttcatcatcttagtgtacttgctgatc

SEQ ID NO: 9 - protein, 112 aa, Artificial Sequence
  CD3 zeta activation domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 336 bp, Artificial Sequence
  CD3 zeta activation domain
agagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 11 - protein, 43 aa, Artificial Sequence
  CD3 zeta activation domain
RVKFSRSADAPAYQQGQNQLYNELNLGRREEYDVLHMQALPPR

SEQ ID NO: 12 - DNA, 129 bp, Artificial Sequence
  CD3 zeta activation domain
agagtgaagttcagcaggagcgcagacgcccccgcgtaccagcagggccagaaccagctctataacgagctcaatctaggacgaagagaggagtacgatgttttgcacatgcaggccctgccccctcgc

SEQ ID NO: 13 - protein, 41 aa, Artificial Sequence
  CD28 co-stimulatory domain
RSKRSRLLHSDYMNMTPRRPGPTRKHYQPYAPPRDFAAYRS

SEQ ID NO: 14 - DNA, 123 bp, Artificial Sequence
  CD28 co-stimulatory domain
aggagcaagcggagcagactgctgcacagcgactacatgaacatgaccccccggaggcctggccccacccggaagcactaccagccctacgcccctcccagggatttcgccgcctaccggagc

SEQ ID NO: 15 - protein, 94 aa, Artificial Sequence
  IL-2-Rbeta intracellular domain
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 16 - DNA, 282 bp, Artificial Sequence
  IL-2-Rbeta intracellular domain
aactgcaggaacaccgggccatggctgaagaaggtcctgaagtgtaacaccccagacccctcgaagttcttttcccagctgagctcagagcatggaggcgacgtccagaagtggctctcttcgcccttcccctcatcgtccttcagccctggcggcctggcacctgagatctcgccactagaagtgctggagagggacaaggtgacgcagctgctccccctgaacactgatgcctacttgtctctccaagaactccagggtcaggacccaactcacttggtg

SEQ ID NO: 17 - protein, 4 aa, Artificial Sequence
  STAT5 recruitment motif
YLSL

SEQ ID NO: 18 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 19 - DNA, 300 bp, Artificial Sequence
  CD3 epsilon intracellular domain
aagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagcgcagaatcggcggaagcaggagcaagcggagcagactgctgcacagcgactacatgaacatgaccccccggaggcctggccccacccggaagcactaccagccctacgcccctcccagggatttcgccgcctaccggagctag

SEQ ID NO: 20 - DNA, 69 bp, Artificial SEquence
  TCR beta transmembrane domain
accatcctctatgagatcttgctagggaaggccaccttgtatgccgtgctggtcagtgccctcgtgctg

SEQ ID NO: 21 - protein, 4 aa, Artificial Sequence
  ITAM motif
  X (2)..(3): X can be any amino acid
YXXL

SEQ ID NO: 22 - DNA, 270 bp, Artificial Sequence
  CD3 gamma intracellular domain
ggacaggatggagttcgccagtcgagagcttcagacaagcagactctgttgcccaatgaccagctctaccagcccctcaaggatcgagaagatgaccagtacagccaccttcaaggaaaccagttgaggaggaatggcggaagcaggagcaagcggagcagactgctgcacagcgactacatgaacatgaccccccggaggcctggccccacccggaagcactaccagccctacgcccctcccagggatttcgccgcctaccggagctag

SEQ ID NO: 23 - protein, 10 aa, Artificial Sequence
  UTY peptide
  X (1)..(1): X is R or G
  X (6)..(6): X is E or A
  X (8)..(8): X is V or P
XESEEXSXSL

SEQ ID NO: 24 - protein, 9 aa, Artificial Sequence
  RPSY peptide
  X (8)..(8): X is V or L
TIRYPDPXI

SEQ ID NO: 25 - protein, 8 aa, Artificial Sequence
  UTY peptide
  X (5)..(5): X is H or R
  X (7)..(7): X is D or N
LPHNXTXL

SEQ ID NO: 26 - protein, 23 aa, Artificial Sequence
  TCR alpha transmembrane domain
VIGFRILLLKVAGFNLLMTLRLW

SEQ ID NO: 27 - DNA, 69 bp, Artificial Sequence
  TCR alpha transmembrane domain
gtgattgggttccgaatcctcctcctgaaagtggccgggtttaatctgctcatgacgctgcggctgtgg

SEQ ID NO: 28 - protein, 23 aa, Artificial Sequence
  TCR beta transmembrane domain
TILYEILLGKATLYAVLVSALVL

SEQ ID NO: 29 - protein, 21 aa, Artificial Sequence
  CD3 zeta transmembrane domain
LCYLLDGILFIYGVILTALFL

SEQ ID NO: 30 - protein, 89 aa, Artificial Sequence
  CD3 delta intracellular domain
GHETGRLSGAADTQALLRNDQVYQPLRDRDDAQYSHLGGNWARNKGGSRSKRSRLLHSDYMNMTPRRPGPTRKHYQPYAPPRDFAAYRS

SEQ ID NO: 31 - DNA, 269 bp, Artificial Sequence
  CD3 delta intracellular domain
ggacatgagactggaaggctgtctggggctgccgacacacaagctctgttgaggaatgaccaggtctatcagcccctccgagatcgagatgatgctcagtacagccaccttggaggaaactgggctcggaacaagggcggaagcaggagcaagcggagcagactgctgcacagcgactacatgaacatgaccccccggaggcctggccccacccggaagcactaccagccctacgcccctcccagggatttcgccgcctaccggagcta

SEQ ID NO: 32 - protein, 99 aa, Artificial Sequence
  CD3 epsilon intracellular domain
KNRKAKAKPVTRGAGAGGRQRGQNKERPPPVPNPDYEPIRKGQRDLYSGLNQRRIGGSRSKRSRLLHSDYMNMTPRRPGPTRKHYQPYAPPRDFAAYRS

SEQ ID NO: 33 - protein, 89 aa, Artificial Sequence
  CD3 gamma intracellular domain
GQDGVRQSRASDKQTLLPNDQLYQPLKDREDDQYSHLQGNQLRRNGGSRSKRSRLLHSDYMNMTPRRPGPTRKHYQPYAPPRDFAAYRS

SEQ ID NO: 34 - protein, 11 aa, Artificial Sequence
  SMCY peptide
  X (3)..(3): X is S or A
  X (8)..(8): X is R or Q
SPXVDKAXAEL

SEQ ID NO: 35 - protein, 10 aa, Artificial Sequence
  TCR beta intracellular domain
MAMVKRKDSR

SEQ ID NO: 36 - DNA, 30 bp, Artificial Sequence
  TCR beta intracellular domain
atggccatggtcaagagaaaggattccaga

SEQ ID NO: 37 - protein, 41 aa, Artificial Sequence; identical to SEQ ID NO: 13
  CD28 intracellular domain

SEQ ID NO: 38 - DNA, 123 bp, Artificial Sequence; identical to SEQ ID NO: 14
  CD28 intracellular domain

SEQ ID NO: 39 - protein, 42 aa, Artificial Sequence
  4-1BB intracellular domain
KRGRKKLLYIFKQPFMRPVQTTQEEDGCSCRFPEEEEGGCEL

SEQ ID NO: 40 - DNA, 126 bp, Artificial Sequence
  4-1BB intracellular domain
aaacggggcagaaagaaactcctgtatatattcaaacaaccatttatgaggccagtacaaactactcaagaggaagatggctgtagctgccgatttccagaagaagaagaaggaggatgtgaactg

SEQ ID NO: 41 - protein, 16 aa, Artificial Sequence
  HLA-A*02 CDR-L1
RSSQSIVHSNGNTYLE

SEQ ID NO: 42 - protein, 12 aa, Artificial Sequence
  HLA-A*02 CDR-L2
KVSNRFSGVPDR

SEQ ID NO: 43 - protein, 9 aa, Artificial Sequence
  HLA-A*02 CDR-L3
FQGSHVPRT

SEQ ID NO: 44 - protein, 12 aa, Artificial Sequence
  HLA-A*02 CDR-H1
ASGYTFTSYHIH

SEQ ID NO: 45 - protein, 18 aa, Artificial Sequence
  HLA-A*02 CDR-H2
WIYPGNVNTEYNEKFKGK

SEQ ID NO: 46 - protein, 9 aa, Artificial Sequence
  HLA-A*02 CDR-H3
EEITYAMDY

SEQ ID NO: 47 - protein, 16 aa, Artificial Sequence
  HLA-A*02 CDR-L1
RSSQSIVHSNGNTYLD

SEQ ID NO: 48 - protein, 12 aa, Artificial Sequence
  HLA-A*02 CDR-L2
KVSNRFSGVPDR

SEQ ID NO: 49 - protein, 9 aa, Artificial Sequence
  HLA-A*02 CDR-L3
MQGSHVPRT

SEQ ID NO: 50 - protein, 11 aa, Artificial Sequence
  HLA-A*02 CDR-H1
SGYTFTSYHMH

SEQ ID NO: 51 - protein, 17 aa, Artificial Sequence
  HLA-A*02 CDR-H2
WIYPGDGSTQYNEKFKG

SEQ ID NO: 52 - protein, 9 aa, Artificial Sequence
  HLA-A*02 CDR-H3
EGTYYAMDY

SEQ ID NO: 53 - protein, 247 aa, Artificial Sequence
  HLA-A*02 ScFv
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSS

SEQ ID NO: 54 - protein, 247 aa, Artificial Sequence
  HLA-A*02 ScFv
QVQLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 55 - protein, 247 aa, Artificial Sequence
  HLA-A*02 ScFv
QVQLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 56 - protein, 247 aa, Artificial Sequence
  HLA-A*02 ScFv
EVQLVESGGGLVKPGGSLRLSCAASGYTFTSYHIHWVRQAPGKGLEWVGWIYPGNVNTEYNEKFKGRFTISRDDSKNTLYLQMNSLKTEDTAVYYCAREEITYAMDYWGQGTTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHVPRTFGGGTKVEIK

SEQ ID NO: 57 - protein, 247 aa, Artificial Sequence
  HLA-A*02 ScFv
QVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVK

SEQ ID NO: 58 - protein, 247 aa, Artificial Sequence
  HLA-A*02 ScFv
QVQLVQSGAEVKKPGSSVKVSCKASGYTFTSYHMHWVRQAPGQGLEWIGYIYPGNVNTEYNEKFKGKATLTADKSTNTAYMELSSLRSEDTAVYFCAREEITYAMDYWGQGTLVTVSSGGGGSGGGGSGGGGSGGDVQMTQSPSTLSASVGDRVTITCSSSQSIVHSNGNTYMEWYQQKPGKAPKLLIYKVSNRFSGVPDRFSGSGSGTEFTLTISSLQPDDFATYYCHQGSHVPRTFGQGTKVEVK

SEQ ID NO: 59 - protein, 247 aa, Artificial Sequence
  HLA-A*02 ScFv
QVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSGGDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 60 - protein, 247 aa, Artificial Sequence
  HLA-A*02 ScFv
QLQLQESGPGLVKPSETLSLTCTVSGYTFTSYHIQWIRQPPGKGLEWIGWIYPGDGSTQYNEKFKGRATISVDTSKNQFSLNLDSVSAADTAIYYCAREGTYYAMDYWGKGSTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTFTISSLQPEDIATYYCFQGSHVPRTFGPGTKVDIK

SEQ ID NO: 61 - protein, 247 aa, Artificial Sequence
  HLA-A*02 ScFv
EVQLVQSGAELKKPGSSVKVSCKASGYTFTSYHIQWVKQAPGQGLEWIGWIYPGDGSTQYNEKFKGKATLTVDKSTNTAYMELSSLRSEDTAVYYCAREGTYYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPSRFSGSGSGTDFTLTISSLQPDDFATYYCFQGSHVPRTFGQGTKVEVK

SEQ ID NO: 62 - protein, 247 aa, Artificial Sequence
  HLA-A*02 ScFv
QVQLVQSGAEVKKPGSSVKVSCKASGYTFTSYHIQWVRQAPGQGLEWMGWIYPGDGSTQYNEKFKGRVTITADKSTSTAYMELSSLRSEDTAVYYCAREGTYYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 63 - protein, 247 aa, Artificial Sequence
  HLA-A*02 ScFv
QVTLKQSGAEVKKPGSSVKVSCTASGYTFTSYHVSWVRQAPGQGLEWLGRIYPGDGSTQYNEKFKGKVTITADKSMDTSFMELTSLTSEDTAVYYCAREGTYYAMDLWGQGTLVTVSSGGGGSGGGGSGGGGSGGEIVLTQSPGTLSLSPGERATLSCRSSQSIVHSNGNTYLAWYQQKPGQAPRLLISKVSNRFSGVPDRFSGSGSGTDFTLTISRLEPEDFAVYYCQQGSHVPRTFGGGTKVEIK

SEQ ID NO: 64 - protein, 247 aa, Artificial Sequence
  HLA-A*02 ScFv
QVQLVQSGAEVKKPGASVKVSCKASGYTFTSYHMHWVRQAPGQRLEWMGWIYPGDGSTQYNEKFKGKVTITRDTSASTAYMELSSLRSEDTAVYYCAREGTYYAMDYWGQGTLVTVSSGGGGSGGGGSGGGGSGGDIVMTQTPLSLPVTPGEPASISCRSSQSIVHSNGNTYLDWYLQKPGQSPQLLIYKVSNRFSGVPDRFSGSGSGTDFTLKISRVEAEDVGVYYCMQGSHVPRTFGGGTKVEIK

SEQ ID NO: 65 - protein, 670 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHPSQEGPSPAVPSIYATLAIH

SEQ ID NO: 66 - DNA, 3229 bp, Homo sapiens
aaatgagttttaaaaaggcttgtccaggaagcacatatgggagctggtcactctgcattttgggccctcctggaggtgtttagaccttccgagagagaaactgagacacatgagagggaagaaatgactcagtggtgagaccctgtggagtcccacccacaaccagcacactgtgacccactgcacaaacctctagcccacagctcacttcctcctttaagaagagaagagaaaagaggagaggagaggaggaacagaaaagaaaagaaaagaaaaagtgggaaacaaataatctaagaatgaggagaaagcaagaagagtgacccccttgtgggcactccattggttttatggcgcctctactttctggagtttgtgtaaaacaaaaatattatggtctttgtgcacatttacatcaagctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggcccccggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacacacagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaaaaaaaaaaaaaaa

SEQ ID NO: 67 - protein, 6 aa, Artificial Sequence
  ITIM
NLYAAV

SEQ ID NO: 68 - protein, 6 aa, Artificial Sequence
  ITIM
VTYAEV

SEQ ID NO: 69 - protein, 6 aa, Artificial Sequence
  ITIM
VTYAQL

SEQ ID NO: 70 - protein, 6 aa, Artificial Sequence
  ITIM
SIYATL

SEQ ID NO: 71 - protein, 35 aa, Artificial Sequence
  ITIM 1-2
NLYAAVKHTQPEDGVEMDTRSPHDEDPQAVTYAEV

SEQ ID NO: 72 - protein, 58 aa, Artificial Sequence
  ITIM 2-3
VTYAEVKHSRPRREMASPPSPLSGEFLDTKDRQAEEDRQMDTEAAASEAPQDVTYAQL

SEQ ID NO: 73 - protein, 36 aa, Artificial Sequence
  ITIM 3-4
VTYAQLHSLTLRREATEPPPSQEGPSPAVPSIYATL

SEQ ID NO: 74 - protein, 87 aa, Artificial Sequence
  ITIM 1-3
NLYAAVKHTQPEDGVEMDTRSPHDEDPQAVTYAEVKHSRPRREMASPPSPLSGEFLDTKDRQAEEDRQMDTEAAASEAPQDVTYAQL

SEQ ID NO: 75 - protein, 88 aa, Artificial Sequence
  ITIM 2-4
VTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 76 - protein, 117 aa, Artificial Sequence
  ITIM 1-4
NLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 77 - protein, 20 aa, Artificial Sequence
  D3D4 domain
YGSQSSKPYLLTHPSDPLEL

SEQ ID NO: 78 - protein, 43 aa, Artificial Sequence
  short hinge
VVSGPSGGPSSPTTGPTSTSGPEDQPLTPTGSDPQSGLGRHLG

SEQ ID NO: 79 - protein, 86 aa, Artificial Sequence
  hinge- transmembrane
YGSQSSKPYLLTHPSDPLELVVSGPSGGPSSPTTGPTSTSGPEDQPLTPTGSDPQSGLGRHLGVVIGILVAVILLLLLLLLLFLIL

SEQ ID NO: 80 - protein, 190 aa, Artificial Sequence
  hinge-transmembrane-intracellular domain
VVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 81 - protein, 167 aa, Artificial Sequence
  LILRB1 intracellular domain
RHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 82 - protein, 253 aa, Artificial Sequence
  LILRB1 hinge-transmemebrane-intracellular domain
YGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 83 - protein, 233 aa, Artificial Sequence
  LILRB1 hinge-TM-intracellular domain
VVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 84 - protein, 63 aa, Artificial Sequence
  LILRB1 hinge domain
YGSQSSKPYLLTHPSDPLELVVSGPSGGPSSPTTGPTSTSGPEDQPLTPTGSDPQSGLGRHLG

SEQ ID NO: 85 - protein, 23 aa, Artificial Sequence
  LILRB1 transmembrane domain
VVIGILVAVILLLLLLLLLFLIL

SEQ ID NO: 86 - protein, 237 aa, Artificial Sequence
  MSLN binding domain
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGDIVMTQSSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 87 - DNA, 711 bp, Artificial Sequence
  MSLN binding domain
caggtgcagctggtgcagtctggggctgaggtggagaagcctggggcctcagtgaaggtctcctgcaaggcttctggatacaccttcaccgactactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgtctggctgggactttgactactggggccagggaaccctggtcaccgtgtcctcaggcggaggtggaagcggagggggaggatctggcggcggaggaagcggaggcgacatcgtgatgacccagtcttcctccctgtctgcatctgtcggagacagagtcaccatcacttgccgggccagtcagagcattaggtactatttaagttggtatcagcagaaaccaggaaaagcccctaagctcctgatctatactgcatccattttacaaaatggggtcccatcaaggttcagtggcagtggatctgggacagatttcactctcaccatcagcagcctgcaacctgaggattttgcaacttattactgcctccagacttacactactccggactttggcccagggaccaaggtggaaatcaaa

SEQ ID NO: 88 - protein, 246 aa, Artificial Sequence
  MSLN binding domain
QVQLVQSGAEVRAPGASVKISCKASGFTFRGYYIHWVRQAPGQGLEWMGIINPSGGSRAYAQKFQGRVTMTRDTSTSTVYMELSSLRSDDTAMYYCARTASCGGDCYYLDYWGQGTLVTVSSGGGGSGGGGSGGGGSGGDIQMTQSPPTLSASVGDRVTITCRASENVNIWLAWYQQKPGKAPKLLIYKSSSLASGVPSRFSGSGSGAEFTLTISSLQPDDFATYYCQQYQSYPLTFGGGTKVEIK

SEQ ID NO: 89 - DNA, 738 bp, Artificial Sequence
  MSLN binding domain
caggtgcagctggtgcagtctggggctgaggtgagggcacctggggcctcagtgaagatttcctgcaaggcttctggattcaccttcagaggctactatatccactgggtgcgacaggcccctggacaagggcttgagtggatgggaatcatcaaccctagtggtggtagcagagcctacgcacagaagttccagggcagggtcaccatgaccagggacacttccacgagcacagtctacatggagctgagcagcctgagatctgacgacacggccatgtattactgtgcgagaaccgcaagttgtggtggtgactgctactaccttgactactggggccagggaaccctggtcaccgtgtcctcaggcggaggtggaagcggagggggaggatctggcggcggaggaagcggaggcgacatccagatgacccagtctcctcccaccctgtctgcatctgtaggagacagagtcaccatcacttgccgggccagtgagaatgttaatatctggttggcctggtatcagcagaaaccagggaaagcccctaagctcctgatctataagtcatccagtttagcaagtggggtcccatcaaggttcagtggcagtggatctggggcagaattcactctcaccatcagcagcctgcagcctgatgattttgcaacttattactgccaacagtatcaaagttaccccctcactttcggcggagggaccaaggtggaaatcaaa

SEQ ID NO: 90 - protein, 242 aa, Artificial Sequence
  MSLN binding domain
QVQLVQSGAEVKKPGASVKVSCKASGYSFTGYTMNWVRQAPGQRLEWMGLITPYNGASSYNQKFRGRVTITRDTSASTAYMELSSLRSEDTAVYYCARGGYDGRGFDYWGQGTTVTVSSGGGGSGGGGSGGGGSGGDIQMTQSPSSLSASVGDRVTITCSASSSVSYMHWYQQKPGKAPKRLIYDTSKLASGVPSRFSGSGSGTEFTLTISSLQPEDFATYYCQQWSGYPLTFGQGTKLEIK

SEQ ID NO: 91 - DNA, 726 bp, Artificial Sequence
  MSLN binding domain
caggtgcagctggtgcagtctggggctgaggtgaagaagcctggggcctcagtgaaggtttcctgcaaggcttctggatactcattcaccggctacaccatgaactgggtgaggcaggcccctggacaaagacttgagtggatgggacttatcaccccttacaatggtgcttctagctacaaccagaagttcaggggcagggtcacaatcactagagacacgtcagccagcacagcctacatggagctctccagcctgagatctgaagacactgcagtctattactgtgcaagggggggttacgacgggaggggttttgactactggggccagggaaccacggtcaccgtgtcctcaggcggaggtggaagcggagggggaggatctggcggcggaggaagcggaggcgacatccagatgacccagtctccttcaagcttgtctgcatctgtaggagacagggtcaccatcacttgcagtgccagctcaagtgtaagttacatgcactggtatcagcagaaaccaggcaaggcccctaagagattgatctatgacacatccaaattagcaagtggggtcccaagtcgcttcagtggcagtggatctgggaccgaattcactctcaccatcagcagcttgcagcctgaggattttgcaacttattactgccagcagtggagtggttaccctctcacgttcggtcaggggacaaagttggaaatcaaa

SEQ ID NO: 92 - protein, 242 aa, Artificial Sequence
  MSLN binding domain
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSGGDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 93 - DNA, 726 bp, Artificial Sequence
  MSLN binding domain
caggtgcagctgcagcagtctgggcctgagctggagaagcctggggcctcagtgaagatttcctgcaaggcttctggatactcattcaccggctacaccatgaactgggtgaagcagagccatggaaaaagccttgagtggattggacttatcaccccttacaatggtgcttctagctacaaccagaagttcaggggcaaggccacattaactgtagacaagtcatccagcacagcctacatggacctcctcagcctgacatctgaagactctgcagtctatttctgtgcaagggggggttacgacgggaggggttttgactactggggccagggaaccacggtcaccgtgtcctcaggcggaggtggaagcggagggggaggatctggcggcggaggaagcggaggcgacatcgagctcacccagtctcctgcaatcatgtctgcatctccaggagagaaggtcaccatgacttgcagtgccagctcaagtgtaagttacatgcactggtatcagcagaaatcaggcacctcccctaagagatggatctatgacacatccaaattggcaagtggggtcccaggtcgcttcagtggcagtggatctgggaactcttactctctcaccatcagcagcgtggaggctgaggatgatgcaacttattactgccagcagtggagtggttaccctctcacgttcggtgctgggacaaagttggaaatcaaa

SEQ ID NO: 94 - protein, 243 aa, Artificial Sequence
  CEA binding domain
QVQLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSGGENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 95 - DNA, 729 bp, Artificial Sequence
  CEA binding domain
caggtccagctgcagcagtctggggcagagcttgtgaggtcagggacctcagtcaagttgtcctgcacagcttctggcttcaacattaaagactcctatatgcactggttgaggcaggggcctgaacagggcctggagtggattggatggattgatcctgagaatggtgatactgaatatgccccgaagttccagggcaaggccacttttactacagacacatcctccaacacagcctacctgcagctcagcagcctgacatctgaggacactgccgtctattactgtaatgaagggacaccgacagggccatactattttgactactggggtcaaggaaccacagtcaccgtgtcctcaggcggaggtggaagcggagggggaggatctggcggcggaggaagcggaggcgagaacgttctcacccagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccgctcacgttcggtgctgggaccaagctggagctgaaa

SEQ ID NO: 96 - protein, 243 aa, Artificial Sequence
  CEA binding domain
QVQLVQSGAEVKKPGASVKVSCKASGFNIKDSYMHWVRQAPGQGLEWMGWIDPENGDTEYAPKFQGRVTMTTDTSTSTAYMELRSLRSDDTAVYYCNEGTPTGPYYFDYWGQGTTVTVSSGGGGSGGGGSGGGGSGGEIVLTQSPATLSLSPGERATLSCSASSSVSYMHWYQQKPGLAPRLLIYSTSNLASGIPDRFSGSGSGTDFTLTISRLEPEDFAVYYCQQRSSYPLTFGQGTKLEIK

SEQ ID NO: 97 - DNA, 729 bp, Artificial Sequence
  CEA binding domain
caggtccagctggtgcagtctggggcagaggtgaagaaaccaggggcctcagtcaaggtgtcctgcaaagcttctggcttcaacattaaagactcctatatgcactgggtgaggcaggcgcctggacagggcctggagtggatgggatggattgatcctgagaatggtgatactgaatatgccccgaagttccagggcagggtcactatgactacagacacatccacctccacagcctacatggagctcaggagcctgagatctgacgacactgccgtctattactgtaatgaagggacaccgacagggccatactattttgactactggggtcaaggaaccacagtcaccgtgtcctcaggcggaggtggaagcggagggggaggatctggcggcggaggaagcggaggcgagatcgttctcacccagtctccagcaaccttgtctctgtctccaggggagagggccaccctaagctgcagtgccagctcaagtgtaagttacatgcactggtaccagcagaagccaggccttgctcccagactcctgatttatagcacatccaacctggcttctggaatccctgatcgcttcagtggcagtggatctgggaccgatttcactctcacaatcagccgactggagcctgaagatttcgccgtttattactgccagcaaaggagtagttacccgctcacgttcggtcaggggaccaagctggagatcaaa

SEQ ID NO: 98 - protein, 243 aa, Artificial Sequence
  CEA binding domain
EVQLAESGGGLVQPGGSLRLSCAASGFTFSSDAMSWVRQAPGKGLEWVSAISGSGGSTYYADSVKGRFTISRDNSKNTLYLQMNSLRAEDTAVYYCAKSNEFLFDYWGQGTLVTVSSGGGGSGGGGSGGGGSGGSSELTQDPAVSVALGQTVRITCQGDSLRSSYASWYRQRPGQAPVLVIYGKNNRPSGIPDRFSGSSSGNTASLTITGAQAEDEADYYWNSSYAWLPYVVFGGGTKLTVLG

SEQ ID NO: 99 - DNA, 729 bp, Artificial Sequence
  CEA binding domain
gaggtgcagctggcggagtctgggggaggcttggtacagcctggggggtccctgagactctcctgtgcagcctctggattcacctttagcagcgatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcaaagtctaatgagtttctttttgactactggggccaaggtaccctggtcaccgtgtcgagtggcggaggtggaagcggagggggaggatctggcggcggaggaagcggaggctcttctgagctgactcaggaccctgctgtgtctgtggccttgggacagacagtcaggatcacatgccaaggagacagcctcagaagctcttatgcaagctggtaccggcagaggccaggacaggcccctgtacttgtcatctatggtaaaaacaaccggccctcagggatcccagaccgattctctggctccagctcaggaaacacagcttccttgaccatcactggggctcaggcggaagatgaggctgactattactggaactccagctacgcttggctgccctacgtggtattcggcggagggaccaagctgaccgtcctaggt

SEQ ID NO: 100 - protein, 243 aa, Artificial Sequence
  CEA binding domain
QVQLEQSGAGVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSGGENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 101 - DNA, 729 bp, Artificial Sequence
  CEA binding domain
caggtccagctggagcagtctggggcaggggttgtgaagccaggggcctcagtcaagttgtcctgcaaagcttctggcttcaacattaaagactcctatatgcactggttgaggcaggggcctggacagcgcctggagtggattggatggattgatcctgagaatggtgatactgaatatgccccgaagttccagggcaaggccacttttactacagacacatccgccaacacagcctacctggggctcagcagcctgagacctgaggacactgccgtctattactgtaatgaagggacaccgacagggccatactattttgactactggggtcaaggaaccctagtcaccgtgtcctcaggcggaggtggaagcggagggggaggatctggcggcggaggaagcggaggcgagaacgttctcacccagtctccaagctctatgtctgtatctgtcggggacagggtcaacatcgcctgcagtgccagctcaagtgtaccttacatgcactggctccagcagaagccaggcaaatctcccaaactcctgatttatctcacatccaacctggcttctggagtccctagccgcttcagtggcagtggatctgggaccgattactctctcacaatcagctcagtgcagcctgaagatgctgccacttattactgccagcaaaggagtagttacccgctcacgttcggtggtgggaccaagctggagatcaaa

SEQ ID NO: 102 - protein, 249 aa, Artificial Sequence
  EGFR ScFv
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSGGGGSGGGGSGGAIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIK

SEQ ID NO: 103 - DNA, 747 bp, Artificial Sequence
  EGFR ScFv
caggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttatatgggatgatggaagttataaatactatggagactccgtgaagggccgattcaccatctccagagacaattccaagaacacgctgtatctgcaaatgaacagcctgagagccgaggacacggctgtgtattactgtgcgagagatggtattactatggttcggggagttatgaaggactactttgactactggggccagggaaccctggtcaccgtctcctcaggcggaggtggaagcggagggggaggatctggcggcggaggaagcggaggcgccatccagttgacccagtctccatcctccctgtctgcatctgtaggagacagagtcaccatcacttgccgggcaagtcaggacattagcagtgctttagtctggtatcagcagaaaccagggaaagctcctaagctcctgatctatgatgcctccagtttggaaagtggggtcccatcaaggttcagcggcagtgaatctgggacagatttcactctcaccatcagcagcctgcagcctgaagattttgcaacttattactgtcaacagtttaatagttacccgctcactttcggcggagggaccaaggtggagatcaaa

SEQ ID NO: 104 - protein, 249 aa, Artificial Sequence
  EGFR ScFv
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGGGGSGGGGSGGQVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSS

SEQ ID NO: 105 - DNA, 747 bp, Artificial Sequence
  EGFR ScFv
gccatccagttgacccagtctccatcctccctgtctgcatctgtaggagacagagtcaccatcacttgccgggcaagtcaggacattagcagtgctttagtctggtatcagcagaaaccagggaaagctcctaagctcctgatctatgatgcctccagtttggaaagtggggtcccatcaaggttcagcggcagtgaatctgggacagatttcactctcaccatcagcagcctgcagcctgaagattttgcaacttattactgtcaacagtttaatagttacccgctcactttcggcggagggaccaaggtggagatcaaaggcggaggtggaagcggagggggaggatctggcggcggaggaagcggaggccaggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttatatgggatgatggaagttataaatactatggagactccgtgaagggccgattcaccatctccagagacaattccaagaacacgctgtatctgcaaatgaacagcctgagagccgaggacacggctgtgtattactgtgcgagagatggtattactatggttcggggagttatgaaggactactttgactactggggccagggaaccctggtcaccgtctcctca

SEQ ID NO: 106 - protein, 247 aa, Artificial Sequence
  EGFR ScFv
QIQLVQSGPELKKPGETVKISCKASGYTFTEYPIHWVKQAPGKGFKWMGMIYTDIGKPTYAEEFKGRFAFSLETSASTAYLQINNLKNEDTATYFCVRDRYDSLFDYWGQGTTLTVSSGGGGSGGGGSGGGGSGGDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 107 - DNA, 741 bp, Artificial Sequence
  EGFR ScFv
cagatccagttggtgcagtctggacctgagctgaagaagcctggagagacagtcaagatctcctgcaaggcctctgggtataccttcacagaatatccaatacactgggtgaagcaggctccaggaaagggtttcaagtggatgggcatgatatacaccgacattggaaagccaacatatgctgaagagttcaagggacggtttgccttctctttggagacctctgccagcactgcctatttgcagatcaacaacctcaagaatgaggacacggctacatatttctgtgtaagagatcgatatgattccctctttgactactggggccaaggcaccactctcacagtctcctcaggcggaggtggaagcggagggggaggatctggcggcggaggaagcggaggcgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 108 - protein, 247 aa, Artificial Sequence
  EGFR ScFv
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKGGGGSGGGGSGGGGSGGQIQLVQSGPELKKPGETVKISCKASGYTFTEYPIHWVKQAPGKGFKWMGMIYTDIGKPTYAEEFKGRFAFSLETSASTAYLQINNLKNEDTATYFCVRDRYDSLFDYWGQGTTLTVSS

SEQ ID NO: 109 - DNA, 741 bp, Artificial Sequence
  EGFR ScFv
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaaggcggaggtggaagcggagggggaggatctggcggcggaggaagcggaggccagatccagttggtgcagtctggacctgagctgaagaagcctggagagacagtcaagatctcctgcaaggcctctgggtataccttcacagaatatccaatacactgggtgaagcaggctccaggaaagggtttcaagtggatgggcatgatatacaccgacattggaaagccaacatatgctgaagagttcaagggacggtttgccttctctttggagacctctgccagcactgcctatttgcagatcaacaacctcaagaatgaggacacggctacatatttctgtgtaagagatcgatatgattccctctttgactactggggccaaggcaccactctcacagtctcctca

SEQ ID NO: 110 - protein, 249 aa, Artificial Sequence
  EGFR ScFv
EMQLVESGGGFVKPGGSLKLSCAASGFAFSHYDMSWVRQTPKQRLEWVAYIASGGDITYYADTVKGRFTISRDNAQNTLYLQMSSLKSEDTAMFYCSRSSYGNNGDALDFWGQGTSVTVSSGGGGSGGGGSGGGGSGGDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVLTFGSGTKLEIK

SEQ ID NO: 111 - DNA, 747 bp, Artificial Sequence
  EGFR ScFv
gaaatgcagctggtggagtctgggggaggcttcgtgaagcctggagggtccctgaaactctcatgtgcagcctctggattcgctttcagtcactatgacatgtcttgggttcgccagactccgaagcagaggctggagtgggtcgcatacattgctagtggtggtgatatcacctactatgcagacactgtgaagggccgattcaccatctccagagacaatgcccagaacaccctgtacctgcaaatgagcagtctgaagtctgaggacacagccatgttttactgttcacgatcctcctatggtaacaacggagatgccctggacttctggggtcaaggtacctcagtcaccgtctcctcaggcggaggtggaagcggagggggaggatctggcggcggaggaagcggaggcgatgttgtgatgacccaaactccactctccctgcctgtcagtcttggagatcaagcctccatctcttgcagatctagtcagagccttgttcacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttctcacgttcggctcggggacaaagttggaaataaaa

SEQ ID NO: 112 - protein, 249 aa, Artificial Sequence
  EGFR ScFv
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVLTFGSGTKLEIKGGGGSGGGGSGGGGSGGEMQLVESGGGFVKPGGSLKLSCAASGFAFSHYDMSWVRQTPKQRLEWVAYIASGGDITYYADTVKGRFTISRDNAQNTLYLQMSSLKSEDTAMFYCSRSSYGNNGDALDFWGQGTSVTVSS

SEQ ID NO: 113 - DNA, 747 bp, Artificial Sequence
  EGFR ScFv
gatgttgtgatgacccaaactccactctccctgcctgtcagtcttggagatcaagcctccatctcttgcagatctagtcagagccttgttcacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttctcacgttcggctcggggacaaagttggaaataaaaggcggaggtggaagcggagggggaggatctggcggcggaggaagcggaggcgaaatgcagctggtggagtctgggggaggcttcgtgaagcctggagggtccctgaaactctcatgtgcagcctctggattcgctttcagtcactatgacatgtcttgggttcgccagactccgaagcagaggctggagtgggtcgcatacattgctagtggtggtgatatcacctactatgcagacactgtgaagggccgattcaccatctccagagacaatgcccagaacaccctgtacctgcaaatgagcagtctgaagtctgaggacacagccatgttttactgttcacgatcctcctatggtaacaacggagatgccctggacttctggggtcaaggtacctcagtcaccgtctcctca

SEQ ID NO: 114 - protein, 243 aa, Artificial Sequence
  EGFR ScFv
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 115 - DNA, 729 bp, Artificial Sequence
  EGFR ScFv
caggtgcagctgaagcagtccggccccggcctggtgcagccctcccagtccctgtccatcacctgcaccgtgtccggcttctccctgaccaactacggcgtgcactgggtgcggcagtcccccggcaagggcctggagtggctgggcgtgatctggtccggcggcaacaccgactacaacacccccttcacctcccggctgtccatcaacaaggacaactccaagtcccaggtgttcttcaagatgaactccctgcagtccaacgacaccgccatctactactgcgcccgggccctgacctactacgactacgagttcgcctactggggccagggcaccctggtgaccgtgtccgccggcggaggtggaagcggagggggaggatctggcggcggaggaagcggaggcgacatcctgctgacccagtcccccgtgatcctgtccgtgtcccccggcgagcgggtgtccttctcctgccgggcctcccagtccatcggcaccaacatccactggtaccagcagcggaccaacggctccccccggctgctgatcaagtacgcctccgagtccatctccggcatcccctcccggttctccggctccggctccggcaccgacttcaccctgtccatcaactccgtggagtccgaggacatcgccgactactactgccagcagaacaacaactggcccaccaccttcggcgccggcaccaagctggagctgaag

SEQ ID NO: 116 - protein, 244 aa, Artificial Sequence
  EGFR ScFv
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSGGGGSGGGGSGGGGSGGDIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIK

SEQ ID NO: 117 - DNA, 731 bp, Artificial Sequence
  EGFR ScFv
aggtgcagctggtgcagtccggcgccgaggtgaagaagcccggcgcctccgtgaaggtgtcctgcaaggcctccggctacaccttcacctcccactggatgcactgggtgcggcaggcccccggccagggcctggagtggatcggcgagttcaacccctccaacggccggaccaactacaacgagaagttcaagtccaaggccaccatgaccgtggacacctccaccaacaccgcctacatggagctgtcctccctgcggtccgaggacaccgccgtgtactactgcgcctcccgggactacgactacgacggccggtacttcgactactggggccagggcaccctggtgaccgtgtcctccggcggaggtggaagcggagggggaggatctggcggcggaggaagcggaggcgacatccagatgacccagtccccctcctccctgtccgcctccgtgggcgaccgggtgaccatcacctgctccgcctcctcctccgtgacctacatgtactggtaccagcagaagcccggcaaggcccccaagctgctgatctacgacacctccaacctggcctccggcgtgccctcccggttctccggctccggctccggcaccgactacaccttcaccatctcctccctgcagcccgaggacatcgccacctactactgccagcagtggtcctcccacatcttcaccttcggccagggcaccaaggtggagatcaag

SEQ ID NO: 118 - protein, 243 aa, Artificial Sequence
  EGFR ScFv
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSGGDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 119 - DNA, 729 bp, Artificial Sequence
  EGFR ScFv
caggtgcagctgcaggagtccggccccggcctggtgaagccctccgagaccctgtccctgacctgcaccgtgtccggcggctccgtgtcctccggcgactactactggacctggatccggcagtcccccggcaagggcctggagtggatcggccacatctactactccggcaacaccaactacaacccctccctgaagtcccggctgaccatctccatcgacacctccaagacccagttctccctgaagctgtcctccgtgaccgccgccgacaccgccatctactactgcgtgcgggaccgggtgaccggcgccttcgacatctggggccagggcaccatggtgaccgtgtcctccggcggaggtggaagcggagggggaggatctggcggcggaggaagcggaggcgacatccagatgacccagtccccctcctccctgtccgcctccgtgggcgaccgggtgaccatcacctgccaggcctcccaggacatctccaactacctgaactggtaccagcagaagcccggcaaggcccccaagctgctgatctacgacgcctccaacctggagaccggcgtgccctcccggttctccggctccggctccggcaccgacttcaccttcaccatctcctccctgcagcccgaggacatcgccacctacttctgccagcacttcgaccacctgcccctggccttcggcggcggcaccaaggtggagatcaag

SEQ ID NO: 120 - protein, 125 aa, Artificial Sequence [sequence omitted]

SEQ ID NO: 121 - protein, 107 aa, Artificial Sequence [sequence omitted]

SEQ ID NO: 122 - protein, 118 aa, Artificial Sequence
  EGFR VH
QIQLVQSGPELKKPGETVKISCKASGYTFTEYPIHWVKQAPGKGFKWMGMIYTDIGKPTYAEEFKGRFAFSLETSASTAYLQINNLKNEDTATYFCVRDRYDSLFDYWGQGTTLTVSS

SEQ ID NO: 123 - protein, 112 aa, Artificial Sequence
  EGFR VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 124 - protein, 121 aa, Artificial Sequence
  EGFR VH
EMQLVESGGGFVKPGGSLKLSCAASGFAFSHYDMSWVRQTPKQRLEWVAYIASGGDITYYADTVKGRFTISRDNAQNTLYLQMSSLKSEDTAMFYCSRSSYGNNGDALDFWGQGTSVTVSS

SEQ ID NO: 125 - protein, 111 aa, Artificial Sequence
  EGFR VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVLTFGSGTKLEIK

SEQ ID NO: 126 - protein, 119 aa, Artificial Sequence
  EGR VH
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 127 - protein, 107 aa, Artificial Sequence
  EGFR VL
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 128 - protein, 121 aa, Artificial Sequence [sequence omitted]

SEQ ID NO: 129 - protein, 106 aa, Artificial Sequence [sequence omitted]

SEQ ID NO: 130 - protein, 119 aa, Artificial Sequence [sequence omitted]

SEQ ID NO: 131 - protein, 107 aa, Artificial Sequence [sequence omitted]

SEQ ID NO: 132 - protein, 5 aa, Artificial Sequence
  EGFR CDR-H1
TYGMH

SEQ ID NO: 133 - protein, 5 aa, Artificial Sequence
  EGFR CDR-H1
EYPIH

SEQ ID NO: 134 - protein, 5 aa, Artificial SEquence
  EGFR CDR-H1
HYDMS

SEQ ID NO: 135 - protein, 5 aa, Artificial Sequence
  EGFR CDR-H1
NYGVH

SEQ ID NO: 136 - protein, 5 aa, Artificial Sequence
  EGFR CDR-H1
SHWMH

SEQ ID NO: 137 - protein, 7 aa, Artificial Sequence
  EGFR CDR-H1
SGDYYWT

SEQ ID NO: 138 - protein, 17 aa, Artificial Sequence
  EGFR CDR-H2
VIWDDGSYKYYGDSVKG

SEQ ID NO: 139 - protein, 17 aa, Artificial Sequence
  EGFR CDR-H2
MIYTDIGKPTYAEEFKG

SEQ ID NO: 140 - protein, 17 aa, Artificial Sequence
  EGFR CDR-H2
YIASGGDITYYADTVKG

SEQ ID NO: 141 - protein, 16 aa, Artificial Sequence
  EGFR CDR-H2
VIWSGGNTDYNTPFTS

SEQ ID NO: 142 - protein, 17 aa, Artificial Sequence
  EGFR CDR-H2
EFNPSNGRTNYNEKFKS

SEQ ID NO: 143 - protein, 16 aa, Artificial Sequence
  EGFR CDR-H2
HIYYSGNTNYNPSLKS

SEQ ID NO: 144 - protein, 16 aa, Artificial Sequence
  EGFR CDR-H3
DGITMVRGVMKDYFDY

SEQ ID NO: 145 - protein, 9 aa, Artificial Sequence
  EGFR CDR-H3
DRYDSLFDY

SEQ ID NO: 146 - protein, 12 aa, Artificial Sequence
  EGFR CDR-H3
SSYGNNGDALDF

SEQ ID NO: 147 - protein, 11 aa, Artificial Sequence
  EGFR CDR-H3
ALTYYDYEFAY

SEQ ID NO: 148 - protein, 12 aa, Artificial Sequence
  EGFR CDR-H3
RDYDYDGRYFDY

SEQ ID NO: 149 - protein, 9 aa, Artificial Sequence
  EGFR CDR-H3
DRVTGAFDI

SEQ ID NO: 150 - protein, 11 aa, Artificial Sequence
  EGFR CDR-L1
RASQDISSALV

SEQ ID NO: 151 - protein, 16 aa, Artificial Sequence
  EGFR CDR-L1
RSSQSLVHSNGNTYLH

SEQ ID NO: 152 - protein, 16 aa, Artificial Sequence
  EGFRR CDR-L1
RSSQSLVHSNGNTYLH

SEQ ID NO: 153 - protein, 11 aa, Artificial Sequence
  EGFR CDR-L1
RASQSIGTNIH

SEQ ID NO: 154 - protein, 10 aa, Artificial Sequence
  EGFR CDR-L1
SASSSVTYMY

SEQ ID NO: 155 - protein, 11 aa, Artificial Sequence
  EGFR CDR-L1
QASQDISNYLN

SEQ ID NO: 156 - protein, 7 aa, Artificial Sequence
  EGFR CDR-L2
DASSLES

SEQ ID NO: 157 - protein, 7 aa, Artificial Sequence
  EGFR CDR-L2
KVSNRFS

SEQ ID NO: 158 - protein, 7 aa, Artificial Sequence
  EGFR CDR-L2
YASESIS

SEQ ID NO: 159 - protein, 7 aa, Artificial Sequence
  EGFR CDR-L2
DTSNLAS

SEQ ID NO: 160 - protein, 7 aa, Artificial Sequence
  EGFR CDR-L2
DASNLET

SEQ ID NO: 161 - protein, 9 aa, Artificial Sequence
  EGFR CDR-L3
QQFNSYPLT

SEQ ID NO: 162 - protein, 9 aa, Artificial Sequence
  EGFR CDR-L3
SQSTHVPWT

SEQ ID NO: 163 - protein, 8 aa, Artificial Sequence
  EGFR CDR-L3
SQSTHVLT

SEQ ID NO: 164 - protein, 9 aa, Artificial Sequence
  EGFR CDR-L3
QQNNNWPTT

SEQ ID NO: 165 - protein, 9 aa, Artificial Sequence
  EGFR CDR-L3
QQWSSHIFT

SEQ ID NO: 166 - protein, 9 aa, Artificial Sequence
  EGFR CDR-L3
QHFDHLPLA

SEQ ID NO: 167 - protein, 246 aa, Artificial Sequence
  pan-HLA ScFv
QVQLKQSGPGLVQPSQSLSLTCTVSGFSLTSYGVHWVRQPPGKGLEWLGVIWSGGSTDYNAAFISRLSIRKDNSKSQVFFKMNSLQADDTAIYYCARTFTTSTSAWFAYWGQGTLVTVSAGGGGSGGGGSGGGGSGGSIVMTQTPKFLLVSAGDRVTITCKASQSVSNDVAWYQQKPGQSPICLLIYYASNRYTGVPDRFTGSGYGTDFTFTISTVQAEDLAVYFCQQDYSSPPWTFGGGTKLEIR

SEQ ID NO: 168 - DNA, 738 bp, Artificial Sequence
  pan-HLA ScFv
caggtgcagctgaagcagtcaggacctggcctagtgcagccctcacagagcctgtccctgacctgcacagtctctggtttctcattaactagttatggcgtacactgggttcgccagcctccaggaaagggtctggagtggctgggagtgatctggagtggtggaagcacagactataatgctgctttcatatccagactgagcatcaggaaggacaactccaagagccaagtcttctttaaaatgaacagtctgcaagctgatgacacagccatatactactgtgccagaacctttactacgtctacctcggcctggtttgcttactggggccaagggactctggtcactgtctctgcaggcggaggtggaagcggagggggaggatctggcggcggaggaagcggaggcagcatcgtgatgacccagactccaaaattcctgcttgtgtctgcgggagacagagtcaccatcacttgcaaggcgagtcagtctgtgagcaacgacgtagcttggtatcagcagaaaccagggcaatctcctatctgtctcctgatctactatgcatctaatcggtatacaggggtccctgataggttcaccggaagtggatatgggacagatttcactttcaccatcagcaccgtgcaggctgaagatcttgcagtatatttctgtcaacaggattatagtagtcctccgtggactttcggcggagggaccaagttggagatcaga

SEQ ID NO: 169 - protein, 245 aa, Artificial Sequence
  pan-HLA ScFv
QVQLQESGPGLVKPSQTLSLTCTVSGFSLTSYGVHWIRQPPGKGLEWIGVIWSGGSTDYNAAFISRVTISVDTSKNQFSLKLSSVTAADTAVYYCARTFTTSTSAWFAYWGQGTLVTVSSGGGGSGGGGSGGGGSGGDIVMTQSPDSLAVSLGERATINCKASQSVSNDVAWYQQKPGQPPKLLIYYASNRYTGVPDRFSGSGSGTDFTLTISSLQAEDVAVYYCQQDYSSPPWTFGGGTKVEIK

SEQ ID NO: 170 - DNA, 735 bp, Artificial Sequence
  pan-HLA ScFv
caggtgcagctgcaggagtccggacctggcctagtgaagccctcacagaccctgtccctgacctgcacagtctctggtttctcattaactagctatggtgtacactggattagacagcctccaggaaagggtctggagtggattggagtgatctggagtggtggaagcacagactataatgctgctttcatatccagagtgaccatcagcgtggacacctccaagaaccaattctcccttaaactgagcagtgtgacagctgccgacacagccgtatactactgtgccagaacctttactacgtctacctcggcctggtttgcttactggggccaagggactctggtcactgtctcttcaggcggaggtggaagcggagggggaggatctggcggcggaggaagcggaggcgacatcgtgatgacccagagtccagattccctggctgtgtctctgggagagagagccaccatcaattgcaaggcgagtcagtctgtgagcaacgacgtagcttggtatcagcagaaaccagggcaacctcctaaactcctgatctactatgcatctaatcggtatacaggggtccctgataggttcagcggaagtggatctgggacagatttcactctcaccatcagcagcctgcaggctgaagatgttgcagtatattactgtcaacaggattatagtagtcctccgtggactttcggcggagggaccaaggtggagatcaaa

SEQ ID NO: 171 - protein, 245 aa, Artificial Sequence
  pan-HLA ScFv
EVQLLESGGGLVQPGGSLRLSCAASGFSLTSYGVHWVRQAPGKGLEWVSVIWSGGSTDYNAAFISRFTISRDNSKNTLYLQMNSLRAEDTAVYYCARTFTTSTSAWFAYWGQGTLVTVSSGGGGSGGGGSGGGGSGGDIQMTQSPSSLSASVGDRVTITCKASQSVSNDVAWYQQKPGKAPKLLIYYASNRYTGVPSRFSGSGSGTDFTFTISSLQPEDIATYYCQQDYSSPPWTFGGGTKVEIK

SEQ ID NO: 172 - DNA, 735 bp, Artificial Sequence
  pan-HLA ScFv
gaggtgcagctgctggagtccggaggtggcctagtgcagcccggagggagcctgcgcctgagctgcgcagcctctggtttctcattaactagctatggtgtacactgggttagacaggctccaggaaagggtctggagtgggttagcgtgatctggagtggtggaagcacagactataatgctgctttcatatccagatttaccatcagccgggacaactccaagaacacactctaccttcaaatgaacagtttgagagctgaagacacagccgtatactactgtgccagaacctttactacgtctacctcggcctggtttgcttactggggccaagggactctggtcactgtctcttcaggcggaggtggaagcggagggggaggatctggcggcggaggaagcggaggcgacatccagatgacccagagtccaagctccctgtctgcgtctgtgggagacagagtcaccatcacttgcaaggcgagtcagtctgtgagcaacgacgtagcttggtatcagcagaaaccagggaaagctcctaaactcctgatctactatgcatctaatcggtatacaggggtccctagtaggttcagcggaagtggatctgggacagatttcactttcaccatcagcagcctgcagcctgaagatattgcaacatattactgtcaacaggattatagtagtcctccgtggactttcggcggagggaccaaggtggagatcaaa

SEQ ID NO: 173 - protein, 245 aa, Artificial Sequence
  pan-HLA ScFv
QVQLQESGPGLVKPSETLSLTCTVSGFSLTSYGVHWIRQPPGKGLEWIGVIWSGGSTDYNAAFISRVTISRDTSKNQFSLKLSSVTAADTAVYYCARTFTTSTSAWFAYWGQGTLVTVSSGGGGSGGGGSGGGGSGGDIVMTQTPLSLSVTPGQPASISCKASQSVSNDVAWYLQKPGQSPQLLIYYASNRYTGVPDRFSGSGSGTDFTLKISRVEAEDVGVYYCQQDYSSPPWTFGGGTKVEIK

SEQ ID NO: 174 - DNA, 735 bp, Artificial Sequence
  pan-HLA ScFv
caggtgcagctgcaggagtccggacctggcctagtgaagccctcagaaaccctgtccctgacctgcacagtctctggtttctcattaactagctatggtgtacactggattagacagcctccaggaaagggtctggagtggattggagtgatctggagtggtggaagcacagactataatgctgctttcatatccagagtgaccatcagcagggacacctccaagaaccaattctcccttaaactgagcagtgtgacagctgccgacacagccgtatactactgtgccagaacctttactacgtctacctcggcctggtttgcttactggggccaagggactctggtcactgtctcttcaggcggaggtggaagcggagggggaggatctggcggcggaggaagcggaggcgacatcgtgatgacccagactccactttccctgtctgtgactccgggacagccagccagcatcagttgcaaggcgagtcagtctgtgagcaacgacgtagcttggtatctgcagaaaccagggcaatctcctcaactcctgatctactatgcatctaatcggtatacaggggtccctgataggttcagcggaagtggatctgggacagatttcactttgaagatcagcagggtggaggctgaagatgttggagtatattactgtcaacaggattatagtagtcctccgtggactttcggcggagggaccaaggtggagatcaaa

SEQ ID NO: 175 - protein, 245 aa, Artificial Sequence
  pan-HLA ScFv
QVQLVESGGGVVQPGRSLRLSCAVSGFSLTSYGMHWVRQAPGKGLEWVAVIWSGGSTDYNAAFISRFTISRDNSKNTLYLQMNSLRAEDTAVYYCAKTFTTSTSAWFAYWGQGTLVTVSSGGGGSGGGGSGGGGSGGEIVLTQSPATLSLSPGERATLSCRASQSVSNDLAWYQQKPGQAPRLLIYYASNRYTGVPDRFSGSGSGTDFTLTISSLEPEDFAVYYCQQDYSSPPWTFGQGTKVEIK

SEQ ID NO: 176 - DNA, 735 bp, Artificial Sequence
  pan-HLA ScFv
caggtgcagctggtggagtccggaggtggcgtagtgcagcccggaaggagcctgcgcctgagctgcgcagtctctggtttctcattaactagctatggtatgcactgggttagacaggctccaggaaagggtctggagtgggttgcagtgatctggagtggtggaagcacagactataatgctgctttcatatccagatttaccatcagccgggacaactccaagaacacactctaccttcaaatgaacagtttgagagctgaagacacagccgtatactactgtgccaaaacctttactacgtctacctcggcctggtttgcttactggggccaagggactctggtcactgtctcttcaggcggaggtggaagcggagggggaggatctggcggcggaggaagcggaggcgagatcgtgctgacccagagtccagctaccctgtctctgtctccgggagagagagccaccctcagttgcagggcgagtcagtctgtgagcaacgacctagcttggtatcagcagaaaccagggcaagctcctagactcctgatctactatgcatctaatcggtatacaggggtccctgataggttcagcggaagtggatctgggacagatttcactctcaccatcagcagcctggagcctgaagattttgcagtatattactgtcaacaggattatagtagtcctccgtggactttcggccaagggaccaaggtggagatcaaa

SEQ ID NO: 177 - protein, 245 aa, Artificial Sequence
  pan-HLA ScFv
QVQLVESGGGVVQPGRSLRLSCAVSGFSLTSYGMHWVRQAPGKGLEWVAVIWSGGSTDYNAAFISRFTISRDNSKNTLYLQMNSLRAEDTAVYYCARTFTTSTSAWFAYWGQGTLVTVSSGGGGSGGGGSGGGGSGGDIQMTQSPSSLSASVGDRVTITCQASQSVSNDLNWYQQKPGKAPKLLIYYASNRYTGVPDRFSGSGSGTDFTFTISSLQPEDIATYYCQQDYSSPPWTFGGGTKVEIK

SEQ ID NO: 178 - DNA, 735 bp, Artificial Sequence
  pan-HLA ScFv
caggtgcagctggtggagtccggaggtggcgtagtgcagcccggaaggagcctgcgcctgagctgcgcagtctctggtttctcattaactagctatggtatgcactgggttagacaggctccaggaaagggtctggagtgggttgcagtgatctggagtggtggaagcacagactataatgctgctttcatatccagatttaccatcagccgggacaactccaagaacacactctaccttcaaatgaacagtttgagagctgaagacacagccgtatactactgtgccagaacctttactacgtctacctcggcctggtttgcttactggggccaagggactctggtcactgtctcttcaggcggaggtggaagcggagggggaggatctggcggcggaggaagcggaggcgacatccagatgacccagagtccaagctccctgtctgcgtctgtgggagacagagtcaccatcacttgccaggcgagtcagtctgtgagcaacgacctaaattggtatcagcagaaaccagggaaagctcctaaactcctgatctactatgcatctaatcggtatacaggggtccctgataggttcagcggaagtggatctgggacagatttcactttcaccatcagcagcctgcagcctgaagatattgcaacatattactgtcaacaggattatagtagtcctccgtggactttcggcggagggaccaaggtggagatcaaa

SEQ ID NO: 179 - DNA, 741 bp, Artificial Sequence
  HLA-A*02 antigen binding domain
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctca

SEQ ID NO: 180 - DNA, 741 bp, Artificial Sequence
  HLA-A*02 antigen binding domain
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatgggatggatctaccctggcaatgttaacacagaatataatgagaagttcaagggcaaagccaccattaccgcggacaaatccacgagcacagcctacatggagctgagcagcctgagatctgaagacacggctgtgtattactgtgcgagggaggaaattacctacgctatggactactggggccagggaaccacagtcaccgtgtcctcaggcggaggtggaagcggagggggaggatctggcggcggaggaagcggaggcgagattgtattgacccagagcccaggcaccctgagcctctctccaggagagcgggccaccctcagttgtagatccagtcagagtattgtacacagtaatgggaacacctatttggaatggtatcagcagaaaccaggtcaagccccaagattgctcatctacaaagtctctaacagatttagtggtattccagacaggttcagcggttccggaagtggtactgatttcaccctcacgatctccaggctcgagccagaagatttcgccgtttattactgttttcaaggttcacatgtgccgcgcacattcggtgggggtactaaagtagaaatcaaa

SEQ ID NO: 181 - DNA, 741 bp, Artificial Sequence
  HLA-A*02 antigen binding domain
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatgggatggatctaccctggcaatgttaacacagaatataatgagaagttcaagggcaaagccaccattaccgcggacaaatccacgagcacagcctacatggagctgagcagcctgagatctgaagacacggctgtgtattactgtgcgagggaggaaattacctacgctatggactactggggccagggaaccacagtcaccgtgtcctcaggcggaggtggaagcggagggggaggatctggcggcggaggaagcggaggcgacattgtaatgacccagaccccactcagcctgcccgtcactccaggagagccggccagcatcagttgtagatccagtcagagtattgtacacagtaatgggaacacctatttggaatggtatctgcagaaaccaggtcaatccccacaattgctcatctacaaagtctctaacagatttagtggtgtaccagacaggttcagcggttccggaagtggtactgatttcaccctcaagatctccagggtcgaggcagaagatgtcggcgtttattactgttttcaaggttcacatgtgccgcgcacattcggtgggggtactaaagtagaaatcaaa

SEQ ID NO: 182 - DNA, 741 bp, Artificial Sequence
  HLA-A*02 antigen binding domain
gaggtgcagctggtggagtctgggggtgggctggtgaagcctgggggctcactgaggctttcctgcgcggcttctggatacaccttcactagctatcatatacattgggtgcgccaggcccccggaaaagggcttgagtgggtgggatggatctaccctggcaatgttaacacagaatataatgagaagttcaagggcagattcaccattagcagggacgattccaagaacacactctacctgcagatgaacagcctgaaaactgaagacacggctgtgtattactgtgcgagggaggaaattacctacgctatggactactggggccagggaaccacagtcaccgtgtcctcaggcggaggtggaagcggagggggaggatctggcggcggaggaagcggaggcgacattcaaatgacccagagcccatccagcctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccaagcaggttcagcggttccggaagtggtactgatttcaccctcacgatctcctctctccagccagaagatttcgccacttattactgttttcaaggttcacatgtgccgcgcacattcggtgggggtactaaagtagaaatcaaa

SEQ ID NO: 183 - DNA, 741 bp, Artificial Sequence
  HLA-A*02 antigen binding domain
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggcggaggtggaagcggagggggaggatctggcggcggaggaagcggaggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaa

SEQ ID NO: 184 - DNA, 741 bp, Artificial Sequence
  HLA-A*02 antigen binding domain
caggtgcagctggtgcagtctggggctgaggtgaagaagcctgggtcctcagtgaaggtttcctgcaaggcttctggatacaccttcactagctatcatatgcattgggtgcgccaggcccccggacaagggcttgagtggatcggatacatctaccctggcaatgttaacacagaatataatgagaagttcaagggcaaagccacccttaccgcggacaaatccacgaacacagcctacatggagctgagcagcctgagatctgaagacacggctgtgtatttctgtgcgagggaggaaattacctacgctatggactactggggccagggaaccctggtcaccgtgtcctcaggcggaggtggaagcggagggggaggatctggcggcggaggaagcggaggcgacgttcaaatgacccagagcccatccaccctgagcgcatctgtaggtgaccgggtcaccatcacttgtagctccagtcagagtattgtacacagtaatgggaacacctatatggaatggtatcagcagaaaccaggtaaagccccaaaattgctcatctacaaagtctctaacagatttagtggtgtaccagacaggttcagcggttccggaagtggtactgaattcaccctcacgatctcctctctccagccagatgatttcgccacttattactgtcatcaaggttcacatgtgccgcgcacattcggtcagggtactaaagtagaagtcaaa

SEQ ID NO: 185 - DNA, 741 bp, Artificial Sequence
  HLA-A*02 antigen binding domain
caggtgcagctgcagcagtctgggcctgagctggtgaagcctggggcctcagtgaagatgtcctgcaaggcttctggatacaccttcactagctatcatatccagtgggtgaagcagaggcctggacaagggcttgagtggatcggatggatctaccctggcgatggtagtacacagtataatgagaagttcaagggcaaaaccacccttaccgcggacaaatcctccagcacagcctacatgttgctgagcagcctgacctctgaagactctgctatctatttctgtgcgagggaggggacctactacgctatggactactggggccagggaacctcagtcaccgtgtcctcaggcggaggtggaagcggagggggaggatctggcggcggaggaagcggaggcgatgttttgatgacccagactccactctccctgcctgtctctcttggagaccaagtctccatctcttgtagatccagtcagagtattgtacacagtaatgggaacacctatttagaatggtatctgcagaaaccaggtcagtctccaaagttgctcatctacaaagtctctaacagatttagtggtgtaccagacaggttcagcggttccggaagtggtactgatttcaccctcaagatctcgagagtggaggctgaggatctgggagtttattactgttttcaaggttcacatgtgccgcgcacattcggtggaggtactaaactggaaatcaaa

SEQ ID NO: 186 - DNA, 741 bp, Artificial Sequence
  HLA-A*02 antigen binding domain
cagctgcagctgcaggagtctgggcccgggctggtgaagccttcggaaacgctgagcctcacctgcacggtttctggatacaccttcaccagctatcatatccagtggatccgacagccccctggaaaagggcttgagtggatcggatggatctaccctggcgatggttcaacacagtacaatgagaagttcaagggcagagccacgattagcgtggacacatccaagaaccaattctccctgaacctggacagcgtgagtgctgcggacacggccatttattactgtgcgagagagggaacttactacgctatggactactggggcaaagggagcacggtcaccgtgtcctcaggcggaggtggaagcggagggggaggatctggcggcggaggaagcggaggcgacatccagatgacccagagcccaagctccctgagtgcgtccgtgggcgaccgcgtgaccatcacttgcagatcctctcagtccatcgtgcactccaacggcaacacgtacctcgagtggtaccagcagaagcccgggaaggccccgaaactgctcatctacaaggtgagcaaccggttctccggcgtccccagccgcttctcagggtccggctcggggacggatttcaccttcacgattagcagcttgcagcccgaagacatcgccacgtactactgctttcagggaagtcacgtgccgcgtaccttcgggccgggcacgaaagtggatattaag

SEQ ID NO: 187 - DNA, 741 bp, Artificial Sequence
  HLA-A*02 antigen binding domain
gaggtgcagctggtgcagtctggggccgagctgaagaagcctgggtcctcggtgaaggtgtcctgcaaggcttctggatacaccttcaccagctatcatatccagtgggtaaaacaggcccctggacaagggcttgagtggatcggatggatctaccctggcgatggttcaacacagtacaatgagaagttcaagggcaaagccacgcttaccgtggacaaatccacgaacacagcctacatggagctgagcagcctgagatctgaggacacggccgtatattactgtgcgagagagggaacttactacgctatggactactggggccaagggaccctggtcaccgtgtcctcaggcggaggtggaagcggagggggaggatctggcggcggaggaagcggaggcgacatccagatgacccagagcccatccaccctgagtgcgtccgtgggcgaccgcgtgaccatcacttgcagatcctctcagtccatcgtgcactccaacggcaacacgtacctcgagtggtaccagcagaagcccgggaaggccccgaaactgctcatctacaaggtgagcaaccggttctccggcgtccccagccgcttctcagggtccggctcggggacggatttcaccctcacgattagcagcttgcagcccgatgacttcgccacgtactactgctttcagggaagtcacgtgccgcgtaccttcgggcagggcacgaaagtggaagttaag

SEQ ID NO: 188 - DNA, 741 bp, Artificial Sequence
  HLA-A*02 antigen binding domain
caggtgcagctggtgcagtctggggccgaggtgaagaagcctgggtcctcggtgaaggtgtcctgcaaggcttctggatacaccttcaccagctatcatatccagtgggtacgacaggcccctggacaagggcttgagtggatgggatggatctaccctggcgatggttcaacacagtacaatgagaagttcaagggcagagtcacgattaccgcggacaaatccacgagcacagcctacatggagctgagcagcctgagatctgaggacacggccgtatattactgtgcgagagagggaacttactacgctatggactactggggccaagggaccacggtcaccgtgtcctcaggcggaggtggaagcggagggggaggatctggcggcggaggaagcggaggcgagatcgtcctgacccagagcccagggaccctgagtttgtccccgggcgagcgcgcgaccctcagttgcagatcctctcagtccatcgtgcactccaacggcaacacgtacctcgagtggtaccagcagaagcccgggcaggccccgcgactgctcatctacaaggtgagcaaccggttctccggcatccccgaccgcttctcagggtccggctcggggacggatttcaccctcacgattagccgcttggagcccgaagacttcgccgtgtactactgctttcagggaagtcacgtgccgcgtaccttcggggggggcacgaaagtggaaattaag

SEQ ID NO: 189 - DNA, 741 bp, Artificial Sequence
  HLA-A*02 antigen binding domain
caggtgaccctgaagcagtctggggccgaggtgaagaagcctgggtcctcggtgaaggtgtcctgcacggcttctggatacaccttcaccagctatcatgtcagctgggtacgacaggcccctggacaagggcttgagtggttgggaaggatctaccctggcgatggttcaacacagtacaatgagaagttcaagggcaaagtcacgattaccgcggacaaatccatggacacatccttcatggagctgaccagcctgacatctgaggacacggccgtatattactgtgcgagagagggaacttactacgctatggacctctggggccaagggaccctggtcaccgtgtcctcaggcggaggtggaagcggagggggaggatctggcggcggaggaagcggaggcgagatcgtcctgacccagagcccagggaccctgagtttgtccccgggcgagcgcgcgaccctcagttgcagatcctctcagtccatcgtgcactccaacggcaacacgtacctcgcgtggtaccagcagaagcccgggcaggccccgcgactgctcatctccaaggtgagcaaccggttctccggcgtccccgaccgcttctcagggtccggctcggggacggatttcaccctcacgattagccgcttggagcccgaagacttcgccgtgtactactgccaacagggaagtcacgtgccgcgtaccttcggggggggcacgaaagtggaaattaag

SEQ ID NO: 190 - DNA, 741 bp, Artificial Sequence
  HLA-A*02 antigen binding domain
caggtgcagctggtgcagtctggggccgaggtgaagaagcctggggcctcggtgaaggtgtcctgcaaggcttctggatacaccttcaccagctatcatatgcactgggtacgacaggcccctggacaaaggcttgagtggatgggatggatctaccctggcgatggttcaacacagtacaatgagaagttcaagggcaaagtcacgattacccgggacacatccgcgagcacagcctacatggagctgagcagcctgagatctgaggacacggccgtatattactgtgcgagagagggaacttactacgctatggactactggggccaagggaccctggtcaccgtgtcctcaggcggaggtggaagcggagggggaggatctggcggcggaggaagcggaggcgacatcgtcatgacccagaccccactgtccctgcctgtgaccccgggcgagcccgcgagcatcagttgcagatcctctcagtccatcgtgcactccaacggcaacacgtacctcgactggtacctgcagaagcccgggcagtccccgcaactgctcatctacaaggtgagcaaccggttctccggcgtccccgaccgcttctcagggtccggctcggggacggatttcaccctcaagattagccgcgtggaggccgaagacgtcggcgtgtactactgcatgcagggaagtcacgtgccgcgtaccttcggggggggcacgaaagtggaaattaag

SEQ ID NO: 191 - protein, 9 aa, Artificial Sequence
  HA-1 variant H peptide
VLHDDLLEA

SEQ ID NO: 192 - protein, 19 aa, Artificial Sequence
  P2A self cleaving peptide
ATNFSLLKQAGDVEENPGP

SEQ ID NO: 193 - protein, 608 aa, Artificial Sequence
  HA-1H TCR alpha and beta
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVRSVSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSIDSFNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 194 - DNA, 1827 bp, Artificial Sequence
  HA-1(H) TCR alpha and beta
atggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagaagcgtgtccggggccggctcctaccagctcacctttgggaaggggaccaaattatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggatccggagccaccaacttcagcctgctgaagcaggccggcgacgtggaggagaaccccggccccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcatcgactccttcaacgagcagttcttcgggccgggcaccaggctcacggtcctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 195 - protein, 440 aa, Artificial Sequence
  Ftcr alpha LIR1 HA-1H
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVRSVSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 196 - DNA, 1279 bp, Artificial Sequence
  Ftcr alpha LIR1 HA-1H
atggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagaagcgtgtccggggccggctcctaccagctcacctttgggaaggggaccaaattatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccct

SEQ ID NO: 197 - protein, 466 aa, Artificial Sequence
  Ftcr beta LIR1 HA-1H
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSIDSFNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 198 - DNA, 1401 bp, Artificial Sequence
  Ftcr beta LIR1 HA-1H
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcatcgactccttcaacgagcagttcttcgggccgggcaccaggctcacggtcctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactag

SEQ ID NO: 199 - protein, 250 aa, Artificial Sequence
  HA-1H TCR alpha
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVRSVSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLS

SEQ ID NO: 200 - protein, 276 aa, Artificial Sequence
  HA-1H TCR beta
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSIDSFNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLS

SEQ ID NO: 201 - DNA, 750 bp, Artificial Sequence
  HA-1H TCR alpha
atggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagaagcgtgtccggggccggctcctaccagctcacctttgggaaggggaccaaattatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtca

SEQ ID NO: 202 - DNA, 828 bp, Artificial Sequence
  HA-1H TCR beta
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcatcgactccttcaacgagcagttcttcgggccgggcaccaggctcacggtcctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtct

SEQ ID NO: 203 - protein, 13 aa, Artificial Sequence
  NY-ESO CDR
CASSLGLGYEQYF

SEQ ID NO: 204 - protein, 20 aa, Artificial Sequence
  NY-ESO CDR
CASSLGGPRGLAGLRGDEQF

SEQ ID NO: 205 - protein, 12 aa, Artificial Sequence
  NY-ESO CDR
CASSLRRDNEQF

SEQ ID NO: 206 - protein, 19 aa, Artificial Sequence
  MAGE-A3 CDR
CASSLEVLLGADFPDTQYF

SEQ ID NO: 207 - protein, 18 aa, Artificial Sequence
  MAGE-A3 CDR
CASSFPAGHGADLDNEQF

SEQ ID NO: 208 - protein, 14 aa, Artificial Sequence
  MAGE-A3 CDR
CASSEITGRIGEQF

SEQ ID NO: 209 - protein, 18 aa, Artificial Sequence
  MAGE-A3 CDR
CASSLGGDELGADGNEQF

SEQ ID NO: 210 - protein, 241 aa, Artificial Sequence
  NY-ESO-1 ScFv
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGGGGSGGGGSGGGGSGGEVQLVESGGGLVQPGGSLRLSCAASGFTVYDYMSWVRQAPGKGLEWVSVIYSGGSTYYADSVKGRFTISRDNSKNTLYLQMNSLRAEDTAVYYCARYSYYYYYMDVWGKGTTVTVSS

SEQ ID NO: 211 - DNA, 723 bp, Artificial Sequence
  NY-ESO-1 ScFv
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagggcggaggtggaagcggagggggaggatctggcggcggaggaagcggaggcgaagtgcagctggtggaaagcggcggaggcctggtgcagcctggcggcagcctgagactgtcttgcgccgccagcggcttcaccgtgtacgactacatgagctgggtccgccaggcccctggcaagggactggaatgggtgtccgtgatctacagcggcggcagcacctactacgccgacagcgtgaagggtcgattcaccatcagccgggacaacagcaagaacaccctgtacctgcagatgaacagcctgcgggccgaggacaccgccgtgtattactgtgcgaggtactcctactactactactacatggacgtctggggcaaagggaccacggtcaccgtgtcctca

SEQ ID NO: 212 - protein, 244 aa, Artificial Sequence
  MAGE-A3 antigen binding domain
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCARIPFGDWWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSFVLTFGGGTKVEIK

SEQ ID NO: 213 - DNA, 732 bp, Artificial Sequence
  MAGE-A3 antigen binding domain
caggtgcagctgcaggaaagcggccctggcctggtgaaacccagcgacaccctgagcctgacctgcgccgtgtccggctacagcatcagcagcagcaattggtggggctggatcagacagccccctggcaagggcctggaatggatcggctacatctactacagcggcagcacctactacaaccccagcctgaagtccagagtgaccatgagcgtggacaccagcaagaaccagttcagcctgaagctgagcagcgtgaccgccgtcgataccgctgtgtattactgtgcgagaataccctttggggattggtggtacttcgatctctggggccgtggcaccctggtcactgtgtcctcaggcggaggtggaagcggagggggaggatctggcggcggaggaagcggaggcgacatccagatgacccagagccccagcagcctgagcgccagcgtgggcgacagagtgaccatcacctgtcgggccagccagtcgatcagcagctacctgaactggtatcagcagaagcccggcaaggcccccaagctgctgatctacgccgccagctccctgcagagcggcgtgccaagcagattcagcggcagcggctccggcaccgacttcaccctgaccatcagcagcctgcagcccgaggacttcgccacctactactgccagcagagttacagtttcgttctcactttcggcggagggaccaaggtggagatcaaa

SEQ ID NO: 214 - protein, 248 aa, Artificial Sequence
  MAGE-A3 antigen binding domain
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDLYSSSWYCDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSWASTPLTFGGGTKVEIK

SEQ ID NO: 215 - DNA, 744 bp, Artificial Sequence
  MAGE-A3 antigen binding domain
caggtgcagctggtgcagtctggcgccgaagtgaagaaacctggcgcctccgtgaaggtgtcctgcaaggtgtccggctacaccctgaccgagctgtcgatgcactgggtccgccaggcacctggcaagggactggaatggatgggcggctttgaccccgaggacggcgagacaatctacgcccagaaattccagggcagagtgaccatgaccgaggacaccagcaccgacaccgcctacatggaactgagcagcctgcggagcgaggacaccgctgtgtattactgtgcaacagatctgtatagcagcagctggtactgtgatgcttttgatatctggggccaagggacaatggtcaccgtgtcctcaggcggaggtggaagcggagggggaggatctggcggcggaggaagcggaggcgacatccagatgacccagagccccagcagcctgagcgccagcgtgggcgacagagtgaccatcacctgtcgggccagccagtcgatcagcagctacctgaactggtatcagcagaagcccggcaaggcccccaagctgctgatctacgccgccagctccctgcagagcggcgtgccaagcagattcagcggcagcggctccggcaccgacttcaccctgaccatcagcagcctgcagcccgaggacttcgccacctactactgccagcagagttgggccagcacccctctcactttcggcggagggaccaaggtggagatcaaa

SEQ ID NO: 216 - protein, 245 aa, Artificial Sequence
  HPV E6 antigen binding domain
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARENGVVKWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLFPFGGGTKVEIK

SEQ ID NO: 217 - DNA, 735 bp, Artificial Sequence
  HPV E6 antigen binding domain
gaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgtgccagggagaacggcgtggtgaagtggtacttcgacctgtggggccgtggcaccctggtcactgtgtcctcaggcggaggtggaagcggagggggaggatctggcggcggaggaagcggaggcgacatccagatgacccagagccccagcagcctgagcgccagcgtgggcgacagagtgaccatcacctgtcgggccagccagtcgatcagcagctacctgaactggtatcagcagaagcccggcaaggcccccaagctgctgatctacgccgccagctccctgcagagcggcgtgccaagcagattcagcggcagcggctccggcaccgacttcaccctgaccatcagcagcctgcagcccgaggacttcgccacctactactgccagcagagttacagtacccctctctttcccttcggcggagggaccaaggtggagatcaaa

SEQ ID NO: 218 - protein, 247 aa, Artificial Sequence
  HPV E6 antigen binding domain
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDGRGSPFYGGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 219 - DNA, 741 bp, Artificial Sequence
  HPV E6 antigen binding domain
gaagtgcagctggtggaaagcggcggaggcctggtgcagcccggtcgaagcctgagactgagctgcgccgccagcggcttcacctttgacgactacgccatgcactgggtccgccaggcccctggcaagggactggaatgggtgtccggcatcagctggaacagcggcagcatcggctacgccgacagcgtgaagggccggttcaccatcagccgggacaacgccaagaacagcctgtacctgcagatgaacagcctgcgggccgaggacaccgccttgtattactgtgccaaggacggcaggggctcccccttctacggcggcgccttcgacatctggggccaagggacaatggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 220 - protein, 251 aa, Artificial Sequence
  HPV E7 antigen binding domain
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTSYDYLLNPYRWNWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 221 - DNA, 753 bp, Artificial Sequence
  HPV E7 antigen binding domain
gaagtgcagctggtggaaagcggcggaggcctggtgaaacctggcggcagcctgagactgagctgcgccgccagcggcttcaccttctcgaacgcctggatgagctgggtccgccaggcccctggcaagggactggaatgggtcggacggatcaagagcaagaccgacggcggcaccaccgactacgctgcccccgtgaagggccggttcaccatcagccgggacgacagcaagaacaccctgtacctgcagatgaacagcctgaaaaccgaggacaccgccgtgtattactgtaccacctcctacgattaccttctcaatccttatcgttggaactggttcgacccctggggccagggaaccctggtcaccgtgtcctcaggcggaggtggaagcggagggggaggatctggcggcggaggaagcggcggagacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 222 - protein, 246 aa, Artificial Sequence
  MAGE-A3 antigen binding domain
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDMDTFSMVTLFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSWPLTFGGGTKVEIK

SEQ ID NO: 223 - DNA, 738 bp, Artificial Sequence
  MAGE-A3 antigen binding domain
caggtgcagctggtgcagtctggcgccgaagtgaagaaacccggcagcagcgtgaaggtgtcctgcaaggccagcggcggcaccttcagcagctacgccatcagctgggtccgccaggctcctggacagggactggaatggatgggcggcatcatccccatcttcggcaccgccaactacgcccagaaattccagggcagagtgaccatcaccgccgacgagagcaccagcaccgcctacatggaactgagcagccttcgaagcgaggacaccgctgtgtattactgtgccagggacatggacaccttctccatggtgaccctgttcgactactggggccagggcaccctggtcaccgtgtcctcaggcggaggtggaagcggagggggaggatctggcggcggaggaagcggaggcgacatccagatgacccagagccccagcagcctgagcgccagcgtgggcgacagagtgaccatcacctgtcgggccagccagtcgatcagcagctacctgaactggtatcagcagaagcccggcaaggcccccaagctgctgatctacgccgccagctccctgcagagcggcgtgccaagcagattcagcggcagcggctccggcaccgacttcaccctgaccatcagcagcctgcagcccgaggacttcgccacctactactgccagcagagttacagttggcctctcactttcggcggagggaccaaggtggagatcaaa

SEQ ID NO: 224 - protein, 245 aa, Artificial Sequence
  KRAS G12V antigen binding domain
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARSYDELYYFDYWGQGTLVTVSSGGGGSGGGGSGGGGSGGDIQMTQSPSSLSASVGDRVTITCRASQSIWTSYLNWYQQKPGKAPKLLIYAASSLQSGVPSRFSGSGSGTDFTLTISSLQPEDFATYYCQQSYSTPLTFGGGTKVEIK

SEQ ID NO: 225 - DNA, 735 bp, Artificial Sequence
  KRAS G12V antigen binding domain
caggtcacactgagagagtccggccctgccctggtgaaacccacccagaccctgaccctgacatgcaccttcagcggcttcagcctgagcaccagcgggatgtgcgtgtcctggattcgacagccccctggcaaggccctggaatggctggccctgattgactgggacgacgacaagtactacagcaccagcctgaaaacccggctgaccatcagcaaggacaccagcaagaaccaggtggtgctgaccatgaccaacatggaccccgtggacaccgccacgtattactgtgcacggagttacgacgagctctactactttgactactggggccagggaaccctggtcaccgtgtcctcaggcggaggtggaagcggagggggaggatctggcggcggaggaagcggaggcgacatccagatgacccagagccccagctccctctctgcatctgtgggcgacagagtgaccatcacctgtcgggccagccagtcgatctggaccagctacctgaactggtatcagcagaagcccggcaaggcccccaagctgctgatctacgccgccagctccctgcagagcggcgtgccaagcagattcagcggcagcggctccggcaccgacttcaccctgaccatcagcagcctgcagcccgaggacttcgccacctactactgccagcagagttacagtacccctctcactttcggcggagggaccaaggtggagatcaaa

SEQ ID NO: 226 - protein, 5 aa, Artificial Sequence
  linker
GGGGS

SEQ ID NO: 227 - protein, 10 aa, Artificial Sequence
  linker
GGGGSGGGGS

SEQ ID NO: 228 - protein, 15 aa, Artificial Sequence
  linker
GGGGSGGGGSGGGGS

SEQ ID NO: 229 - protein, 15 aa, Artificial Sequence
  linker
GGGGSGGGGSGGGGS

SEQ ID NO: 230 - protein, 20 aa, Artificial Sequence
  linker
GGGGSGGGGSGGGGSGGGGS

SEQ ID NO: 231 - protein, 4 aa, Artificial Sequence
  linker
GGGS

SEQ ID NO: 232 - protein, 247 aa, Artificial Sequence
  KRAS ScFv
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDFTRDYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 233 - DNA, 741 bp, Artificial Sequence
  KRAS ScFv
caggtgcagctggtggaaagcggcggaggcctggtgaaacctggcggcagcctgagactgagctgcgccgccagcggcttcaccttcagcgactactacatgagctggatcagacaggcccctggcaagggactggaatgggtgtcctacatcagcagcagcggctcgaccatctactacgccgacagcgtgaagggccggttcaccatcagccgggacaacgccaagaacagcctgtacctgcagatgaacagcctgcgggccgaggacaccgccgtgtattactgtgccagggacttcaccagggactactactactactactacatggacgtgtggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 234 - protein, 244 aa, Artificial Sequence
  KRAS ScFv
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARSYDELYYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 235 - DNA, 732 bp, Artificial Sequence
  KRAS ScFv
caggtcacactgagagagtccggccctgccctggtgaaacccacccagaccctgaccctgacatgcaccttcagcggcttcagcctgagcaccagcgggatgtgcgtgtcctggattcgacagccccctggcaaggccctggaatggctggccctgattgactgggacgacgacaagtactacagcaccagcctgaaaacccggctgaccatcagcaaggacaccagcaagaaccaggtggtgctgaccatgaccaacatggaccccgtggacaccgccacgtattactgtgcacggagttacgacgagctctactactttgactactggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 236 - protein, 245 aa, Artificial Sequence; identical to SEQ ID NO: 224
  KRAS ScFv

SEQ ID NO: 237 - DNA, 735 bp, Artificial Sequence; identical to SEQ ID NO: 225
  KRAS ScFv

SEQ ID NO: 238 - protein, 244 aa, Artificial Sequence
  KRAS ScFv
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARSYDELYYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTRLTFGGGTKVEIK

SEQ ID NO: 239 - DNA, 732 bp, Artificial Sequence
  KRAS ScFv
caggtcacactgagagagtccggccctgccctggtgaaacccacccagaccctgaccctgacatgcaccttcagcggcttcagcctgagcaccagcgggatgtgcgtgtcctggattcgacagccccctggcaaggccctggaatggctggccctgattgactgggacgacgacaagtactacagcaccagcctgaaaacccggctgaccatcagcaaggacaccagcaagaaccaggtggtgctgaccatgaccaacatggaccccgtggacaccgccacgtattactgtgcacggagttacgacgagctctactactttgactactggggccagggaaccctggtcaccgtgtcctcaggcggaggtggaagcggagggggaggatctggcggcggaggaagcggaggcgacatccagatgacccagagccccagctccctctctgcatctgtgggcgacagagtgaccatcacctgtcgggccagccagtcgatcagcagctacctgaactggtatcagcagaagcccggcaaggcccccaagctgctgatctacgccgccagctccctgcagagcggcgtgccaagcagattcagcggcagcggctccggcaccgacttcaccctgaccatcagcagcctgcagcccgaggacttcgccacctactactgccagcagagttacagtacccggctcactttcggcggagggaccaaggtggagatcaaa

SEQ ID NO: 240 - protein, 244 aa, Artificial Sequence
  KRAS TCR alpha
MQRNLGAVLGILWVQICWVRGDQVEQSPSALSLHEGTDSALRCNFTTTMRSVQWFRQNSRGSLISLFYLASGTKENGRLKSAFDSKERRYSTLHIRDAQLEDSGTYFCAADSSNTGYQNFYFGKGTSLTVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLS

SEQ ID NO: 241 - DNA, 732 bp, Artificial Sequence
  KRAS TCR alpha
atgcagaggaacctgggagctgtgctggggattctgtgggtgcagatttgctgggtgagaggggatcaggtggagcagagtccttcagccctgagcctccacgagggaaccgattctgctctgagatgcaattttacgaccaccatgaggagtgtgcagtggttccgacagaattccaggggcagcctcatcagtttgttctacttggcttcaggaacaaaggagaatgggaggctaaagtcagcatttgattctaaggagcggcgctacagcaccctgcacatcagggatgcccagctggaggactcaggcacttacttctgtgctgctgactcttcgaacacgggttaccagaacttctattttgggaaaggaacaagtttgactgtcattccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaatgtgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctgaactttcaaaacctgtct

SEQ ID NO: 242 - protein, 283 aa, Artificial Sequence
  KRAS TCR beta
MSNTAFPDPAWNTTLLSWVALFLLGTSSANSGVVQSPRYIIKGKGERSILKCIPISGHLSVAWYQQTQGQELKFFIQHYDKMERDKGNLPSRFSVQQFDDYHSEMNMSALELEDSAVYFCASSLTDPLDSDYTFGSGTRLLVIEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLS

SEQ ID NO: 243 - DNA, 849 bp, Artificial Sequence
  KRAS TCR beta
atgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctctctcacagatccgctagactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtctgcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtct

SEQ ID NO: 244 - protein, 283 aa, Artificial Sequence; identical to SEQ ID NO: 242
  KRAS TCR beta

SEQ ID NO: 245 - DNA, 849 bp, Artificial Sequence; identical to SEQ ID NO: 243
  KRAS TCR beta

SEQ ID NO: 246 - protein, 248 aa, Artificial Sequence
  KRAS TCRa
MKTVTGPLFLCFWLQLNCVSRGEQVEQRPPHLSVREGDSAVITCTYTDPNSYYFFWYKQEPGASLQLLMKVFSSTEINEGQGFTVLLNKKDKRLSLNLTAAHPGDSAAYFCAVSGGTNSAGNKLTFGIGTRVLVRPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLS

SEQ ID NO: 247 - DNA, 744 bp, Artificial Sequence
  KRAS TCRa
atgaagacggtgactggacctttgttcctgtgcttctggctgcagctgaactgtgtgagcagaggcgagcaggtggagcagcgccctcctcacctgagtgtccgggagggagacagtgccgttatcacctgcacctacacagaccctaacagttattacttcttctggtacaagcaagagccgggggcaagtcttcagttgcttatgaaggttttctcaagtacggaaataaacgaaggacaaggattcactgtcctactgaacaagaaagacaaacgactctctctgaacctcacagctgcccatcctggggactcagccgcgtacttctgcgcagtcagtggagggactaacagtgcagggaacaagctaacttttggaattggaaccagggtgctggtcaggccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaatgtgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctgaactttcaaaacctgtct

SEQ ID NO: 248 - protein, 272 aa, Artificial Sequence
  KRAS TCRb
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSRDWGPAEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLS

SEQ ID NO: 249 - DNA, 816 bp, Artificial Sequence
  KRAS TCRb
atgggctgtaggctcctaagctgtgtggccttctgcctcttgggaataggccctttggagacagctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagtcgggactgggggcctgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtctgcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtct

SEQ ID NO: 250 - protein, 272 aa, Artificial Sequence; identical to SEQ ID NO: 248
  KRAS TCRb

SEQ ID NO: 251 - DNA, 816 bp, Artificial Sequence; identical to SEQ ID NO: 249
  KRAS TCRb

SEQ ID NO: 252 - protein, 244 aa, Artificial Sequence; identical to SEQ ID NO: 240
  TCRa control

SEQ ID NO: 253 - DNA, 732 bp, Artificial Sequence; identical to SEQ ID NO: 241
  TCRa control

SEQ ID NO: 254 - protein, 248 aa, Artificial Sequence; identical to SEQ ID NO: 246
  TCRa control

SEQ ID NO: 255 - DNA, 744 bp, Artificial Sequence; identical to SEQ ID NO: 247
  TCRa control

SEQ ID NO: 256 - protein, 9 aa, Artificial Sequence
  mH-Y-2Db peptide
KCSRNRQYL

SEQ ID NO: 257 - protein, 614 aa, Artificial Sequence
  H-Y TCR alpha and beta
MFPVTILLLSAFFSLRGNSAQSVDQPDAHVTLSEGASLELRCSYSYSAAPYLFWYVQYPGQSLQFLLKYITGDTVVKGTKGFEAEFRKSNSSFNLKKSPAHWSDSAKYFCALEGQDQGGSAKLIFGEGTKLTVSSPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMSNTAFPDPAWNTTLLSWVALFLLGTKHMEAAVTQSPRNKVAVTGGKVTLSCNQTNNHNNMYWYRQDTGHGLRLIHYSYGAGSTEKGDIPDGYKASRPSQENFSLILELATPSQTSVYFCASGDNSAETLYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 258 - DNA, 1845 bp, Artificial Sequence
  H-Y TCR alpha and beta
atgttccccgttaccatattgctcctgagtgctttcttcagtttgcgagggaactctgcccaatctgtcgatcagccagatgcacacgttaccctttccgagggtgcgtcacttgaactgaggtgttcctatagctactcagcagccccttatctgttttggtacgtacaatacccagggcagagtctccaatttctccttaaatatataaccggggacactgtagtgaaggggactaaaggttttgaagcagagtttaggaagagcaacagctccttcaaccttaagaagtcacccgcacactggtcagactccgcgaaatacttctgtgctctggaagggcaagaccagggtggaagtgccaaattgatatttggtgagggtactaaattgactgttagctcaccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtcgggccaagcggtccggatccggagccaccaacttcagcctgctgaagcaggccggcgacgtggaggagaaccccggccccatgtctaatactgcatttccagatccggcctggaatactacactccttagttgggttgccctcttcctccttggcacgaagcacatggaagccgccgtcacccagagtccgagaaacaaagtagcggtcaccggcggtaaggtaacattgagttgtaaccaaacgaacaaccataacaatatgtactggtatagacaagacacaggccatggcttgcgcctgattcactacagctatggagcggggagtacggagaagggtgatattcccgatggctataaagcctctcggcctagccaggagaattttagtttgatcctggagctcgcaacccccagtcagactagcgtctacttttgtgcctcaggcgacaatagtgcggaaaccctttacttcggccctgggacaagacttacagttctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctag

SEQ ID NO: 259 - protein, 619 aa, Artificial Sequence
  H-Y TCR alpha and beta
MFPVTILLLSAFFSLRGNSAQSVDQPDAHVTLSEGASLELRCSYSYSAAPYLFWYVQYPGQSLQFLLKYITGDTVVKGTKGFEAEFRKSNSSFNLKKSPAHWSDSAKYFCALEGQDQGGSAKLIFGEGTKLTVSSPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMSNTAFPDPAWNTTLLSWVALFLLGTKHMEAAVTQSPRNKVAVTGGKVTLSCNQTNNHNNMYWYRQDTGHGLRLIHYSYGAGSTEKGDIPDGYKASRPSQENFSLILELATPSQTSVYFCASGDNSAETLYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 260 - DNA, 1860 bp, Artificial Sequence
  H-Y TCR alpha and beta
atgttccccgttaccatattgctcctgagtgctttcttcagtttgcgagggaactctgcccaatctgtcgatcagccagatgcacacgttaccctttccgagggtgcgtcacttgaactgaggtgttcctatagctactcagcagccccttatctgttttggtacgtacaatacccagggcagagtctccaatttctccttaaatatataaccggggacactgtagtgaaggggactaaaggttttgaagcagagtttaggaagagcaacagctccttcaaccttaagaagtcacccgcacactggtcagactccgcgaaatacttctgtgctctggaagggcaagaccagggtggaagtgccaaattgatatttggtgagggtactaaattgactgttagctcaccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggatccggagccaccaacttcagcctgctgaagcaggccggcgacgtggaggagaaccccggccccatgtctaatactgcatttccagatccggcctggaatactacactccttagttgggttgccctcttcctccttggcacgaagcacatggaagccgccgtcacccagagtccgagaaacaaagtagcggtcaccggcggtaaggtaacattgagttgtaaccaaacgaacaaccataacaatatgtactggtatagacaagacacaggccatggcttgcgcctgattcactacagctatggagcggggagtacggagaagggtgatattcccgatggctataaagcctctcggcctagccaggagaattttagtttgatcctggagctcgcaacccccagtcagactagcgtctacttttgtgcctcaggcgacaatagtgcggaaaccctttacttcggccctgggacaagacttacagttctggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 261 - protein, 614 aa, Artificial Sequence
  H-Y TCR alpha and beta
MFPVTILLLSAFFSLRGNSAQSVDQPDAHVTLSEGASLELRCSYSYSAAPYLFWYVQYPGQSLQFLLKYITGDTVVKGTKGFEAEFRKSNSSFNLKKSPAHWSDSAKYFCALEGQDQGGSAKLIFGEGTKLTVSSPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMSNTAFPDPAWNTTLLSWVALFLLGTKHMEAAVTQSPRNKVAVTGGKVTLSCNQTNNHNNMYWYRQDTGHGLRLIHYSYGAGSTEKGDIPDGYKASRPSQENFSLILELATPSQTSVYFCASGDNSAETLYFGPGTRLLVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 262 - DNA, 1845 bp, Artificial Sequence
  H-Y TCR alpha and beta
atgttccccgttaccatattgctcctgagtgctttcttcagtttgcgagggaactctgcccaatctgtcgatcagccagatgcacacgttaccctttccgagggtgcgtcacttgaactgaggtgttcctatagctactcagcagccccttatctgttttggtacgtacaatacccagggcagagtctccaatttctccttaaatatataaccggggacactgtagtgaaggggactaaaggttttgaagcagagtttaggaagagcaacagctccttcaaccttaagaagtcacccgcacactggtcagactccgcgaaatacttctgtgctctggaagggcaagaccagggtggaagtgccaaattgatatttggtgagggtactaaattgactgttagctcaccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtcgggccaagcggtccggatccggagccaccaacttcagcctgctgaagcaggccggcgacgtggaggagaaccccggccccatgtctaatactgcatttccagatccggcctggaatactacactccttagttgggttgccctcttcctccttggcacgaagcacatggaagccgccgtcacccagagtccgagaaacaaagtagcggtcaccggcggtaaggtaacattgagttgtaaccaaacgaacaaccataacaatatgtactggtatagacaagacacaggccatggcttgcgcctgattcactacagctatggagcggggagtacggagaagggtgatattcccgatggctataaagcctctcggcctagccaggagaattttagtttgatcctggagctcgcaacccccagtcagactagcgtctacttttgtgcctcaggcgacaatagtgcggaaaccctttacttcggccctgggacaagacttttggttctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctag

SEQ ID NO: 263 - protein, 619 aa, Artificial Sequence
  H-Y TCR alpha and beta
MFPVTILLLSAFFSLRGNSAQSVDQPDAHVTLSEGASLELRCSYSYSAAPYLFWYVQYPGQSLQFLLKYITGDTVVKGTKGFEAEFRKSNSSFNLKKSPAHWSDSAKYFCALEGQDQGGSAKLIFGEGTKLTVSSPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMSNTAFPDPAWNTTLLSWVALFLLGTKHMEAAVTQSPRNKVAVTGGKVTLSCNQTNNHNNMYWYRQDTGHGLRLIHYSYGAGSTEKGDIPDGYKASRPSQENFSLILELATPSQTSVYFCASGDNSAETLYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 264 - DNA, 1860 bp, Artificial Sequence
  H-Y TCR alpha and beta
atgttccccgttaccatattgctcctgagtgctttcttcagtttgcgagggaactctgcccaatctgtcgatcagccagatgcacacgttaccctttccgagggtgcgtcacttgaactgaggtgttcctatagctactcagcagccccttatctgttttggtacgtacaatacccagggcagagtctccaatttctccttaaatatataaccggggacactgtagtgaaggggactaaaggttttgaagcagagtttaggaagagcaacagctccttcaaccttaagaagtcacccgcacactggtcagactccgcgaaatacttctgtgctctggaagggcaagaccagggtggaagtgccaaattgatatttggtgagggtactaaattgactgttagctcaccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggatccggagccaccaacttcagcctgctgaagcaggccggcgacgtggaggagaaccccggccccatgtctaatactgcatttccagatccggcctggaatactacactccttagttgggttgccctcttcctccttggcacgaagcacatggaagccgccgtcacccagagtccgagaaacaaagtagcggtcaccggcggtaaggtaacattgagttgtaaccaaacgaacaaccataacaatatgtactggtatagacaagacacaggccatggcttgcgcctgattcactacagctatggagcggggagtacggagaagggtgatattcccgatggctataaagcctctcggcctagccaggagaattttagtttgatcctggagctcgcaacccccagtcagactagcgtctacttttgtgcctcaggcgacaatagtgcggaaaccctttacttcggccctgggacaagacttttggttctggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 265 - protein, 9 aa, Artificial Sequence
  NY-ESO-1
SLLMWITQV

SEQ ID NO: 266 - protein, 9 aa, Artificial Sequence
  HA-1(R) peptide
VLRDDLLEA

SEQ ID NO: 267 - protein, 607 aa, Artificial Sequence
  NY-ESO-1 TCR alpha and beta
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 268 - DNA, 1824 bp, Artificial Sequence
  NY-ESO-1 TCR alpha and beta
atggagacactcttgggcctgcttatcctttggctgcagctgcaatgggtgagcagcaaacaggaggtgacgcagattcctgcagctctgagtgtcccagaaggagaaaacttggttctgaactgcagtttcactgatagcgctatttacaacctccagtggtttaggcaggaccctgggaaaggactcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctctacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggatccggagccaccaacttcagcctgctgaagcaggccggcgacgtggaggagaaccccggccccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 269 - protein, 612 aa, Artificial Sequence
  KRAS TCR
MQRNLGAVLGILWVQICWVRGDQVEQSPSALSLHEGTDSALRCNFTTTMRSVQWFRQNSRGSLISLFYLASGTKENGRLKSAFDSKERRYSTLHIRDAQLEDSGTYFCAADSSNTGYQNFYFGKGTSLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMSNTAFPDPAWNTTLLSWVALFLLGTSSANSGVVQSPRYIIKGKGERSILKCIPISGHLSVAWYQQTQGQELKFFIQHYDKMERDKGNLPSRFSVQQFDDYHSEMNMSALELEDSAVYFCASSLTDPLDSDYTFGSGTRLLVIEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 270 - DNA, 1839 bp, Artificial Sequence
  KRAS TCR
atgcagaggaacctgggagctgtgctggggattctgtgggtgcagatttgctgggtgagaggggatcaggtggagcagagtccttcagccctgagcctccacgagggaaccgattctgctctgagatgcaattttacgaccaccatgaggagtgtgcagtggttccgacagaattccaggggcagcctcatcagtttgttctacttggcttcaggaacaaaggagaatgggaggctaaagtcagcatttgattctaaggagcggcgctacagcaccctgcacatcagggatgcccagctggaggactcaggcacttacttctgtgctgctgactcttcgaacacgggttaccagaacttctattttgggaaaggaacaagtttgactgtcattccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctgaactttcaaaacctgtcagttatgggactccgaatcctcctgctgaaagtagcgggatttaacctgctcatgacgctgaggctgtggtccagtcgggccaagcggtccggatccggagccaccaacttcagcctgctgaagcaggccggcgacgtggaggagaaccccggccccatgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctctctcacagatccgctagactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcatag

SEQ ID NO: 271 - protein, 18 aa, Artificial Sequence
  T2A self cleaving peptide
EGRGSLLTCGDVEENPGP

SEQ ID NO: 272 - protein, 20 aa, Artificial Sequence
  E2A self cleaving peptide
QCTNYALLKLAGDVESNPGP

SEQ ID NO: 273 - protein, 22 aa, Artificial Sequence
  F2A self cleaving peptide
VKQTLNFDLLKLAGDVESNPGP

SEQ ID NO: 274 - protein, 6 aa, Artificial Sequence
  ITIM
  Z (1)..(1): Z is S, I V or L
  X (2)..(2): X is any amino acid
  X (4)..(5): X is any amino acid
  Z (6)..(6): Z is I, V or L
ZXYXXZ

SEQ ID NO: 275 - protein, 242 aa, Artificial Sequence
  CD19 ScFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 276 - DNA, 726 bp, Artificial Sequence
  CD19 ScFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgtcctca

SEQ ID NO: 277 - protein, 244 aa, Artificial Sequence
  CD19 ScFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 278 - DNA, 732 bp, Artificial Sequence
  CD19 ScFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggaggtggaagcggagggggaggatctggcggcggaggaagcggaggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgtcctca

SEQ ID NO: 279 - protein, 509 aa, Artificial Sequence
  CD19 CAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 280 - DNA, 1527 bp, Artificial Sequence
  CD19 CAR
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgtcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 281 - protein, 509 aa, Artificial Sequence; identical to SEQ ID NO: 279
  CD19 CAR

SEQ ID NO: 282 - protein, 246 aa, Artificial Sequence
  CEA ScFv
QVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFAYYVEAMDYWGQGTTVTVSSGGGGSGGGGSGGGGSGGDIQMTQSPSSLSASVGDRVTITCKASQNVGTNVAWYQQKPGKAPKLLIYSASYRYSGVPSRFSGSGSGTDFTLTISSLQPEDFATYYCHQYYTYPLFTFGQGTKLEIK

SEQ ID NO: 283 - DNA, 1542 bp, Artificial Sequence
  CEA ScFv
caggtgcagctggtgcaatctgggtctgagttgaagaagcctggggcctcagtgaaggtttcctgcaaggcttctggatacaccttcactgagtttggaatgaactgggtgcgacaggcccctggacaagggcttgagtggatgggatggataaacaccaaaactggagaggcaacatatgttgaagagtttaagggacggtttgtcttctccttggacacctctgtcagcacggcatatctgcagatcagcagcctaaaggctgaagacactgccgtgtattactgtgcgagatgggacttcgcttattacgtggaggctatggactactggggccaagggaccacggtcaccgtctcctcaggcggaggtggaagcggagggggaggatctggcggcggaggaagcggaggcgatatccagatgacccagtctccatcctccctgtctgcatctgtgggagacagagtcaccatcacttgcaaggccagtcagaatgtgggtactaatgttgcctggtatcagcagaaaccagggaaagcacctaagctcctgatctattcggcatcctaccgctacagtggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaagatggacatgagggtccccgctcagctcctggggctcctgctactctggctccgaggtgccagatgtcaggtgcagctggtgcaatctgggtctgagttgaagaagcctggggcctcagtgaaggtttcctgcaaggcttctggatacaccttcactgagtttggaatgaactgggtgcgacaggcccctggacaagggcttgagtggatgggatggataaacaccaaaactggagaggcaacatatgttgaagagtttaagggacggtttgtcttctccttggacacctctgtcagcacggcatatctgcagatcagcagcctaaaggctgaagacactgccgtgtattactgtgcgagatgggacttcgcttattacgtggaggctatggactactggggccaagggaccacggtcaccgtctcctcaggcggaggtggaagcggagggggaggatctggcggcggaggaagcggaggcgatatccagatgacccagtctccatcctccctgtctgcatctgtgggagacagagtcaccatcacttgcaaggccagtcagaatgtgggtactaatgttgcctggtatcagcagaaaccagggaaagcacctaagctcctgatctattcggcatcctaccgctacagtggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaag

SEQ ID NO: 284 - protein, 246 aa, Artificial Sequence
  CEA ScFv
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSGGGGSGGGGSGGGGSGGDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 285 - DNA, 738 bp, Artificial Sequence
  CEA ScFv
caggtgcagctggtgcagtctggcgccgaagtgaagaaacctggagctagtgtgaaggtgtcctgcaaggccagcggctacaccttcaccgagttcggcatgaactgggtccgacaggctccaggccagggcctcgaatggatgggctggatcaacaccaagaccggcgaggccacctacgtggaagagttcaagggcagagtgaccttcaccacggacaccagcaccagcaccgcctacatggaactgcggagcctgagaagcgacgacaccgccgtgtactactgcgccagatgggacttcgcttattacgtggaagccatggactactggggccagggcaccaccgtgaccgtgtctagcggcggaggtggaagcggagggggaggatctggcggcggaggaagcggaggcgatatccagatgacccagtctccatcctccctgtctgcatctgtgggagacagagtcaccatcacttgcaaggccagtgcggctgtgggtacgtatgttgcgtggtatcagcagaaaccagggaaagcacctaagctcctgatctattcggcatcctaccgcaaaaggggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaag

SEQ ID NO: 286 - protein, 246 aa, Artificial Sequence
  CEA ScFv
QVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFAHYFQTMDYWGQGTTVTVSSGGGGSGGGGSGGGGSGGDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 287 - DNA, 738 bp, Artificial Sequence
  CEA ScFv
caggtgcagctggtgcaatctgggtctgagttgaagaagcctggggcctcagtgaaggtttcctgcaaggcttctggatacaccttcactgagtttggaatgaactgggtgcgacaggcccctggacaagggcttgagtggatgggatggataaacaccaaaactggagaggcaacatatgttgaagagtttaagggacggtttgtcttctccttggacacctctgtcagcacggcatatctgcagatcagcagcctaaaggctgaagacactgccgtgtattactgtgcgagatgggactttgctcattactttcagactatggactactggggccaagggaccacggtcaccgtctcctcaggcggaggtggaagcggagggggaggatctggcggcggaggaagcggaggcgatatccagatgacccagtctccatcctccctgtctgcatctgtgggagacagagtcaccatcacttgcaaggccagtgcggctgtgggtacgtatgttgcgtggtatcagcagaaaccagggaaagcacctaagctcctgatctattcggcatcctaccgcaaaaggggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaag

SEQ ID NO: 288 - protein, 535 aa, Artificial Sequence
  CEA CAR
MDMRVPAQLLGLLLLWLRGARCQVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFAYYVEAMDYWGQGTTVTVSSGGGGSGGGGSGGGGSGGDIQMTQSPSSLSASVGDRVTITCKASQNVGTNVAWYQQKPGKAPKLLIYSASYRYSGVPSRFSGSGSGTDFTLTISSLQPEDFATYYCHQYYTYPLFTFGQGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 289 - DNA, 1608 bp, Artificial Sequence
  CEA CAR
atggacatgagggtccccgctcagctcctggggctcctgctactctggctccgaggtgccagatgtcaggtgcagctggtgcaatctgggtctgagttgaagaagcctggggcctcagtgaaggtttcctgcaaggcttctggatacaccttcactgagtttggaatgaactgggtgcgacaggcccctggacaagggcttgagtggatgggatggataaacaccaaaactggagaggcaacatatgttgaagagtttaagggacggtttgtcttctccttggacacctctgtcagcacggcatatctgcagatcagcagcctaaaggctgaagacactgccgtgtattactgtgcgagatgggacttcgcttattacgtggaggctatggactactggggccaagggaccacggtcaccgtctcctcaggcggaggtggaagcggagggggaggatctggcggcggaggaagcggaggcgatatccagatgacccagtctccatcctccctgtctgcatctgtgggagacagagtcaccatcacttgcaaggccagtcagaatgtgggtactaatgttgcctggtatcagcagaaaccagggaaagcacctaagctcctgatctattcggcatcctaccgctacagtggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaagacaacgacgccagctccccgcccgccaacccctgcacctacgattgcatcacaaccgctgtccctgcggcctgaagcttgtcgcccagccgcaggtggcgccgtacatacacgggggctggattttgcctgtgatttctgggtgctggtcgttgtgggcggcgtgctggcctgctacagcctgctggtgacagtggccttcatcatcttttgggtgaggagcaagcggagtcgactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 290 - protein, 537 aa, Artificial Sequence
  CEA CAR
MDMRVPAQLLGLLLLWLRGARCQVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSGGGGSGGGGSGGGGSGGDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 291 - DNA, 1614 bp, Artificial Sequence
  CEA CAR
atggacatgagggtccccgctcagctcctggggctcctgctactctggctccgaggtgccagatgtcaggtgcagctggtgcagtctggcgccgaagtgaagaaacctggagctagtgtgaaggtgtcctgcaaggccagcggctacaccttcaccgagttcggcatgaactgggtccgacaggctccaggccagggcctcgaatggatgggctggatcaacaccaagaccggcgaggccacctacgtggaagagttcaagggcagagtgaccttcaccacggacaccagcaccagcaccgcctacatggaactgcggagcctgagaagcgacgacaccgccgtgtactactgcgccagatgggacttcgcttattacgtggaagccatggactactggggccagggcaccaccgtgaccgtgtctagcggcggaggtggaagcggagggggaggatctggcggcggaggaagcggaggcgatatccagatgacccagtctccatcctccctgtctgcatctgtgggagacagagtcaccatcacttgcaaggccagtgcggctgtgggtacgtatgttgcgtggtatcagcagaaaccagggaaagcacctaagctcctgatctattcggcatcctaccgcaaaaggggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaagcgtacgacaacgacgccagctccccgcccgccaacccctgcacctacgattgcatcacaaccgctgtccctgcggcctgaagcttgtcgcccagccgcaggtggcgccgtacatacacgggggctggattttgcctgtgatttctgggtgctggtcgttgtgggcggcgtgctggcctgctacagcctgctggtgacagtggccttcatcatcttttgggtgaggagcaagcggagtcgactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 292 - protein, 536 aa, Artificial Sequence
  CEA CAR
MDMRVPAQLLGLLLLWLRGARCQVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFAHYFQTMDYWGQGTTVTVSSGGGGSGGGGSGGGGSGGDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 293 - DNA, 1611 bp, Artificial Sequence
  CEA CAR
atggacatgagggtccccgctcagctcctggggctcctgctactctggctccgaggtgccagatgtcaggtgcagctggtgcaatctgggtctgagttgaagaagcctggggcctcagtgaaggtttcctgcaaggcttctggatacaccttcactgagtttggaatgaactgggtgcgacaggcccctggacaagggcttgagtggatgggatggataaacaccaaaactggagaggcaacatatgttgaagagtttaagggacggtttgtcttctccttggacacctctgtcagcacggcatatctgcagatcagcagcctaaaggctgaagacactgccgtgtattactgtgcgagatgggactttgctcattactttcagactatggactactggggccaagggaccacggtcaccgtctcctcaggcggaggtggaagcggagggggaggatctggcggcggaggaagcggaggcgatatccagatgacccagtctccatcctccctgtctgcatctgtgggagacagagtcaccatcacttgcaaggccagtgcggctgtgggtacgtatgttgcgtggtatcagcagaaaccagggaaagcacctaagctcctgatctattcggcatcctaccgcaaaaggggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaagcgtacaacgacgccagctccccgcccgccaacccctgcacctacgattgcatcacaaccgctgtccctgcggcctgaagcttgtcgcccagccgcaggtggcgccgtacatacacgggggctggattttgcctgtgatttctgggtgctggtcgttgtgggcggcgtgctggcctgctacagcctgctggtgacagtggccttcatcatcttttgggtgaggagcaagcggagtcgactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 294 - protein, 5 aa, Artificial Sequence
  CEA CDR-H1
EFGMN

SEQ ID NO: 295 - protein, 17 aa, Artificial Sequence
  CEA CDR-H2
WINTKTGEATYVEEFKG

SEQ ID NO: 296 - protein, 12 aa, Artificial Sequence
  CEA CDR-H3
WDFAYYVEAMDY

SEQ ID NO: 297 - protein, 12 aa, Artificial Sequence
  CEA CDR-H3
WDFAHYFQTMDY

SEQ ID NO: 298 - protein, 11 aa, Artificial Sequence
  CEA CDR-L1
KASQNVGTNVA

SEQ ID NO: 299 - protein, 11 aa, Artificial Sequence
  CEA CDR-L1
KASAAVGTYVA

SEQ ID NO: 300 - protein, 7 aa, Artificial Sequence
  CEA CDR-L2
SASYRYS

SEQ ID NO: 301 - protein, 7 aa, Artificial Sequence
  CEA CDR-L2
SASYRKR

SEQ ID NO: 302 - protein, 10 aa, Artificial Sequence
  CEA CDR-L3
HQYYTYPLFT

SEQ ID NO: 303 - protein, 10 aa, Artificial Sequence
  LB-CYBA-1Y peptide
  X (10)..(10): X is Y or H
STMERWGQKX

SEQ ID NO: 304 - protein, 11 aa, Artificial Sequence
  LB-OAS1-1R
  X (7)..(7): X is R or T
ETDDPRXYQKY

SEQ ID NO: 305 - protein, 9 aa, Artificial Sequence
  HA-2 peptide
  X (9)..(9): X is V or M
YIGEVLVSX

SEQ ID NO: 306 - protein, 9 aa, Artificial Sequence
  HA-8 peptide
  X (1)..(1): X is R or P
XTLDKVLEV

SEQ ID NO: 307 - protein, 9 aa, Artificial Sequence
  HA-3 peptide
  X (2)..(2): X is T or M
VXEPGTAQY

SEQ ID NO: 308 - protein, 11 aa, Artificial Sequence
  HwA11-S peptide
  X (6)..(6): X is S or T
CIPPDXLLFPA

SEQ ID NO: 309 - protein, 11 aa, Artificial Sequence
  LB-ADIR-1F peptide
  X (9)..(9): X is F or S
SVAPALALXPA

SEQ ID NO: 310 - protein, 9 aa, Artificial Sequence
  LB-HIVEP1-1S peptide
  X (6)..(6): X is S or N
SLPKHXVTI

SEQ ID NO: 311 - protein, 9 aa, Artificial Sequence
  LB-NISCH-1A peptide
  X (7)..(7): X is A or V
ALAPAPXEV

SEQ ID NO: 312 - protein, 9 aa, Artificial Sequence
  LB-SSR1-1S peptide
  X (1)..(1): X is S or L
XLAVAQDLT

SEQ ID NO: 313 - protein, 10 aa, Artificial Sequence
  LB-WNK1-1I peptide
  X (7)..(7): X is I or M
RTLSPEXITV

SEQ ID NO: 314 - protein, 10 aa, Artificial Sequence
  T4A peptide
  X (10)..(10): X is A or E
GLYTYWSAGX

SEQ ID NO: 315 - protein, 9 aa, Artificial Sequence
  UTA2-1 peptide
  X (3)..(3): X is L or P
QLXNSVLTL

SEQ ID NO: 316 - protein, 9 aa, Artificial Sequence
  LB-CLYBL-1Y peptide
  X (5)..(5): X is Y or D
SLAAXIPRL

SEQ ID NO: 317 - protein, 10 aa, Artificial Sequence
  TRIM22 peptide
  X (7)..(7): X is C or R
MAVPPCXIGV

SEQ ID NO: 318 - protein, 11 aa, Artificial Sequence
  PARP10-1L peptide
  X (3)..(3): X is L or P
GLXGQEGLVEI

SEQ ID NO: 319 - protein, 9 aa, Artificial Sequence
  FAM119A-1T peptide
  X (8)..(8): X is T or I
AMLERQFXV

SEQ ID NO: 320 - protein, 9 aa, Artificial Sequence
  GLRX3-1S peptide
  X (3)..(3): X is S or P
FLXSANEHL

SEQ ID NO: 321 - protein, 9 aa, Artificial Sequence
  HNF4G-1M peptide
  X (2)..(2): X is M or I
MXYKDILLL

SEQ ID NO: 322 - protein, 9 aa, Artificial Sequence
  HMMR-1V peptide
  X (6)..(6): X is V or A
SLQEKXAKA

SEQ ID NO: 323 - protein, 9 aa, Artificial Sequence
  BCL2A1 peptide
  X (4)..(4): X is N or K
VLQXVAFSV

SEQ ID NO: 324 - protein, 10 aa, Artificial Sequence
  CDC26-1F peptide
  X (1)..(1): X is F or S
XVAGTQEVFV

SEQ ID NO: 325 - protein, 10 aa, Artificial Sequence
  APOBEC3F-1S/A peptide
  X (3)..(3): X is S or A
FLXEHPNVTL

SEQ ID NO: 326 - protein, 9 aa, Artificial Sequence
  LB-PRCP-1D peptide
  X (8)..(8): X is D or E
FMWDVAEXL

SEQ ID NO: 327 - protein, 11 aa, Artificial Sequence
  LB-PRCP-1D peptide
  X (8)..(8): X is D or E
FMWDVAEXLKA

SEQ ID NO: 328 - protein, 9 aa, Artificial Sequence
  LB-CCL4-1T peptide
  X (7)..(7): X is T or S
CADPSEXWV

SEQ ID NO: 329 - protein, 9 aa, Artificial Sequence
  LB-NCAPD3-1Q peptide
  X (3)..(3): X is Q or R
WLXGVVPVV

SEQ ID NO: 330 - protein, 9 aa, Artificial Sequence
  LB-NDC80-1P peptide
  X (7)..(7): X is P or A
HLEEQIXKV

SEQ ID NO: 331 - protein, 9 aa, Artificial Sequence
  LB-TTK-1D peptide
  X (4)..(4): X is D or E
RLHXGRVFV

SEQ ID NO: 332 - protein, 9 aa, Artificial Sequence
  WDR27-1L peptide
  X (2)..(2): X is L or P
SXDDHVVAV

SEQ ID NO: 333 - protein, 11 aa, Artificial Sequence
  MIIP peptide
  X (8)..(8): X is K or E
SEESAVPXRSW

SEQ ID NO: 334 - protein, 10 aa, Artificial Sequence
  MIIP peptide
  X (7)..(7): X is K or E
EESAVPXRSW

SEQ ID NO: 335 - protein, 9 aa, Artificial Sequence
  LB-DHX33-1C peptide
  X (9)..(9): X is C or R
YLYEGGISX

SEQ ID NO: 336 - protein, 10 aa, Artificial Sequence
  PANE1 peptide
RVWDLPGVLK

SEQ ID NO: 337 - protein, 10 aa, Artificial Sequence
  SP110 peptide
  X (4)..(4): X is R or G
SLPXGTSTPK

SEQ ID NO: 338 - protein, 9 aa, Artificial Sequence
  ACC-1C/Y
  X (5)..(5): X is Y or C
DYLQXVLQI

SEQ ID NO: 339 - protein, 9 aa, Artificial Sequence
  P2RX7 peptide
  X (6)..(6): X is H or R
WFHHCXPKY

SEQ ID NO: 340 - protein, 9 aa, Artificial Sequence
  ACC-4 peptide
  X (9)..(9): X is R or G
ATLPLLCAX

SEQ ID NO: 341 - protein, 10 aa, Artificial Sequence
  ACC-5
  X (10)..(10): X is R or G
WATLPLLCAX

SEQ ID NO: 342 - protein, 9 aa, Artificial Sequence
  AKAP13 peptide
  X (9)..(9): X is M or T
APAGVREVX

SEQ ID NO: 343 - protein, 10 aa, Artificial Sequence
  LB-APOBEC3B-1K peptide
  X (1)..(1): X is K or E
XPQYHAEMCF

SEQ ID NO: 344 - protein, 9 aa, Artificial Sequence
  APOBEC3H peptide
  X (5)..(5): X is K or E
KPQQXGLRL

SEQ ID NO: 345 - protein, 8 aa, Artificial Sequence
  LB-ARHGDIB-1R peptide
  X (6)..(6): X is R or P
PRACWXEA

SEQ ID NO: 346 - protein, 10 aa, Artificial Sequence
  LB-BCAT2-1R peptide
  X (3)..(3): X is R or T
QPXRALLFVI

SEQ ID NO: 347 - protein, 11 aa, Artificial Sequence
  BFAR peptide
  X (11)..(11): X is M or R
APNTGRANQQX

SEQ ID NO: 348 - protein, 11 aa, Artificial Sequence
  C14orf169 peptide
  X (4)..(4): X is A or V
RPRXPTEELAL

SEQ ID NO: 349 - protein, 11 aa, Artificial Sequence
  LB-C16ORF-1R peptide
  X (1)..(1): X is R or W
XPCPSVGLSFL

SEQ ID NO: 350 - protein, 9 aa, Artificial Sequence
  C18orf21 peptide
  X (6)..(6): X is A or T
NPATPXSKL

SEQ ID NO: 351 - protein, 10 aa, Artificial Sequence
  LB-EBI3-1I peptide
  X (8)..(8): X is I or V
RPRARYYXQV

SEQ ID NO: 352 - protein, 9 aa, Artificial Sequence
  POP1 peptide
  X (7)..(7): X is N or K
LPQKKSXAL

SEQ ID NO: 353 - protein, 9 aa, Artificial Sequence
  SCRIB peptide
  X (7)..(7): X is L or P
LPQQPPXSL

SEQ ID NO: 354 - protein, 9 aa, Artificial Sequence
  MTRR peptide
  X (5)..(5): X is S or L
SPASXRTDL

SEQ ID NO: 355 - protein, 9 aa, Artificial Sequence
  LLGL2
  X (5)..(5): X is R or H
SPSLXILAI

SEQ ID NO: 356 - protein, 11 aa, Artificial Sequence
  LB-ECGF-1H peptide
  X (3)..(3): X is H or R
RPXAIRRPLAL

SEQ ID NO: 357 - protein, 11 aa, Artificial Sequence
  LB-ERAP1-1R peptide
  X (3)..(3): X is R or P
HPXQEQIALLA

SEQ ID NO: 358 - protein, 9 aa, Artificial Sequence
  LB-ERAP1-1R peptide
  X (3)..(3): X is R or P
HPXQEQIAL

SEQ ID NO: 359 - protein, 9 aa, Artificial Sequence
  LB-FUCA2-1V peptide
  X (5)..(5): X is V or M
RLRQXGSWL

SEQ ID NO: 360 - protein, 9 aa, Artificial Sequence
  LB-GEMIN4-1V peptide
  X (9)..(9): X is V or E
FPALRFVEX

SEQ ID NO: 361 - protein, 11 aa, Artificial Sequence
  HDGF peptide
  X (11)..(11): X is L or P
LPMEVEKNSTX

SEQ ID NO: 362 - protein, 11 aa, Artificial Sequence
  LB-PDCD11-1F peptide
  X (8)..(8): X is F or L
GPDSSKTXLCL

SEQ ID NO: 363 - protein, 9 aa, Artificial Sequence
  LB-PFAS-1P peptide
  X (2)..(2): X is P or S
AXGHTRRKL

SEQ ID NO: 364 - protein, 10 aa, Artificial Sequence
  LB-TEP1-1S peptide
  X (9)..(9): X is S or P
APDGAKVAXL

SEQ ID NO: 365 - protein, 11 aa, Artificial Sequence
  LB-TMEM8A-1I peptide
  X (7)..(7): X is I or V
RPRSVTXQPLL

SEQ ID NO: 366 - protein, 10 aa, Artificial Sequence
  LB-USP15-1I peptide
  X (8)..(8): X is I or T
MPSHLRNXLL

SEQ ID NO: 367 - protein, 9 aa, Artificial Sequence
  LRH-1 peptide
TPNQRQNVC

SEQ ID NO: 368 - protein, 9 aa, Artificial Sequence
  LB-MOB3A-1C peptide
  X (1)..(1): X is C or S
XPRPGTWTC

SEQ ID NO: 369 - protein, 11 aa, Artificial Sequence
  LB-ZDHHC6-1Y peptide
  X (4)..(4): X is Y or H
RPRXWILLVKI

SEQ ID NO: 370 - protein, 10 aa, Artificial Sequence
  ZAPHIR peptide
IPRDSWWVEL

SEQ ID NO: 371 - protein, 9 aa, Artificial Sequence
  HEATR1 peptide
  X (7)..(7): X is E or G
ISKERAXAL

SEQ ID NO: 372 - protein, 9 aa, Artificial Sequence
  LB-GSTP1-1V peptide
  X (7)..(7): X is V or I
DLRCKYXSL

SEQ ID NO: 373 - protein, 9 aa, Artificial Sequence
  HA-1/B60 peptide
  X (6)..(6): X is H or R
KECVLXDDL

SEQ ID NO: 374 - protein, 9 aa, Artificial Sequence
  LB-SON-1R peptide
  X (6)..(6): X is R or C
SETKQXTVL

SEQ ID NO: 375 - protein, 9 aa, Artificial Sequence
  LB-SWAP70-1Q peptide
  X (6)..(6): X is Q or E
MEQLEXLEL

SEQ ID NO: 376 - protein, 9 aa, Artificial Sequence
  LB-TRIP10-1EPC peptide
  X (2)..(2): X is E or G
  X (3)..(3): X is P or S
  X (7)..(7): X is C or G
GXXQDLXTL

SEQ ID NO: 377 - protein, 9 aa, Artificial Sequence
  LB-NUP133-1R peptide
  X (8)..(8): X is R or Q
SEDLILCXL

SEQ ID NO: 378 - protein, 11 aa, Artificial Sequence
  LB-ZNFX1-1Q peptide
  X (8)..(8): X is Q or H
NEIEDVWXLDL

SEQ ID NO: 379 - protein, 11 aa, Artificial Sequence
  SLC1A5 peptide
  X (3)..(3): X is A or P
AEXTANGGLAL

SEQ ID NO: 380 - protein, 10 aa, Artificial Sequence
  ACC-2 peptide
  X (6)..(6): X is D or G
KEFEDXIINW

SEQ ID NO: 381 - protein, 11 aa, Artificial Sequence
  ACC-6 peptide
MEIFIEVFSHF

SEQ ID NO: 382 - protein, 10 aa, Artificial Sequence
  HB-1H/Y peptide
  X (8)..(8): X is H or Y
EEKRGSLXVW

SEQ ID NO: 383 - protein, 9 aa, Artificial Sequence
  DPH1 peptide
  X (2)..(2): X is V or L
SXLPEVDVW

SEQ ID NO: 384 - protein, 9 aa, Artificial Sequence
  UGT2B17/A02 peptide
CVATMIFMI

SEQ ID NO: 385 - protein, 10 aa, Artificial Sequence
  UGT2B17/A29
AELLNIPFLY

SEQ ID NO: 386 - protein, 10 aa, Artificial Sequence
  UGT2B17/B44 peptide
AELLNIPFLY

SEQ ID NO: 387 - protein, 9 aa, Artificial Sequence
  DFFRY peptide
  X (4)..(4): X is C or S
IVDXLTEMY

SEQ ID NO: 388 - protein, 9 aa, Artificial Sequence
  SMCY peptide
FIDSYICQV

SEQ ID NO: 389 - protein, 11 aa, Artificial Sequence
  TMSB4Y peptide
EVLLRPGLHFR

SEQ ID NO: 390 - DNA, 1533 bp, Artificial Sequence
  CD19 CAR
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggaggtggaagcggagggggaggatctggcggcggaggaagcggaggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgtcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 391 - protein, 245 aa, Artificial Sequence
  EGFR ScFv
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSGGGGSGGGGSGGGGSGGEIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIK